(19) United States
(12) Patent Application Publication
(10) Pub. No.: US 2023/0295347 A1
(43) Pub. Date: Sep. 21, 2023

(54) WNT RECEPTOR-SPECIFIC COMPOUND AND METHOD RELATING THERETO

(71) Applicant: Surrozen Operating, Inc., South San Francisco, CA (US)

(72) Inventors: Hui CHEN, Foster City, CA (US); Sungjin LEE, Palo Alto, CA (US); Yang LI, Mountain View, CA (US); Wen-Chen YEH, Belmont, CA (US)

(21) Appl. No.: 18/148,448

(22) Filed: Dec. 30, 2022

Related U.S. Application Data
(60) Provisional application No. 63/295,805, filed on Dec. 31, 2021.
(60) Provisional application No. 63/298,570, filed on Jan. 11, 2022.
(60) Provisional application No. 63/398,754, filed on Aug. 17, 2022.

(51) Int. Cl. C07K 16/46; C12N 15/63; A61P 27/02

(52) U.S. Cl. CPC C07K 16/468; C12N 15/63; A61P 27/02; C07K 2317/31; C07K 2317/565; C07K 2317/569; C07K 2317/52

(57) ABSTRACT

The present invention provides compounds which specifically bind to one or more Wnt receptors and methods relating thereto. In some aspects, compositions and methods for treating ocular disorders with modulators of the Wnt signaling pathway are provided. In particular, the ocular disorders are retinopathies. Also provided are methods of dosing and pharmaceutical compositions.

### CROSS-REFERENCE TO RELATED APPLICATIONS

This application claims priority to: U.S. Provisional Application No. 62/295,805, filed on Dec. 31, 2021; U.S. Provisional Application No. 63/298,570, filed on Jan. 11, 2022; and U.S. Provisional Application No. 63/398,754, filed on Aug. 17, 2022. The contents of said applications are incorporated by reference in their entirety herein.

### FIELD OF THE INVENTION

The present invention relates to compounds which specifically bind to one or more Wnt receptors and methods relating thereto. Specifically the present invention provides Wnt signal modulators for use in treating various ocular disorders. In particular, treatments for vascular diseases of the eye, including retinopathies, are provided.

### BACKGROUND OF THE INVENTION

The vertebral retina is a thin layer of nerve tissue in the back of the eye. It is responsible for detecting visual stimuli and is the first station for visual information processing. For its proper function, the retinal vasculature is an indispensable source of nutrients and oxygen. The retina is metabolically highly active. Due to the photoreceptors which consume the vast amount of oxygen, a gram of retina shows the highest oxygen consumption rate than any other organs in body. To effectively deliver nutrients and oxygen, the retinal vasculature is positioned in the retina as a stereotyped architecture consisting of three planal vascular plexuses on one side and the choriocapillaries on the other. The inner vascularization initially begins on the vitreal surface of the retina, giving rise to a primary vascular plexus. After the superficial radial expansion of the vascular plexus, vertical penetration of vessels into the retina forms two additional intraretinal capillary plexuses at the inner plexiform layer (IPL) and the outer plexiform layer (OPL). Due to the functional and structural relationship between blood vessels and the retina, aberrant vessel development and/or vascular damage are directly linked to the aberrant or impaired function of the retina, which may result in various types of retinopathy and retinal degeneration.

Wnt signaling has been implicated as an important pathway for vascular development in the retina. Growing genetic evidence from human and rodent studies further supports the importance of Wnt signaling in retinal vasculature (Wang et al., 2018, Prog Retin Eye Res. 2018 Dec. 1. pii: S1350-9462(18)30046-6). Mutations in human genes encoding various receptors (Fzd4, Lrp5, Tspan12) and a ligand (norrin) involved in Wnt signaling result in a variety of inherited vitreoretinopathies. Specific genetic mutations in the murine orthologs of these genes (Fzd4, Lrp5, Tspan12, norrin) have also been observed to cause the characteristic phenotypes of aberrant vasculature seen in human retinopathy. This discovery has facilitated researchers to obtain a better understanding of retinopathy disease progression, as well as opening the possibility of retinopathy treatment through Wnt signal modulation.

Retinopathy is any damage to the retina of the eyes, which may cause vision impairment. Retinopathy encompasses retinal vascular disease, and damage to the retina caused by abnormal blood flow. Age-related macular degeneration is considered a retinopathy but is often discussed as a separate entity. Retinopathy, including diabetic retinopathy, can be divided into early and late stages. In the early stage, also known as non-proliferative retinopathy, there may be a slight deterioration in the small blood vessels of the retina, and portions of the vessels may swell and leak fluid into the surrounding retinal tissue. Late-stage retinopathy involves significant neovascularization as well as microaneurysms and hemorrhages in the retinal area (see, e.g., Grading Diabetic Retinopathy from Stereoscopic Color Fundus Photographs—An Extension of the Modified Airlie House Classification. (1991) Ophthalmology, 98(5), 786-806). Retinopathy may be an ocular manifestation of systemic or metabolic diseases, as seen with diabetes and hypertension. Diabetes is a common cause of retinopathy, and diabetic retinopathy is a leading cause of blindness in working-aged people and has been designated a priority eye disease by the World Health Organization.

Familial Exudative Vitreoretinopathy (FEVR) is a genetic eye disease characterized by poor formation of intraocular vasculature. Over 50% of FEVR patients show mutations in one of the genes encoding Fzd4, Lrp5, Tspan12, or norrin. Norrin, a Wnt signal ligand, transmits a signal to the endothelial cells through a receptor complex composed of Fzd4/Lrp5/Tspan12 for normal development of retinal vascularization in the eye. However, in FEVR patients, mutations in one or more of norrin, Fzd4, Lrp5, or/and Tspan12 genes result in immature vascular development in the retina. The resulting formation of the avascular region creates a retinal ischemic area, which results in primary damage to the retina. This ischemic condition induces the production of vascular endothelial growth factor (VEGF) and angiopoietin2 (Ang2), leading to neovascularization and vascular tuft formation. The newly generated abnormal blood vessels can be easily broken, leading to secondary damage of the retina due to exudation and hemorrhage. Disease progression of diabetic retinopathy (DR) is similar to that of FEVR and other genetic vascular malformation or insufficiency diseases. Hyperglycemia induces retinal vessel damage, leading to vaso-obliteration, ischemia, neovascularization, and hemorrhage, eventually leading to retinopathy.

While genetic data has suggested the importance of Wnt signaling in establishing the proper vascular structure in the eye, whether activation of Wnt signaling post-developmentally would lead to improvement in vascular structure is unknown. Certain reports have even suggested that antagonizing rather than agonizing Wnt signaling would be beneficial in the treatment of retinopathy. Therefore, a greater understanding of retinopathy, disease progression, and Wnt signal involvement therein would be beneficial as this potentially could result in new treatments. For effective treatment of retinopathy in particular, there is a need for modulating Wnt agonist and antagonist signaling during specific stages of disease. The present invention addresses these needs and provides methods for modulating Wnt signaling agonism and antagonism during different stages of retinopathy and disease progression.

### SUMMARY OF THE INVENTION

In one aspect, the present disclosure provides multispecific antibodies or antibody fragments.

A multispecific antibody or antibody fragment according to the present disclosure may comprise at least: (A) a first antigen-binding region which specifically binds to frizzled class receptor 4 (Fzd4) and comprises a first heavy chain variable domain (VH1) and a first light chain variable domain (VL1), (B) a second antigen-binding region which specifically binds to low density lipoprotein receptor-related protein 5 and/or 6 (LRP5 and/or LRP6) and comprises at least a second heavy chain variable domain (VH2).

In some embodiments, the VH1 comprises: (i) a heavy chain complementarity-determining region 1 (CDRH1) which has an amino acid sequence which: (i-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 121 or to the CDRH1 amino acid sequence contained in SEQ ID NO: 181, optionally defined according to Kabat; (i-2) comprises or consists of the amino acid sequence of SEQ ID NO: 120, wherein X₁ is any amino acid, optionally S, A, Q, or N; or (i-3) comprises or consists of the amino acid sequence of SEQ ID NO: 121, 122, 123, or 124, or comprises or consists of the CDRH1 amino acid sequence contained in any one of SEQ ID NOS: 181-188, optionally defined according to Kabat; (ii) a heavy chain complementarity-determining region 2 (CDRH2) has an amino acid sequence which: (ii-1) has at least 90%, at least 91%, at least 92%, at least 93%, at least 94%, at least 95%, at least 96%, at least 97%, at least 98%, at least 99%, or 100% identity to the amino acid sequence of SEQ ID NO: 141 or to the CDRH2 amino acid sequence contained in SEQ ID NO: 181, optionally defined according to Kabat; (ii-2) comprises or consists of the amino acid sequence of SEQ ID NO: 140, wherein X₁ is any amino acid, optionally E, A, S, or D; or (ii-3) comprises or consists of: the amino acid sequence of any one of SEQ ID NO: 141, or 142, 143, or 144; or comprises or consists of the CDRH2 amino acid sequence contained in any one of SEQ ID NOS: 181-188, optionally defined by Kabat; and (iii) a heavy chain complementarity-determining region 3 (CDRH3) has an amino acid sequence which: (iii-1) has at least 90%, at least 91%, at least 92%, at least 93%, at least 94%, at least 95%, at least 96%, at least 97%, at least 98%, at least 99%, or 100% identity to the amino acid sequence of SEQ ID NO: 161 or to the CDRH3 amino acid sequence contained in SEQ ID NO: 181, optionally defined by Kabat; (iii-2) comprises or consists of the amino acid sequence of SEQ ID NO: 160, wherein X₁ is any amino acid, optionally D or any amino acid which is not S, E, A, R, T, L, H, Y, Q, or K; (iii-3) comprises or consists of the amino acid sequence of SEQ ID NO: 161, or comprises or consists of the CDRH3 amino acid sequence contained in any one of SEQ ID NOS: 181-188, optionally defined by Kabat.

In some embodiments, the VL1 comprises: (iv) a light chain complementarity-determining region 1 (CDRL1) which has an amino acid sequence which: (iv-1) has at least 90%, at least 91%, at least 92%, at least 93%, at least 94%, at least 95%, at least 96%, at least 97%, at least 98%, at least 99%, or 100% identity to the amino acid sequence of SEQ ID NO: 221 or to the CDRL1 amino acid sequence contained in SEQ ID NO: 281, optionally defined by Kabat; or (iv-2) comprises or consists of the amino acid sequence of SEQ ID NO: 221, or comprises or consists of the CDRL1 amino acid sequence contained in SEQ ID NO: 281, optionally defined according to Kabat; (v) a light chain complementarity-determining region 2 (CDRL2) which has an amino acid sequence which comprises or consists of: (v-1) the amino acid sequence of SEQ ID NO: 241; or (v-2) the CDRL2 amino acid sequence contained in SEQ ID NO: 281, optionally defined according to Kabat; and (vi) a light chain complementarity-determining region 3 (CDRL3) which has an amino acid sequence which comprises or consists of: (vi-1) the amino acid sequence of SEQ ID NO: 261; or (vi-2) the CDRL3 amino acid sequence contained in SEQ ID NO: 281, optionally defined according to Kabat.

In some embodiments, the VH2 comprises: (i) a CDRH1 which has an amino acid sequence which comprises or consists of: (i-1) the amino acid sequence of SEQ ID NO: 321; or (i-2) the CDRH1 amino acid sequence contained in any one of SEQ ID NOS: 381, 386, and 393-396, optionally defined according to IMGT or Kabat; (ii) a CDRH2 which has an amino acid sequence which: (ii-1) has at least 90%, at least 91%, at least 92%, at least 93%, at least 94%, at least 95%, at least 96%, at least 97%, at least 98%, at least 99%, or 100% identity to the amino acid sequence of SEQ ID NO: 341 or to the CDRH3 amino acid sequence contained in SEQ ID NO: 381, optionally defined according to IMGT or Kabat; (ii-2) comprises or consists of the amino acid sequence of SEQ ID NO: 341; or (ii-3) comprises or consists of the CDRH2 amino acid sequence contained in any one of SEQ ID NOS: 381, 386, and 393-396, optionally defined according to IMGT or Kabat; and (iii) a CDRH3 which has an amino acid sequence which: (iii-1) has at least 90%, at least 91%, at least 92%, at least 93%, at least 94%, at least 95%, at least 96%, at least 97%, at least 98%, at least 99%, or 100% identity to the amino acid sequence of SEQ ID NO: 361 or to the CDRH3 amino acid sequence contained in SEQ ID NO: 381, optionally defined according to IMGT or MGT or Kabat; (iii-2) comprises or consists of the amino acid sequence of SEQ ID NO: 360, wherein X₁, X₂, and X₃ are individually any amino acids provided that (a) X₁ and X₂ are D and E, respectively, (b) X₁ and X₃ are D and D, respectively, and/or (c) X₂ and X₃ are E and D, respectively, optionally wherein X₁, X₂, and X₃ are D, E, and D, respectively, D, D, and D, respectively, D, E, and E, respectively, D, E, and S, respectively, D, E, and A, respectively, or D, E, and T, respectively; or (iii-3) comprises or consists of the amino acid sequence of any one of SEQ IDS NO: 361, 362, 363, 364, 365, and 366, or comprises or consists of the CDRH3 amino acid sequence contained in any one of SEQ ID NOS: 381, 382, 388, 393, 394, 395, and 396, optionally defined according to IMGT or Kabat.

In some cases, the multispecific antibody or antibody fragment may comprise the VH1 described above, the VL1 described above, and the VH2 described above.

In some cases, the VH2 is, comprises, or is comprised in a nanobody.

In some cases, the multispecific antibody or antibody fragment is a bispecific antibody or antibody fragment.

In certain embodiments, the VH1 comprises a CDRH1, a CDRH2, and a CDRH3 having the amino acid sequences of: (i) SEQ ID NOS: 121, 141, and 161, respectively, (ii) SEQ ID NOS: 122, 142, and 161, respectively, (iii) SEQ ID NOS: 121, 142, and 161, respectively, (iv) SEQ ID NOS: 123, 142, and 161, respectively, (v) SEQ ID NOS: 124, 142, and 161, respectively, (vi) SEQ ID NOS: 122, 141, and 161, respectively, (vii) SEQ ID NOS: 122, 143, and 161, respectively, (viii) SEQ ID NOS: 122, 144, and 161, respectively; (ix) SEQ ID NOS: 121, 143, and 161, respectively, (x) SEQ ID NOS: 121, 144, and 161, respectively, (xi) SEQ ID NOS: 123, 141, and 161, respectively, (xii) SEQ ID NOS: 123, 143, and 161, respectively, (xiii) SEQ ID NOS: 123, 144, and 161, respectively, (xiv) SEQ ID NOS: 124, 141, and 161, respectively, (xv) SEQ ID NOS: 124, 143, and 161, respectively, or (xvi) SEQ ID NOS: 124, 144, and 161, respectively.

In certain embodiments, the VL1 comprises a CDRL1, a CDRL2, and a CDRL3 having the amino acid sequences of SEQ ID NOS: 221, 241, and 261, respectively.

In certain embodiments, the VH2 comprises a CDRH1, a CDRH2, and a CDRH3 having the amino acid sequences of: (i) SEQ ID NOS: 321, 341, and 361, respectively, (ii) SEQ ID NOS: 321, 341, and 362, respectively, (iii) SEQ ID NOS: 321, 341, and 363, respectively, (iv) SEQ ID NOS: 321, 341, and 364, respectively, (v) SEQ ID NOS: 321, 341, and 365, respectively, or (vi) SEQ ID NOS: 321, 341, and 366, respectively.

In some cases, the multispecific antibody or antibody fragment may comprise the VH1 described above, the VL1 described above, and the VH2 described above.

In certain embodiments, the VH1 comprises: (i) the CDRH1, the CDRH2, and the CDRH3 of the VH1 comprising the amino acid sequence contained in any one of SEQ ID NOS: 181-188, optionally according to Kabat; or (ii) a CDRH1, a CDRH2, and a CDRH3 comprising the amino acid sequences of: (ii-1) SEQ ID NOS: 121, 141, and 161, respectively, (ii-2) SEQ ID NOS: 122, 142, and 161, respectively, (ii-3) SEQ ID NOS: 121, 142, and 161, respectively, (ii-4) SEQ ID NOS: 123, 142, and 161, respectively, (ii-5) SEQ ID NOS: 124, 142, and 161, respectively, (ii-6) SEQ ID NOS: 122, 141, and 161, respectively, (ii-7) SEQ ID NOS: 122, 143, and 161, respectively, or (ii-8) SEQ ID NOS: 122, 144, and 161, respectively.

In certain embodiments, the VL1 comprises: (i) the CDRL1, the CDRL2, and the CDRL3 of the VL1 comprising the amino acid sequence contained in SEQ ID NO: 281, optionally according to Kabat, (ii) a CDRL1, a CDRL2, and a CDRL3 comprising the amino acid sequences of SEQ ID NOS: 221, 241, and 261, respectively.

In certain embodiments, the VH2 comprises: (i) the CDRH1, the CDRH2, and the CDRH3 of the VH2 comprising the amino acid sequence contained in any one of SEQ ID NOS: 381, 386, 393, 394, 395, and 396, optionally according to IMGT or Kabat; or (ii) a CDRH1, a CDRH2, and a CDRH3 comprising the amino acid sequences of: (ii-1) SEQ ID NOS: 321, 341, and 361, respectively, (ii-2) SEQ ID NOS: 321, 341, and 362, respectively, (ii-3) SEQ ID NOS: 321, 341, and 363, respectively, (ii-4) SEQ ID NOS: 321, 341, and 364, respectively, (ii-5) SEQ ID NOS: 321, 341, and 365, respectively, (ii-6) SEQ ID NOS: 321, 341, and 366, respectively.

In some cases, the multispecific antibody or antibody fragment may comprise the VH1 described above, the VL1 described above, and the VH2 described above.

In particular embodiments, (A) the VH1 comprises: (i) the CDRH1, the CDRH2, and the CDRH3 of the VH1 comprising the amino acid sequence contained in SEQ ID NO: 181, optionally according to Kabat; or (ii) a CDRH1, a CDRH2, and a CDRH3 comprising the amino acid sequences of SEQ ID NOS: 121, 141, and 161, respectively, and the VL1 comprises: (i) the CDRL1, the CDRL2, and the CDRL3 of the VL1 comprising the amino acid sequence contained in SEQ ID NO: 281, optionally according to Kabat, or (ii) a CDRL1, a CDRL2, and a CDRL3 comprising the amino acid sequences of SEQ ID NOS: 221, 241, and 261, respectively; and (B) the VH2 comprises: (i) the CDRH1, the CDRH2, and the CDRH3 of the VH2 comprising the amino acid sequence contained in any one of SEQ ID NO: 381, 393, 394, 395, or 396, optionally according to IMGT or Kabat; or (ii) a CDRH1, a CDRH2, and a CDRH3 comprising the amino acid sequences of SEQ ID NOS: 321, 341, and 361, respectively, SEQ ID NOS: 321, 341, and 363, respectively, SEQ ID NOS: 321, 341, and 364, respectively, SEQ ID NOS: 321, 341, and 365, or SEQ ID NOS: 321, 341, and 366, respectively.

In certain embodiments, the VH1 comprises: (i) a heavy chain framework region 1 (FRH1), which has an amino acid sequence which: (i-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 111 or to the FRH1 amino acid sequence contained in SEQ ID NO: 181, optionally according to Kabat; (i-2) comprises or consists of the amino acid sequence of SEQ ID NO: 110, wherein X₁ is any amino acid, optionally Q or K; (i-3) comprises or consists of the amino acid sequence of SEQ ID NO: 111 or 112, or comprises or consists of the FRH1 amino acid sequence contained in any one of SEQ ID NOS: 181-188, optionally according to Kabat; and/or (i-4) consists of, or is derived from the FRH1 encoded by the human germline IGHV3-23*04; (ii) a heavy chain framework region 2 (FRH2), which has an amino acid sequence which: (ii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 131 or to the FRH2 amino acid sequence contained in SEQ ID NO: 181, optionally according to Kabat; (ii-2) comprises or consists of the amino acid sequence of SEQ ID NO: 130 or 131, or comprises or consists of the FRH2 amino acid sequence contained in any of SEQ ID NOS: 181-188, optionally according to Kabat; and/or (ii-3) consists of, or is derived from the FRH2 encoded by the human germline IGHV3-23*04; (iii) a heavy chain framework region 3 (FRH3), which has an amino acid sequence which: (iii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 151 or to the FRH3 amino acid sequence contained in SEQ ID NO: 181, optionally according to Kabat; (iii-2) comprises or consists of the amino acid sequence of SEQ ID NO: 150, wherein X₁ is any amino acid, optionally N or D, X₂ is any amino acid, optionally R or K, and X₃ is any amino acid, optionally A or T; (iii-3) comprises or consists of the amino acid sequence of SEQ ID NO: 151 or 152, comprises or consists of or the FRH3 amino acid sequence contained in any of SEQ ID NOS: 181-188, optionally according to Kabat; and/or (iii-4) consists of, or is derived from the FRH3 encoded by the human germline IGHV3-23*04; and/or (iv) a heavy chain framework region 4 (FRH4), which has an amino acid sequence which: (iv-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 171 or to the FRH4 amino acid sequence contained in SEQ ID NO: 181, optionally according to Kabat; (iv-2) comprises or consists of the amino acid sequence of SEQ ID NO: 170 or 171, or comprises or consists of the FRH4 amino acid sequence contained in any of SEQ ID NOS: 181-188, optionally according to Kabat; and/or (iv-3) consists of, or is derived from the FRH4 encoded by the human germline IGHJ4*01.

In certain embodiments, the VL1 comprises: (v) a light chain framework region 1 (FRL1), which has an amino acid sequence which: (v-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 211 or to the FRL1 amino acid sequence contained in SEQ ID NO: 281, optionally according to Kabat; or (v-2) comprises or consists of the amino acid sequence of SEQ ID NO: 211, or comprises or consists of the FRL1 amino acid sequence contained in SEQ ID NO: 281, optionally according to Kabat; (vi) a light chain framework region 2 (FRL2), which has an amino acid sequence which: (vi-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 231 or to the FRL2 amino acid sequence contained in SEQ ID NO: 281, optionally according to Kabat; or (vi-2) comprises or consists of the amino acid sequence of SEQ ID NO: 231, or comprises or consists of the FRL2 amino sequence contained in SEQ ID NO: 281, optionally according to Kabat; (vii) a light chain framework 3 (FRL3), which has an amino acid sequence which: (vii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 251 or to the FRL3 amino acid sequence contained in SEQ ID NO: 281, optionally according to Kabat; or (vii-2) comprises or consists of the amino acid sequence of SEQ ID NO: 251, or comprises or consists of the FRL3 amino acid sequence contained in SEQ ID NO: 281, optionally according to Kabat; and/or (viii) a light chain framework 4 (FRL4), which has an amino acid sequence which: (viii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 271 or to the FRL4 amino acid sequence contained in SEQ ID NO: 281, optionally according to Kabat; or (viii-2) comprises or consists of the amino acid sequence of SEQ ID NO: 271, or comprises or consists of the FRL4 amino acid sequence contained in SEQ ID NO: 281, optionally according to Kabat.

In certain embodiments, the VH2 comprises: (i) a FRH1, which has an amino acid sequence which: (i-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 311 or to the FRH1 amino acid sequence contained in SEQ ID NO: 381, optionally according to IMGT or Kabat; (i-2) comprises or consists of the amino acid sequence of SEQ ID NO: 310, wherein X₁ is any amino acid, optionally E or D, X₂ is any amino acid, optionally A or T, and X₃ is any amino acid, optionally S, optionally wherein the FRH1 does not consist of the amino acid sequence of SEQ ID NO: 310 wherein X₁, X₂, and X₃ are E, A, and A, respectively; (i-3) comprises or consists of the amino acid sequence of SEQ ID NO: 311 or 312, or comprises or consists of the FRH1 amino acid sequence contained in any one of SEQ ID NOS: 381, 382-386, and 393-396, optionally according to IMGT or Kabat; and/or (i-4) consists of, or is derived from the FRH1 encoded by the human germline IGHV3-66*01; (ii) a FRH2, which has an amino acid sequence which: (ii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 331 or to the FRH2 amino acid sequence contained in SEQ ID NO: 381, optionally according to IMGT or Kabat; (ii-2) comprises or consists of the amino acid sequence of SEQ ID NO: 330, wherein X₁ is any amino acid, optionally Y, X₂ is any amino acid, optionally Q, X₃ is any amino acid, optionally R, X₄ is any amino acid, optionally L, X₅ is any amino acid, optionally I, and X₆ is any amino acid, optionally A, optionally wherein the FRH2 does not consist of the amino acid sequence of SEQ ID NO: 330 wherein X₁, X₂, X₃, X₄, X₅, and X₆ are V, G, L, W, V, and S, respectively; (ii-3) comprises or consists of the amino acid sequence of SEQ ID NO: 331, 332, or 333, or comprises or consists of the FRH2 amino acid sequence contained in any one of SEQ ID NOS: 381, 382-386, and 393-396, optionally according to IMGT or Kabat; and/or (ii-4) consists of, or is derived from the FRH2 encoded by the human germline IGHV3-66*01; (iii) a FRH3, which has an amino acid sequence which: (iii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 351 or to the FRH3 amino acid sequence contained in SEQ ID NO: 381, optionally according to IMGT or Kabat; (iii-2) comprises or consists of the amino acid sequence of SEQ ID NO: 350, wherein X₁ is any amino acid, optionally D or G; X₂ is any amino acid, optionally L; X₃ is any amino acid, optionally M; X₄ is any amino acid, optionally T; X₅ is any amino acid, optionally D or E; X₆ is any amino acid, optionally N or S; X₇ is any amino acid, optionally S or A; X₈ is any amino acid, optionally M; X₉ is any amino acid, optionally R or K; and X₁₀ is any amino acid, optionally A or P, optionally wherein the FRH3 does not consist of the amino acid sequence of SEQ ID NO: 350 wherein X₁, X₂, X₃, X₄, X₅, X₆, X₇, X₈, X₉, and X₁₀ are D, V, I, R, D, N, S, L, R, and A, respectively; (iii-3) comprises or consists of the amino acid sequence of SEQ ID NO: 351 or 352, or comprises or consists of the FRH3 amino acid sequence contained in any one of SEQ ID NOS: 381, 382-386, and 393-396, optionally according to IMGT or Kabat; and/or (iii-4) consists of, or is derived from the FRH3 encoded by the human germline IGHV3-66*01; and/or (iv) a FRH4, which has an amino acid sequence which: (iv-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 371 or to the FRH4 amino acid sequence contained in SEQ ID NO: 381, optionally according to IMGT or Kabat; (iv-2) comprises or consists of the amino acid sequence of SEQ ID NO: 370, wherein X₁ is any amino acid, optionally R or W; and X₂ is any amino acid, optionally Q or L; (iv-3) comprises or consists of the amino acid sequence of SEQ ID NO: 351 or 352, or comprises or consists of the FRH4 amino acid sequence contained in any of SEQ ID NOS: 381, 382-386, and 393-396, optionally according to IMGT or Kabat; and/or (iv-4) consists of, or is derived from the FRH4 encoded by the human germline IGHV3-66*01.

In some cases, the multispecific antibody or antibody fragment may comprise the VH1 described above, the VL1 described above, and the VH2 described above.

In certain embodiments, the VH1 comprises an amino acid sequence which: (i) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of any one of SEQ ID NOS: 181-188; and/or (ii) comprises or consists of the amino acid sequence of SEQ ID NO: 180, wherein: X₁ is any amino acid, optionally Q or K; X₂ is any amino acid, optionally S, A, Q, or N; X₃ is any amino acid, optionally E, A, S, or D; X₄ is any amino acid, optionally N or D; X₅ is any amino acid, optionally R or K; X₆ is any amino acid, optionally E or T; and X₇ is any amino acid, optionally D or any amino acid which is not S, E, A, R, T, L, H, Y, Q, or K.

In certain embodiments, the VL1 comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 281.

In certain embodiments, the VH2 comprises an amino acid sequence which: (i) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of any one of SEQ ID NOS: 381, 386, and 393-396; and/or (ii) comprises or consists of the amino acid sequence of SEQ ID NO: 380, wherein: X₁ is any amino acid, optionally E or D; X₂ is any amino acid such as A or T; and X₃ is any amino acid such as S; X₄ is Q; X₅ is any amino acid, such as Y; X₆ is any amino acid, such as Q or G; X₇ is any amino acid, such as R; X₈ is any amino acid, such as L or W; X₉ is any amino acid, such as I; and X₁₀ is any amino acid, such as A; X₁₁ is M; X₁₂ is M; X₁₃ is any amino acid, such as D or G; X₁₄ is any amino acid, such as L; X₁₅ is any amino acid, such as M; X₁₆ is any amino acid, such as T; X₁₇ is any amino acid, such as D or E; X₁₈ is any amino acid, such as N or S; X₁₉ is any amino acid, such as S or A; X₂₀ is any amino acid, such as M; X₂₁ is any amino acid, such as R or K; and X₂₂ is any amino acid, such as A or P; X₂₃, X₂₄, and X₂₅ are individually any amino acids as long as (a) X₂₃ and X₂₄ are D and E, respectively, (b) X₂₃ and X₂₅ are D and D, respectively, and/or (c) X₂₄ and X₂₅ are E and D, respectively, optionally wherein X₂₃, X₂₄, and X₂₅ are D, E, and D, respectively, D, D, and D, respectively, D, E, and E, respectively, D, E, and S, respectively, D, E, and A, respectively, or D, E, and T, respectively; X₂₆ is any amino acid, such as R or W; and X₂₇ is any amino acid, such as Q or L.

In some cases, the multispecific antibody or antibody fragment may comprise the VH1 described above, the VL1 described above, and the VH2 described above.

In some particular embodiments, (A) the VH1 comprises an amino acid sequence which comprises or consists of the amino acid sequence of any one of SEQ ID NOS: 181-188: and/or the VL1 comprises an amino acid sequence which comprises or consists of the amino acid sequence of SEQ ID NO: 281; and/or (B) the VH2 comprises an amino acid sequence which comprises or consists of the amino acid sequence of any one of SEQ ID NOS: 381, 386, and 393-396.

In particular embodiments, (A) the VH1 comprises: (i) the CDRH1, the CDRH2, and the CDRH3 of the VH1 comprising the amino acid sequence contained in SEQ ID NO: 181, optionally according to Kabat; or (ii) a CDRH1, a CDRH2, and a CDRH3 which comprise the amino acid sequences of SEQ ID NOS: 121, 141, and 161, respectively, and the VL1 comprises: (i) the CDRL1, the CDRL2, and the CDRL3 of the VL1 comprising the amino acid sequence contained in SEQ ID NO: 281, optionally according to Kabat, or (ii) a CDRL1, a CDRL2, and a CDRL3 which comprise the amino acid sequences of SEQ ID NOS: 221, 241, and 261, respectively; and (B) the VH2 comprises: (i) the CDRH1, the CDRH2, and the CDRH3 of the VH2 comprising the amino acid sequence contained in SEQ ID NO: 381, optionally according to IMGT or Kabat; or (ii) a CDRH1, a CDRH2, and a CDRH3 which comprise the amino acid sequences of SEQ ID NOS: 321, 341, and 361, respectively.

In certain embodiments, the VH1 comprises: (i) a FRH1 which has an amino acid sequence which: (i-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 111 or to the FRH1 amino acid sequence contained in SEQ ID NO: 181, optionally according to Kabat; (i-2) comprises or consists of the amino acid sequence of SEQ ID NO: 111, or comprises or consists of the FRH1 amino acid sequence contained in SEQ ID NO: 181, optionally according to Kabat; and/or (i-3) consists of, or is derived from the FRH1 encoded by the human germline IGHV3-23*04; (ii) a FRH2 which has an amino acid sequence which: (ii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 131 or to the FRH2 amino acid sequence contained in SEQ ID NO: 181, optionally according to Kabat; (ii-2) comprises or consists of the amino acid sequence of SEQ ID NO: 131, or comprises or consists of the FRH2 amino acid sequence contained in SEQ ID NO: 181, optionally according to Kabat; and/or (ii-3) consists of, or is derived from the FRH2 encoded by the human germline IGHV3-23*04; (iii) a FRH3 which has an amino acid sequence which: (iii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 151 or to the FRH3 amino acid sequence contained in SEQ ID NO: 181, optionally according to Kabat; (iii-2) comprises or consists of the amino acid sequence of SEQ ID NO: 151, or comprises or consists of the FRH3 amino acid sequence contained in SEQ ID NO: 181, optionally according to Kabat; and/or (iii-3) consists of, or is derived from the FRH3 encoded by the human germline IGHV3-23*04; and/or (iv) a FRH4 which has an amino acid sequence which: (iv-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 171 or to the FRH4 amino acid sequence contained in SEQ ID NO: 181, optionally according to Kabat; (iv-2) comprises or consists of the amino acid sequence of SEQ ID NO: 171, or comprises or consists of the FRH4 amino acid sequence contained in SEQ ID NO: 181, optionally according to Kabat; and/or (iv-3) consists of, or is derived from the FRH4 encoded by the human germline IGHJ4*01.

In certain embodiments, the VL1 comprises: (v) a FRL1 which has an amino acid sequence which: (v-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 211 or to the FRL1 amino acid sequence contained in SEQ ID NO: 281, optionally according to Kabat; or (v-2) comprises or consists of the amino acid sequence of SEQ ID NO: 211, or comprises or consists of the FRL1 amino acid sequence contained in SEQ ID NO: 281, optionally according to Kabat; (vi) a FRL2 which has an amino acid sequence which: (vi-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 231 or to the FRL2 amino acid sequence contained in SEQ ID NO: 281, optionally according to Kabat; or (vi-2) comprises or consists of the amino acid sequence of SEQ ID NO: 231, or comprises or consists of the FRL2 amino acid sequence contained in SEQ ID NO: 281, optionally according to Kabat; (vii) a FRL3 which has an amino acid sequence which: (vii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 251 or to the FRL3 amino acid sequence contained in SEQ ID NO: 281, optionally according to Kabat; or (vii-2) comprises or consists of the amino acid sequence of SEQ ID NO: 251, or comprises or consists of the FRL3 amino acid sequence contained in SEQ ID NO: 281, optionally according to Kabat; and/or (viii) a FRL4 which has an amino acid sequence which: (viii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 271 or to the FRL4 amino acid sequence contained in SEQ ID NO: 281, optionally according to Kabat; or (viii-2) comprises or consists of the amino acid sequence of SEQ ID NO: 271, or comprises or consists of the FRL4 amino acid sequence contained in SEQ ID NO: 281, optionally according to Kabat.

In certain embodiments, the VH2 comprises: (i) a FRH1 which has an amino acid sequence which: (i-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 311 or to the FRH1 amino acid sequence contained in SEQ ID NO: 381, optionally according to IMGT or Kabat; (i-2) comprises or consists of the amino acid sequence of SEQ ID NO: 311, or comprises or consists of the FRH1 amino acid sequence contained in SEQ ID NO: 381, optionally according to IMGT or Kabat; and/or (i-3) consists of, or is derived from the FRH1 encoded by the human germline IGHV3-66*01; (ii) a FRH2 which has an amino acid sequence which: (ii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 331 or to the FRH2 amino acid sequence contained in SEQ ID NO: 381, optionally according to IMGT or Kabat; (ii-2) comprises or consists of the amino acid sequence of SEQ ID NO: 331, or comprises or consists of the FRH2 amino acid sequence contained in SEQ ID NO: 381, optionally according to IMGT or Kabat; and/or (ii-3) consists of, or is derived from the FRH2 encoded by the human germline IGHV3-66*01; (iii) a FRH3 which has an amino acid sequence which: (iii-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 351 or to the FRH3 amino acid sequence contained in SEQ ID NO: 381, optionally according to IMGT or Kabat; (iii-2) comprises or consists of the amino acid sequence of SEQ ID NO: 351, or comprises or consists of the FRH3 amino acid sequence contained in SEQ ID NO: 381, optionally according to IMGT or Kabat; and/or (iii-3) consists of, or is derived from the FRH3 encoded by the human germline IGHV3-66*01; and/or (iv) a FRH4 which has an amino acid sequence which: (iv-1) has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 371 or to the FRH4 amino acid sequence contained in SEQ ID NO: 381, optionally according to IMGT or Kabat; (iv-2) comprises or consists of the amino acid sequence of SEQ ID NO: 351, or comprises or consists of the FRH4 amino acid sequence contained in SEQ ID NO: 381, optionally according to IMGT or Kabat; and/or (iv-3) consists of, or is derived from the FRH4 encoded by the human germline IGHV3-66*01.

In some cases, the multispecific antibody or antibody fragment may comprise the VH1 described above, the VL1 described above, and the VH2 described above.

In some particular embodiments, (A) the VH1 comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of any one of SEQ ID NOS: 181; and/or the VL1 comprises an amino acid sequence which at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 281; and/or (B) the VH2 comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 381.

In some particular embodiments, (A) the VH1 comprises an amino acid sequence which comprises or consists of the amino acid sequence of SEQ ID NO: 181: and/or the VL1 comprises an amino acid sequence which comprises or consists of the amino acid sequence of SEQ ID NO: 281; and/or (B) the VH2 comprises an amino acid sequence which comprises or consists of the amino acid sequence of any of SEQ ID NOS: 381.

In particular embodiments, (A) the VH1 comprises an amino acid sequence which consists of the amino acid sequence of SEQ ID NO: 181: and the VL1 comprises an amino acid sequence which consists of the amino acid sequence of SEQ ID NO: 281; and (B) the VH2 comprises an amino acid sequence which consists of the amino acid sequence of SEQ ID NO: 381.

In some particular embodiments, a multispecific antibody or antibody fragment according to the present disclosure may comprise: (A) a first antigen-binding region which specifically binds to Fzd4 and comprises a VH1 and a VL1; and (B) a second antigen-binding region which specifically binds to LRP5 and/or LRP6 and comprises at least a VH2,

wherein: (i) the CDRH1, the CDRH2, and the CDRH3 of the VH1 are identical to those of the heavy chain variable domain (VH) having the amino acid sequence of SEQ ID NO: 181; (ii) the CDRL1, the CDRL2, and the CDRL3 of the VL1 are identical to those of the light chain variable domain (VL) having the amino acid sequence of SEQ ID NO: 281; and (iii) the CDRH1, the CDRH2, and the CDRH3 of the VH2 are identical to those of the VH having the amino acid sequence of SEQ ID NO: 381. In particular embodiments: (i) the VH1 comprises or consists of the amino acid sequence of SEQ ID NO: 181; (ii) the VL1 comprises or consists of the amino acid sequence of SEQ ID NO: 281; and (iii) the VH2 comprises or consists of the amino acid sequence of SEQ ID NO: 381.

In some embodiments, (A) the first antigen-binding region is, comprises, or is comprised in a fragment antigen-binding (Fab), a Fab′ fragment, a F(ab′)₂, a single-chain Fab (scFab), a variable fragment (Fv), a single-chain Fv (scFv), a diabody, or a triabody; and/or (B) the second antigen-binding region: (i) is, comprises, or is comprised in a nanobody or a Fd fragment; or (ii) further comprises a second light chain variable domain (VL2) and is, comprises, or is comprised in a Fab, a Fab′ fragment, a F(ab′)₂, a scFab, a Fv, a scFv, a diabody, or a triabody.

In some embodiments, the multispecific antibody or antibody fragment comprises one or more of the following: (a) an immunoglobulin heavy chain constant domain 1 (CH1) or a variant thereof; (b) an immunoglobulin hinge or a variant thereof, (c) an immunoglobulin heavy chain constant domain 2 (CH2) or a variant thereof; (d) an immunoglobulin heavy chain constant domain 3 (CH3) or a variant thereof, and/or (e) an immunoglobulin light chain constant domain (CL) or a variant thereof.

In certain embodiments, the CH1 or a variant thereof: (a-1) is of an IgG, an IgA, an IgE, an IgD, an IgM, optionally of an IgG1, an IgG4, an IgG2, or an IgG3, further optionally of human; and/or (a-2) comprises an amino acid sequence which comprises or consists of the amino acid sequence of SEQ ID NO: 21 or 22 or which is at least 80%, at least 85%, at least 90%, at least 91%, at least 92%, at least 93%, at least 94%, at least 95%, at least 96%, at least 97%, at least 98%, at least 99%, or is 100% identical thereto;

In certain embodiments, the hinge or a variant thereof: (b-1) is of an IgG, an IgA, an IgE, an IgD, an IgM, optionally of an IgG1, an IgG4, an IgG2, or an IgG3, further optionally of human; and/or (b-2) comprises an amino acid sequence which comprises or consists of the amino acid sequence of SEQ ID NO: 31 or which is at least 80%, at least 85%, at least 90%, at least 91%, at least 92%, at least 93%, at least 94%, at least 95%, at least 96%, at least 97%, at least 98%, at least 99%, or is 100% identical thereto.

In certain embodiments, the CH2 or a variant thereof: (c-1) is of an IgG, an IgA, an IgE, an IgD, an IgM, optionally of an IgG1, an IgG4, an IgG2, or an IgG3, further optionally of human; and/or (c-2) comprises an amino acid sequence which comprises or consists of the amino acid sequence of any of SEQ ID NOS: 41-46 or which is at least 80%, at least 85%, at least 90%, at least 91%, at least 92%, at least 93%, at least 94%, at least 95%, at least 96%, at least 97%, at least 98%, at least 99%, or is 100% identical thereto.

In certain embodiments, the CH3 or a variant thereof: (d-1) is of an IgG, an IgA, an IgE, an IgD, an IgM, optionally of an IgG1, an IgG4, an IgG2, or an IgG3, further optionally of human; and/or (d-2) comprises an amino acid sequence which comprises or consists of the amino acid sequence of any of SEQ ID NOS: 51-54, 61-64, and 66-69 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or 100% identical thereto.

In certain embodiments, the CL or a variant thereof: (e-1) is of a kappa light chain constant domain (CLκ), optionally human CLκ and optionally comprising an amino acid sequence which comprises or consists of the amino acid sequence of SEQ ID NO: 11 or which is at least 80%, at least 85%, at least 90%, at least 91%, at least 92%, at least 93%, at least 94%, at least 95%, at least 96%, at least 97%, at least 98%, at least 99%, or 100% identity thereto; and/or (e-2) is of a lambda light chain constant domain (CLλ), optionally human CLλ, and optionally comprising an amino acid sequence which comprises or consists of the amino acid sequence of SEQ ID NO: 12 or which is at least 80%, at least 85%, at least 90%, at least 91%, at least 92%, at least 93%, at least 94%, at least 95%, at least 96%, at least 97%, at least 98%, at least 99%, or 100% identity thereto.

In some particular embodiments, the multispecific antibody or antibody fragment comprises the CH2 and CH3 sequences of SEQ ID NOS: 42 and 51, respectively, SEQ ID NOS: 43 and 51, respectively, SEQ ID NOS: 45 and 61, respectively, SEQ ID NO: 45 and 66, respectively, SEQ ID NOS: 46 and 61, respectively, or SEQ ID NO: 46 and 66, respectively.

In some embodiments, the multispecific antibody or antibody fragment comprises a fragment crystallizable (Fc) region.

In certain embodiments, the Fc region is of an IgG, an IgA, an IgE, an IgD, an IgM, optionally of an IgG1, an IgG4, an IgG2, or an IgG3, further optionally of human, or a variant thereof.

In certain embodiments, the Fc region of an IgG1 or a variant thereof may comprise one or more of the following amino acid substitutions: N297A, N297Q, D265A, L234A, L235A, C226S, C229S, P238S, E233P, L234V, G236-deleted, P238A, A327Q, A327G, P329A, P329G, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, H310A, L328F, M252Y, I253A, S254T, T256E, H435Q, H435A, and/or any combination thereof, according to EU numbering;

In certain embodiments, the Fc region of an IgG2 or a variant thereof may comprise one or more of the following amino acid substitutions: P238S, V234A, G237A, H268A, H268Q, H268E, V309L, N297A, N297Q, A330S, P331S, C232S, C233S, M252Y, S254T, T256E, and/or any combination thereof, according to EU numbering.

In certain embodiments, the Fc region of an IgG3 or a variant thereof may comprise the amino acid substitution E235Y, according to EU numbering.

In certain embodiments, the Fc region of an IgG4 or a variant thereof may comprise one or more of the following amino acid substitutions: E233P, F234V, L235A, G237A, E318A, S228P, L236E, S241P, L248E, T394D, M252Y, S254T, T256E, N297A, N297Q, and/or any combination thereof, according to EU numbering.

In certain embodiments, the Fc region may comprise one or more amino acid substitutions that reduce an Fc effector function.

In some particular embodiments, the one or more amino acid substitutions are at position(s) selected from the group consisting of positions 234, 235, 236, 237, 265, 297 and 329, according to EU numbering.

In some particular embodiments, the one or more amino acid substitutions comprises at least the following amino acid substitutions according to EU numbering: (ii-1) L234A, L235A, and P329G; (ii-2) L234A and L235A; (ii-3) I253A, H310A, and H435Q; (ii-4) I253A, H310A, and H435A; (ii-5) D265A and P329A; (ii-6) M252Y, S254T, and T256E; and/or (ii-7) M428L and N434S. Optionally, such substitutions may be relative to a human IgG1 Fc region and/or to any of SEQ ID NOS: 71-74 (optionally not including the C-terminal K).

In some particular embodiments, the Fc region may comprise an amino acid sequence which comprises or consists of the amino acid sequence of any one of SEQ ID NOS: 71-74, 81-84, 86-89, 91-94, 96-99, 471-474, 476-479, 481-484, and 486-489 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or is 100% identical thereto.

In some embodiments, the multispecific antibody or antibody fragment according to the present disclosure may comprise a structure as depicted in any one of FIGS. 24-35, optionally any of the structures depicted in FIG. 24.

In some embodiments, the multispecific antibody or antibody fragment according to the present disclosure may comprise: (a) a first polypeptide comprising said VH1; and (b) a second polypeptide comprising said VH2 and said VL1, wherein the first and second polypeptides interact with each other, optionally via one or more disulfide bonds, permitting said VH1 and VL1 to form said first antigen-binding region.

In certain embodiments, (a) the first polypeptide comprises the following in the order from the N-terminus to the C-terminus: (a-1) said VH1 and (a-2) a CH1 or a variant thereof, optionally wherein the CH1 or a variant thereof: (i) is of an IgG, optionally of an IgG1 or an IgG4, further optionally of human IgG1 or IgG4; and/or (ii) comprises an amino acid sequence which comprises or consists of the amino acid sequence of SEQ ID NO: 21 or 22 or which is at least 80%, at least 85%, at least 90%, at least 91%, at least 92%, at least 93%, at least 94%, at least 95%, at least 96%, at least 97%, at least 98%, at least 99%, or is 100% identical thereto; (a-3) an immunoglobulin hinge or a variant thereof, optionally wherein the hinge or a variant thereof: (i) is of an IgG, optionally of an IgG1 or an IgG4, further optionally of human IgG1 or IgG4; and/or (ii) comprising an amino acid sequence which comprises or consists of the amino acid sequence of SEQ ID NO: 31 or which is at least 80%, at least 85%, at least 90%, at least 91%, at least 92%, at least 93%, at least 94%, at least 95%, at least 96%, at least 97%, at least 98%, at least 99%, or is 100% identical thereto; (a-4) a CH2 or a variant thereof, optionally wherein the CH2 or a variant thereof: (i) is of an IgG, optionally of an IgG1 or an IgG4, further optionally of human IgG1 or IgG4; and/or (ii) comprises an amino acid sequence which comprises or consists of the amino acid sequence of any of SEQ ID NOS: 41-46 or which is at least 80%, at least 85%, at least 90%, at least 91%, at least 92%, at least 93%, at least 94%, at least 95%, at least 96%, at least 97%, at least 98%, at least 99%, or is 100% identical thereto; and (a-5) a CH3 or a variant thereof, optionally wherein the CH3 or a variant thereof: (i) is of an IgG, optionally of an IgG1 or an IgG4, further optionally of human IgG1 or IgG4; and/or (ii) comprises an amino acid sequence which comprises or consists of the amino acid sequence of any of SEQ ID NOS: 51-54, 61-64, and 66-69 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or is 100% identical thereto.

In certain embodiments, (b) the second polypeptide comprises the following in the order from the N-terminus to the C-terminus: (b-1) said VH2; (b-2) optionally a linker, which is optionally a peptide linker, further optionally a flexible linker, optionally wherein the linker: (i) comprises one or more amino acids, optionally one, two, three, four, five, six, seven, eight, nine, ten, eleven, or twelve amino acids; (ii) consists of small amino acids consisting of G, S, and/or A; (iii) comprises an amino acid sequence which comprises or consists of the amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG; and/or (iv) comprises an amino acid sequence which comprises or consists of multiple repeats of the amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG; (b-3) said VL1; and (b-4) an CL or a variant thereof, optionally wherein the CL or a variant thereof: (i) is of a CLκ, optionally human CLκ, and optionally comprising an amino acid sequence comprising or consisting of the amino acid sequence of SEQ ID NO: 11 or having at least 80%, at least 85%, at least 90%, at least 91%, at least 92%, at least 93%, at least 94%, at least 95%, at least 96%, at least 97%, at least 98%, at least 99%, or 100% identity thereto; or (ii) is of a CLλ, optionally human CLλ, and optionally comprising an amino acid sequence comprising or consisting of the amino acid sequence of SEQ ID NO: 12 or having at least 80%, at least 85%, at least 90%, at least 91%, at least 92%, at least 93%, at least 94%, at least 95%, at least 96%, at least 97%, at least 98%, at least 99%, or 100% identity thereto.

In some cases, the multispecific antibody or antibody fragment may comprise both the first polypeptide described above and the second polypeptide described above.

In some particular embodiments, the multispecific antibody or antibody fragment comprises: two of said first polypeptides forming a dimer with each other optionally via one or more disulfide bonds; and two of said second polypeptides, wherein one of the second polypeptides is interacting with one of the first polypeptides optionally via a disulfide bond and the other of the second polypeptides is interacting with the other of the first polypeptides optionally via a disulfide bond.

In certain embodiments, the multispecific antibody or antibody fragment comprises a Fc region, wherein the Fc region: (I) is of an IgG, optionally of an IgG1 or an IgG4, further optionally of human, or a variant thereof; (II) comprises at least the following amino acid substitutions according to EU numbering: (ii-1) L234A, L235A, and P329G; (ii-2) L234A and L235A; (ii-3) I253A, H310A, and H435Q; (ii-4) I253A, H310A, and H435A; (ii-5) D265A and P329A; (ii-6) M252Y, S254T, and T256E; and/or (ii-7) M428L and N434S, optionally relative to a human IgG1 Fc region and/or to any of SEQ ID NOS: 71-74 (optionally not including the C-terminal K); and/or (III) comprises an amino acid sequence which comprises or consists of the amino acid sequence of any one of SEQ ID NOS: 71-74, 81-84, 86-89, 91-94, 96-99, 471-474, 476-479, 481-484, and 486-489 (optionally not including the C-terminal K) or comprises an amino acid sequence which is at least 80%, at least 85%, at least 90%, at least 91%, at least 92%, at least 93%, at least 94%, at least 95%, at least 96%, at least 97%, at least 98%, at least 99%, or 100% identical thereto.

In some particular embodiments, (a) the first polypeptide; (i) comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOS: 2, 7, 8, 9, 450, 451, 452, and 453 (optionally without the C-terminal K); (ii) comprises an amino acid sequence which comprises or consists of the amino acid sequence of any one of SEQ ID NOS: 402, 407, 408, 409, 460, 461, 462, and 463 (optionally without the C-terminal K), wherein: X₁ is any amino acid, optionally Q or K; X₂ is any amino acid, optionally S, A, Q, or N; X₃ is any amino acid, optionally E, A, S, or D; X₄ is any amino acid, optionally N or D; X₅ is any amino acid, optionally R or K; X₆ is any amino acid, optionally E or T; and X₇ is any amino acid, optionally D or any amino acid which is not S, E, A, R, T, L, H, Y, Q, or K; and/or (iii) comprises an amino acid sequence which comprises or consists of the amino acid sequence of any one of SEQ ID NOS: 2, 7, 8, 9, 450, 451, 452, and 453 (optionally without the C-terminal K).

In some particular embodiments, (b) the second polypeptide (i) comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to SEQ ID NO: 1; and/or (ii) comprises an amino acid sequence which comprises or consists of the amino acid sequence of SEQ ID NO: 401, wherein: X₁ is any amino acid, optionally E or D; X₂ is any amino acid such as A or T; and X₃ is any amino acid such as S; X₄ is Q; X₅ is any amino acid, such as Y; X₆ is any amino acid, such as Q or G; X₇ is any amino acid, such as R; X₈ is any amino acid, such as L or W; X₉ is any amino acid, such as I; and X₁₀ is any amino acid, such as A; X₁₁ is M; X₁₂ is M; X₁₃ is any amino acid, such as D or G; X₁₄ is any amino acid, such as L; X₁₅ is any amino acid, such as M; X₁₆ is any amino acid, such as T; X₁₇ is any amino acid, such as D or E; X₁₈ is any amino acid, such as N or S; X₁₉ is any amino acid, such as S or A; X₂₀ is any amino acid, such as M; X₂₁ is any amino acid, such as R or K; and X₂₂ is any amino acid, such as A or P; X₂₃, X₂₄, and X₂₅ are individually any amino acids as long as (a) X₂₃ and X₂₄ are D and E, respectively, (b) X₂₃ and X₂₅ are D and D, respectively, and/or (c) X₂₄ and X₂₅ are E and D, respectively, optionally wherein X₂₃, X₂₄, and X₂₅ are D, E, and D, respectively, D, D, and D, respectively, D, E, and E, respectively, D, E, and S, respectively, D, E, and A, respectively, or D, E, and T, respectively; X₂₆ is any amino acid, such as R or W; and X₂₇ is any amino acid, such as Q or L; and/or (iii) comprises an amino acid sequence which comprises or consists of the amino acid sequence of SEQ ID NO: 1.

In particular embodiments, (a) the first polypeptide comprises an amino acid sequence which comprises or consists of the amino acid sequence of SEQ ID NOS: 2, 7, 450, and 451 (optionally without the C-terminal K); and (b) the second polypeptide comprises an amino acid sequence which comprises or consists of the amino acid sequence of SEQ ID NO: 1.

In further particular embodiments, the multispecific antibody or antibody fragment comprises: two of said first polypeptides forming a dimer with each other via one or more disulfide bonds; and two of said second polypeptides, wherein one of the second polypeptides is interacting with one of the first polypeptides via a disulfide bond and the other of the second polypeptides is interacting with the other of the first polypeptides via a disulfide bond.

In further embodiments, a multispecific antibody or antibody fragment of according to the present disclosure may comprise: (A) two or more Fzd4-binding regions, wherein at least one of said Fzd4-binding regions is, comprises, or is comprised in said first antigen-binding region; and/or (B) two or more LRP5 and/or LRP6-binding regions, wherein at least one of said LRP5 and/or LRP6-binding regions is, comprises, or is comprised in said second antigen-binding region.

In some cases, the ratio of the number of said one or more Fzd4-binding regions and the number of said one or more LRP5 and/or LRP6-binding regions comprised in the multispecific antibody or antibody fragment may be selected from the group consisting of 2:1, 1:2, 2:2, 3:1, 1:3, 3:2, 2:3, 3:3, 4:1, 1:4, 4:2, 2:4, 4:3, 3:4, and 4:4.

In some cases, the number of said one or more Fzd4-binding regions and the number of said one or more LRP5 and/or LRP6-binding regions comprised in the multispecific antibody or antibody fragment may be: 2 and 1, respectively; 1 and 2, respectively; 2 and 2, respectively; 3 and 1, respectively; 1 and 3, respectively; 3 and 2, respectively; 2 and 3, respectively; 3 and 3, respectively; 4 and 1, respectively; 1 and 4, respectively; 4 and 2, respectively; 2 and 4, respectively; 4 and 3, respectively; 3 and 4, respectively; or 4 and 4 respectively.

In certain cases, a multispecific antibody or antibody fragment according to the present disclosure may comprise: two identical Fzd4-binding regions, each of which is, comprises, or is comprised in said first antigen-binding region; and one LRP5 and/or LRP6-binding region which is, comprises, or is comprised in said second antigen-binding region.

In certain cases, a multispecific antibody or antibody fragment according to the present disclosure may comprise: two Fzd4-binding regions, which are different from each other and (i) at least one of which is, comprises, or is comprised in or (ii) each of which is, comprises, or is comprised in said first antigen-binding region; and one LRP5 and/or LRP6-binding region which is, comprises, or is comprised in said second antigen-binding region.

In certain cases, a multispecific antibody or antibody fragment according to the present disclosure may comprise: one Fzd4-binding region which is, comprises, or is comprised in said first antigen-binding region; and two identical LRP5 and/or LRP6-binding regions, each of which is, comprises, or is comprised in said second antigen-binding region.

In certain cases, a multispecific antibody or antibody fragment according to the present disclosure may comprise: one Fzd4-binding region which is, comprises, or is comprised in said first antigen-binding region; and two LRP5 and/or LRP6-binding regions, which are different from each other and (i) at least one of which is, comprises, or is comprised in or (ii) each of which is, comprises, or is comprised in said second antigen-binding region.

In certain cases, a multispecific antibody or antibody fragment according to the present disclosure may comprise: two identical Fzd4-binding regions, each of which is, comprises, or is comprised in said first antigen-binding region; and two identical LRP5 and/or LRP6-binding region, each of which is, comprises, or is comprised in said second antigen-binding region.

In certain cases, a multispecific antibody or antibody fragment according to the present disclosure may comprise: two Fzd4-binding regions, which are different from each other and (i) at least one of which is, comprises, or is comprised in or (ii) each of which is, comprises, or is comprised in said first antigen-binding region; and two identical LRP5 and/or LRP6-binding region, each of which is, comprises, or is comprised in said second antigen-binding region.

In certain cases, a multispecific antibody or antibody fragment according to the present disclosure may comprise: two identical Fzd4-binding regions, each of which is, comprises, or is comprised in said first antigen-binding region; and two LRP5 and/or LRP6-binding regions, which are different from each other and (i) at least one of which is, comprises, or is comprised in or (ii) each of which is, comprises, or is comprised in said second antigen-binding region.

In certain cases, a multispecific antibody or antibody fragment according to the present disclosure may comprise: two Fzd4-binding regions, which are different from each other and (i) at least one of which is, comprises, or is comprised in or (ii) each of which is, comprises, or is comprised in said first antigen-binding region; and two LRP5 and/or LRP6-binding region, which are different from each other and (i) at least one of which is, comprises, or is comprised in or (ii) each of which is, comprises, or is comprised in said second antigen-binding region.

In another aspect, the present disclosure provides nucleic acids optionally wherein a nucleic acid or a combination of nucleic acids according to the present disclosure may encode any of the multispecific antibodies and antibody fragments described herein.

In some embodiments, the nucleic acid(s) may be DNA, cDNA, RNA, mRNA, modified mRNA, or a DNA/RNA hybrid.

In some embodiments, a combination of nucleic acids nucleic acid or a combination of nucleic acids may comprise: (a) a first nucleic acid encoding any of the first polypeptides described herein; and (b) a second nucleic acids encoding any of the second polypeptides described herein.

In another aspect, the present disclosure provides vectors comprising these nucleic acids.

In some embodiments a vector or a combination of vectors according to the present disclosure may comprise any of the nucleic acids or any of the combinations of nucleic acids described herein.

In some embodiments, the vector(s) comprise(s) one or more promoters operably linked to the nucleic acid(s).

In some embodiments, the vector(s) is/are an expression vector.

In some embodiments, the vector(s) comprise(s) a plasmid, a viral vector (optionally adeno-associated viral, adenoviral, lentiviral, or retroviral), a lipid-based vector, a self-replicating RNA vector, a virus-like particle, a polymer-based vector, and/or a nanoparticle, optionally a lipid-based nanoparticle.

In some embodiments, a combination of vectors may comprise: (a) a first vector comprising a first nucleic acid encoding the first polypeptide; and (b) a second vector comprising a second nucleic acid encoding the second polypeptide.

In another aspect, the present disclosure provides host cells and populations of cells comprising such nucleic acids or vectors.

A host cell according to the present disclosure may comprise: (A) any of the multispecific antibodies and antibody fragments described herein; (B) any of the nucleic acids or any of the combinations of nucleic acids described herein; and/or (C) any of the vectors or any of the combinations of vectors described herein.

In some embodiments, the host cell is mammalian, optionally human, non-human primate, monkey, rabbit, rodent, hamster, rat, or mouse.

In some embodiments, the host cell is non-mammalian, optionally plant, bacterial, fungal, yeast, protozoa, or insect.

In certain embodiments, the host cell is: (i) a human embryonal kidney (HEK) cell, optionally a HEK293 cell, or a variant thereof, further optionally Expi293F™ cell; (ii) a CHO (Chinese Hamster Ovary) cell; (iii) an immune cell or (iv) a hybridoma.

In some embodiments, a population of cells according to the present disclosure may comprise two or more of any of the host cells described herein.

In another aspect, the present disclosure provides pharmaceutical compositions containing any of the foregoing. A pharmaceutical composition according to the present disclosure may comprise: (A) any of the multispecific antibodies and antibody fragments described herein; (B) any of the nucleic acids or any of the combinations of nucleic acids described herein; (C) any of the vectors or any of the combinations of vectors described herein; and/or (D) any of the host cells or the populations of cells described herein; and (II) a pharmaceutically acceptable carrier and/or excipient.

In another aspect, the present disclosure provides in vivo methods, such as methods of treating a subject, methods of treating or preventing a disease, disorder, or a condition in a subject, and methods of inducing, promoting, stimulating, enhancing, and/or supporting a Wnt signaling in a target cell of a subject.

In some aspects a method of treating a subject in need of such treatment according to the present disclosure may comprise administering to the subject an effective amount of: (A) any of the multispecific antibodies or antibody fragments described herein; (B) any of the nucleic acids or combinations of nucleic acids described herein; (C) any of the vectors or combinations of vectors described herein; (D) any of the host cells or the populations of cells described herein; and/or (E) any of the pharmaceutical compositions described herein.

In some embodiments, the subject comprises or has a risk of developing a disease, disorder, or a condition, e.g., because of heredity or an underlying disease or comorbidity.

In some embodiments, the invention provides methods of treating or preventing a disease, disorder, or a condition in a subject in need of such treatment which may comprise administering an effective amount of: (A) any of the multispecific antibodies or antibody fragments described herein; (B) any of the nucleic acids or combination of nucleic acids described herein; (C) any of the vectors or the combinations of vectors described herein; (D) any of the host cells or the populations of cells described herein; and/or (E) any of the pharmaceutical compositions described herein.

In some aspects a method of inducing, promoting, stimulating, enhancing, and/or supporting Wnt signaling in a target cell of a subject according to the present disclosure may comprise administering to the subject an effective amount of: (A) any of the multispecific antibodies or antibody fragments described herein; (B) any of the nucleic acids or combinations of nucleic acids described herein; (C) any of the vectors or combinations of vectors described herein; (D) any of the host cells or the populations of cells described herein; and/or (E) any of the pharmaceutical compositions described herein.

In some embodiments, the Wnt signaling is or comprises Wnt/p-catenin signaling.

In some embodiments, the method promotes, stimulates, enhances, and/or supports recruitment of multiple receptor complexes each comprising (i) Fzd4 and (ii) LRP5 or LRP6.

In any of the in vivo methods disclosed herein, the subject typically is a mammal, optionally a human, a non-human primate, a monkey, a horse, a cow, sheep, a goat, a pig, a dog, a cat, a rabbit, a rodent, a hamster, a rat, or a mouse.

In any of the in vivo methods disclosed herein, the subject may comprise a non-mammalian vertebrate, optionally a bird, fish, an amphibian, or a reptile.

In any of the in vivo methods disclosed herein, in some cases, the subject comprises or has a risk of developing a disease, disorder, or a condition, e.g., because of heredity or another underlying disease or comorbidity.

In any of the in vivo methods disclosed herein, the method may further comprise administering to the subject an additional agent, optionally an adjuvant or a therapeutic agent.

In any of the in vivo methods disclosed herein, the disease, disorder, or condition may comprise a retinopathy, optionally a retinal vascular disease (optionally caused by inhibition of vascular development or excessive angiogenesis) and/or optionally selected from the group consisting of exudative vitreoretinopathy, familiar exudative vitreoretinopathy (FEVR), retinopathy of prematurity, Norrie disease, diabetic retinopathy (DR), diabetic macular edema, diabetic macular ischemia, age-related macular degeneration (AMD) (including wet AMD and dry AMD), retinopathy of prematurity (ROP), osteoporosis-pseudoglioma syndrome (OPPG), retinal vein occlusion, and Coats disease.

In any of the in vivo methods disclosed herein, the disease, disorder, or condition may comprise a vascular disorder, optionally vascular malformation or vascular insufficiency, further optionally associated with ischemia-induced neovascularization.

In any of the in vivo methods disclosed herein, the disease, disorder, or condition may comprise a bone disease or bone injury, optionally selected from the group consisting of osteoporosis (including osteoporosis pseudoglioma (OPPG) syndrome and juvenile primary osteoporosis), bone mineral density variability, and cancer-associated osteolysis.

In any of the in vivo methods disclosed herein, the disease, disorder, or condition may comprise a muscle wasting disease, optionally selected from the group consisting of sarcopenia, cachexia, and muscular dystrophies.

In any of the in vivo methods disclosed herein, the disease, disorder, or condition may comprise a neural or neurodegenerative disease, optionally selected from the group consisting of stroke, traumatic brain injury, epilepsy, Alzheimer's disease, Parkinson's disease, Huntington's disease, amyotrophic lateral sclerosis, Friedreich ataxia, Lewy body disease, spinal muscular atrophy, motor neuron disease, multiple sclerosis, Batten disease, and Creutzfeldt-Jakob disease.

In any of the in vivo methods disclosed herein, administration of the active agent may be to one or more of the subject's eyes, optionally intravitreally or via ocular drops.

In any of the in vivo methods disclosed herein, administration of the active agent may be effected locally, optionally to the eye, ear, nose (optionally intranasally), skin (optionally transdermally or epicutaneously), mucosa, skin, or vagina, or by inhalation.

In any of the in vivo methods disclosed herein, administration of the active agent may be effected parenterally administering, optionally by injection (optionally intravenous, intramuscular, subcutaneous, intradermal, intrathecal, intra-arterial, intraarticular, intraosseous, or intraperitoneal administration) or by inhalation.

In any of the in vivo methods disclosed herein, administration of the active agent may be effected enterally, optionally orally, sublingually, buccally, or rectally.

In any of the in vivo methods disclosed herein, administration of the active agent may comprise administering any of the multispecific antibodies and antibody fragments described herein to one or more of the subject's eyes at about 0.1-100000 ng per eye, about 1-100000 ng per eye, about 1-10000 ng per eye, about 10-10000 ng per eye, about 1-100 ng per eye, about 200-300 ng per eye, about 300-400 ng per eye, about 400-500 ng per eye, about 500-600 ng per eye, about 600-700 ng per eye, about 700-800 ng per eye, about 800-900 ng per eye, about 900-1000 ng per eye, about 1000-2000 ng per eye, about 2000-3000 ng per eye, about 3000-4000 ng per eye, about 4000-5000 ng per eye, about 5000-6000 ng per eye, about 6000-7000 ng per eye, about 7000-8000 ng per eye, about 8000-9000 ng per eye, about 9000-10000 ng per eye, about 10000-20000 ng per eye, about 20000-30000 ng per eye, about 30000-40000 ng per eye, about 40000-50000 ng per eye, about 50000-60000 ng per eye, about 60000-70000 ng per eye, about 70000-80000 ng per eye, about 80000-90000 ng per eye, or about 90000-100000 ng per eye.

In another aspect, the present disclosure provides methods of inducing, promoting, stimulating, enhancing, and/or modulating Wnt signaling in a target cell.

In some aspects such a method of inducing, promoting, stimulating, enhancing, and/or supporting Wnt signaling in a target cell may comprise contacting the target cell with an effective amount of: (A) any of the multispecific antibodies or antibody fragments described herein; (B) any of the nucleic acids or combinations of nucleic acids described herein; (C) any of the vectors or combinations of vectors described herein; (D) any of the host cells or the populations of cells described herein; and/or (E) any of the pharmaceutical compositions described herein.

In some embodiments, the contacting occurs in vitro.

In some embodiments, the contacting occurs ex vivo.

In some embodiments, the contacting occurs in vivo.

In some embodiments, the method comprises (i) preparing a cell and/or tissue for implantation, (ii) differentiation of a stem cell, and/or (iii) preparation of an organoid.

In some embodiments, the method elicits or promotes bone formation, vascular formation, and/or neural formation and/or differentiation.

In a further aspect, the present disclosure provides methods of manufacturing any of the multispecific antibodies and antibody fragments described herein.

In some aspects a method of manufacturing any of the multispecific antibodies and antibody fragments described herein may comprise: (a) culturing cells comprising any of the nucleic acids or combinations of nucleic acids encoding the multispecific antibody or antibody fragment under conditions that permit the expression of said multispecific antibody or antibody fragment; and (b) harvesting and purifying the resultant multispecific antibody or antibody fragment from the cell culture from (a).

In a further aspect, the present disclosure provides methods of manufacturing any of the host cells or any of the populations of cells described herein.

In some aspects a method of manufacturing any of the host cells or the populations of such cells described herein may comprise introducing any of the nucleic acids or the combinations of nucleic acids described herein and/or the vectors or combinations of vectors described herein into one or more cells.

In some embodiments, the introducing occurs in vitro.

In some embodiments, the introducing occurs ex vivo.

In some embodiments, the introducing occurs in vivo.

Any of the multispecific antibodies or antibody fragments described herein, any of the nucleic acids or combinations of nucleic acids described herein, any of the vectors or combinations of vectors described herein, any of the host cells or populations of cells described herein, and/or pharmaceutical compositions described herein may be for use in medicine.

Any of the multispecific antibodies or antibody fragments described herein, any of the nucleic acids or combinations of nucleic acids described herein, any of the vectors or combinations of vectors described herein, any of the host cells or populations of cells described herein, and/or pharmaceutical compositions described herein may be for use in treating a disease, disorder, or condition, e.g., one involving Wnt signaling.

In some embodiments, the disease, disorder, or condition comprises one or more of any of the diseases described herein.

The present disclosure further encompasses use of any of the multispecific antibodies or antibody fragments described herein, any of the nucleic acids or combinations of nucleic acids described herein, any of the vectors or combinations of vectors described herein, any of the host cells or populations of cells described herein, and/or pharmaceutical compositions described herein for the manufacture of a medicament for treatment of a disease, disorder, or condition.

In some embodiments, the disease, disorder, or condition comprises one or more of any of the diseases described herein.

The present invention is based, in part, upon the use of Wnt signaling agonists and antagonists to regulate aberrant vascular formation in retinopathy indications.

In some embodiments the present invention provides methods of treating a subject suffering from a retinopathy comprising administering the subject, an engineered Wnt signaling agonist. In further embodiments, the engineered Wnt agonist comprises binding compositions that bind to one or more Fzd receptors and binding compositions that bind to one or more LRP receptors.

In one aspect, the disclosure provides an engineered Wnt signaling agonist, wherein the Wnt signaling agonist comprises: two light chains, each comprising a polypeptide sequence having at least 95% identity to SEQ ID NO: 1, and two heavy chains, each comprising a polypeptide sequence having at least 95% identity to SEQ ID NO: 2, wherein the two heavy chains are bound to each other, and each of the two light chains is bound to a different heavy chain of the Wnt signaling agonist. In certain embodiments, each of the two light chains comprises a VHH domain comprising one or more of the following amino acid residue modifications: N29Q and/or hVHH3-H4 DDD (amino acids 101-103) substituted with DED. In certain embodiments, each of the two heavy chains comprises one or more of the following amino acid residues: a CDR1 S31, a CDR2 E62, and/or CDR3 D108.

In another aspect, the disclosure provides a nucleic acid comprising a sequence encoding one or more light chain and/or one or more heavy chain of the engineered Wnt signaling agonist disclosed herein, e.g., hp4SD1-03, or a fragment or variant thereof. In certain embodiments, the nucleic acid is an mRNA, e.g., a modified mRNA.

In a related aspect, the disclosure provides a vector comprising a nucleic acid disclosed herein, e.g., a nucleic acid comprising a sequence encoding one or more light chain and/or one or more heavy chain of hp4SD1-03, or a fragment or variant thereof. In particular embodiments, the vector is an expression vector comprising a promoter operatively linked to the nucleic acid, such as, e.g., a viral vector.

In a related embodiment, the disclosure provides a host cell comprising a vector or nucleic acid disclosed herein, e.g., a vector or nucleic acid comprising a sequence encoding one or more light chain and/or one or more heavy chain of hp4SD1-03, or a fragment or variant thereof. In certain embodiments, the host cell is a recombinant cell. In certain embodiments, the host cells is a mammalian cell or an insect cell.

In another aspect, the disclosure provides a pharmaceutical composition comprising a pharmaceutically acceptable diluent, excipient or carrier, and:
a) an engineered Wnt signaling agonist disclosed herein;
b) a nucleic acid disclosed herein; and/or
c) a vector disclosed herein.

In certain embodiments, the pharmaceutical composition comprises hp4SD1-03, or a fragment or variant thereof, or the nucleic acid encodes one or more light chain and/or one or more heavy chain of hp4SD1-03, or a fragment or variant thereof, or the vector comprises a nucleic acid comprising a sequence encoding one or more light chain and/or one or more heavy chain of hp4SD1-03, or a fragment or variant thereof.

In a further aspect, the disclosure provides a method of treating a retinopathy in a subject, comprising administering to the subject:
a) an engineered Wnt signaling agonist disclosed herein;
b) a nucleic acid disclosed herein;
c) a vector disclosed herein, and/or
d) a pharmaceutical composition disclosed herein.

In certain embodiments, the engineered Wnt signaling agonist is hp4SD1-03, or a fragment or variant thereof, or the pharmaceutical composition comprises hp4SD1-03, or a fragment or variant thereof, or the nucleic acid encodes one or more light chains and/or one or more heavy chains of hp4SD1-03, or a fragment or variant thereof, or the vector comprises a nucleic acid comprising a sequence encoding one or more light chains and/or one or more heavy chains of hp4SD1-03, or a fragment or variant thereof.

In certain embodiments of the disclosed treatment methods, the treated retinopathy is a retinal vascular disease. In certain embodiments, the retinal vascular disease is caused by inhibition of vascular development. In certain embodiments, the retinopathy is caused by excessive angiogenesis. In certain embodiments, the retinopathy is an early-stage retinopathy or a late-stage retinopathy. In some embodiments, the retinal vascular disease is selected from the group consisting of: familiar exudative vitreoretinopathy (FEVR), exudative vitreoretinopathy, Norrie disease, diabetic retinopathy (DR), diabetic macular edema, diabetic macular ischemia, age-related macular degeneration (AMD) (including wet AMD and dry AMD), retinopathy of prematurity (ROP), osteoporosis-pseudoglioma syndrome (OPPG), retinal vein occlusion, and Coats disease. The engineered Wnt signaling agonist may be administered via various routes of administration, including but not limited to systemically or locally, such as intravenously, subcutaneously, orally, topically, nasally, intravitreally, suprachoroidally, or subretinally. In certain embodiments, the engineered Wnt signaling agonist is administered to one or more of the subject's eyes, e.g., intravitreally.

### DESCRIPTION OF THE DRAWINGS

FIG. 1 provides the amino acid sequences of a tetravalent, bi-specific Wnt signaling agonist, 4SD1-03_LALAPG, which comprises two heavy chains and two light chains of an anti-Fzd4 antibody, 4SD1, wherein an anti-LRP5/6 VHH domain is fused to the N-terminus of each anti-Fzd4 antibody light chain via the linker sequence, GGSGS (SEQ ID NO: 102), and its associated IgG-like antibody structure wherein the two heavy chains are joined via disulfide bonds, and each light chain is joined to a heavy chain via a disulfide bond. The VHH domain is shown in italics; the linker sequence is GGSGS (SEQ ID NO: 102); the CDRs are underlined and in bold; the LALAPG mutations in the Fc domain of the heavy chain are shown in gray; and the Fc domain is highlighted in gray; the anti-Fzd4 VL and VH domains each comprise three CDRs and are also highlighted (anti-Fzd4 CDRs defined based on Kabat numbering).

FIG. 2A shows various amino acid modification made to the VHH3 domain of 4SD1-03_LALAPG to humanize the VHH camelid sequences. VHH represents the parental camelid sequence; 3-66*01 represents the closest human sequence identified in human genome databases; and hVHH3-H1 to hVHH3-H4 include the indicated amino acid substitutions. Amino acid residue numbers based on the actual residue number within the VH (“seq”) and based on Martin numbering “AbM” are shown above the sequences.

FIG. 2B shows the SEC (size exclusion chromatography) profiles of various humanized forms of VHH3 shown in FIG. 2A following Protein A purification. The x-axis shows elution volume in ml; the y-axis shows milli absorbance units (mAU). hVHH3-H4 showed the best expression and highest purity, as indicated by the single peak and high mAU value. Arrows indicate expected retention volume.

FIG. 3 shows the SEC profiles of VHHs associated with the modification of a potential deamidation motif, NS, in hVHH3-H4. The x-axis shows elution volume in ml; the y-axis shows milli absorbance units (mAU). The graphs show that N29 in hVHH3-H4 can be replaced for example by Q without affecting purity.

FIG. 4 provides SEC profiles of VHHs associated with the modification of a potential isomerization motif, DDD, in hVHH3-H4. The x-axis shows elution volume in ml; the y-axis shows milli absorbance units (mAU). The chromatograms show the effects on purity following Protein A elution from the various amino acid substitutions made to the DDD motif.

FIG. 5 shows modification of potential oxidation sensitive residues, M51 and M57, in VHH3. The table to the left shows various amino acid substitutions made to the M51 or M57 residue and shows that substitution of M51 or M57 with any tested amino acid was not tolerated and resulted in very poor yield. The chromatograms to the right show examples of the resulting purity of different M51 and M57 mutations. The x-axis shows elution volume in ml; the y-axis shows milli absorbance units (mAU).

FIG. 6 provides a diagram of modifications to the VHH3 domain of 4SD1-03_LALAPG (VHH3 was humanized and N29Q and D102E substitutions were incorporated) and modifications to the VH domain of 4SD1-03_LALAPG (N31S and DF62E substitutions were incorporated and the framework region sequence was altered to further simulate to a human germline-encoded sequence) made to derive hp4SD1-03.

FIG. 7 shows various amino acid substitutions made to the heavy chain of 4SD1-03_LALAPG to modify potentially liable amino acids; N31 in CDR1 was modified to Q, S, or A; D62 in CDR2 was modified to E, S, or A; and D108 in CDR3 was modified to E, S, or A. The graphs show that mutation of CDR1 N31 and CDR2 D62 have similar or higher Wnt signaling activity as compared to the parental molecule. The N31S mutation resulted in a five-fold increase in potency. However, CDR3 D108 could not be replaced without losing potency. Additional mutations were tested but resulted in reduced Wnt signaling activity, as shown in the graph to the right. The combination of N31S, D62E and D108 was tested by mass spectrometry, and showed no isomerization upon accelerated stress conditions.

FIG. 8 shows an alignment between the sequences of the VH region of 4SD1 and its closest human germline IGHV3-23*04 a. Based on this alignment a framework mutant of 4SD1-03_LALAPG containing changes in the FR sequences to simulate to human germline-encoded sequences (as shown in the table on upper right) was constructed. Framework-mutated hp4SD1-03 retained good expression and purification profile as shown in the SEC (size exclusion chromatography) profile. The framework-mutated hp4SD1-03 showed five-fold higher potency in the cell-based STF assay than the parental molecule 4SD1-03_LALAPG. Further, hp4SD1-03 showed no isomerization liability when analyzed by mass spectrometry after thermal stress at 42° C. for 2-weeks at pH 5.5.

FIG. 9 provides the amino acid sequences of the resulting tetravalent, bi-specific Wnt signaling agonist, hp4SD1-03, comprising the modifications noted above to the parental 4SD1-03_LALAPG sequences. This construct has the same general structure as the parental construct. The VHH domain is shown in italics; the linker sequence is GGGGS (SEQ ID NO: 101); the CDRs are underlined and in bold; the LALAPG mutations in the Fc domain of the heavy chain are shown in gray; and the Fc domain is highlighted in gray; the anti-Fzd4 VL and VH domains each comprise three CDRs and are also highlighted (anti-Fzd4 CDRs defined based on Kabat numbering).

FIG. 10A is a graph showing the Wnt signaling activity of the modified construct, hp4SD1-03, as compared to the parental construct, 4SD1-03_LALAPG, both constructs comprising the LALAPG Fc mutant. The EC₅₀ₛ shown in the table indicate hp4SD1-03 having approximately ten-fold greater Wnt signaling activity.

FIG. 10B shows an exemplary result of Wnt/β-catenin signaling activity assessment based on Axin2 expression as described in Example 5, comparing hp4SD1-03 and 4SD1-03_LALAPG, both comprising the LALAPG Fc mutant with Norrin as a control. As shown in the table, hp4SD1-03 showed an approximately 11.5-fold reduction in EC₅₀.

FIG. 11 shows the effect of treatment with the modified construct, hp4SD1-03, as compared to the parental construct, 4SD1-03_LALAPG in a mouse model of retinal injury. The timeline at the top outlines the induction of retinal damage via 75% oxygen, followed by intravitreal injection of either construct, and examination of the retinal tissue by staining for avascular (AV) and tuft areas. The retinal flatmount images stained with isolectin B4 (IB4) below show non-injured retinal, injured retina (negative control), and retina from animals treated with Eylea® (positive control), the modified construct, hp4SD1-03, or the parental construct, 4SD1-03_LALAPG with dosage as shown in the table to the right. The graphs to the right show the percentage of avascular tissue or tuft area over total area resulting from each treatment, and demonstrate a better outcome with hp4SD1-03 as compared to 4SD1-03_LALAPG. For each graph, from left to right, the bars correspond to: no treatment; Eylea®, 4SD1-03_LALAPG, and hp4SD1-03. * indicates p<0.05, ** indicates p<0.01, *** indicates p<0.001, **** indicates p<0.0001 in ANOVA test.

FIG. 12 shows the effect of treatment with the indicated amounts of the modified construct, hp4SD1-03, in a VEGF-induced retinal vascular leakage rabbit model of retinal injury. Dosages are shown in the table, and VEGF-induced retinal leakage scores are shown in the accompanying graphs. Treatment with 10 ug/eye or 2 ug/eye of hp4SD1-03 resulted in an about 75% reduction in leakage, and treatment with 0.4 ug/eye or 0.08 ug/eye of hp4SD1-03 also significantly reduced vascular leakage. * indicates p<0.05, and ** indicates p<0.01 in ANOVA test. Retinal vascular leakage comparison between the treatment of vehicle and hp4SD1-03 at Day 3.

FIG. 13 provides fluorescein angiography images of rabbit retinal tissue treated as indicated. Efficacy was observed in all tested animals.

FIG. 14 shows the activity of various mutants with amino acid substitutions within the hVHH3-H4 sequence to replace the D at position 103 or the DED at positions 101-103. The upper graphs and lower table show the STF activity of the various mutants as compared to the activity of hp4SD1-03, which contains DED at positions 101-103. The chromatograms show the purity following Protein A purification associated with the modification of DED in hVHH3-H4. The x-axis shows elution volume in ml; the y-axis shows milli absorbance units (mAU). These data show both D101 and D103 of hp4SD1-03 may be replaced without reducing the molecule's activity.

FIGS. 15A-15C show the effect of treatment with high concentrations of hp4SD1-03 in the mouse OIR model. FIG. 15A depicts the OIR study design; FIG. 15B shows staining of the retinal avascular (AV) and neovascular tuft (NV) areas. The retinal flatmount images stained with isolectin B4 (IB4) show untreated and treated with 40 nM, 400 nM, and 4000 nM of hp4SD1-03. FIG. 15C provides quantitative graphs of the imaging results in FIG. 15B. * indicates p<0.05, ** indicates p<0.01, **** indicates p<0.0001 in ANOVA test.

FIGS. 16A-16B show the increased expression of selected genes and proteins upon treatment with parental 4SD1-03_LALAPG. FIG. 16A is mRNA expression of LEF1 and MFSD2A and FIG. 16B is protein expression of ZO-1 and CLDN5, two tight junction proteins.

FIG. 16C provides an exemplary western blot analysis result from Example 7. Treatment of human retinal microvascular endothelial cells (HRMEC) with hp4SD1-03 increased phosphorylation of DVL3 (increased ratio of phosphorylated DVL3 to unphosphorylated DVL3), a key cytoplasmic molecule in Wnt signaling.

FIGS. 17A-17C provide exemplary results from assessment of binding kinetics as described in Example 8. Monovalent binding of human Fzd4 CRD to 4SD1-03_LALAPG or hp4SD1-03 was measured by BLI. FIG. 17A provides an exemplary binding sensorgram of human Fzd4 CRD to 4SD1-03_LALAPG. Red lines represent the global fits generated by fitting the real-time binding sensorgrams (blue lines) using a 1:1 Langmuir binding model. FIG. 17B provides an exemplary binding sensorgram of human Fzd4 CRD to hp4SD1-03. Red lines represent the global fits generated by fitting the real-time binding sensorgrams (green lines) using a 1:1 Langmuir binding model. FIG. 17C provides a table showing the KD, kₒₙ, and k_{dis} (also called k_{off}) values observed in binding of human Fzd4 CRD to 4SD1-03_LALAPG or hp4SD1-03 at the indicated human Fzd4 CRD concentrations.

FIGS. 18A-18C provide exemplary results from assessment of binding kinetics as described in Example 8. Bind of 4SD1-03_LALAPG or hp4SD1-03 to human Fzd4 CRD was measured by SPR. FIG. 18A provides an exemplary binding sensorgram of human Fzd4 CRD to 4SD1-03_LALAPG. FIG. 18B provides an exemplary binding sensorgram of human Fzd4 CRD to hp4SD1-03. FIG. 18C provides a table showing the K_{D}, kₐ (also called kₒₙ), and k_{d} (also called k_{off}) values observed in binding of 4SD1-03_LALAPG or hp4SD1-03 to human Fzd4 CRD. Indicated are the average of the values observed at four different capture concentrations.

FIGS. 19A-19B provide the amino acid sequences of the heavy and light chains of hp4SD1-03 AAQ and hp4SD1-03 AAA. The additional amino acid substitutions introduced relative to hp4SD1-03 are highlighted.

FIG. 20 provides exemplary SEC chromatograms obtained in Example 9 after Protein A affinity resin purification of hp4SD1-03, hp4SD1-03 AAQ, and hp4SD1-03 AAA produced in transiently transfected Expi293F™ cells. Highlighted fraction was analyzed on SDS-PAGE gel.

FIG. 21 provides exemplary results from melting temperature evaluation as described in Example 9. The three top graphs are exemplary melting curves for hp4SD1-03, hp4SD1-03 AAQ, and hp4SD1-03 AAA, each analyzed in duplicate. The table (top right) provides the average Tm1 values from each sample ran in duplicate. The bottom graph shows an overlay of Tagg values from each sample analyzed in duplicate.

FIG. 22 provides exemplary results from the Wnt signaling assay in Example 9. The graph (left) shows exemplary dose-response curves for hp4SD1-03, hp4SD1-03 AAQ, and hp4SD1-03 AAA obtained using a luciferase-reporter system in bEnd.3 cells natively expressing Fzd4 and LRP5. The table (right) shows EC50 values from the fit curve.

FIG. 23 provides exemplary results from assessment of binding to the neonatal Fc-receptor FcRn in Example 9. Graphs are exemplary sensorgrams for hp4SD1-03, hp4SD1-03-AAQ, and hp4SD1-03-AAA. hp4SD1-03 shows binding to FcRn as expected, and in contrast no binding was observed with the hp4SD1-03-AAQ or hp4SD1-03-AAA mutants.

FIGS. 24-34 provide exemplary and non-limiting embodiments of various multispecific antibody structures which may comprise any of the variable domain sequences of anti-Fzd4 and anti-LRP5/6 antibodies disclosed herein. In FIGS. 24-34, the following applies unless otherwise indicated: (1) Each domain is presented as a rectangle with the text therein showing the domain name (e.g., CH3, VH1, etc); (2) a set of multiple domains connected with each other represents a polypeptide (e.g., a heavy chain polypeptide, a light chain polypeptide, etc); (3) the direction of domains within a polypeptide is according to the direction of the text showing domain names, from the N-terminus to the C-terminus; (4) a linker or a hinge may be used between domains as necessary and a disulfide bond(s) may exist between polypeptides (and/or within a domain), perhaps to allow correct formation of the antigen-binding site(s), even when the figures do not explicitly show a linker, a hinge, or a disulfide bond; (5) a line extending from a VH2 to another domain means a linker may or may not be present between the VH2 and the other domain; (6) a CH1, a CH2, a CH3 and/or CL domain(s) shown in figures may be omitted whenever possible and, when appropriate, may be replaced with a hinge or a linker; (7) CH1, CH2, and CH3 domains may individually be of any (heavy chain) isotype; (8) when more than one CH1 domains are present in a structure, the CH1 domain sequences may or may not be identical to each other or may or may not be of the same isotype, when more than one CH2 domains are present in a structure, the CH2 domain sequences may or may not be identical to each other or may or may not be of the same isotype, and when more than one CH3 domains are present in a structure, the CH3 domain sequences may or may not be identical to each other or may or may not be of the same isotype; (9) light chain constant (CL) domain may be a kappa CL domain or a lambda CL domain; (10) when more than one CL domains are present in a structure, all CL domains may be kappa CLs or all CL domains may be lambda CLs, or alternatively one CL may be a kappa CL and another CL may be a lambda CL domain, and the CL domain sequences may or may not be identical to each other; (11) when both kappa and lambda CL domains are present, the CH1 domains paired to the CL domains may, in some instances, be variant CH1 domains, one of which may be a variant CH1 that preferentially binds to a kappa CL and another CH1 domain may be a variant CH1 that preferentially binds to a lambda CL (having kappa and lambda CLs and kappa-preferring CH1 and lambda-preferring CH1 in a molecule potentially allows for efficient manufacturing); (12) in general VH1 and VL1 (if VL1 is present) form an antigen-binding region for a first antigen (e.g., Fzd4), and VH2 forms an antigen-binding region for a second antigen (e.g., LRP5 and/or LRP6); (13) in a VH1-VL1 pair, the VL1 may be omitted even if VL1 is shown in figures if the VH1 alone gives sufficient specificity to the first antigen (i.e., nanobody); and (14) even when VL2 is not explicitly shown in figures, the antigen-binding region comprising VH2 may further comprise VL2, as long as the VH2-VL2 pair retains binding to the second antigen.

FIG. 24 provides some exemplary and non-limiting embodiments of various multi-specific antibody structures with which any of the variable domain sequences of anti-Fzd4 and anti-LRP5/6 antibodies disclosed herein may be used. The structure on the top left (boxed) is an exemplary structure possessed by some of the specific bispecific antibodies disclosed herein (e.g., hp4SD1-03, hp4SD1-03 AAQ, hp4SD1-03 AAA). The boxed structure has; two first polypeptides (which may also be referred to as heavy chains herein) each comprising a VH1, a CH1, a CH2, and a CH3 from the N-terminus to the C-terminus; and two second polypeptides (which may also be referred to as light chains herein) each comprising a VH2, an optional linker, a VL1, and a CL from the N-terminus to the C-terminus. Each pair of VH1 and VL1 forms an antigen-binding region specific for a first antigen, and each VH2 forms an antigen-binding region specific for a second antigen. Although hinges or disulfide bonds are not explicitly shown in the boxed structure, the boxed structure may, for example, comprise a hinge between CH1 and CH2 within a single polypeptide and a disulfide bond(s) may be present between the hinges and between CH1 and CL domains as shown in the structure on the bottom left (disulfide bonds shown as dotted lines). Alternatively, the boxed antibody may, for example, comprise a hinge between CH1 and CH2 within a single polypeptide and a disulfide bond(s) may be present between hinges and between CL and a hinge as shown in the structure on the bottom right (disulfide bonds shown as dotted lines). Hinges and disulfide bonds, such as those shown in the bottom left and right structures may be present, even if not explicitly shown, in any structures shown in figures and described herein.

FIG. 25 provides exemplary variations of the antibody structures shown in FIG. 24. In some variants of the boxed antibody, a VH2 may be relocated to a different position, for example N-terminal to the VH1 in a first polypeptide (top right), C-terminal to the CL in a second polypeptide (bottom left), or C-terminal to the CH3 in a first polypeptide (bottom right). Equivalent variations (relocating a VH2) depicted in FIG. 25 may be further applied to any structures shown in FIGS. 24-34 or variations thereof as appropriate, even if not explicitly shown or stated.

FIG. 26 provides further exemplary variations of the antibody structures shown in FIG. 24. In some variants of the boxed antibody, a VH2 may be further included, for example N-terminal to another VH2 in a second polypeptide (top left), N-terminal to the VH1 in a first polypeptide (top right), C-terminal to the CL in a second polypeptide (bottom left), or C-terminal to the CH3 in a first polypeptide (bottom right). Equivalent variations (adding a VH2 to the N- or C-terminus of a first and/or second polypeptide) depicted in FIG. 26 may be further applied to any structures shown in FIGS. 24-34 or variations thereof as appropriate, even if not explicitly shown or stated.

FIG. 27 provides some exemplary variations of the antibody structures shown in FIG. 25. Specifically, the positions of VH1 and VL1 are swapped relative to the antibody structures shown in FIG. 25. Equivalent variations (Swapping VH1 and VL1) depicted in FIG. 27 may be further applied to any structures shown in FIGS. 24-34 or variations thereof as appropriate, even if not explicitly shown.

FIG. 28 provides some exemplary variations of the antibody structures shown in FIG. 25. Specifically, the positions of CH1 and CL are swapped relative to the antibody structures shown in FIG. 25. Equivalent variations (Swapping CH1 and CL) depicted in FIG. 28 may be further applied to any structures shown in FIGS. 24-34 or variations thereof as appropriate, even if not explicitly shown.

FIG. 29 provides some exemplary variations of the antibody structures shown in FIG. 25. Specifically, CH3 domains are absent relative to the antibody structures shown in FIG. 25. Equivalent variations (removing CH3) depicted in FIG. 29 may be further applied to any structures shown in FIGS. 24-34 or variations thereof as appropriate, even if not explicitly shown.

FIG. 30 provides some exemplary variations of the antibody structures shown in FIG. 25. Specifically, CH2 domains are absent relative to the antibody structures shown in FIG. 25. Equivalent variations (removing CH2) depicted in FIG. 30 may be further applied to any structures shown in FIGS. 24-34 or variations thereof as appropriate, even if not explicitly shown.

FIG. 31 provides some exemplary variations of the antibody structures shown in FIG. 25. Specifically, CH2 and CH3 domains are absent relative to the antibody structures shown in FIG. 25. In some cases, hinges and disulfide bonds may be present to allow dimerization of the two first polypeptides, as shown (disulfide bonds shown as dotted lines). Equivalent variations (removing CH2 and CH3) depicted in FIG. 31 may be further applied to any structures shown in FIGS. 24-34 or variations thereof as appropriate, even if not explicitly shown.

FIG. 32A provides some exemplary variations of the antibody structures shown in FIG. 25. Specifically, the structures shown are half the antibody structures shown in FIG. 25, i.e., comprising one first polypeptide and one second polypeptide, and contains one first antigen-binding region and one second antigen-binding region. Equivalent variations (half structure or half antibody) depicted in FIG. 32A may be further applied to any structures shown in FIGS. 24-34 or variations thereof as appropriate, even if not explicitly shown.

FIG. 32B provides further exemplary variations of half antibody structures shown in FIG. 32A, which do not comprise CH2 and CH3 but comprises CH1. The structures may comprise a hinge or a partial hinge C-terminal to the CH1 (four structures on the top) and the VH2 may be located N-terminal to the VL1 (top left), N-terminal to the VH1 (top second from left), C-terminal to the CL (top second from right), or C-terminal to the hinge or partial hinge (top right). Alternatively, the structures may not comprise a hinge (four structures on the bottom) and the VH2 may be located N-terminal to the VL1 (top left), N-terminal to the VH1 (top second from left), C-terminal to the CL (top second from right), or C-terminal to the CH1 (top right).

FIG. 33 provides further exemplary and non-limiting embodiments of various multispecific antibody structures which may comprise any of the variable domain sequences of anti-Fzd4 and anti-LRP5/6 antibodies disclosed herein. In the structures shown on the top, VH1 and VL1 form a scFv and are further fused to VH2. The scFv may comprise a VH1, an optional linker, and a VL1 or a VL1, an optional linker, and a VH1 in the direction from the N-terminus to the C-terminus and a VH2 may be place N-terminal to the scFv (top left and top second from left) or C-terminal to the scFv (top right and top second from right). Such structures may be further linked or dimerized. For example, as shown in the structures on the bottom, the boxed structure may be fused to another boxed structure (bottom left) or to each of the other three variants on the top (bottom left, bottom right, and bottom second from right).

FIG. 34 provides further exemplary variations of the boxed structure of FIG. 24. Specifically, VH1 and VL1 form a scFv and are further fused to VH2. Therefore, antibody structures may comprise two first polypeptides, each comprising a VH2, a VL1, an optional linker, a VH1, CH1, CH2, and CH3 from the N-terminus to the C-terminus (top left, boxed). In further variants of the boxed structure, CH3 domains may be absent (middle left), CH2 domains may be absent (middle center), or CH2 and CH3 domains may be absent (middle right). Hinges and disulfide bonds may be present, for example as shown in the structures on the bottom (disulfide bonds shown as dotted lines).

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference. Nothing herein is to be construed as an admission that the present invention is not entitled to antedate such disclosure by virtue of prior invention.

### I. Definitions

Unless defined otherwise, all technical and scientific terms used herein have the same meaning as commonly understood by one of ordinary skill in the art to which this disclosure belongs.

It is to be understood that the terminology used in the description is for the purpose of describing the particular versions or embodiments only and is not intended to limit the scope of the present invention which will be limited only by the appended claims.

As used herein, the term “about,” when used in reference to a particular recited numerical value, means that the value may vary from the recited value by no more than 10%. For example, as used herein, the expression “about 100” includes 90 and 110 and all values in between (e.g., 91, 92, 93, 99, 99.1, 99.2, 99.3, 99.4, 100, 100.8, 100.9, 101, 106, 107, 108, 109, etc.).

“Activity” of a molecule may describe or refer to the binding of the molecule to a ligand or to a receptor, to catalytic activity, to the ability to induce, promote, enhance, and/or support multimerization (e.g., dimerization, trimerization, tetramerization, etc) of one or more target molecules (e.g., antigen), to the ability to induce, promote, enhance, and/or support signal transduction in a cell expressing one or more target molecules (e.g., antigen), to the ability to alter (e.g., stimulate or suppress) gene expression, to antigenic activity, to the modulation of activities of other molecules such as activities of a ligand specific to the one or more target molecules (e.g., antigen)), and the like. “Activity” of a molecule may also refer to activity in modulating or maintaining cell-to-cell interactions, e.g., adhesion, or activity in maintaining a structure of a cell, e.g., cell membranes or cytoskeleton, or therapeutic effects. “Activity” may also mean specific activity, e.g., [catalytic activity]/[mg protein], or [immunological activity]/[mg protein], or the like.

The terms “administering” or “introducing”, as used herein, refer to delivery of a compound or a composition according to the present invention to a cell, to cells, tissues and/or organs of a subject, or to a subject. Such administering or introducing may take place in vivo, in vitro or ex vivo.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length or intact monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and F(ab′)2, so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, Fab′-SH, and Fv fragments; diabodies; linear antibodies (e.g., Zapata et al., Protein Eng. 8(10): 1057-1062 (1995)); single-chain antibody molecules (e.g., scFv); half antibody; nanobody or VH only, or VL only; and multispecific antibodies formed from antibody fragments. The term “half molecule” or “half antibody” when referring to IgG, IgE, or IgD, which may also be referred to as “half IgG”, “half IgE”, or “half IgD”, respectively, refers to a set of one heavy chain and one light chain of the referenced antibody. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The terms “intact antibody,” “full length antibody,” and “whole antibody” or the like are used herein interchangeably and refer to an antibody having a structure substantially similar to a native antibody. In some instances, an antibody comprises heavy (H) and light (L) chains interconnected by disulfide bonds. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. For example, an intact IgG (or IgD or IgE) antibody comprises two immunoglobulin heavy chains and two immunoglobulin light chains. Therefore, in some instances, an antibody according to the present disclosure may comprise two pairs of heavy and light chains interconnected by disulfide bonds, or an antigen-binding fragment(s) thereof. Some intact antibody comprises multiple units each comprising two pairs of heavy and light chains interconnected by disulfide bonds. For example, an intact IgA comprises two units and an intact IgM comprises five units. Therefore, in other instances, an antibody according to the present disclosure may instead comprise multiple (e.g., two, three, four, five, and so on) units each comprising two pairs of heavy and light chains interconnected by disulfide bonds, or an antigen-binding fragment(s) thereof.

In an intact antibody, each heavy chain is comprised of: a heavy chain variable domain (VH); and a heavy chain constant region (CH), which is typically comprised of domains CH1, CH2 and CH3. Each light chain is comprised of: a light chain variable domain (VL); and a light chain constant domain (CL). Typically (with some exceptions such as nanobodies, camelid heavy chain antibodies, IgNARs, and the like), one VH and one VL may form an antigen-binding region. The VH and VL can be further subdivided into regions of hypervariability, termed complementarity determining regions (CDRs), interspersed with regions that are more conserved, termed framework regions (FRs). Each VH and VL polypeptide is composed of three CDRs and four FRs, arranged from amino-terminus to carboxy-terminus in the following order: FR1, CDR1, FR2, CDR2, FR3, CDR3, FR4. CDRs in a heavy chain are designated “CDRH1,” “CDRH2,” and “CDRH3,” respectively, and the CDRs in a light chain are designated “CDRL1,” “CDRL2,” and “CDRL3.” FRs in a heavy chain are designated “FRH1,” “FRH2,” “FR3,” and “FRH4,” respectively, and the FRs in a light chain are designated “FRL1,” “FRL2,” “FRL3,” and “FRL4.” In certain embodiments of the disclosure, the FRs of the antibody (or antigen-binding fragment thereof) may be identical to the human germline-encoded sequences (e.g., heavy chain FR sequences encoded by the VH1-03 germline; and/or light chain FR sequences encoded by the VK4-01 germline) or may be naturally or artificially modified. An amino acid consensus sequence may be defined based on a side-by-side analysis of two or more CDRs.

The phrase “heavy chain” or “HC” is used in its broadest sense and encompasses a polypeptide comprising at least one immunoglobulin heavy chain domain or part (i.e., at least one of VH, CH1, hinge, CH2, and CH3). Similarly, the phrase “light chain” or “LC” is used in its broadest sense and encompasses a polypeptide comprising at least one immunoglobulin light chain domain (i.e., at least one of VL and CL).

The numbering of amino acid residues in antibody variable and/or constant domains may be performed by any appropriate numbering schemes, methods, and definitions. For example, CDR sequences may be determined based on Kabat numbering or IMGT numbering, or alternatively Chothia numbering, Martin numbering (also known as AbM numbering), Gelfand numbering, Honneger's numbering (also known as AHo's numbering), or Chemical Computing Group (CCG) numbering for variable regions or may be determined structurally (see e.g., IgBlast (https://www.ncbi.nlm.nih.govfigblast/); Dondelinger et al., Front Immunol. 2018 Oct. 16; 92278; http://www.bioinf.org.uk/abs/info.html#cdrid; http://opig.stats.ox.ac.uk/webapps/newsabdab/sabpred/anarci/). For constant domains, EU numbering, IMGT numbering, or Kabat numbering may for example be used. Unless otherwise indicated, EU numbering is used for constant domains and regions.

According to IMGT (the international ImMunoGeneTics information system for immunoglobulins or antibodies, T cell receptors, MH, immunoglobulin superfamily IgSF and MhSF), the CH1 domain, the hinge region, the CH2 domain, and the CH3 domain correspond to the amino acid positions 118-215, 216-230, 231-340, and 341-446, respectively (EU numbering). The terms “CH1 domain”, “hinge”, “CH2 domain”, and “CH3” are used in a broad sense herein to encompass any naturally occurring, corresponding heavy chain constant domain and/or region allotypes and variants thereof, which may comprise fewer or more amino acids (e.g., a CH1 domain may comprise a portion of a hinge region) and/or amino acid modification(s).

An exemplary CH1 domain of a human IgG1 may comprise the amino acid sequence of SEQ ID NO: 21 or 22; an exemplary hinge of a human IgG1 may comprise the amino acid sequence of SEQ ID NO: 31; and a CH2 domain of a human IgG1 may comprise the amino acid sequences of SEQ ID NOS: 41. An exemplary CH3 domain of a human IgG1 may comprise the amino acid sequence of SEQ ID NO: 51, 52, 53, or 54, and the C-terminal K may be removed such CH3 sequences. Any variants of such exemplary sequences may be used in conjunction with CDRs and/or variable domain sequences described herein.

“Fc region” is a C-terminal region of an immunoglobulin heavy chain that contains at least a portion of the constant region, including native sequence Fc regions and variant Fc regions. A human IgG heavy chain Fc region can extend from Asp221, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The phrase “effector function” of an antibody refers to biological activities attributable to the Fc region of an antibody, which varies by antibody isotype. Exemplary effector functions include: complement (e.g., C1q) binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

There are two major light chain isotypes, kappa (κ) and lambda (λ), and the corresponding light chain constant domains are called kappa CL domain (CLκ) and lambda CL domain (CLλ), respectively.

According to IMGT, the CLκ domain is the amino acid positions 108-214 (EU numbering). An exemplary CLκ domain of a human IgG may comprise the amino acid sequence of SEQ ID NO: 11. According to IMGT, the CLX domain is the amino acid positions 107-215 (EU numbering). An exemplary CLX domain of a human IgG may comprise the amino acid sequence of SEQ ID NO: 12.

The terms “CLκ” and “CLλ” are used in a broad sense herein to encompass any naturally occurring, corresponding light chain constant domain and/or region allotypes and variants thereof, which may comprise fewer or more amino acids and/or amino acid modification(s).

Various standard sequences (corresponding to different allotypes) of the constant domains of human IgG1, IgG2, IgG3, and IgG4 are known in the field and may be found for example in Vidarsson et al., Front Immunol. 2014 Oct. 20; 5:520 and U.S. Pat. No. 9,150,663, the disclosures of which are hereby incorporated by reference herein in their entirety herein. Again these reference sequences are intended to be exemplary as Applicant intends for human IgG1, IgG2, IgG3, and IgG4 sequences to include any naturally occurring human IgG1, IgG2, IgG3, and IgG4 allotype.

An “antigen-binding region” refers to a portion of an antibody or antigen-binding fragment with specificity for an antigen. In some cases, an antigen-binding region may comprise a VH and a VL. In some cases, an antigen-binding region may comprise a VH and no VL, e.g., in case of a nanobody.

The term “antigen” refers to a molecule or a portion of a molecule capable of being bound by a selective binding agent, such as an antibody, and additionally capable of being used in an animal to produce antibodies capable of binding to an epitope of that antigen. In certain embodiments, a binding agent (e.g., a Wnt surrogate molecule or binding region thereof, or a Wnt antagonist) is said to specifically bind an antigen when it preferentially recognizes its target antigen in a complex mixture of proteins and/or macromolecules.

The phrase “different antigens” may refer to different and/or distinct proteins, polypeptides, or molecules; as well as different and/or distinct epitopes, which epitopes may be contained within one protein, one polypeptide, or one molecule. An “antigen-binding fragment” or “antigen-binding antibody fragment” refers to a portion of an intact antibody or to a combination of portions derived from one or more intact antibody that binds the antigen to which the intact antibody binds. The term “antigen-binding fragment” as used herein may refer to a polypeptide fragment that contains at least one CDR of an immunoglobulin heavy and/or light chain, or of a VHH/sdAb (single domain antibody) or Nanobody® (Nab), that binds to the antigen of interest, in particular to one or more Fzd receptors, or to LRP5 and/or LRP6. In this regard, an antigen-binding fragment of the herein described antibodies may comprise 1, 2, 3, 4, 5, or all 6 CDRs of a VH and VL from antibodies that bind one or more Fzd receptors or LRP5 and/or LRP6. An antigen-binding fragment of an antibody includes any naturally occurring, enzymatically obtainable, synthetic, or genetically engineered polypeptide or glycoprotein that specifically binds an antigen to form a complex.

As used herein, the terms “biological activity” and “biologically active” refer to the activity attributed to a particular biological element in a cell. For example, the “biological activity” of a Wnt agonist, or fragment or variant thereof refers to the ability to mimic or enhance Wnt signals. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g., a promoter, enhancer, Kozak sequence, and the like, refers to the ability of the regulatory element or functional fragment or variant thereof to regulate, i.e., promote, enhance, or activate the translation of, respectively, the expression of the gene to which it is operably linked.

The term “bifunctional antibody,” as used herein, refers to an antibody that comprises a first arm having a specificity for one antigenic site and a second arm having a specificity for a different antigenic site, i.e., the bifunctional antibodies have a dual specificity.

With regard to multispecific antibodies (e.g., bispecific, trispecific, tetraspecific, and so on), such antibodies comprise at least two different antigen binding regions which recognize and specifically bind to at least two different antigens or epitopes. The different epitopes may or may not be within the same antigen. A “bispecific antibody” is a type of multispecific antibody and comprises two different antigen binding regions which recognize and specifically bind to two different antigens or two epitopes. A bispecific antibody may target, for example, two different surface receptors on the same or different cells. “Bispecific antibody” is used herein to encompass a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences).

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g., poly-adenylation sequence, enhancer elements, other genes, linker domains, etc.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. (See, e.g., Pearson (1994) Methods Mol. Biol. 24: 307-331). Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine. Alternatively, in some embodiments, a conservative replacement comprises any change having a positive value in the PAM250 log-likelihood matrix disclosed in Gonnet et al. (1992) Science 256: 1443 45. In some embodiments, a “moderately conservative” replacement comprises any change having a nonnegative value in a PAM250 log-likelihood matrix.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g., 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “effective amount” of an antibody disclosed herein or a composition (e.g., pharmaceutical composition), is at least the minimum amount required to achieve the desired therapeutic or prophylactic result, e.g., a measurable improvement or prevention of a particular disorder, e.g., retinopathy, preferably with minimal or no toxic or detrimental effects. An effective amount may vary according to inter alia disease state, age, sex, and weight of the patient, and the ability of the antibody (or antigen-binding fragment thereof) to elicit a desired response in the individual and, in some instances, by co-administering one or more additional therapeutic agents.

An “expression vector” is a vector, e.g., plasmid, minicircle, viral vector, liposome, and the like as discussed herein or as known in the art, comprising a region which encodes a gene product of interest, and is used for effecting the expression of the gene product in an intended target cell. An expression vector also comprises control elements, e.g., promoters, enhancers, UTRs, miRNA targeting sequences, etc., operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The frizzled family is a family of frizzled class receptors, which are G protein-coupled receptors that serve as receptors in the Wingless/Integrated (Wnt) and other signaling pathways, often with a co-receptor such as LRP5, LRP6, or ROR1/2. The frizzed family members include frizzled class receptors 1-10 (Fzd1, Fzd2, Fzd3, Fzd4, Fzd5, Fzd6, Fzd7, Fzd8, Fzd9, and Fzd10).

Frizzled class receptor 4 (Fzd4) is a member of the frizzled family, a family of Fzd4 may also referred to as CD344, EVR1, FEVR, frizzled 4, frizzled family receptor 4, frizzled homolog 4, Fz4, Fz-4, Fzd4S, FzE4, MGC34390, or Wnt receptor frizzled-4. In humans, Fzd4 is encoded by the Fzd4 gene on chromosome 11, with gene location 11q14.2 (NCBI, Gene ID: 8322). In some embodiments, human Fzd4 may have the amino acid sequence provided as GenBank: AAR23924.1 or SEQ ID NO: 4 or the equivalent residues from a non-human species, e.g., mouse, rodent, monkey, ape and the like.

Several diseases and/or phenotypes are caused by one or more alterations in the Fzd4 gene or altered expression the Fzd4 gene. According to the OMIM® database (https://www.omim.org/), genetic diseases of the Fzd4 gene include: Exudative vitreoretinopathy 1 (Phenotype MIM number 133780, autosomal dominant); and Retinopathy of prematurity (Phenotype MIM number 133780, autosomal dominant)). According to MedlinePlus (NIH, https://medlineplus.gov/genetics/gene/), diseases involving genetic changes in the Fzd4 gene include: Familial exudative vitreoretinopathy.

The term “host cell” refers to cells into which an exogenous nucleic acid sequence has been introduced, including the progeny of such cells. Host cells include transformants and transformed cells, which include the primary transformed cell and progeny derived therefrom without regard to the number of passages.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

Low density lipoprotein receptors (LDLRs) are involved in a variety of cellular functions including endocytosis of low-density lipoproteins (LDLs), a primary carriers of cholesterol in the blood. The LDLR family members include LDL receptor, low density lipoprotein receptor-related proteins 1, 1b, 2, 3, 4, 5, 6, and 8 (LRP1, LRP1b, LRP2, LRP3, LRP4, LRP5, LRP6, and LRP8), LR11, and very-low-density lipoprotein (VLDL) receptor (Ren et al., Front Cell Dev Biol. 2021 May 6; 9:670960).

Among the LDLRs, LRP5 and LRP6 are unique in their structure and function. For example, both LRP5 and LRP6 can also individually function as a co-receptor of Wnt and other ligands by forming a receptor complex with another receptor such as another Wnt receptor (e.g., a Fzd receptor) and mediate signaling.

LRP5 may also referred to as BMND1, EVR1, EVR4, HBM, LR3, low density lipoprotein receptor-related protein 7 (LRP7), OPPG, OPS, OPTA1, or VBCH2. In humans, LRP5 is encoded by the LRP5 gene on chromosome 11, with gene location 11q13.2 (NCBI, Gene ID: 4041). In some embodiments, human LRP5 may have the amino acid sequence provided as NCBI Reference Sequence: NP_002326.2 or SEQ ID NO: 5 or the equivalent residues from a non-human species, e.g., mouse, rodent, monkey, ape and the like.

Several diseases and/or phenotypes are caused, mediated, and/or exacerbated by one or more alterations in the LRP5 gene or altered expression thereof. A loss-of-function mutation in LRP5 was shown to cause Osteoporosis pseudoglioma (OPPG), a rare syndrome associated with premature, generalized osteoporosis leading to bone fracture; familial exudative vitreoretinopathy (FEVR) is also associated with mutations in LRP5; upregulated LRP5 is associated with calcific aortic valve disease (e.g., bone matrix protein expression in the aortic valve and vasculature in the presence of hypercholesterolemia is dependent on LRP5, and increased LRP5 histological staining and whole tissue protein levels have been observed in degenerative human mitral valve and calcified aortic valve specimens); LRP5 gene expression has been found up-regulated in hip and knee tissue from human patients with osteoarthritis (OA); and truncated LRP5 generated by alternative splicing is associated with parathyroid tumors and breast cancer (Joiner et al., Trends Endocrinol Metab. 2013 January; 24(1):3-9). In colorectal cancer (CRC), tissue LRP5 expression is upregulated and associated with clinical stages of CRC, and LRP5 is shown to promote cancer stem-like traits (Nie et al, J Cell Mol Med 2022 February; 26(4):1095-1112.). While LRP5 expression in osteosarcoma cells is also associated with disease progression (Hoang et al., Int J Cancer. 2004 March; 109(1):106-11), overexpression of LRP5 in osteocytes was shown to enhance anti-breast cancer effects of osteocytes in bone (Liu et al., Bone Res. 2021 Jul. 6; 9(1):32.).

According to the OMIM® database (https://www.omim.org/), genetic diseases of the LRP5 gene include: exudative vitreoretinopathy 4 (Phenotype MIM number 601813, autosomal dominant and autosomal recessive); hyperostosis, endosteal (Phenotype MIM number 144750, autosomal dominant); osteopetrosis, autosomal dominant 1 (Phenotype MIM number 607634, autosomal dominant); osteoporosis-pseudoglioma syndrome (Phenotype MIM number 259770, autosomal recessive); osteosclerosis (Phenotype MIM number 144750, autosomal dominant); polycystic liver disease 4 with or without kidney cysts (Phenotype MIM number 617875, autosomal dominant); van Buchem disease, type 2 (Phenotype MIM number 607636); Bone mineral density variability 1 (Phenotype MIM number 601884, autosomal dominant); and Osteoporosis (Phenotype MIM number 166710, autosomal dominant)). According to MedlinePlus (NIH, https.//medlineplus.gov/genetics/gene/), diseases involving genetic changes in the LRP5 gene include: familial exudative vitreoretinopathy; juvenile primary osteoporosis; and osteoporosis-pseudoglioma syndrome.

LRP6 may also be referred to as ADCAD2 or STHAG7. In humans, LRP6 is encoded by the LRP6 gene on chromosome 12, with gene location 12p13.2 (NCBI, Gene ID: 4040). In some embodiments, human LRP6 may have the amino acid sequence provided as NCBI Reference Sequence: NP_002327.2 or SEQ ID NO: 6 or the equivalent residues from a non-human species, e.g., mouse, rodent, monkey, ape and the like.

Several diseases and/or phenotypes are caused, mediated, and/or exacerbated by one or more alterations in the LRP6 gene or altered expression thereof. For example, a single nucleotide polymorphism (SNP) in exon 18 of LRP6 has been associated with Alzheimer disease; a causal link between a mutation in LRP6 (R611C) and early coronary artery disease has been observed; and LRP6 gene expression has been found to be up-regulated in hip and knee tissue in human patients with osteoarthritis (OA) (Joiner et al., Trends Endocrinol Metab. 2013 January; 24(1):31-9.). Furthermore, LRP6 is upregulated in and reportedly promotes growth of various cancers including breast cancer (e.g., triple negative breast cancer (TNBC)), primary chronic lymphocytic leukemia (CLL), non-small cell lung cancer (NSCL), lung squamous cell carcinoma (LSCC), and hepatocellular carcinoma (HCC) (Li et al., Oncogene 2004 Dec. 2; 23(56):9129-35.; Roslan et al., J Oncol. 2019 Mar. 26; 2019:4536302). Related thereto multiple antagonistic anti-LRP6 antibodies have been reported to inhibit Wnt-driven tumors in vivo (Joiner et al., Trends Endocrinol Metab. 2013 January; 24(1):31-9.).

According to the OMIM® database (https://www.omim.org/), genetic diseases of the LRP6 gene include: tooth agenesis, selective, 7 (Phenotype MIM number 616724, autosomal dominant); and coronary artery disease, autosomal dominant, 2 (Phenotype MIM number 610947, autosomal dominant).

The term “native” or “wild-type” as used herein refers to a nucleotide sequence, e.g., gene, or gene product, e.g., RNA or protein, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full length native polynucleotide sequence, e.g., an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g., an identity of 75% or 80% or more, such as 85%, 90%, or 95% or more, for example, 98% or 99% identity with the full length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

“Norrin” is a secreted protein with a cystine-knot motif that activates the Wnt/p-catenin signaling pathway. In humans, Norrin is encoded by the NDP gene on X chromosome, with gene location Xp11.3 (NCBI, Gene ID: 4693). Norrin may also referred to as Norrie disease protein, NDP, ND, X-linked exudative vitreoretinopathy 2 protein, or EVR2. In some embodiments, human LRP5 may have the amino acid sequence provided as NCBI Reference Sequence: NP_000257.1 or SEQ ID NO: 3 or the equivalent residues from a non-human species, e.g., mouse, rodent, monkey, ape and the like.

Several diseases and/or phenotypes are caused, mediated, and/or exacerbated by one or more alterations in the NDP gene or altered expression thereof. According to the OMIM® database (https://www.omim.org/), genetic diseases of the NDP gene include: exudative vitreoretinopathy 2, X-linked (Phenotype MIM number 305390, X-linked dominant or X-linked recessive); and Norrie disease (Phenotype MIM number 310600, X-linked recessive). According to MedlinePlus (NIH, https://medlineplus.gov/genetics/gene/), diseases involving genetic changes in the NDP gene include: familial exudative vitreoretinopathy; Norrie disease; and retinal dystrophies.

Norrin binds to one or more Wnt receptors, e.g., Fzd4, LRP5 or LRP6 (often referred to as LRP5/6), and TSPAN12, and induces the canonical Wnt signaling pathway, and the downstream effects thereof include angiogenesis, e.g., via activation of transcription factor Sox17 (Junge et al., Cell. 2009 Oct. 16; 139(2):299-311.; and Ye et al., Cell 2009 Oct. 16; 139(2):285-98.). Mutations in Fz4, Lrp5, Norrin, or TSPAN12 are further known to cause retinal hypovascularization. Also blocking Norrin-induced, Fzd4-mediated Wnt signaling reportedly causes defects in central nervous system (CNS) angiogenesis and the blood-CNS barrier (Zhang et al., Nat Commun. 2017 Jul. 4; 8:16050).

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

A “pharmaceutically acceptable carrier” refers to an ingredient in a pharmaceutical formulation or composition, other than an active ingredient, which is largely nontoxic to a subject. A pharmaceutically acceptable carrier includes, but is not limited to, a buffer, excipient, stabilizer, or preservative. In some embodiments, a pharmaceutical formulation comprises any of the antibodies provided herein and at least one additional therapeutic agent.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” or “nucleic acid” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. The terms “identical” or “identity” when used in the context of two or more nucleic acids or polypeptide sequences, refer to the number or percentage of residues that are the same in a sequence of interest and a reference sequence. The percentage can be calculated by optimally aligning the sequence of interest to the reference sequence; comparing the two sequences over the entire length of the reference sequence; determining the number of positions at which the identical amino acid residue or nucleic acid base occurs in both sequences to yield the number of matched positions; dividing the number of matched positions by the total number of positions in the reference sequence adjusted by adding the number of gap positions introduced into the reference sequence in generating the alignment; and multiplying the result by 100 to yield the percentage of sequence identity. When comparing DNA and RNA, thymine (T) and uracil (U) can be considered equivalent. Sequence identity may be determined by using the stand-alone executable BLAST engine program for blasting two sequences (bl2seq), which can be retrieved from the National Center for Biotechnology Information (NCBI) ftp site or over the worldwide web at ncbi.nlm.nih.gov/BLAST/, using the default parameters (Tatusova and Madden, FEMS Microbiol Lett., 1999, 174, 247-250; which is incorporated herein by reference in its entirety).

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

“Recombinant,” as applied to a polynucleotide means that the polynucleotide is the product of various combinations of cloning, restriction or ligation steps, and other procedures that result in a construct that is distinct from a polynucleotide found in nature.

The terms “treatment”, “treating” and the like are used herein to generally mean obtaining a desired pharmacologic and/or physiologic effect. The effect may be prophylactic in terms of completely or partially inhibiting or preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) inhibiting the disease, i.e., arresting its development; or (b) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues, e.g., the eye(s). The subject therapy will desirably be administered during the symptomatic stage of the disease, and in some cases after the symptomatic stage of the disease.

As used herein, the phrase “retinal vascular disease” is a disease of the eye, in particular, the retinal caused by aberrant vasculature formation. In some aspects, the aberrant vasculature is caused by an inhibition of vasculature development, and in other aspects the aberrant vasculature is cause by excessive angiogenesis.

A “vector” is a compound or a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, viruses, and virus-like particles (VLPs). Thus, the term “vector” includes an autonomously replicating plasmid, a self-replicating RNA, or viral particles. Therefore, the term “vector” encompasses expression vectors. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

“Wnt signaling pathways” comprise a group of signal transduction pathways that may be induced by binding of a Wnt ligand (also called Wnt) or another ligand to one or more Wnt receptors. Wnt ligands are a diverse family of cysteine-rich and highly hydrophobic, secreted glycoproteins that are usually 350-400 amino acids in length. In humans, Wnt ligands include WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, and WNT16. Wnt receptors are receptors that can be bound by a Wnt ligand and include frizzled class receptors and co-receptors such as LRP5, LRP6, related to tyrosine kinase (Ryk), and receptor tyrosine kinase-like orphan receptor (ROR).

Wnt signaling pathways regulate complex normal cellular processes such as cell differentiation, development, tissue homeostasis, and wound healing. When Wnt signaling is aberrantly regulated, it can be associated with developmental disorders, oncogenesis, and other diseases. Primary Wnt signaling pathways include: the canonical Wnt signaling pathway (also referred to as the Wnt/β-catenin signaling pathway); the Wnt/planar cell polarity (PCP) pathway; and the Wnt/Ca2+ pathway (Sharma and Pruitt. Int J Mol Sci. 2020 Oct. 28; 21(21):8018.). These pathways are described below.

“Canonical Wnt signaling pathway”: In the absence of a Wnt ligand, cytoplasmic β-catenin generally forms a complex (often called “destruction complex”) with Axin and adenomatosis polyposis coli (APC) and is phosphorylated by casein kinase 1 (CK1) and glycogen synthase kinase 3 (GSK3). Phosphorylated β-catenin is recognized by the E3 ubiquitin ligase β-Trcp, which targets β-catenin for proteosomal degradation, and thus does not enter the nucleus. In the presence of a Wnt ligand, a Fzd and a LRP (e.g., LRP5 or LRP6), once bound by a Wnt ligand, form a receptor complex, which becomes phosphorylated by CK1 and GSK3. This causes binding of Axin to the LRP cytoplasmic tail and binding of Dishevelled (DVL) to the Fzd cytoplasmic tail, which disrupts the destruction complex, allowing cytoplasmic β-catenin to enter the nucleus. In the nucleus β-catenin causes direct and indirect transcriptional activation, for example by binding as coactivator to transcription factors of the TCF/LEF family (Wiese et al., Development. 2018 Jun. 26; 145(12):dev165902.; Sharma and Pruitt. Int J Mol Sci. 2020 Oct. 28; 21(21):8018.; Ren et al., Front Cell Dev Biol. 2021 May 6; 9:670960.). The canonical Wnt signaling pathway may also be initiated by binding of a non-Wnt ligand to one or more Wnt receptors, e.g., by the binding of Norrin (NDP) to Fzd4, LRP5 or LRP6, (often referred to as LRP5/6), and TSPAN12 (Junge et al., Cell. 2009 Oct. 16; 139(2):299-311.; and Ye et al., Cell. 2009 Oct. 16; 139(2):285-98.).

“Wnt/planar cell polarity (PCP) pathway”: A Fzd and a co-receptor (e.g., ROR1 or ROR2), once bound by a Wnt ligand, form a receptor complex and recruit DVL to the plasma membrane. DVL interacts with small GTPases such as RHO and RAC to further trigger activation of ROCK and/or JNK. This results in activation of cytoskeletal rearrangements by transcriptional responses activating transcription factors such as JUN and ATF2 (Sharma and Pruitt. Int J Mol Sci. 2020 Oct. 28; 21(21):8018.).

“Wnt/Ca2+ pathway”: A Fzd and a co-receptor (e.g., ROR1 or ROR2), once bound by a Wnt ligand, form a receptor complex and activates phospholipase C (PLC), resulting in intracellular Ca2+ influx. This further activates CDC42 and triggers Ca2+-dependent cell movement and polarity via various transcriptional factors such as NFAT (Sharma and Pruitt. Int J Mol Sci 2020 Oct. 28; 21(21):8018.).

The practice of the present invention will employ, unless otherwise indicated, conventional techniques of cell biology, molecular biology techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terms “including”, “includes”, “having”, “has”, “with”, or variants thereof are used in either the detailed description and/or the claims, such terms are intended to be inclusive in a manner similar to the term “comprising”.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### II. General

The present disclosure provides Wnt signal modulators and methods of use thereof for treating diseases, disorders, and conditions, e.g., involving aberrant Wnt signaling, such as but not limited to various ocular disorders, including methods of modulating Wnt signals to treat retinopathy, including but limited to, FEVR and other genetic disorders, DR, and AMD. In particular embodiments, the present invention provides Wnt/0-catenin agonists to inhibit aberrant neovascularization in the progression of retinopathy.

Wnt (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea, lung, and many other tissues (reviewed, e.g., by Clevers, Loh, and Nusse, Science 2014; 346:1248012). Modulation of Wnt signaling pathways has potential for treatment of degenerative diseases and tissue injuries.

One of the challenges for modulating Wnt signaling as a therapeutic is the existence of multiple Wnt ligands and Wnt receptors, Frizzled 1-10 (Fzd1-10), with many tissues expressing multiple and overlapping Fzds. Canonical Wnt signals also involve Low-density lipoprotein (LDL) receptor-related protein 5 (LRP5) or Low-density lipoprotein (LDL) receptor-related protein 6 (LRP6) as co-receptors, which are broadly expressed in various tissues, in addition to Fzds.

R-spondins 1-4 are a family of ligands that amplify Wnt signals. Each of the R-spondins work through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight and Hankenson 2014, Matrix Biology; 37: 157-161). R-spondins might also work through additional mechanisms of action. ZNRF3 and RNF43 are two membrane-bound E3 ligases specifically targeting Wnt receptors (Fzd1-10 and LRP5 or LRP6) for degradation. Binding of an R-spondin to ZNRF3/RNF43 and LGR4-6 causes clearance or sequestration of the ternary complex, which removes E3 ligases from Wnt receptors and stabilizes Wnt receptors, resulting in enhanced Wnt signals. Each R-spondin contains two furin domains (1 and 2), with Furin domain 1 binding to ZNRF3/RNF43, and Furin domain 2 binding to LGR4-6. Fragments of R-spondins containing Furin domains 1 and 2 are sufficient for amplifying Wnt signaling. While R-spondin effects depend on Wnt signals, since both LGR4-6 and ZNRF3/RNF43 are widely expressed in various tissues, the effects of R-spondins are not tissue-specific.

In certain embodiments, the Wnt/0-catenin agonist (or Wnt signaling agonist) comprises an antibody, antibody fragment, and/or antibody domain that binds to one or more Fzd and an antibody domain that binds to LRP5 and/or Lrp6. In certain embodiments, the antibody domain preferentially binds to Fzd4 as compared to other Fzds. In certain embodiments, a Fzd binding domain preferentially binds to one or more Fzd(s) where it binds to those Fzd(s) with a K_{D} at least 10-fold lower than the K_{D} with which it binds to other Fzds. In certain embodiments, a Fzd binding domain preferentially binds to one or more Fzd(s) where it binds to those Fzd(s) with a K_{D} at least 100-fold lower than the K_{D} with which it binds to other Fzds. In certain embodiments, a Fzd binding domain preferentially binds to one or more Fzd(s) where it binds to those Fzd(s) with a K_{D} at least 1000-fold lower than the K_{D} with which it binds to other Fzds. In certain embodiments, the antibody domains comprise the corresponding CDRs present in the Wnt signaling agonist disclosed herein, e.g., hp4SD1-03, or comprises a fragment or variant of hp4SD1-03, e.g., a variable heavy chain or variable light chain region. Non-limiting, exemplary sequences may be found in APPENDIX Tables A-J.

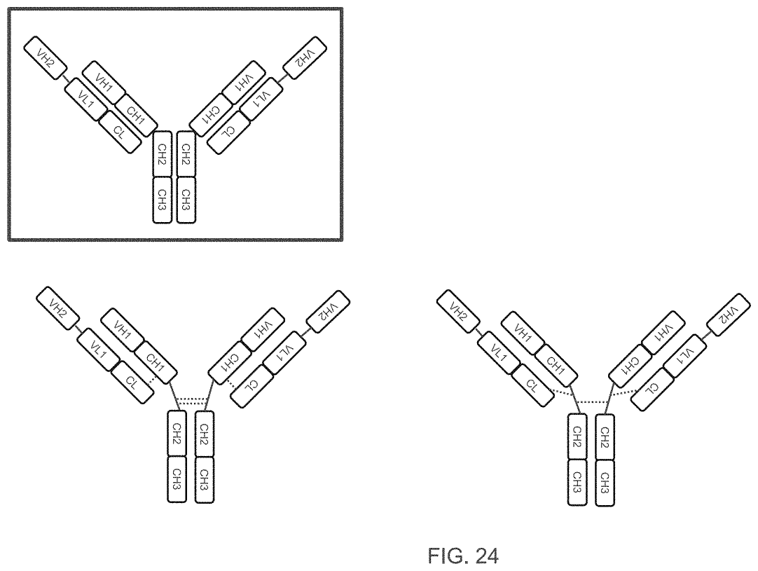

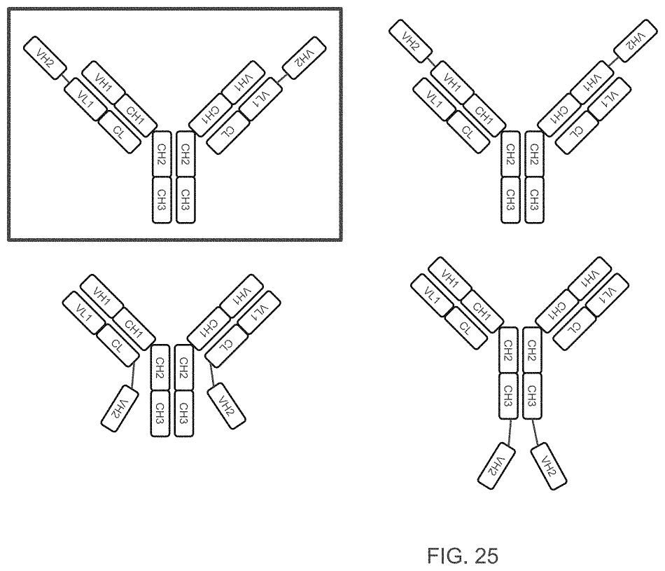

In certain embodiments, the Wnt/0-catenin agonist comprises an IgG antibody like structure comprising two antibody light chains and two antibody heavy chains, and further comprising two VHH domain fused to any of the antibody chains (see e.g., FIGS. 24-25). In particular embodiments, a VHH domain is fused to the N-terminus of each antibody light chain (see e.g., FIG. 24). In particular embodiments, the antibody heavy and light chains bind one or more Fzd, or preferentially bind to Fzd4, and the VHH domains bind to LRP5 and/or LRP6. In certain embodiments, the VHH domain preferentially binds to LRP5 as compared to LRP6. In certain embodiments, a LRP5/6 binding domain preferentially binds to LRP5 or LRP6 where it binds to that LRP with a K_{D} at least 100-fold lower than the K_{D} with which it binds to other LRP. In certain embodiments, a LRP5/6 binding domain preferentially binds to LRP5 or LRP6 where it binds to that LRP with a K_{D} at least 1000-fold lower than the K_{D} with which it binds to the other LRP.

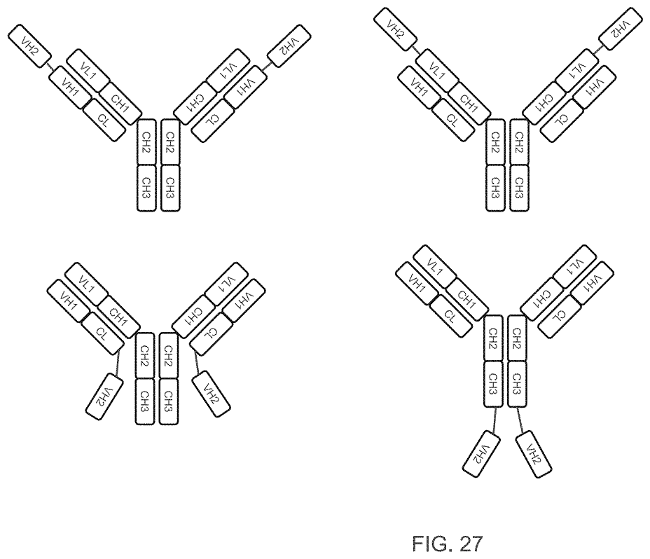

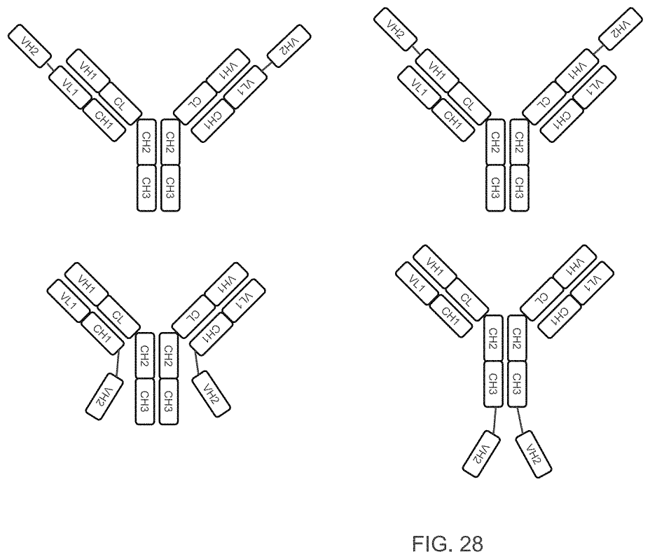

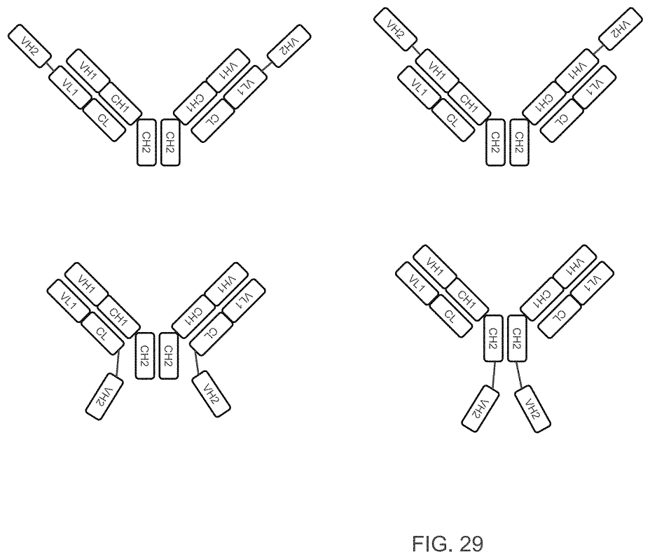

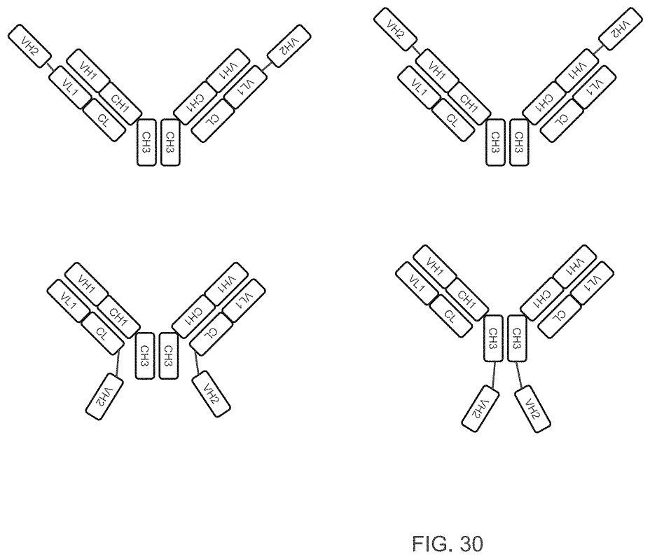

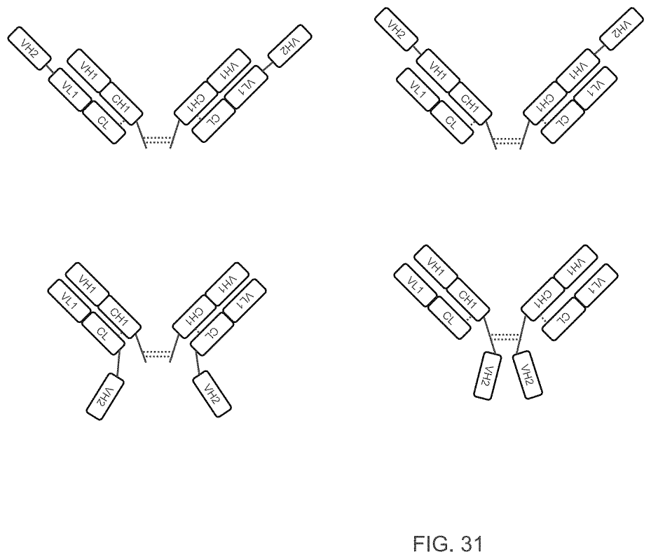

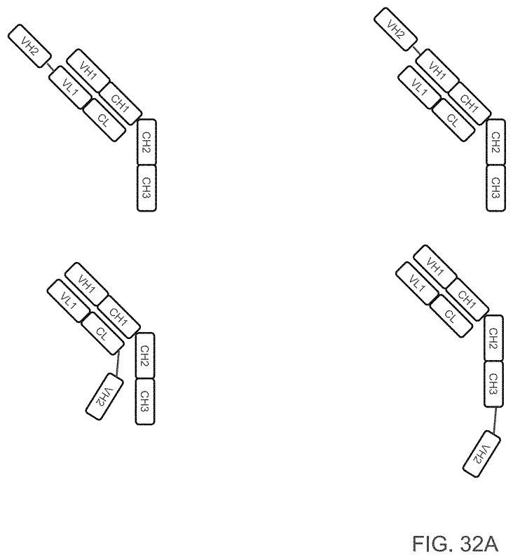

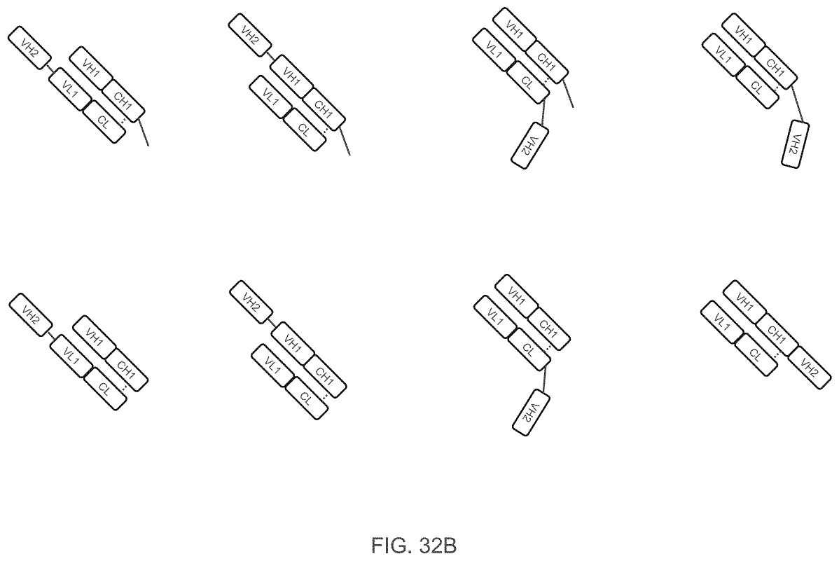

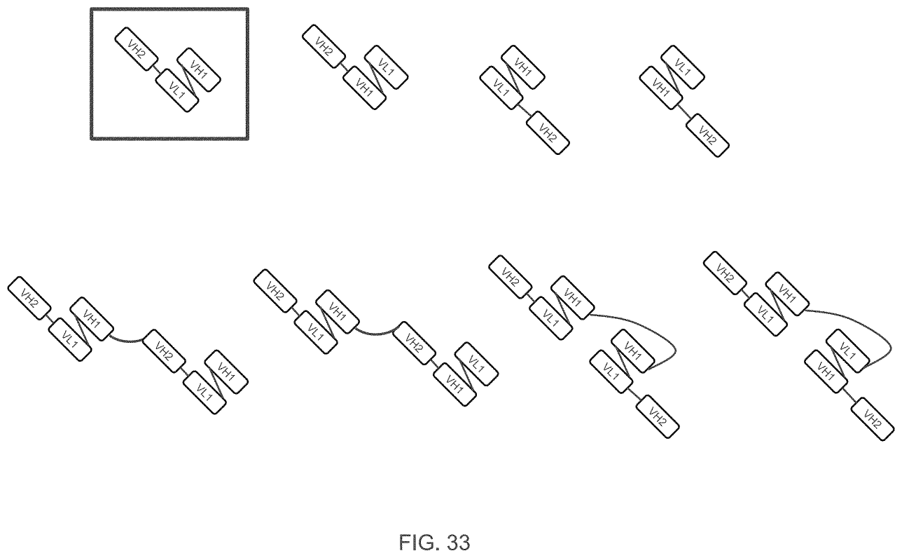

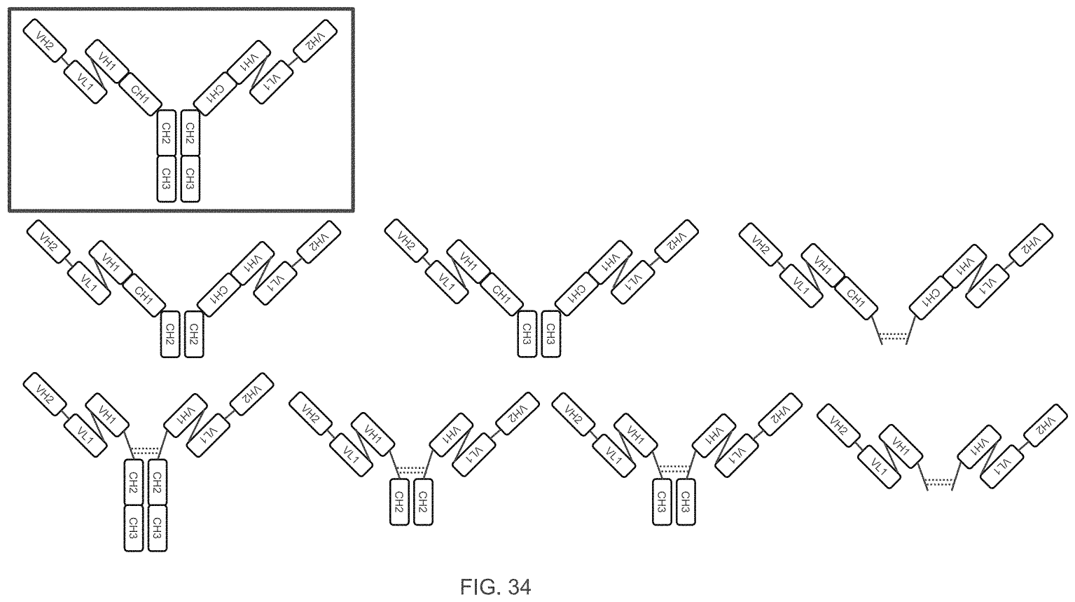

In related embodiments, the Wnt/β-catenin agonist may adopt a different bi-specific binding structure, including but not limited to any disclosed herein (including but not limited to the structures depicted in FIGS. 24-34). As is well known in the art, an antibody is an immunoglobulin molecule capable of specific binding to a target such as a carbohydrate, polynucleotide, lipid, polypeptide, etc., through at least on epitope binding domain, located on the variable region of the immunoglobulin molecule. As used herein, the term encompasses not only intact polyclonal or monoclonal antibodies, but also fragments thereof containing epitope binding domains (e.g., dAb, Fab, Fab′, (F(ab′)₂, Fv, single chain (scFv), VHH or single domain antibodies (sdAb), DVD-Igs, synthetic variants thereof, naturally occurring variants, fusion proteins comprising and epitope binding domain, humanized antibodies, chimeric antibodies, and any other modified configuration of the immunoglobulin molecule that comprises an antigen-binding site or fragment (epitope recognition site) of the required specificity. “Diabodies,” multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., Proc. Natl. Acad. Sci. USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH:VL heterodimer including an antigen-binding site which retains much of the antigen recognition and binding capabilities of the native antibody molecule. Inbar et al. (1972) Proc. Natl. Acad. Sci. USA 69:2659-2662; Hochman et al. (1976) Biochem 15:2706-2710; and Ehrlich et al. (1980) Biochem 19:4091-4096.

In certain embodiments, single chain Fv or scFV antibodies are contemplated. For example, Kappa bodies (Ill et al., Prot. Eng. 10: 949-57 (1997)); minibodies (Martin et al., EMBO J 13: 5305-9 (1994)); diabodies (Holliger et al., PNAS 90: 6444-8 (1993)); or Janusins (Traunecker et al., EMBO J 10: 3655-59 (1991) and Traunecker et al., Int. J. Cancer Suppl. 7: 51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more Fzd receptors through one binding domain and to a second molecule through a second binding domain. These antibodies may be produced through recombinant molecular biological techniques or may be physically conjugated together. A single chain Fv (scFv) polypeptide is a covalently linked VH:VL heterodimer which is expressed from a gene fusion including VH- and VL-encoding genes linked by a peptide-encoding linker. Exemplary linkers that may be used to connect VH and VL in scFvs may comprise a flexible linker. In some embodiments, the linker may comprise one or more amino acids, optionally between 1-50 amino acids, such as one, two, three, four, five, six, seven, eight, nine, ten, eleven, twelve, thirteen, fourteen, fifteen, sixteen, seventeen, eighteen, nineteen, or twenty amino acids. In some embodiments, the linker may consists of small amino acids consisting of G, S, and/or A. In some embodiments, the linker may comprise an amino acid sequence which comprises or consists of the amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG. In some embodiments, the linker may comprise an amino acid sequence which comprises or consists of multiple repeats (e.g., two, three, four, or five repeats) of the amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG. In certain embodiments, the linker may comprise three or four repeats of SEQ ID NO: 101, which may optionally be called (G4S)₃ linker and (G4S)₄ linker, respectively. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

When bispecific antibodies are used, they may comprise conventional bispecific antibodies, which can be manufactured by various known methods. e.g., as described in (Holliger, P. and Winter G., Current Opinion Biotechnol. 4, 446-449 (1993)), or thy may be prepared chemically or from hybrid hybridomas, or they may comprise any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful in part because they can be readily constructed and expressed in E. coli. Diabodies (and many other polypeptides such as antibody fragments) of appropriate binding specificities can be readily selected using phage display (see e.g., WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al., Protein Eng., 9, 616-621 (1996)).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino acid residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

A “monoclonal antibody” refers to a homogeneous antibody population (the individual antibodies comprising the population are identical and bind the same epitope, except for possible variant antibodies (e.g., containing naturally occurring mutations or arising during production of a monoclonal antibody preparation), such variants generally being present in minor amounts.) wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)₂, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including Wnt surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

In certain embodiments, the antibodies of the present disclosure may take the form of a Nanobody®. Nanobody® technology was originally developed following the discovery and identification that Camelidae (e.g., camels and llamas) possess fully functional antibodies that consist of heavy chains only and therefore lack light chains. These heavy-chain only antibodies contain a single variable domain (VHH) and two constant domains (CH2, CH3). The cloned and isolated single variable domains have full antigen binding capacity and are very stable. These single variable domains, with their unique structural and functional properties, form the basis of “Nanobodies®”. Nanobodies® are encoded by single genes and are efficiently produced in almost all prokaryotic and eukaryotic hosts, e.g., E. coli (see, e.g., U.S. Pat. No. 6,765,087), molds (for example Aspergillus or Trichoderma) and yeast (for example Saccharomyces, Kluyvermyces, Hansenula or Pichia (see, e.g., U.S. Pat. No. 6,838,254). The production process is scalable and multi-kilogram quantities of Nanobodies® have been produced. Nanobodies® may be formulated as a ready-to-use solution having a long shelf life. The Nanoclone® method (see, e.g., WO 06/079372) is a proprietary method for generating Nanobodies® against a desired target, based on automated high-throughput selection of B-cells. Nanobodies® are single-domain antigen-binding fragments of camelid-specific heavy-chain only antibodies. Nanobodies®, also referred to as VHH antibodies, typically have a small size of around 15 kDa.

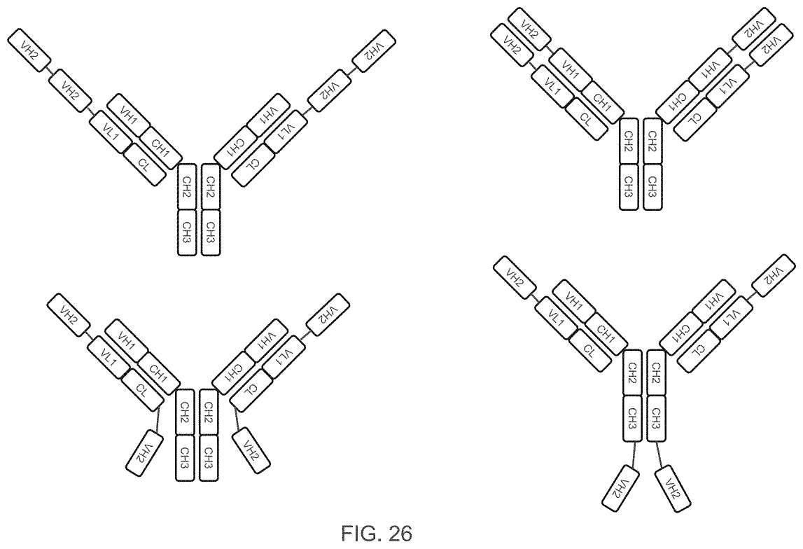

Another antibody fragment contemplated is a dual-variable domain-immunoglobulin (DVD-Ig) which is an engineered protein that combines the function and specificity of two monoclonal antibodies in one molecular entity. A DVD-Ig is designed as an IgG-like molecule, except that each light chain and heavy chain contains two variable domains in tandem through a short peptide linkage, instead of one variable domain in IgG. The fusion orientation of the two variable domains and the choice of linker sequence are critical to functional activity and efficient expression of the molecule. A DVD-Ig can be produced by conventional mammalian expression systems as a single species for manufacturing and purification. A DVD-Ig has the specificity of the parental antibodies, is stable in vivo, and exhibits IgG-like physicochemical and pharmacokinetic properties. DVD-Igs and methods for making them are described in Wu, C., et al., Nature Biotechnology, 25:1290-1297 (2007)). The DVD-Ig structure may be applied to cases in which at least one of the antigen-binding regions comprises a VHH domain (for example, see FIG. 26 (e.g., top right).

In certain embodiments, the antibodies or antigen-binding fragments thereof as disclosed herein are humanized. This refers to a chimeric molecule, generally prepared using recombinant techniques, having an antigen-binding site (i.e., the complete set of CDRs) derived from an immunoglobulin from a non-human species and the remaining immunoglobulin structure of the molecule based upon the structure and/or sequence of a human immunoglobulin. In some cases, for example when the FR sequences derived from a non-human species match or closely match corresponding human FR sequences, a humanized antibody or antigen-binding antibody fragment may comprise complete variable domains derived from a non-human species fused onto constant domains. In some cases, for example when the FR sequences derived from a non-human species differ from or significantly differ from corresponding human FR sequences, a humanized antibody or antigen-binding antibody fragment may comprise CDRs derived from a non-human species grafted onto appropriate framework regions in the variable domains fused onto constant domains. In some cases, appropriate residues in FRs are substituted to the corresponding human residue or a residue similar thereto (e.g., a conservative amino acid). In certain cases, some residues in CDRs may also be substituted to the corresponding human residue or a residue similar thereto (e.g., a conservative amino acid). Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327). Illustrative methods for humanization of the anti-Fzd or LRP antibodies disclosed herein include the methods described in U.S. Pat. No. 7,462,697 and other well-known humanization methods.

Such further modifications to the CDRs may be to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856; Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous constant or Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

In some embodiments, antibodies or antibody fragments according to the present disclosure comprises a Fc region.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9(1): 63-73; Dall'Acqua W. F. et al., J Biol Chem. 2006 Aug. 18; 281(33):23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289(6): 3571-3590).

In some embodiments, an antibody or antibody fragment such as a multispecific antibody or antibody fragment according to the present disclosure may comprise an Fc region comprising one or more amino acid substitutions that reduce or mitigate an effector function compared to the corresponding wild-type Fc region (also called “Fc silencing mutations or substitutions). In certain embodiments, an Fc region may comprise one or more amino acid substitutions at positions selected from the group consisting of positions 234, 235, 236, 237, 265, 297 and 329, according to EU numbering. In certain embodiments, an Fc region may comprise L234A, L235A, and P329G, according to EU numbering (also referred to as “LALAPG” herein). In certain embodiments, an Fc region may comprise L234A and L235A, according to EU numbering (also referred to as “LALA” herein). In certain embodiments, an Fc region may comprise D265A and P329A, according to EU numbering (also referred to as “DAPA” herein). In certain embodiments, an Fc region may comprise N297A, according to EU numbering.

In some embodiments, an antibody or antibody fragment such as a multispecific antibody or antibody fragment according to the present disclosure may comprise an Fc region comprising one or more amino acid substitutions that reduce the half-life of the antibody or antibody fragment compared to the corresponding wild-type Fc region. In certain embodiments, an Fc region may comprise I253A, H310A, and H435Q, according to EU numbering (also referred to as “IAHAHQ” or “AAQ” herein). In certain embodiments, an Fc region may comprise I253A, H310A, and H435A, according to EU numbering (also referred to as “IAHAHA” or “AAA” herein).

In some embodiments, an antibody or antibody fragment such as a multispecific antibody or antibody fragment according to the present disclosure may comprise an Fc region comprising one or more amino acid substitutions that increase FcRn binding and/or increase the half-life of the antibody or antibody fragment compared to the corresponding wild-type Fc region. In certain embodiments, an Fc region may comprise M252Y, S254T, and T256E, according to EU numbering (also referred to as “YTE” herein). In certain embodiments, an Fc region may comprise M428L and N434S, according to EU numbering (also referred to as “LS” herein).

In some embodiments, when an antibody comprises an Fc region of or derived from IgG1, the Fc region may comprise one or more amino acid substitutions. The substitution may be, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y, M428L, N434S, L328F, M252Y, S254T, T256E, and/or any combination thereof (the residue numbering is according to EU numbering) (Dall'Acqua W. F. et al., J Biol Chem. 2006 Aug. 18; 281(33):23514-24. Epub 2006 Jun. 21; Wang X. et al., Protein Cell. 2018 January; 9(1): 63-73). The Fc region may further comprise one or more additional amino acid substitutions. The substitution may be, for example, but is not limited to, A330L, L234F, L235E, P331S, and/or any combination thereof (the residue numbering is according to EU numbering).

In some embodiments, when an antibody comprises an Fc region of or derived from IgG2, the Fc region may comprise one or more amino acid substitutions. The substitution may be, for example, but is not limited to, P238S, V234A, G237A, H268A, H268Q, H268E, V309L, N297A, N297Q, A330S, P331S, C232S, C233S, M252Y, S254T, T256E, and/or any combination thereof (the residue numbering is according to EU numbering). The Fc region may further comprise one or more additional amino acid substitutions. The substitution may be, for example, but is not limited to, M252Y, S254T, T256E, and/or any combination thereof (the residue numbering is according to EU numbering).

In some embodiments, when an antibody comprises an Fc region of or derived from IgG3, the Fc region may comprise one or more amino acid substitutions. The substitution may be, for example, but is not limited to, E235Y (the residue numbering is according to EU numbering).

In some embodiments, when an antibody comprises an Fc region of or derived from IgG4, the Fc region may comprise one or more amino acid substitutions. The substitution may be, for example, but is not limited to, E233P, F234V, L235A, G237A, E318A, S228P, L236E, S241P, L248E, T394D, M252Y, S254T, T256E, N297A, N297Q, and/or any combination thereof (the residue numbering is according to EU numbering). The substitution may be, for example, S228P (the residue numbering is according to EU numbering).

In some embodiments, the glycan of the human-like Fc region may be engineered to modify the effector function (for example, see Li T. et al., Proc Natl Acad Sci USA. 2017 Mar. 28; 114(13):3485-3490. doi: 10.1073/pnas.1702173114. Epub 2017 Mar. 13).

In some embodiments, the structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

In certain embodiments, the antagonist or agonist binding agent binds to one or more Fzd and/or LRP5/6 with a dissociation constant (K_{D}) of about 1 μM or less, about 100 nM or less, about 40 nM or less, about 20 nM or less, or about 10 nM or less. For example, in certain embodiments, a Fzd binding domain or antibody described herein that binds to one or more Fzds, binds to those Fzds with a K_{D} of about 100 nM or less, about 20 nM or less, or about 10 nM or less. In certain embodiments, the binding domain binds to one or more its target antigen with an EC50 of about 1 μM or less, about 100 nM or less, about 40 nM or less, about 20 nM or less, about 10 nM or less, or about 1 nM or less.

The antibodies or other agents of the present invention can be assayed for specific binding by any method known in the art. The immunoassays which can be used include, but are not limited to, competitive and non-competitive assay systems using techniques such as biolayer interferometry (BLI) analysis, surface plasmon resonance (SPR), FACS analysis, immunofluorescence, immunocytochemistry, Western blots, radioimmunoassays, ELISAs, “sandwich” immunoassays, immunoprecipitation assays, precipitation reactions, gel diffusion precipitation reactions, immunodiffusion assays, agglutination assays, complement-fixation assays, immunoradiometric assays, fluorescent immunoassays, and protein A immunoassays. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety).

For example, the specific binding of an antibody to a target antigen may be determined using ELISA. An ELISA assay comprises preparing antigen, coating wells of a 96 well microtiter plate with antigen, adding the antibody or other binding agent conjugated to a detectable compound such as an enzymatic substrate (e.g., horse-radish peroxidase or alkaline phosphatase) to the well, incubating for a period of time and detecting the presence of the antigen. In some embodiments, the antibody or agent is not conjugated to a detectable compound, but instead a second conjugated antibody that recognizes the first antibody or agent is added to the well. In some embodiments, instead of coating the well with the antigen, the antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art (see e.g., Ausubel et al, eds, 1994, Current Protocols in Molecular Biology, Vol. 1, John Wiley & Sons, Inc., New York at 11.2.1).

The binding affinity of an antibody or other agent to a target antigen and the off-rate of the antibody-antigen interaction can be determined by competitive binding assays. One example of a competitive binding assay is a radioimmunoassay comprising the incubation of labeled antigen (e.g., Fzd, LRP), or fragment or variant thereof, with the antibody of interest in the presence of increasing amounts of unlabeled antigen followed by the detection of the antibody bound to the labeled antigen. The affinity of the antibody and the binding off-rates can be determined from the data by Scatchard plot analysis. In some embodiments, BLI analysis is used to determine the binding on and off rates of antibodies or agents. BLI kinetic analysis comprises analyzing the binding and dissociation of antibodies from sensors with immobilized antigens on their surface.

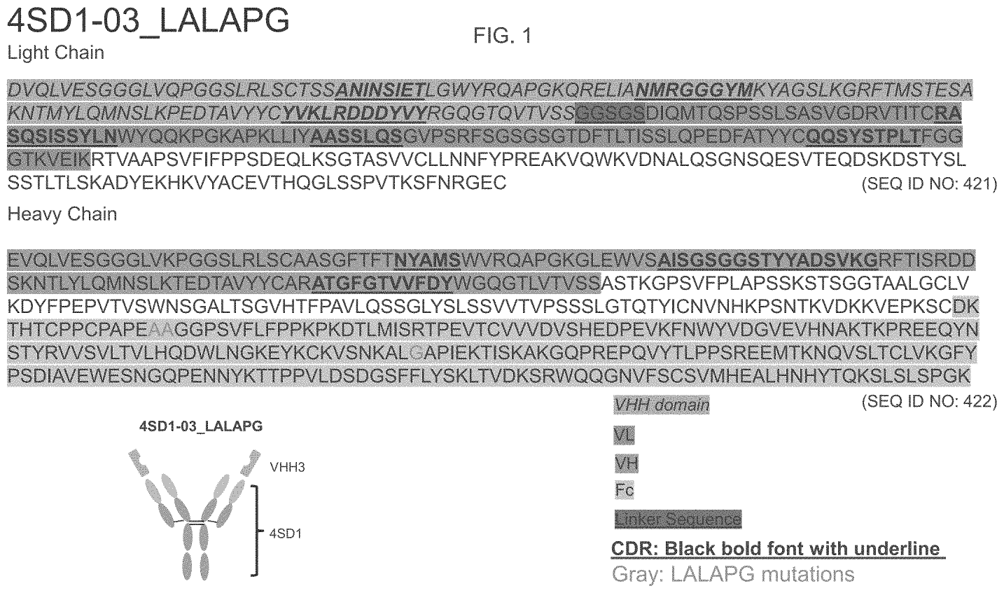

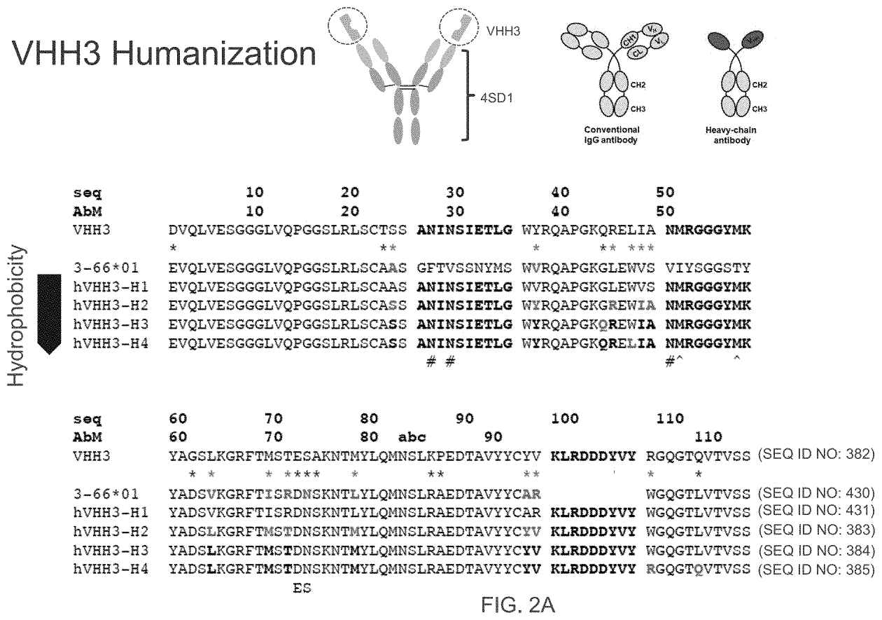

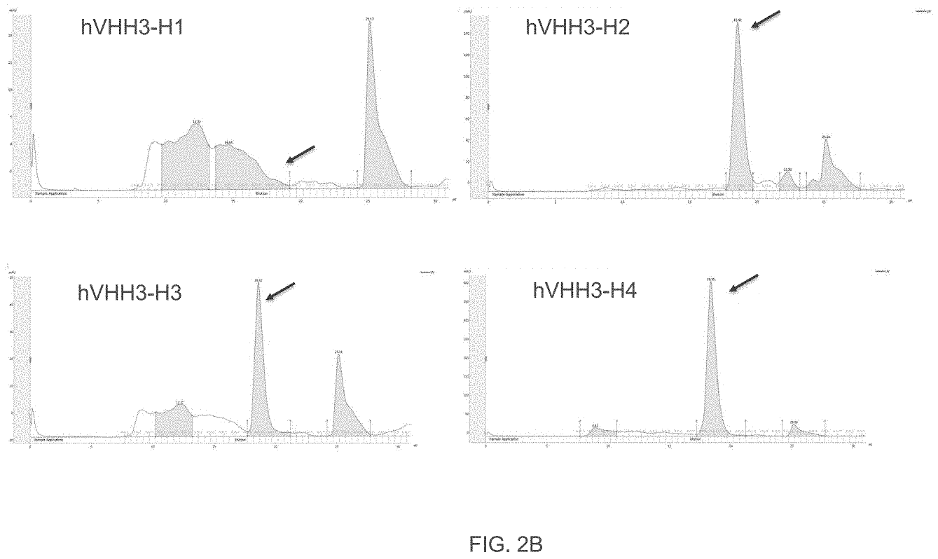

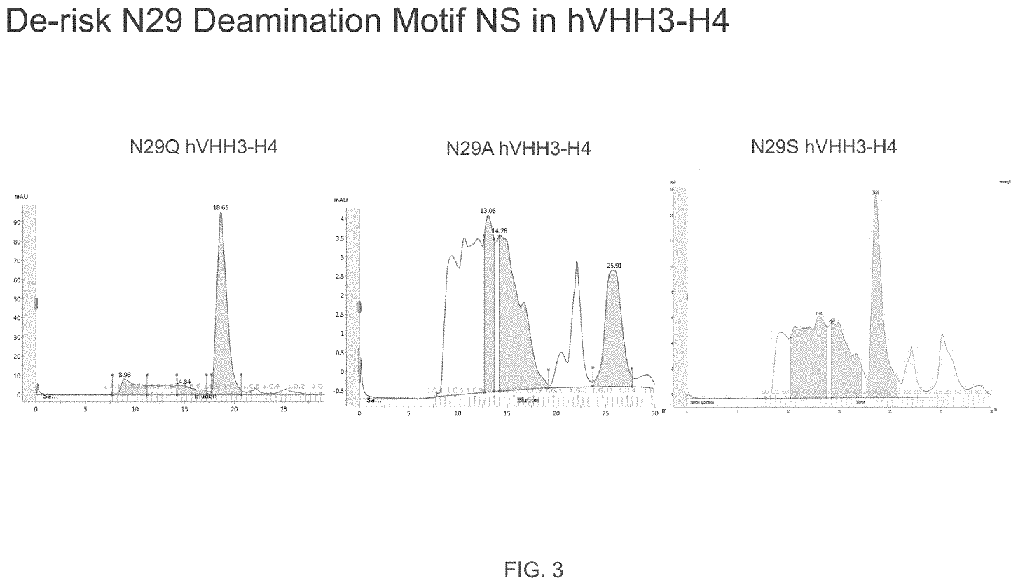

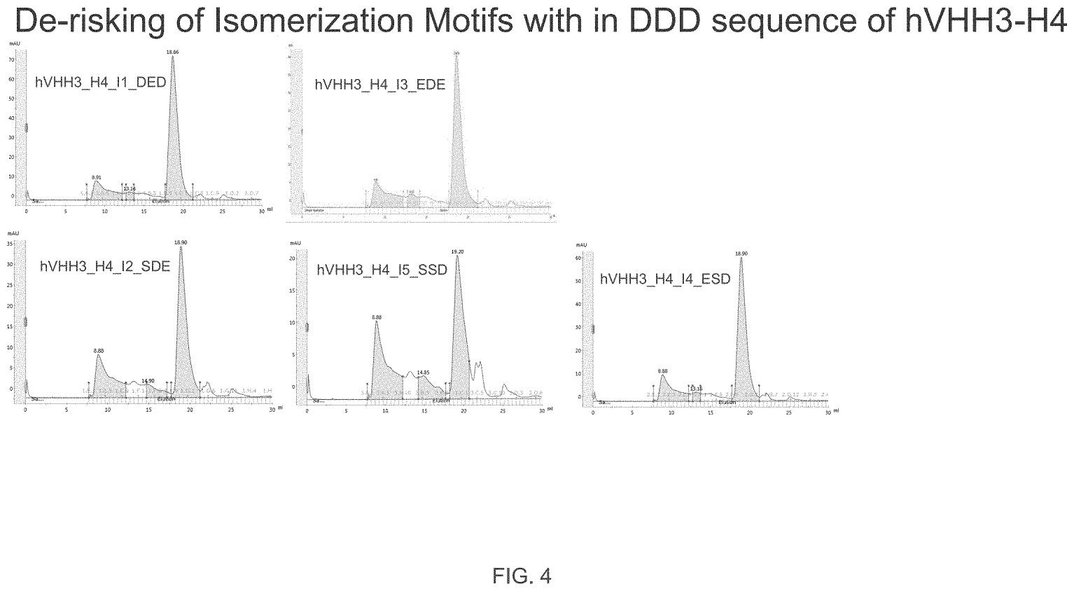

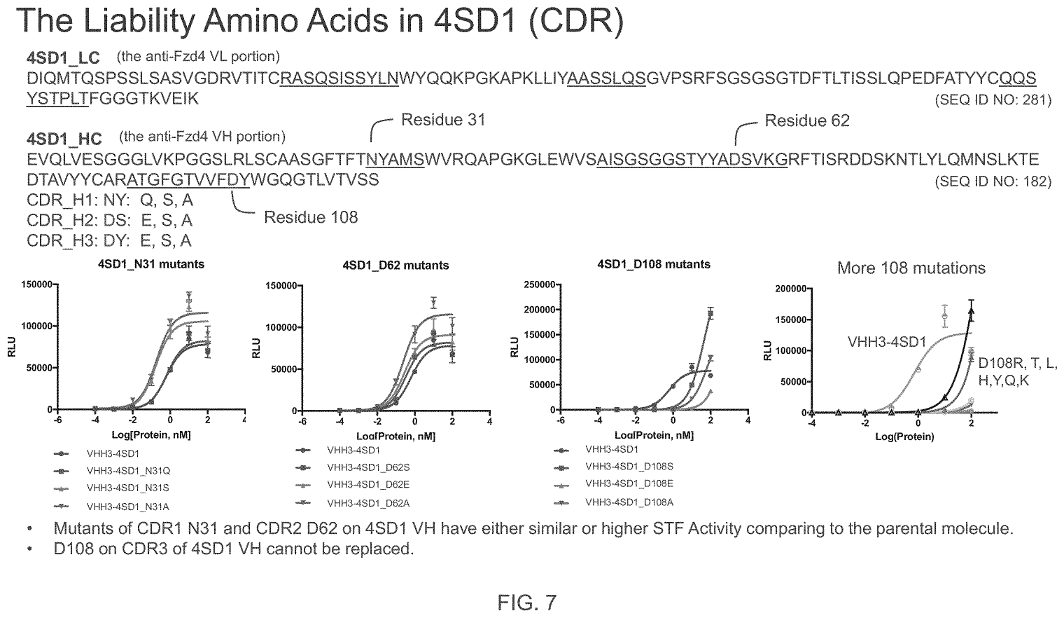

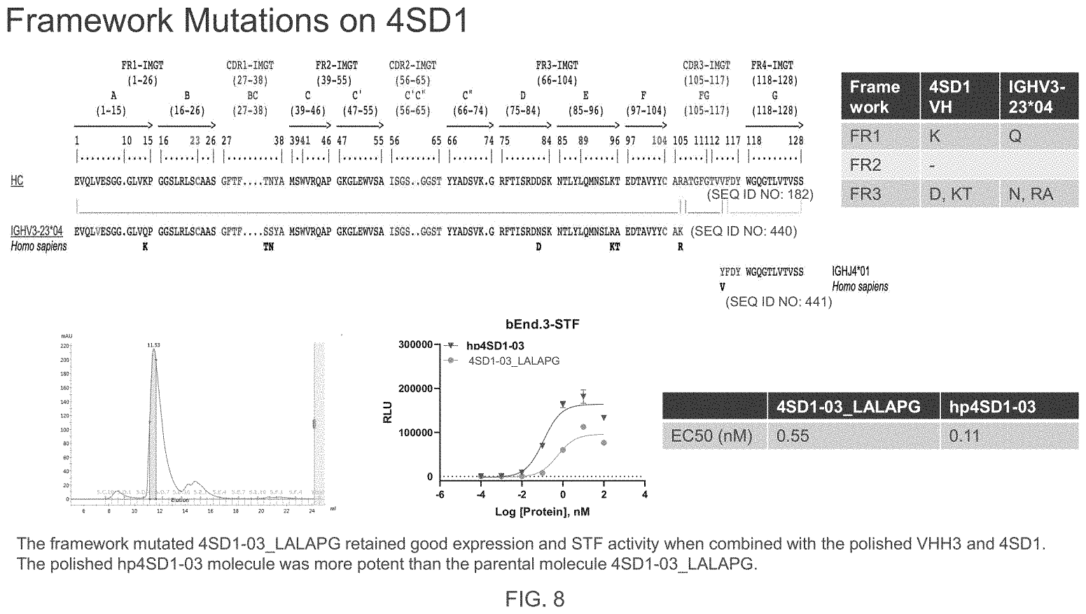

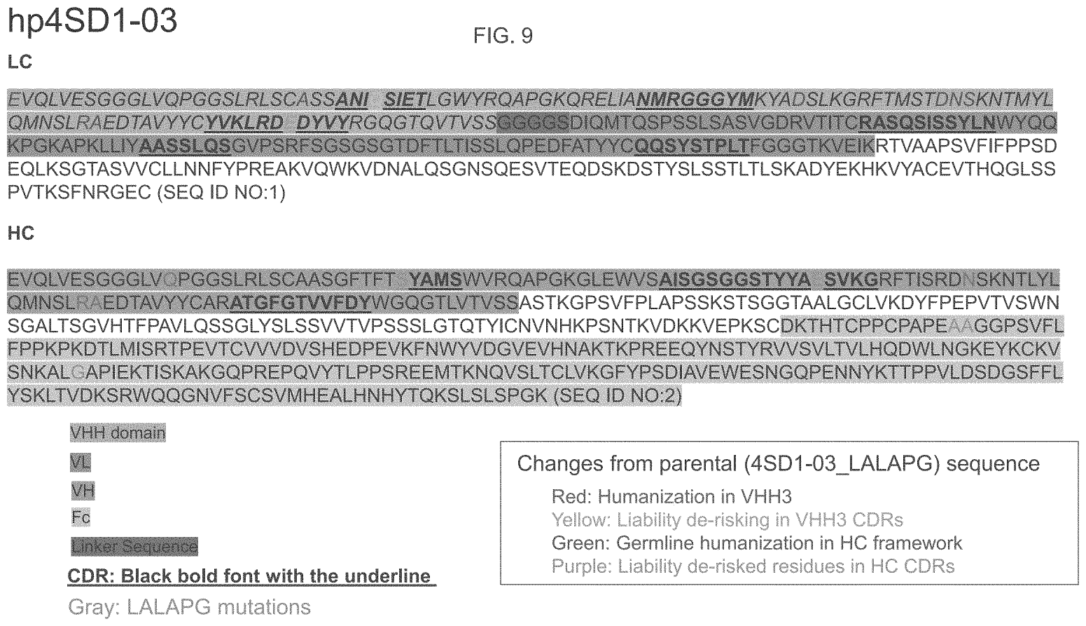

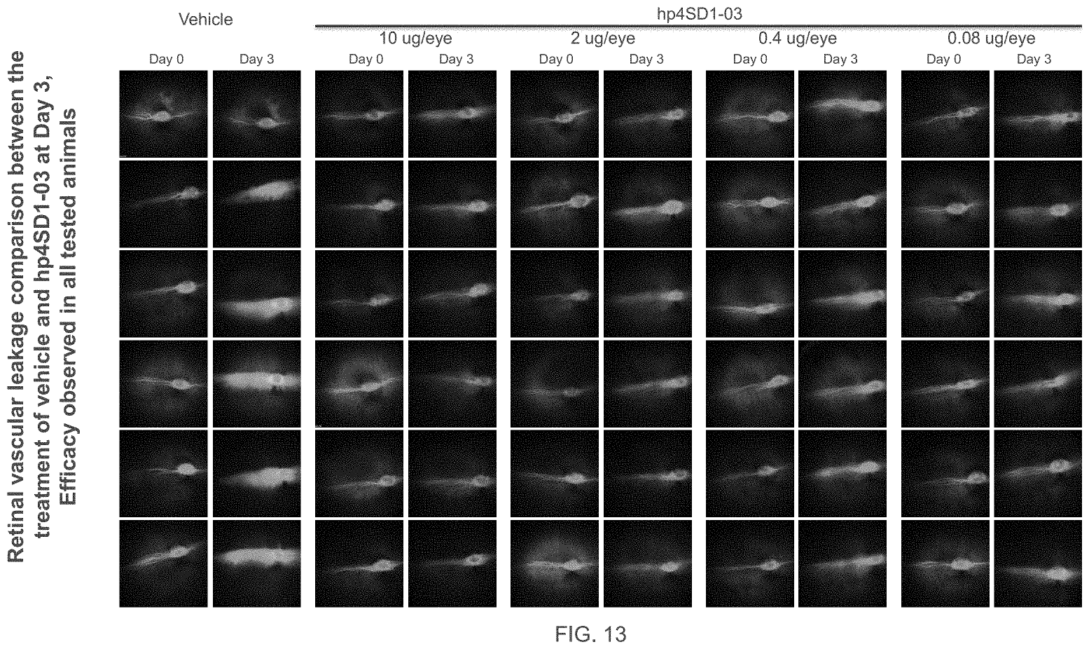

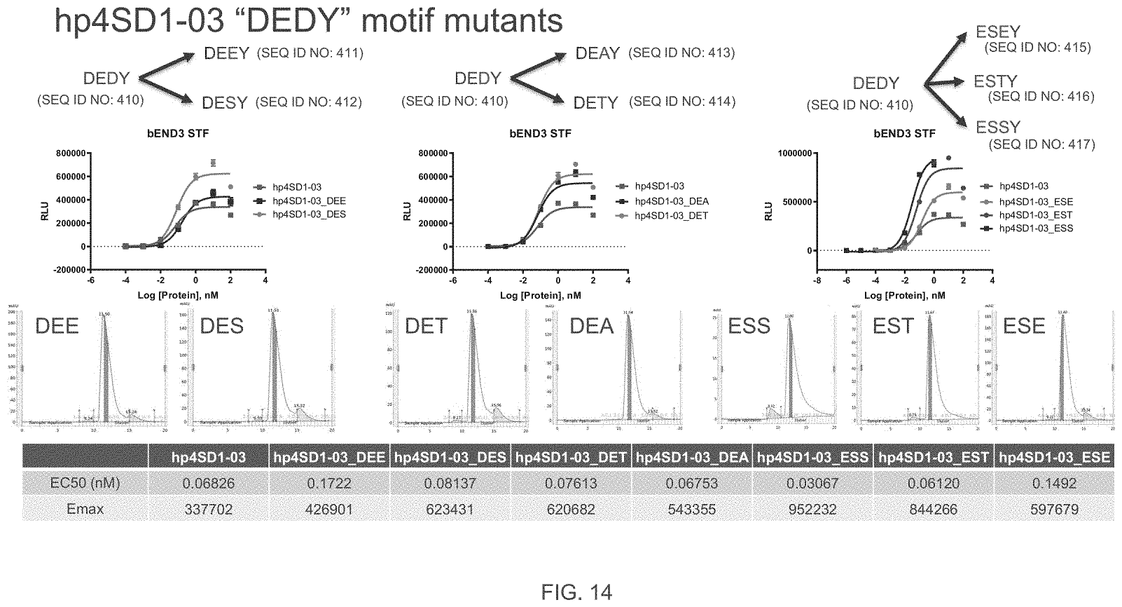

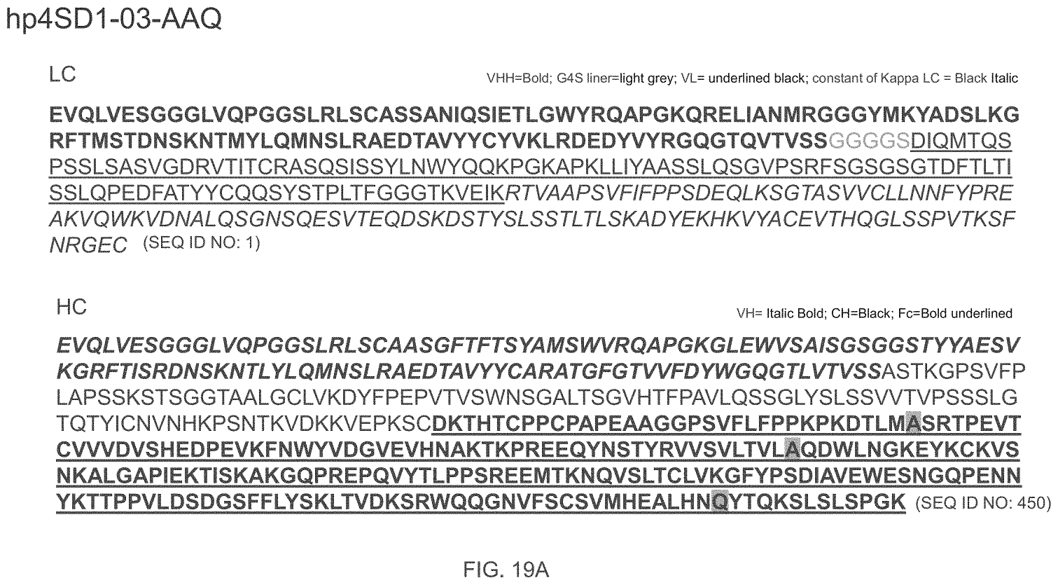

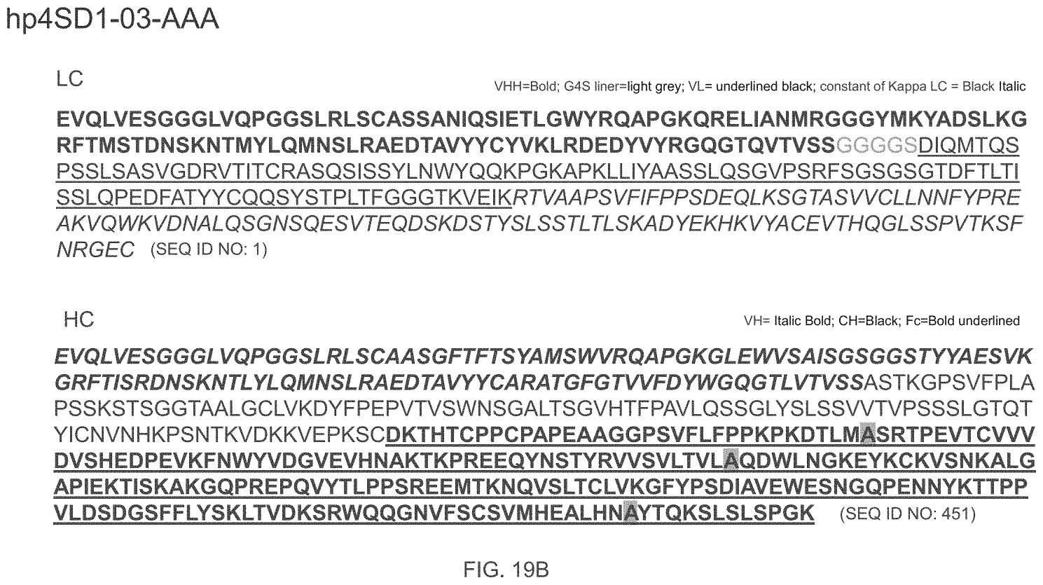

In certain embodiments, the disclosure provides Wnt signaling agonists, including but not limited to hp4SD1-03, hp4SD1-03 AAQ, hp4SD1-03 AAA, and also fragments and variants thereof. In particular embodiments, a Wnt signaling agonist has a structure diagrammed in FIG. 1 or the boxed structure in FIG. 24 and/or comprises one or both of the sequences disclosed in FIG. 9 or FIG. 19A or 19B, or any functional fragment or variant thereof, e.g., an antigen-binding fragment thereof, such as a VHH domain, VL domain, or VH domain, or an Fc domain, or a variant of any of these functional domains, e.g., a variant having at least 90%, at least 95%, at least 98%, or at least 99% sequence identity to any functional fragment of antibody sequence disclosed herein. In some embodiments, a Wnt signaling agonist comprises a variant sequence having at least 90% identity (e.g., 95%, 98% or 100% identity) to a sequence disclosed in FIG. 9 or FIG. 19A or 19B, or set forth in SEQ ID NOs: 1 and/or 2, SEQ ID NOs: 1 and/or 7, SEQ ID NOs: 1 and/or 450, SEQ ID NOs: 1 and/or 451, SEQ ID NOs: 1 and/or 452, SEQ ID NOs: 1 and/or 453, or any functional fragment thereof, e.g., an antigen-binding fragment thereof. In some embodiments, a Wnt signaling agonist comprises a wild type or variant sequence having at least 90% identity (e.g., 95%, 98% or 100% identity) to a light chain sequence disclosed in FIG. 9 or FIG. 19A or 19B, or set forth in SEQ ID NO: 1, and a wild type or variant sequence having at least 90% identity (e.g., 95%, 98% or 100% identity) to a heavy chain sequence disclosed in FIG. 9 or FIG. 19A or 19B, or set forth in SEQ ID NO:2, 7, 450, 451, 452, or 453. In some embodiments, a Wnt signaling agonist comprises two wild type or variant sequences, each having at least 90% identity (e.g., 95%, 98% or 100% identity) to a light chain sequence disclosed in FIG. 9 or FIG. 19A or 19B, or set forth in SEQ ID NO: 1, and two wild type or variant sequences, each having at least 90% identity (e.g., 95%, 98% or 100% identity) to a heavy chain sequence disclosed in FIG. 9 or FIG. 19A or 19B, or set forth in SEQ ID NO: 2, 7, 450, 451, 452, or 453, wherein the two heavy chains are bound to each other and each light chain is bound to a different heavy chain, e.g., by disulfide bonds. In particular embodiments, one or more of the amino acid residues are introduced into the parental molecule, 4SD1-03_LALAPG, to generate hp4SD1-03, including but not limited to, e.g., those in hVHH3-H4 (FIG. 2), hVHH3-H4 N29Q (FIG. 3), hVHH3-H4 DDD substituted with DED (FIG. 4), 4SD1 HC CDR1 N31S, and CDR2 D62E (FIG. 7), and/or those in the humanized 4SD1 framework, as shown in FIG. 8. In certain embodiments, VHH3 CDR3 D108 is maintained. In certain embodiments, VHH3 M51 is maintained. In certain embodiments, the disclosure provides a variant of hp4SD1-03 (or a variant of a fragment or fragment or chain thereof) comprising one or more amino acid substitutions, which may optionally comprise an amino acid substitution within the DDDY (SEQ ID NO: 418) motif of hVHH3-H4 at positions 101-104, which corresponds to the DEDY (SEQ ID NO: 410) motif of the VHH domain of hp4SD1-03 at positions 101-104, optionally wherein the D at position 102 is substituted with another amino acid, e.g., E, S, A, or T, or optionally wherein DEDY (SEQ ID NO: 410) is modified to DEEY (SEQ ID NO: 411), DESY (SEQ ID NO: 412), DEAY (SEQ ID NO: 413), DETY (SEQ ID NO: 414), ESEY (SEQ ID NO: 415), ESTY (SEQ ID NO: 416), or ESSY (SEQ ID NO: 417) (FIG. 14). The sequences of hp4SD1-03 are shown below.

Light chain:
(SEQ ID NO: 1)
EVQLVESGGGLVQPGGSLRLSCASS [symbol] LGWYRQAPGKQRELI
A [symbol] KYADSLKGRFTMSTDNSKNTMYLQMNSLRAEDTAVYYC [symbol]
RGQGTQVTVSS [symbol] [symbol] DIQMTQSPSSLSASVGDR
VTITCRASQSISSYLNWYQQKPGKAPKLLIYAASSLQSGVPSRFSGSG
SGTDFTLTISSLQPEDFATYYCQQSYSTPLTFGGGTKVEIKRTVAAPS
VFIFPPSDEQLKSGTASVVCLLNNFYPREAKVQWKVDNALQSGNSQES
VTEQDSKDSTYSLSSTLTLSKADYEKHKVYACEVTHQGLSSPVTKSFN
RGEC
Heavy chain:
(SEQ ID NO: 2)
EVQLVESGGGLVQPGGSLRLSCAASGFTFTSYAMSWVRQAPGKGLEWVS
AISGSGGSTYYAESVKGRFTISRDNSKNTLYLQMNSLRAEDTAVYYCAR
ATGFGTVVFDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALGAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

The VHH domain of hp4SD1-03 has the sequence:

(SEQ ID NO: 381)
EVQLVESGGGLVQPGGSLRLSCASSANIQSIETLGWYRQAPGKQRELIA
NMRGGGYMKYADSLKGRFTMSTDNSKNTMYLQMNSLRAEDTAVYYCYVK
LRDEDYVYRGQGTQVTVSS, with the CDRs shown under-
lined and in bold.

GGGGS (SEQ ID NO: 101) is the linker sequence between the VHH domain and VL domain.

The VL domain of hp4SD1-03 has the sequence:

(SEQ ID NO: 281)
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPLTFGG
GTKVEIK, with the CDRs shown underlined and in
bold.

The VH domain of hp4SD1-03 has the sequence:

(SEQ ID NO: 181)
EVQLVESGGGLVQPGGSLRLSCAASGFTFTSYAMSWVRQAPGKGLEWVSA
ISGSGGSTYYAESVKGRFTISRDNSKNTLYLQMNSLRAEDTAVYYCARAT
GFGTVVFDYWGQGTLVTVSS, with the CDRs shown under-
lined and in bold.

The Fc domain of hp4SD1-03 has the sequence:

(SEQ ID NO: 81)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

Further particularly encompassed are some Fc variants of hp4SD1-03, which comprise the same light chain as hp4SD1-03 (i.e., SEQ ID NO: 1) and a heavy chain which differs in the Fc region sequence from the heavy chain of hp4SD1-03 (hp4SD1 HC contains an Fc region comprising the LALAPG substitutions, SEQ ID NO: 81).

For example, the LALAPG substitution may be introduced to another Fc region reference sequence to result in SEQ ID NOS: 82-84.

In another example, “hp4SD1-03 AAQ” comprises the light chain of SEQ ID NO: 1 and a heavy chain which is similar to the heavy chain of hp4SD1-03 but comprises an Fc region comprising the IAHAHQ substitutions (also called AAQ substitutions herein) in addition to the LALAPG substitutions (Fc region of e.g., SEQ ID NOS: 471-474). Such a heavy chain may for example comprise the amino acids sequence of SEQ ID NO: 450.

In another example, “hp4SD1-03 AAA” comprises the light chain of SEQ ID NO: 1 and a heavy chain which is similar to the heavy chain of hp4SD1-03 but comprises an Fc region comprising the IAHAHA substitutions (also called AAA substitutions herein) in addition to the LALAPG substitutions (Fc region of e.g., SEQ ID NOS: 476-479). Such a heavy chain may for example comprise the amino acids sequence of SEQ ID NO: 451.

Other non-limiting examples of Fc variants of hp4SD1-03 include: (I) a bispecific antibody comprising the light chain of SEQ ID NO: 1 and the heavy chain of SEQ ID NO: 7, which comprises an Fc region comprising the LALA substitutions (such a Fc region sequence may be, e.g., SEQ ID NOS: 86-89); (II) a bispecific antibody comprising the light chain of SEQ ID NO: 1 and the heavy chain of SEQ ID NO: 8, which comprises an Fc region comprising the IAHAHQ substitutions but no LALAPG substitutions (such a Fc region sequence may be, e.g., SEQ ID NOS: 91-94); (III) a bispecific antibody comprising the light chain of SEQ ID NO: 1 and the heavy chain of SEQ ID NO: 9, which comprises an Fc region comprising the IAHAHA substitutions but no LALAPG substitutions (such a Fc region sequence may be, e.g., SEQ ID NOS: 96-99); (IV) a bispecific antibody comprising the light chain of SEQ ID NO: 1 and the heavy chain of SEQ ID NO: 452, which comprises an Fc region comprising the IAHAHQ substitutions in addition to LALA substitutions (such a Fc region sequence may be, e.g., SEQ ID NOS: 481-484); and (V) a bispecific antibody comprising the light chain of SEQ ID NO: 1 and the heavy chain of SEQ ID NO: 453, which comprises an Fc region comprising the IAHAHA substitutions in addition to LALA substitutions (such a Fc region sequence may be, e.g., SEQ ID NOS: 486-489).

Additional non-limiting Fc variants of hp4SD1-03 may include: (I) a hp4SD1-03 variant in which the Fc region is replaced with a Fc region comprising one or more amino acid substitutions at positions selected from positions 234, 235, 236, 237, 265, 297 and 329, according to EU numbering; (II) a hp4SD1-03 variant in which the Fc region is replaced with a Fc region comprising D265A and P329A substitutions, according to EU numbering (also referred to as “DAPA” herein); (III) a hp4SD1-03 variant in which the Fc region is replaced with a Fc region comprising N297A substitution, according to EU numbering; (IV) a hp4SD1-03 variant in which the Fc region is replaced with a Fc region comprising M252Y, S254T, and T256E, according to EU numbering (also referred to as “YTE” herein); and (V) a hp4SD1-03 variant in which the Fc region is replaced with a Fc region comprising M428L and N434S, according to EU numbering (also referred to as “LS” herein). Such substations may be made to any Fc region sequences including but not limited to Fc region reference sequence of SEQ ID NOS: 71-74.

Also particularly encompassed are variants of hp4SD1-03, in which: one or more of the anti-Fzd4 VH and/or VL and/or the anti-LRP5/6 VH sequences are replaced with any of the corresponding variants contained in APPENDIX Tables B, C, and E; one or more of the anti-Fzd4 VH CDRs and/or VL CDRs and/or the anti-LRP5/6 VH CDRs sequences are replaced with any of the corresponding variants contained in APPENDIX Tables A, C, and D or any of the corresponding variants contained in the anti-Fzd4 VH and/or VL and/or the anti-LRP5/6 VH sequences contained in APPENDIX Tables B, C, and E; one or more of the anti-Fzd4 VH FRs and/or VL FRs and/or the anti-LRP5/6 VH FRs sequences are replaced with any of the corresponding variants contained in APPENDIX Tables A, C, and D or any of the corresponding variants contained in the anti-Fzd4 VH and/or VL and/or the anti-LRP5/6 VH sequences contained in APPENDIX Tables B, C, and E; the linker (linker between the anti-LRP5/6 VH and the anti-Fzd4 VL) sequence is replaced with any of the corresponding variants contained in APPENDIX Table F; one or more of the CH1, hinge, CH2, CH3, and/or CL sequences are replaced with any of the corresponding variants contained in APPENDIX Table G; one or more of the Fc region sequences are replaced with any of the corresponding variants contained in APPENDIX Table H; and/or one or more of the heavy chain (HC) and/or light chain (LC) sequences are replaced with any of the corresponding variants contained in APPENDIX Tables I-J. In further embodiments, the disclosure provides a nucleic acid sequence encoding a Wnt agonist molecule described herein, or a polypeptide component thereof, e.g., encoding a polypeptide of SEQ ID NO:1 and/or a polypeptide of SEQ ID NO: 2, 7, 8, 9, 450, 451, 452, or 453, or a variant or fragment of either polypeptide. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail.

In other embodiments, the polynucleotides are expression cassettes comprising a promoter operatively linked to the coding sequences. In further embodiments, the polynucleotide(s) are present in an expression vector, e.g., a viral vector, comprising a polynucleotide comprising a nucleic acid sequence encoding a Wnt agonist polypeptide sequence described herein, or a variant or fragment thereof.

The present disclosure further contemplates a cell comprising an expression vector comprising a polynucleotide comprising a promoter operatively linked to a nucleic acid encoding a Wnt agonist polypeptide sequence, or variant or fragment thereof. In particular embodiments, the cell comprises an expression vector comprising a polynucleotide comprising a promoter operatively linked to a nucleic acid sequence encoding a Wnt agonist polypeptide. In certain embodiments, the host cell is a mammalian cell, an inset cell, or a yeast cell. A variety of cells are known in the art that may be used to express and produce mammalian polypeptides. In particular embodiments, the cell is a heterologous cell, an allogeneic cell, or an autologous cell obtained from a subject to be treated. In particular embodiments, the cell is a stem cell, e.g., an adipose-derived stem cell or a hematopoietic stem cell.

The disclosure also provides methods of producing a Wnt agonist or polypeptide sequence thereof disclosed herein, the method comprising culturing a cell (e.g., a host cell) comprising one or more expression vector(s) encoding the Wnt agonist polypeptide(s) under conditions suitable for expression of the Wnt agonist polypeptide(s). In certain embodiments, a polynucleotide encoding the heavy chain polypeptide is present in a first expression vector, and a polynucleotide encoding the light chain polypeptide is present in a second expression vector. In certain embodiments, the polynucleotide encoding the heavy chain polypeptide and the polynucleotide encoding the light chain polypeptide are present in the same expression vector. In certain embodiments, the Wnt agonist or polypeptide sequence thereof is secreted and/or purified from the host cells following expression.

### III. Pharmaceutical Compositions

Pharmaceutical compositions comprising a Wnt agonist molecule described herein and one or more pharmaceutically acceptable diluent, carrier, or excipient are also disclosed.

In further embodiments, pharmaceutical compositions comprising a polynucleotide comprising a nucleic acid sequence encoding a Wnt agonist molecule described herein and one or more pharmaceutically acceptable diluent, carrier, or excipient are also disclosed. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences.

In further embodiments, pharmaceutical compositions comprising an expression vector, e.g., a viral vector, comprising a polynucleotide comprising a nucleic acid sequence encoding a Wnt agonist molecule described herein and one or more pharmaceutically acceptable diluent, carrier, or excipient are also disclosed. In certain embodiments, the nucleic acid sequence encoding the Wnt antagonist molecule and the nucleic acid sequence encoding the Wnt agonist are in the same polynucleotide, e.g., expression cassette.

The present disclosure further contemplates a pharmaceutical composition comprising a cell comprising an expression vector comprising a polynucleotide comprising a promoter operatively linked to a nucleic acid encoding a Wnt antagonist/agonist molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a Wnt agonist. In particular embodiments, the cell is a heterologous cell or an autologous cell obtained from the subject to be treated. In particular embodiments, the cell is a stem cell, e.g., an adipose-derived stem cell or a hematopoietic stem cell.

The subject molecules, alone or in combination, can be combined with pharmaceutically acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerol, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; surfactants such as Tween 20® to prevent aggregation and/or absorption to materials; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the Wnt antagonist/agonist antibody or antigen-binding fragment thereof (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms may be dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g., a syringe, e.g., a prefilled syringe, together with instructions for administration.

The pharmaceutical compositions of the present disclosure encompass any pharmaceutically acceptable salts, esters, or salts of such esters, or any other compound which, upon administration to a mammal, is capable of providing (directly or indirectly) the biologically active antibody or antigen-binding fragment thereof.

The present disclosure includes pharmaceutically acceptable salts of a Wnt agonist molecule described herein. The term “pharmaceutically acceptable salt” refers to physiologically and pharmaceutically acceptable salts of the compounds of the present disclosure: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, PA, USA, 1985 (and more recent editions thereof), in the “Encyclopedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002). Pharmaceutically acceptable base addition salts are formed with metals or amines, such as alkali and alkaline earth metals or organic amines.

Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In some embodiments, the pharmaceutical composition provided herein comprise a therapeutically effective amount of a Wnt agonist molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone and polyethyleneglycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### IV. Methods of Use

The present disclosure also provides methods for using the Wnt agonist molecules, e.g., to modulate a Wnt signaling pathway, e.g., to increase Wnt signaling, and the administration of a Wnt agonist molecule in a variety of therapeutic settings. Provided herein are methods of treatment using a Wnt agonist molecule. In one embodiment, a Wnt agonist molecule is provided to a subject having a disease involving inappropriate or deregulated Wnt signaling.

In certain embodiments, a Wnt agonist molecule may be used to enhance a Wnt signaling pathway in a tissue or a cell. Agonizing the Wnt signaling pathway may include, for example, increasing Wnt signaling or enhancing Wnt signaling in a tissue or cell. Thus, in some aspects, the present disclosure provides a method for agonizing a Wnt signaling pathway in a cell, comprising contacting the tissue or cell with an effective amount of a Wnt agonist molecule or pharmaceutically acceptable salt thereof disclosed herein, wherein the Wnt agonist molecule is a Wnt signaling pathway agonist. In some embodiments, contacting occurs in vitro, ex vivo, or in vivo. In particular embodiments, the cell is a cultured cell, and the contacting occurs in vitro. In certain embodiments, such a method may be useful for inducing cell and/or tissue differentiation of interest. Therefore, such a method maybe for preparing a cell and/or tissue for implantation of transplantation (e.g., neural cells or tissues, which may for example be useful for treating a neural or neurodegenerative disease), differentiating stem cells (e.g., differentiating stem cells into neural progenitors or neurons), and/or preparing an organoid (e.g., neural organoid).

The Wnt agonist molecule may be used for the treatment of various diseases, disorders, and conditions, such as a retinopathy. In particular, activation of Wnt signaling is necessary for retinal vascularization during vessel development in eye. Genetic deletion of norrin, Fzd4, Lrp5, or Tspan12 significantly regresses not only vascular development on superficial retina surface, but also vascular penetration into deeper layers of retina. Additionally, the generated avascular area due to immature vascularization causes ischemia-induced neovascularization. Therefore, the timely controlled administrations of Wnt agonist or/and antagonist not only will regress retinopathy disease progression but also would also lead to an improvement of the illness. In the particular embodiments, Wnt agonist will be administered in either an earlier or later phase of retinopathy disease progression in the subjects.

Retinal vascular diseases that may be treated can include, but are not limited to: familiar exudative vitreoretinopathy (FEVR), exudative vitreoretinopathy, Norrie disease, diabetic retinopathy (DR), diabetic macular edema, diabetic macular ischemia, age-related macular degeneration (AMD) (including wet and dry AMD and dry AMD), retinopathy of prematurity (ROP), osteoporosis-pseudoglioma syndrome (OPPG), retinal vein occlusion, and Coats disease.

In particular embodiments, a retinal disease or retinopathy is treated with a Wnt signaling agonist, including but not limited to hp4SD1-03, hp4SD1-03 AAQ, or hp4SD1-03 AAA, or a Wnt signaling agonist comprising a fragment or variant of hp4SD1-03 or a polypeptide component thereof. In particular embodiments, fragments or variants are functional fragments or variants that retain Fzd and/or LRP5/6 binding activity and the capacity to induce Wnt signaling pathways in the context of the Wnt signaling agonist, e.g., in certain embodiments, at least 100%, at least 90%, at least 80%, at least 70%, or at least 60% of the Fzd and/or LRP5/6 binding activity and/or at least 100%, at least 90%, at least 80%, at least 70%, or at least 60% of the capacity to induce Wnt signaling pathways, as compared to the parental molecule, which may be, e.g., hp4SD1-03, hp4SD1-03 AAQ, or hp4SD1-03 AAA.

In particular embodiments, the Wnt signaling agonist has a structural format as diagrammed in FIG. 1 and/or comprises one or both of the sequences disclosed in FIG. 9 or FIG. 19A or 19B, or any functional fragment or variant thereof, e.g., an antigen-binding fragment thereof, such as a VHH domain, VL domain, or VH domain, or an Fc domain, or a variant of any of these functional domains, e.g., a variant having at least 90%, at least 95%, at least 98%, or at least 99% sequence identity to any functional fragment of antibody sequence disclosed herein. In some embodiments, the Wnt signaling agonist comprises a variant sequence having at least 90% identity (e.g., 95%, 98% or 100% identity) to a sequence disclosed in FIG. 9 or FIG. 19A or 19B or set forth in SEQ ID NOs: 1 and/or 2, SEQ ID NOs: 1 and/or 7, SEQ ID NOs: 1 and/or 8, SEQ ID NOs: 1 and/or 9, SEQ ID NOs: 1 and/or 450, SEQ ID NOs: 1 and/or 451, SEQ ID NOs: 1 and/or 452, and/or SEQ ID NOs: 1 and/or 453 or any functional fragment thereof, e.g., an antigen-binding fragment thereof. In some embodiments, the Wnt signaling agonist comprises a wild type or variant sequence having at least 90% identity (e.g., 95%, 98% or 100% identity) to a light chain sequence disclosed in FIG. 9 or set forth in SEQ ID NO: 1, and a wild type or variant sequence having at least 90% identity (e.g., 95%, 98% or 100% identity) to a heavy chain sequence disclosed in FIG. 9 or FIG. 19A or 19B or set forth in any one of SEQ ID NOS:2, 7-9, and 450-453. In some embodiments, the Wnt signaling agonist comprises two wild type or variant sequences, each having at least 90% identity (e.g., 95%, 98% or 100% identity) to a light chain sequence disclosed in FIG. 9 or FIG. 19A or 19B or set forth in SEQ ID NO: 1, and two wild type or variant sequences, each having at least 90% identity (e.g., 95%, 98% or 100% identity) to a heavy chain sequence disclosed in FIG. 9 or FIG. 19A or 19B or set forth in any one of SEQ ID NO: 2, 7-9, and 450-453, wherein the two heavy chains are bound to each other and each light chain is bound to a different heavy chain, e.g., by disulfide bonds. In particular embodiments, amino acid modifications may be introduced into the parental molecule, 4SD1-03_LALPG to generate hp4SD1-03 or its variants, including, e.g., those in hVHH3-H4 (FIG. 2), hVHH3-H4 N29Q, hVHH3-H4 DDD substituted with DED (FIG. 4), hVHH3-H4 DDDY (SEQ ID NO: 418) (amino acids 101-104) substituted with DEEY (SEQ ID NO: 411), DESY (SEQ ID NO: 412), DEAY (SEQ ID NO: 413), DETY (SEQ ID NO: 414), ESEY (SEQ ID NO: 415), ESTY (SEQ ID NO: 416), or ESSY (SEQ ID NO: 417) (FIG. 14), 4SD1 HC CDR1 N31S, and 4SD1 HC CDR2 D62E (FIG. 7), and/or those in the humanized 4DS1 framework, as shown in FIG. 8. In certain embodiments, CDR3 D108 is maintained. In certain embodiments, VHH3 M51 is maintained.

In particular embodiments, the methods of treatment disclosed herein result in one or more of: decreased retinal vascular leakage, decreased retinal ischemia, or decreased retinal neovascular lesion size. In particular embodiments, the methods of treatment disclosed herein result in improvement in one or more symptom or pathology of any of the diseases or disorders disclosed herein.

The present invention also provides for combination treatment with known treatments for FEVR and/or DR. For example, the Wnt agonist can be administered in combination with another therapy for a retinopathy, including, but not limited to, anti-VEGF agent or VEGF inhibitors or VEGF antagonists, such as an anti-VEGF antibody. In some embodiments, anti-Ang2 antibody will also be administered to subjects in combination with the Wnt agonist. Hypoxia-induced VEGF and Ang2 expression are important cues for pathological neovascularization, and indeed, an antagonist Ang2 antibody has been considered for retinopathy patient treatment (Gadkar et al., Invest Ophthalmol Vis Sci. 2015 August; 56(9):5390-400). The anti-VEGF antibody or anti-Ang2 antibody can be administered sequentially or concurrently with the molecules of the present invention. VEGF antagonists can include, but are not limited to: bevacizumab, ranibizumab, aflibercept, ramucirumab, and tanibirumab; and Ang2 antagonists can include but are not limited to: nesvacumab, AMG780, and MEDI3617.

In a further embodiment, the agonist molecule may also incorporate a tissue targeting moiety, e.g., an antibody or fragment thereof that recognizes a retinal tissue specific receptor or cell surface molecule.

The therapeutic agent (e.g., a Wnt agonist) may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. In some embodiments, the subject method results in a therapeutic benefit, e.g., preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified and will recognize the appropriate detection methods to use to measure therapeutic efficacy.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the present disclosure have been described herein for purposes of illustration, various modifications may be made without deviating from the spirit and scope of the present disclosure. Accordingly, the present disclosure is not limited except as by the appended claims.

The broad scope of this invention is best understood with reference to the following example, which is not intended to limit the inventions to the specific embodiments.

### EXAMPLES

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd}ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, and glycosylation sites, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### Example 1

### Production and Characterization of Novel Wnt Signaling Agonist

A humanized and optimized Wnt signaling agonist exhibiting enhanced activity was developed and characterized as described.

### Protein Production

All recombinant proteins were produced in Expi293F™ cells (Thermo Fisher Scientific) by transient transfection. The proteins were first purified using a MabSelect SuRe column (often referred herein as Protein A) then further polished with Superdex 200 Increase 10/300 GL (GE Healthcare Life Sciences) size-exclusion chromatography (SEC) using 1×HBS buffer (20 mM HEPES pH 7.4, 150 mM NaCl) or HiLoad 16/600 Superdex 200 using 2×HBS buffer. After that, the proteins were examined by SDS-polyacrylamide gel electrophoresis and estimated to be >90% purity.

### SuperTop Flash (STF) Assay (Wnt Signaling Assay)

Wnt signaling activity was measured using bEND3 cells containing a luciferase gene controlled by a Wnt-responsive promoter (Super Top Flash reporter assay, STF) as previously reported (Chen et al., STAR Protoc. 2020 Jun. 3; 1(1):100043.). In brief, cells were seeded at a density of 10,000 per well in 96-well plates 24 hours prior to treatment in the presence of 3 μM IWP2 to inhibit the production of endogenous Wnt ligands. Varying concentrations of the recombinant proteins were then added to the cells overnight. Cells were lysed with Luciferase Cell Culture Lysis Reagent (Promega) and luciferase activity was measured with Luciferase Assay System (Promega) using the vendor suggested procedures.

### Example 2

### Oxygen-Induced Retinopathy (OIR) Mouse Study

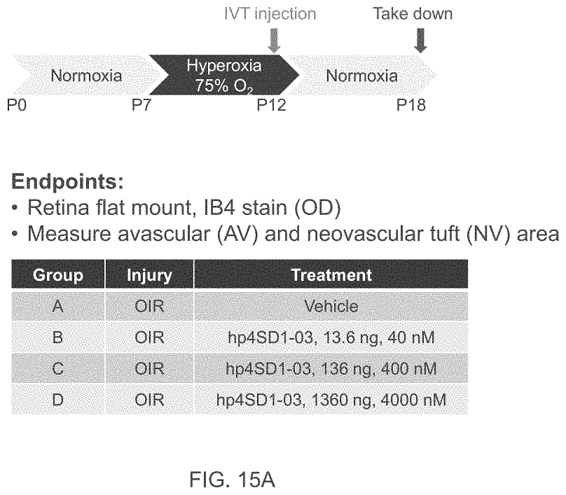

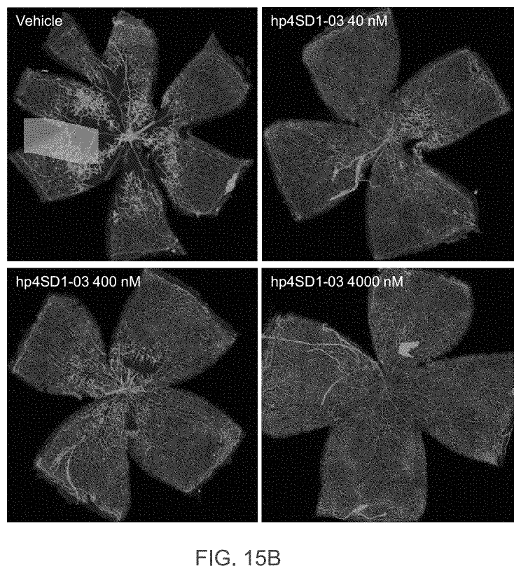

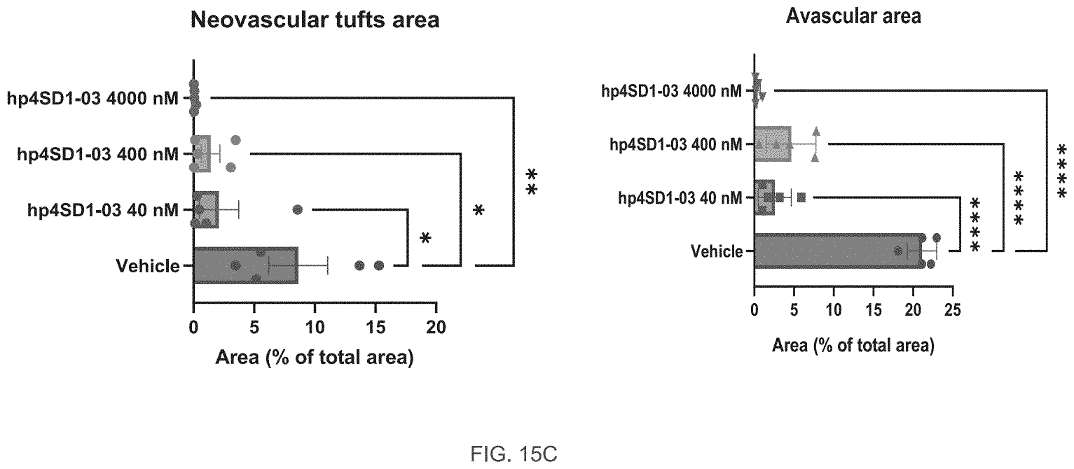

Mouse model of oxygen-induced retinopathy (OIR) was carried out similarly to previously described (Smith et al., Oxygen-induced retinopathy in the mouse, Investigative Ophthalmology & Visual Science January 1994, Vol. 35, 101-111) with some experimental changes. Briefly, newborn pups at postnatal day 7 (P7) were exposed to a hyperoxic environment with 75% inspired oxygen level for 5 days. As a result, pups developed avascular (AV) retina from vaso-obliteration of newly formed capillaries in the center retina from P7 to P12. After pups were moved to room air at P12, avascular central retina became hypoxic and intravitreal neovascular (NV) tufts were formed at the junctions of vascular and avascular retina from P12 to P17. For treatment, P12 pups received an intravitreal injection of one of the following test articles: Eylea® (aflibercept, a decoy VEGF receptor) at 60 μg/eye, 4SD1-03_LALAPG at 68 ng/eye, hp4SD1-03 at 13.6 ng/eye, or vehicle control. For evaluating the efficacy of test articles on reducing AV and NV areas, whole mount retinas from pups at P17 were stained with isolectin B4 and imaged. AV, NV, and total retinal areas were manually scored and presented as percentage of either AV area or NV area over total retinal area. To evaluate dose-efficacy relationship, a second OIR study was performed as described above (FIG. 15A-15C) with doses 13.6 ng, 136 ng, 1360 ng per eye of hp4SD1-03 or vehicle control injected. For evaluating the efficacy of test articles on reducing AV and NV areas, whole mount retinas from pups at P17 were stained with isolectin B4 and imaged. AV, NV, and total retinal areas were manually scored and presented as a percentage of either AV or NV area over total retinal area. Statistical significance between each treatment group and the vehicle control group was determined using a one-way ANOVA test followed by Tukey's multiple comparison test. hp4SD1-03 from 40 nM to 4000 nM was efficacious in reducing NV tufts and AV area in OIR model.

All tested doses of hp4SD1-03 showed significant reductions of both pathologic NV and retinal AV areas to a larger degree than the vehicle-treated group (FIGS. 15B and 15C). These results suggest that hp4SD1-03, not only potently inhibits pathologic NV tuft formation in retinal ischemic conditions, but also potentially promotes proper vessel regeneration to re-perfuse the damaged retina.

### Example 3

### VEGF-Induced Retinal Vascular Leakage Rabbit Study

Rabbit model of VEGF-induced retinal vascular leakage was carried out similarly to previously described (Arana, L. A., et al. Fluorescein angiography, optical coherence tomography, and histopathologic findings in a VEGF animal model of retinal angiogenesis. Graefes Arch Clin Exp Ophthalmol 250, 1421-1428 (2012)) with some experimental changes. On day 0, rabbit eyes were dosed intravitreally with the test articles followed by the 1.0 μg of recombinant human VEGF165 (R&D Systems) for retinal leakage. Fluorescein angiography (FA) was done on both eyes at day0_pre-VEGF, day3 and day5. Early-phase retinal photography was performed 1-2 min after intravenous sodium fluorescein injection (12 mg kg-1). Animals were imaged using the RetCam3 (Natus) imaging system. Test articles and collected images were assessed (masked) and scored for retinal leakage as following: Score Retinal Leakage 0: major blood vessels appear very straight with limited tortuosity of smaller vessels; 1: major blood vessels present increased tortuosity and/or vessel dilation; 2: leakage is present between major vessels, no leakage between minor vessels; 3: leakage is present between major and minor vessels; and 4: leakage is present between major and minor vessels, and minor vessels are not visible.

### Example 4

### VHH3 Humanization

In order to humanize the camelid antibody (VHH3) sequence, the sequence database of human germline IgG heavy chain (HC) was searched. 3-66*01 was found as the closest match. Four different mutants (H1-H4) were generated and purified. The “hVHH3-H4” which showed the best yield of production was picked as a humanized VHH3 (FIG. 3).

### Example 5

### Polishing VHH3

To de-risk potential liabilities of deamidation, isomerization, and oxidation in VHH3 CDR regions, VHH3 mutants were generated and tested as below (FIGS. 3-5).

### N290 Mutation at CDR1 (FIG. 3)

Three different N29 mutants (N29Q, N29A, N29S) of hVHH3-H4 were generated and purified. For example, N29Q VHH3 showed a good yield for high purity expression. The asparagine in CDR1 was replaced to glutamine to mitigate the potential liability of deamidation.

### D102E Mutation at CDR3 (FIG. 4)

Five different mutants (DED, SDE, EDE, ESD, SSD) in hVHH3-H4 CDR3 were generated and purified to de-risk “DD” motif isomerization in VHH3. All mutants showed good STF response and expression. The DED mutant which may have minimal structural change was selected. The aspartic acid in CDR3 was replaced to glutamic acid to mitigate the potential liability of isomerization.

### D103 Mutation at CDR3 (FIG. 14)

In hp4SD1-03, various amino acid substitutions were made within amino acids 101-104 of CDR3 of the VHH domain, including the following: DEEY (SEQ ID NO: 411), DESY (SEQ ID NO: 412), DEAY (SEQ ID NO: 413), DETY (SEQ ID NO: 414), ESEY (SEQ ID NO: 415), ESTY (SEQ ID NO: 416), and ESSY (SEQ ID NO: 417). These mutants showed good expression and stability. The STF response magnitude (Emax) was increased with all the tested mutants. In some mutants, the EC50 value was reduced (e.g., ESSY (SEQ ID NO: 417)).

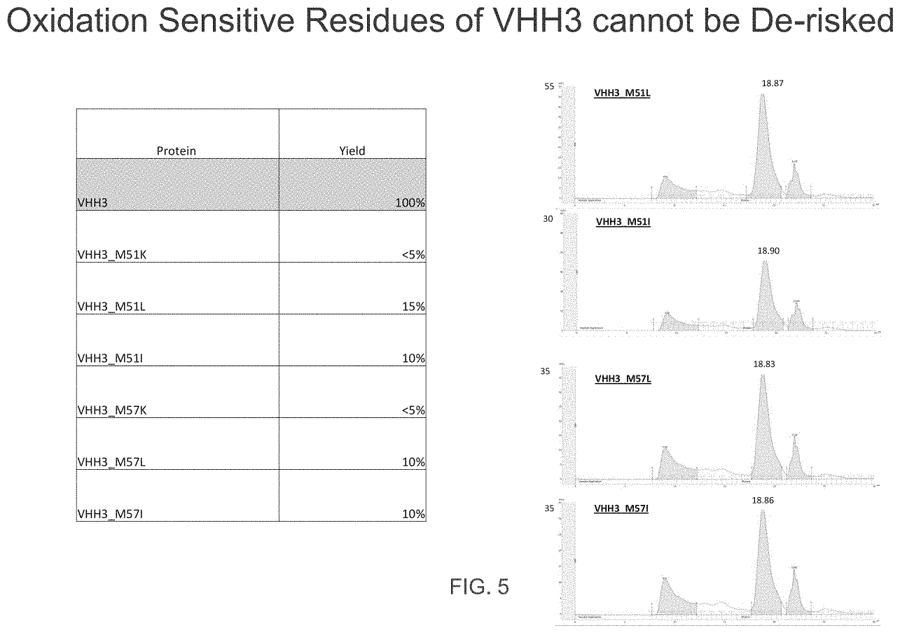

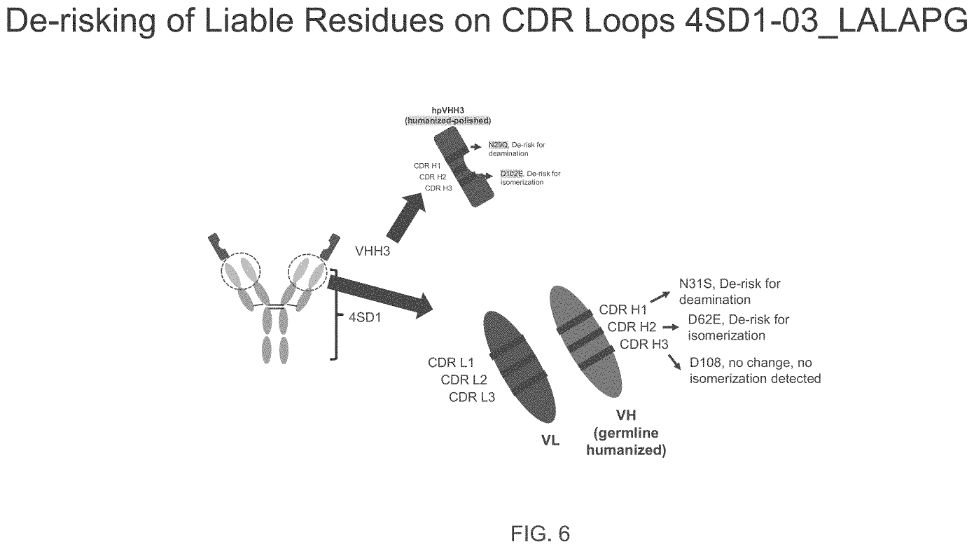

### No Changes of M51 and M57 in VHH3 CDR2 (FIG. 5)

Three different mutants at each methionine residue in VHH3 CDR2 (M51K, M51L, M51I. M57K, M57L, M57I) were generated as VHHs and purified to de-risk potential oxidation liability. All mutants showed a large decrease of expression and it was decided to keep both methionine residues. Oxidation was tested by Mass-spec analyses, which indicated no oxidation (data not shown).

### Polishing 4SD1 IgG LALAPG

To de-risk potential liabilities of deamidation, isomerization, and oxidation in 4SD1 variable domains, HC mutants were generated and tested as below. There was no amino acid for potential liabilities in light chain CDR regions.

### N31S Mutation at 4SD1 HC CDR1 (FIG. 7)

Bispecific antibodies comprising 4SD1 LC and one of three different N31 mutants (N31Q, N31A, N31S) of 4SD1 HC were generated and purified. All mutants showed no decrease of activity in STF assay, compared with their parental form. N31S and N31A mutants even showed potency improvement in the STF assay, and N31S was picked for the polished molecule.

### D62E Mutation at 4SD1 HC CDR2 (FIG. 7)

Bispecific antibodies comprising 4SD1 LC and one of three different D62 mutants (D62E, D62A, D62S) of 4SD1 HC were generated and purified. All mutants showed comparable or improved STF activity (e.g., increased response magnitude) relative to their parental form. D62E was picked for a polished molecule due to the amino acid similarity between D and E.

### No Changes of D108 at 4SD1 HC CDR3 (FIG. 7)

Bispecific antibodies comprising 4SD1 LC and one of ten different D108 mutants (D108E, D108A, D108S, D108R, D108T, D108L, D108H, D108Y, D108Q, D108K) of 4SD1 HC were generated and purified. All mutants showed significant potency loss in the STF activity, compared to their parental form. The decision was made that D108 should be kept unchanged and the isomerization liability evaluated by mass-spec analyses.

### 4SD1 Framework Humanization (FIG. 8)

4SD1 antibody sequence was obtained from a synthetic human antibody library containing some mutations which mismatch with human germline sequences. The mutations in the framework were corrected based on the human IgG germline sequence as indicated in FIG. 8. hp4SD1-03, which incorporates the above sequence modifications as shown in FIG. 9, showed improved STF activity, i.e., reduced EC50 and increased Emax, relative to the parent, 4SD1-03_LALAPG.

### Example 6

Biological Activity of hp4SD1-03

### Wnt Signaling Activity

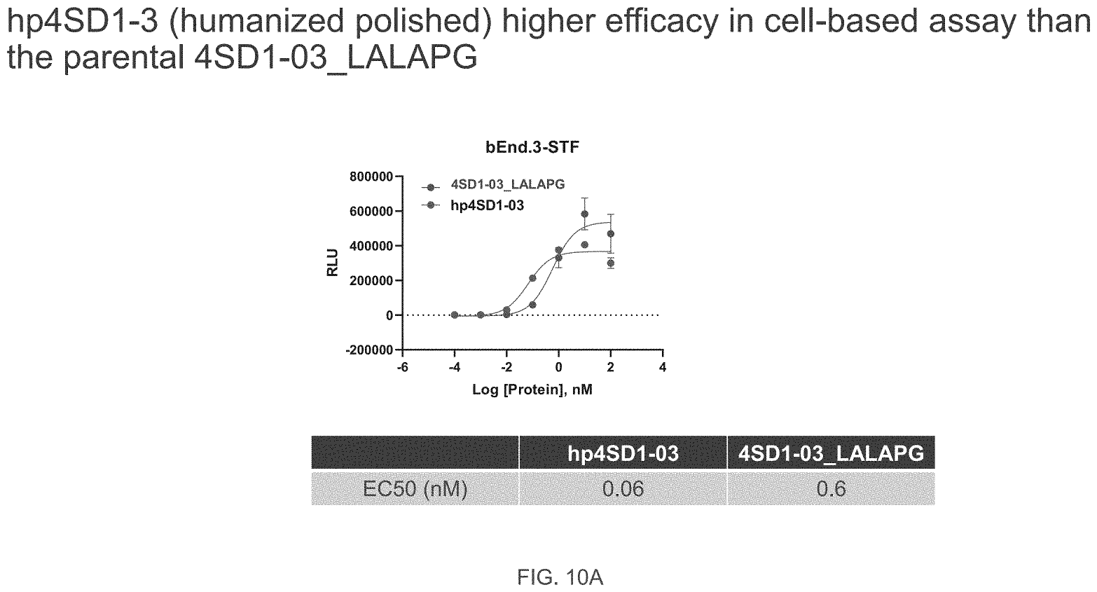

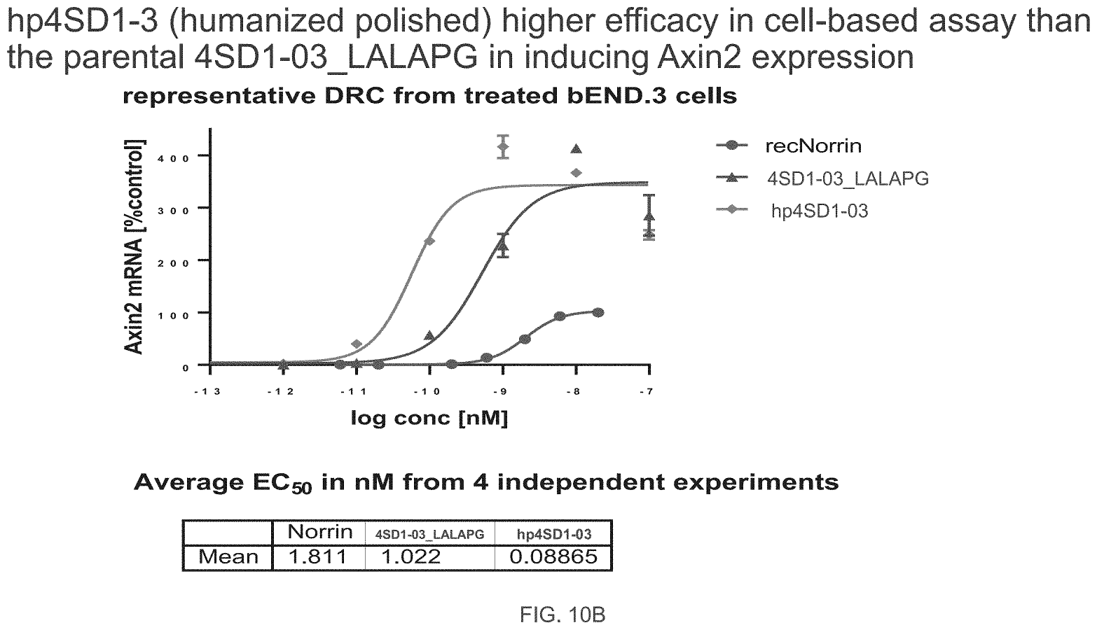

The final humanized and polished 4SD1-03_LALAPG, hp4SD1-03 (FIG. 9), showed about a 10-fold potency increase (as evaluated based on reduction in EC50) than its parental form 4SD1-03_LALAPG in STF assay as performed in Example 1 (one exemplary side-by-side dose-response comparison shown in FIG. 10A). More specifically, the average EC50±SEM for hp4SD1-03 from seven independent experiments was 0.055±0.014 nM, and the average EC50±SEM for 4SD1-03_LALAPG from ten independent experiments was 0.477±0.069 nM.

Similarly, when another assay for measuring Wnt/β-catenin signaling similar to the STF assay but using bEND3 cells not containing an exogenous reporter gene controlled by a Wnt-responsive promoter (i.e., using a less artificial setting) was performed (method details described below), a reduction in EC50 by 11.5 fold without losing response magnitude (Emax) was observed with hp4SD1-03 relative to 4SD1-03_LALAPG (FIG. 10B).

Materials and Methods

Wnt/β-catenin signaling activity was measured in mouse brain microvascular endothelial cells (bEnd.3 cells) by quantifying the amount of the gene expression of Axin2, a gene induced by active Wnt signaling and acts in a negative feedback loop (Yan, PNAS, 98(26):14973-14978, (2001); Lustig, Mol. Cell. Biol. 22(4): 1184-1193 (2002); Jho, Mol. Cell. Biol. 22(4): 1172-1183 (2002)).

Cells were seeded at a density of 500000 cells per well in 6-well plates and 24 hours prior to treatment starved in DMEM with 0.5% BSA. 4SD1-03_LALAPG or hp4SD1-03 protein was then added to the cells for 24 hours. Starvation was performed to eliminate unspecific signaling effects of FCS. Recombinant human Norrin (R&D Systems, Lot NS00717041, 3014-NR-025) was used as a response comparator. Cells were lysed and RNA was extracted with 350 μl RLT Buffer exactly following the manufacturer protocol (Qiagen RNeasy 96 Kit, catalog number 74181, lot 172036621). cDNA was synthesized and Axin 2 cDNA was amplified according to manufacturer procedures (Applied Biosystems High Capacity cDNA Reverse Transcription Kit catalog number 4368813, lot 01216654; Applied Biosystems TaqMan Universal PCR Master Mix catalog number 4304437, lot 2208187; Axin2 Primer: Mm00443610_m1Applied Biosystems).

Dose-response curves (DRC) were generated using GraphPad Prism 9.3.1; for normalization the maximal response of recombinant Norrin was set to 100%.

### Mouse Retinal Injury Model

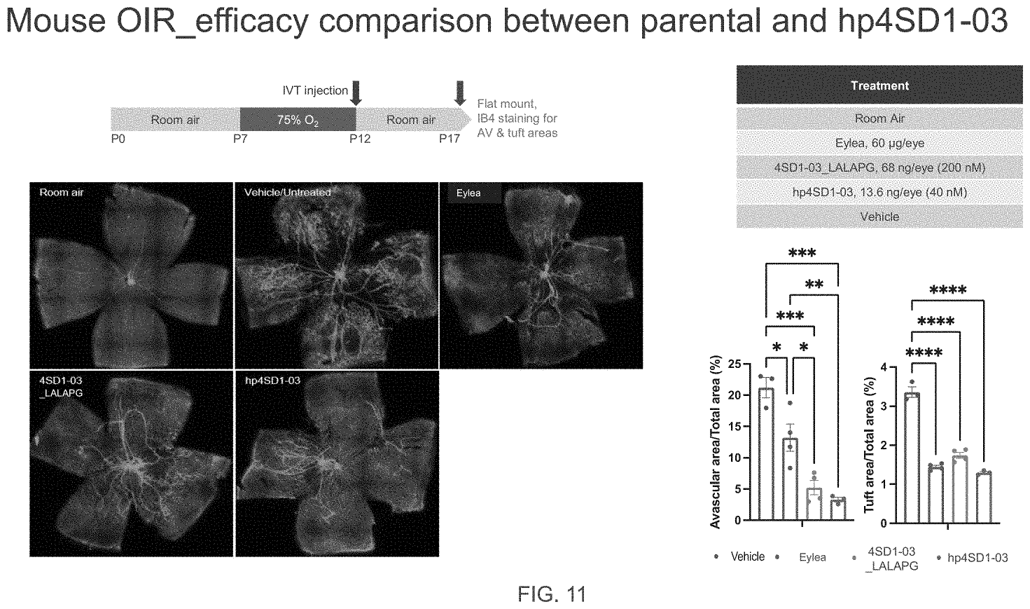

As shown in FIG. 11, treatment with the modified construct, hp4SD1-03, as compared to the parental construct, 4SD1-03_LALAPG, in the mouse model of retinal injury demonstrated a better outcome with hp4SD1-03 as compared to 4SD1-03_LALAPG.

### Rabbit Retinal Vascular Leakage Model

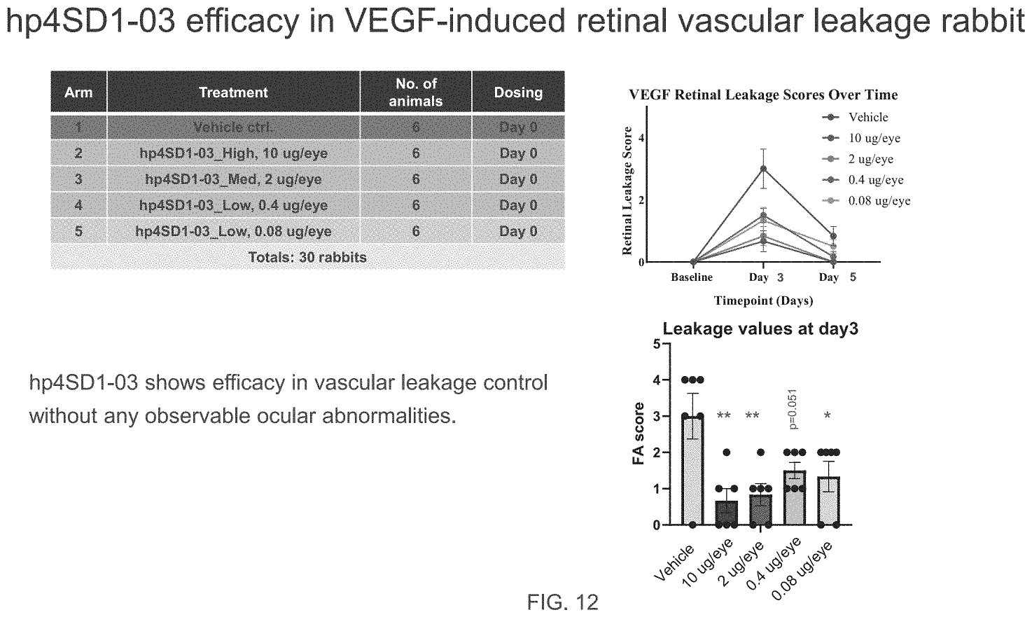

As shown in FIG. 12, treatment with the modified construct, hp4SD1-03, in the VEGF-induced retinal vascular leakage rabbit model of retinal injury reduced vascular leakage. Treatment with 2 ug/eye of hp4SD1-03 resulted in a 44% reduction in leakage, and treatment with 0.4 ug/eye of hp4SD1-03 also significantly reduced vascular leakage.

### Example 7

### Western Blot Analysis—4SD1-03 LALAPG

Western Blot analysis was performed as previously reported, with minor modifications. In brief, human retinal microvascular endothelial Cells (HRMEC) were treated with 10 nM 4SD1-03_LALAPG, 24 hrs before harvesting for cell lysis. The anti-ZO-1 (Abcam: ab216880), anti-CLDN5 (Abcam; ab131259), and anti-β actin (Sigma-Aldrich: A5441) primary antibodies were used, followed by horseradish peroxidase (HRP)-conjugated anti-rabbit or anti-mouse secondary antibodies for chemiluminescent signals.

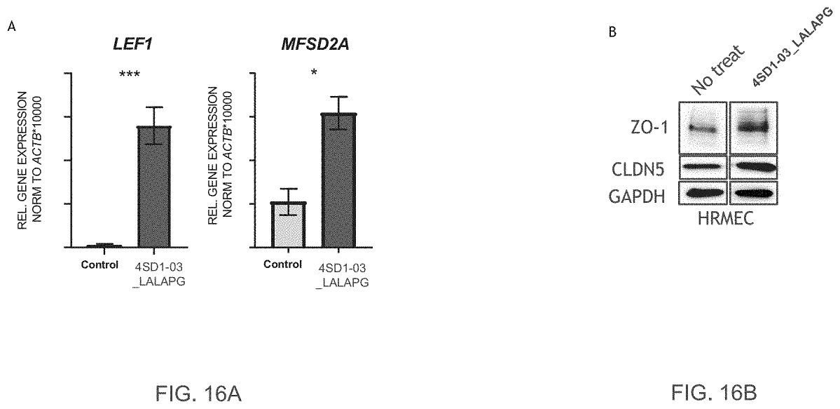

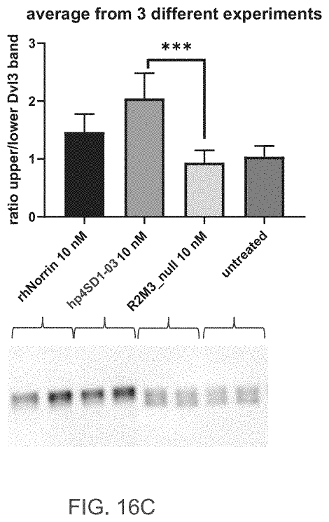

Accumulation of lymphoid enhancer binding factor 1 (Lef1) and Mfsd2a have been observed in the blood-brain barrier (BBB) vessels with specialized endothelial cell (EC) tight junctions, which are under Wnt/β-catenin signaling regulation (see, e.g., Wang Z et al. (2020) Sci Adv 2020; 6:eaba7457; Wang Y et al (2019) Elife 8; and Ben-Zvi A, et al. (2014) Nature 509:507-511). Expression of LEF1 and MFSD2A mRNA were significantly upregulated by 4SD1-03_LALAPG treatment in HRMEC (FIG. 16A). Furthermore, expression of EC tight junction proteins, CLDN5 and ZO-1, were upregulated in the retinal vascular ECs post 4SD1-03_LALAPG treatment (FIG. 16B). These results suggest the bioengineered tetravalent antibody targeting Fzd4 and LRP5 is active in vascular ECs and can increase vascular integrity through upregulation of barrier function proteins.

Western Blot Analysis—hp4SD1-03

Similarly, treatment of HRMEC with hp4SD1-03 at 10 nM (material and method details below) was confirmed to induce Wnt/β-catenin signaling as measured by increased phosphorylation of Dishevelled Segment Polarity Protein 3 (DVL3), which is a key component of Wnt signaling and becomes phosphorylated upon activation of the Wnt signaling pathway (Gonzalez-Sancho et al., Mol Cell Biol. 2004 June; 24(11):4757-68.). (FIG. 16C).

Materials and Methods

HRMEC cells were seeded at a density of 500000 cells per well in 6-well plates and 24 hours prior to treatment starved in DMEM with 0.5% BSA. Cells were added with recombinant Norrin, R2M3_null control, or hp4SD1-03 protein and incubated for 1 hour. Recombinant human Norrin (R&D Systems, Lot NSO0717041, 3014-NR-025) was used as response comparator. Cells were solubilized with 300 μl Cell Lysis Buffer (Cell Signaling 9803S) with Complete (Roche 11873580001) or Pefabloc SC (Roche 505648900) for 30 min at 4° C., extracts were centrifuged for 15 min at 14000 g at 4° C., and supernatant digested with 3 μl DNAse I (Qiagen 79254). Lysates were boiled in SDS sample buffer separated by SDS-polyacrylamide gel electrophoresis, transferred onto nitrocellulose membrane and subjected to Western blot analyses using indicated antibodies (NuPage7% Tris Acetate Gel, Invitrogen EA03585BOX according to manufacturer's protocol, iBlot® NC Transfer Stacks Invitrogen IB 23001, rabbit anti Dvl3 antibody Cell Signaling 3218, Anti-Rabbit IgG (H+L), HRP Conjugate Promega, W4011). Protein levels were quantified by measuring the band intensities using Fuji LumiImager LAS 300, AIDA Software (Advanced Data Image Analyzer Version 4.15.025). Intensity ratios between the upper band (corresponding to phosphorylated DVL3) and the lower band (corresponding to unphosphorylated DVL3) were calculated.

### Example 8

### Binding Kinetics by BLI

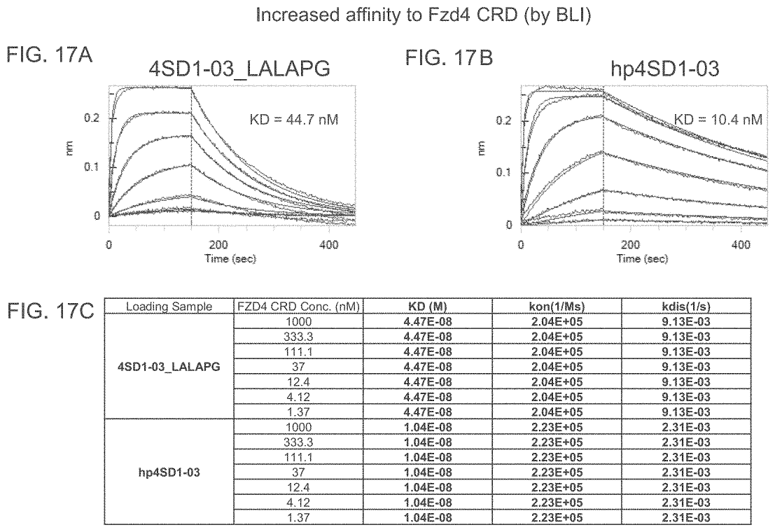

An increase in binding affinity measured by bio-layer interferometry (BLI) (material and method details below) for hp4SD1-03 to the CRD of human Fzd4 relative to 4SD1-03_LALAPG was observed (FIGS. 17A-17C). In all tested concentrations, the sensorgrams of human Fzd4 CRD with hp4SD1-03 showed slower dissociations than with 4SD1-03_LALAPG (FIG. 17C). The kinetic analyses in the two graphs (FIGS. 17A-17B) also clearly show that the slowed k_{off} (k_{dis}) majorly impacted the reduced K_{D} value of hp4SD1-03. Without wishing to be bound by theory, the slowed k_{off} and the reduced K_{D} may at least partially explain the reduced EC50 values observed in the STF and Wnt/β-catenin signaling measurement assays described above.

Materials and Methods

Kinetics of monovalent binding by 4SD1-03_LALAPG or hp4SD1-03 to the cysteine-rich domain (CRD) of human Fzd4 was determined by BLI using an OCTET® Red 96 (PALL ForteBio, Fremont, CA) instrument at 30° C., 1000 rpm with AHC biosensors (Sartorius). 4SD1-03_LALAPG or hp4SD1-03 was diluted to 50 nM in the running buffer (PBS, 0.05% Tween-20@, 0.5% BSA, pH 7.2) and captured to the AHC biosensor, followed by dipping into wells containing the CRD of human Fzd4 at different concentrations (1000, 333.3, 111.1, 37, 12.4, 4.12, and 1.37 nM) in a running buffer or into a well with only the running buffer as a reference channel. The dissociation of the interaction was followed with the running buffer. The monovalent K_{D} for each binder was calculated by OCTET® System software, based on fitting to a 1:1 binding model.

### Binding Kinetics by SPR

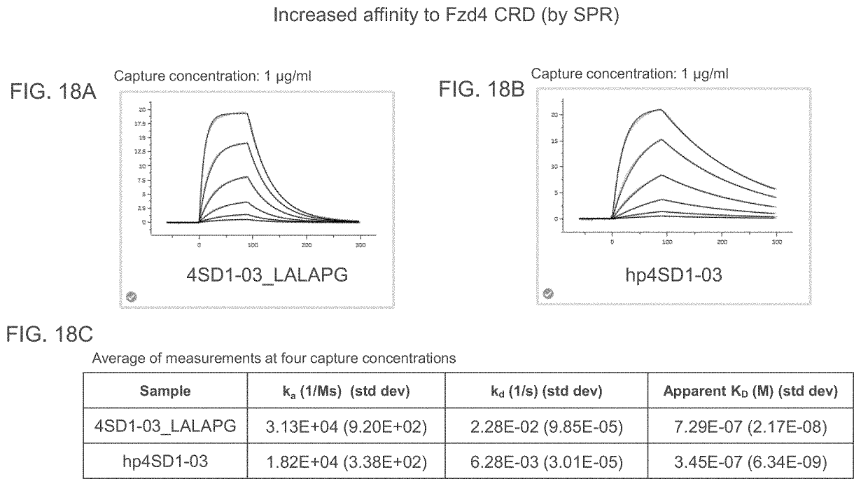

An increase in binding affinity measured by surface plasmon resonance (SPR) (material and method details below) for hp4SD1-03 to the CRD of human Fzd4 relative to 4SD1-03_LALAPG was observed (FIGS. 18A-18C). As shown in the sensorgrams (FIGS. 18A-18B) and the kinetic values (FIG. 18C), hp4SD1-03 showed slower dissociation and increased affinity (reduced K_{D} values) than with 4SD1-03_LALAPG. Again, without wishing to be bound by theory, the slowed k_{off} and the reduced K_{D} may at least partially explain the reduced EC50 values observed in the STF and Wnt/β-catenin signaling measurement assays described above.

Materials and Methods

Binding of hp4SD1-03 or 4SD1-03_LALAPG to human Fzd4 CRD (TPP-48723, PPB-51327) were determined by SPR using a Biacore® 8K instrument using HBS-EP+(Biacore #BR-100668) as the running buffer. Following manufacturer's recommendations, a CM5 biosensor chip (Biacore #BR100530) was activated with a 50:50 solution of EDC/NHS (amine coupling kit Biacore #BR-100633). Protein AG at 10 μg/ml in 10 mM acetate pH 4.5 was immobilized on the surface followed by deactivation with 1M ethanolamine HCl.

hp4SD1-03 or 4SD1-03_LALAPG was captured at four concentrations (4, 2, 1, and 0.5 μg/ml in HBS-EP+) for 30 seconds at a flowrate of 10 μl/min. Serial dilutions of human Fzd CRD were prepared in HBS-EP+(2.5 μM-10.29 nM) and injected for 90 seconds association at 30 μl/min followed by a dissociation of 200 seconds. The surface was regenerated by injecting 0.85% phosphoric acid for 30 sec at a flowrate of 30 μl/min.

The analyte interaction with sensor surface (flow cell 1) and blank (HBS-EP+ or 0 nM analyte) were subtracted from the raw data. Sensorgrams were then fit using 1:1 binding model to provide kinetic constants (kₐ and k_{d}) and affinity (K_{D}) values. The results from the different capture concentrations were averaged, and standard deviations were calculated in excel to obtain the values reported here. Since the highest concentration of analyte used was 2.5 μM, K_{D} values above 250 nM are apparent K_{D}s.

### Example 9

Designing Fc Mutants of hp4SD1-03

In an effort to generate variants of hp4SD1-03 which may have shorter in vivo circulation time, the Fc region of hp4SD1-03 (which already has L234A, L235A, and P329G (also referred to as LALAPG herein)) was modified to further have the following amino acid substitutions: I253A, H310A, and H435Q (also referred to as IAHAHQ or AAQ herein); or I253A, H310A, and H435A (also referred to as IAHAHA or AAA herein). The hp4SD1-03 variant which additionally comprises IAHAHQ was named hp4SD1-03 AAQ, and the hp4SD1-03 variant which additionally comprises IAHAHA was named hp4SD1-03 AAA. No changes were made to the light chain of hp4SD1-03. Amino acid sequences of the heavy and light chains of hp4SD1-03 AAQ and hp4SD1-03 AAA are shown in FIGS. 19A and 19B, respectively. Without wishing to be bound by theory, reduced in vivo circulation time (half-life, T_{1/2}) may provide reduced toxicity.

Production of hp4SD1-03 AAQ and hp4SD1-03 AAA

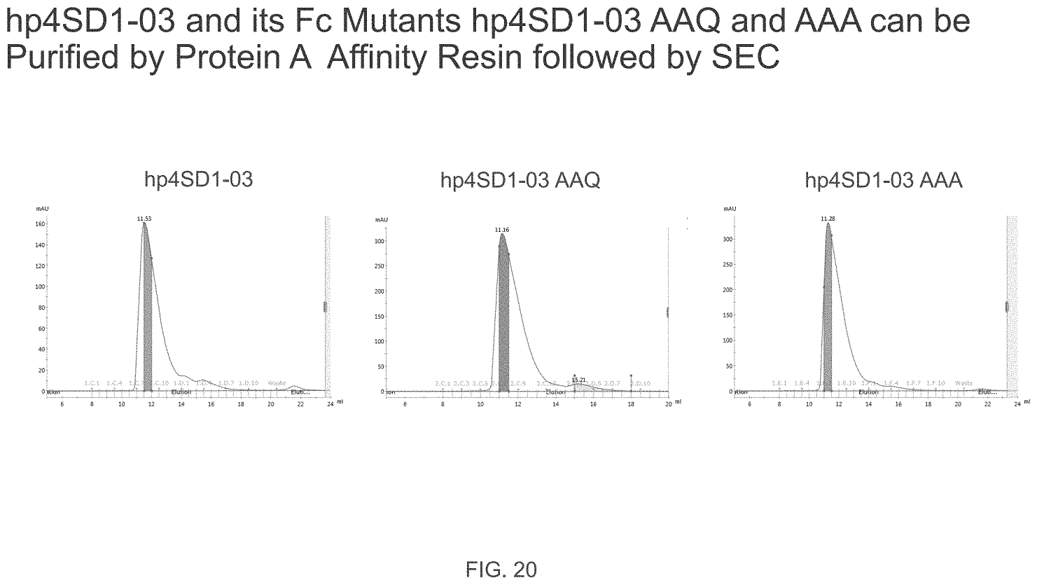

Both hp4SD1-03 AAQ and hp4SD1-03 AAA were successfully purified using Protein A affinity resin (FIG. 20).

Materials and Methods

The recombinant proteins were produced in Expi293F™ cells (Thermo Fisher Scientific) by transient transfection. The proteins were captured by Protein A affinity chromatography and eluted under acidic conditions, then polished and buffer exchanged by Superdex 200 Increase 10/300 GL (Cytiva) size-exclusion chromatography (SEC) using HBS buffer. Purified proteins were examined by SDS-polyacrylamide electrophoresis, based on which the proteins were estimated to have >90% purity. SEC chromatograms are shown in FIG. 20.

Protein Stability of hp4SD1-03 AAQ and hp4SD1-03 AAA

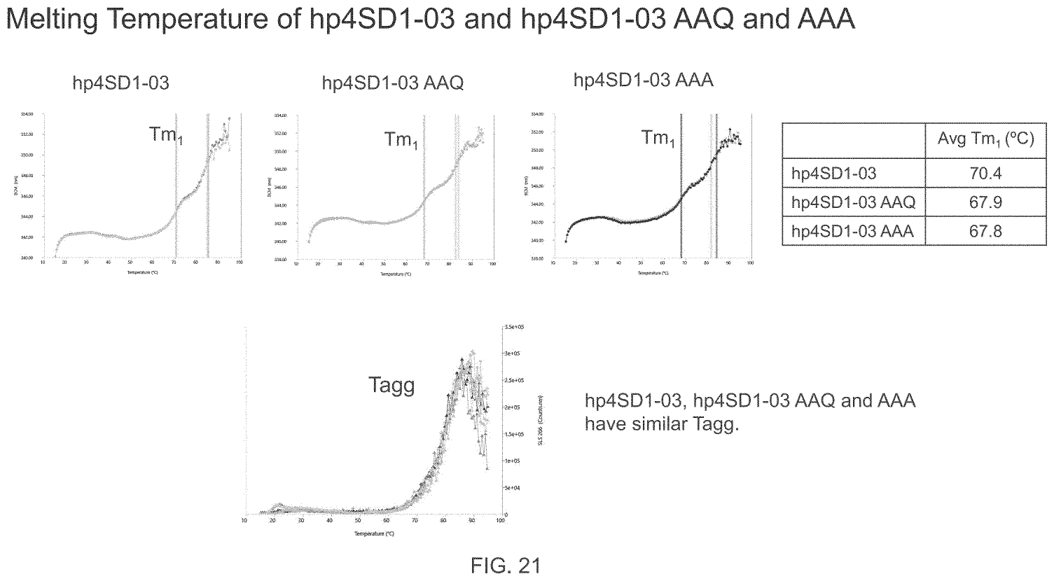

No significant changes in the first melting temperature (Tm1) were found with hp4SD1-03 AAQ and hp4SD1-03 AAA relative to hp4SD1-03 (FIG. 21).

Materials and Methods

Melting curves were determined and melting temperatures (Tm1 and Tm2) were measured using an UNcle Instrument (UNchained Labs) monitoring the intrinsic fluorescence. The protein concentration was 0.5 mg/mL in HBS, pH 7.4 with the temperature ramping from 15° C. to 95° C., with the ramp rate of 0.6° C./min. In the same experiment, temperature of the onset of aggregation (Tagg) was determined by static light scattering of increasing particle size. Each sample was analyzed in duplicate.

STF Activity of hp4SD1-03 AAQ and hp4SD1-03 AAA

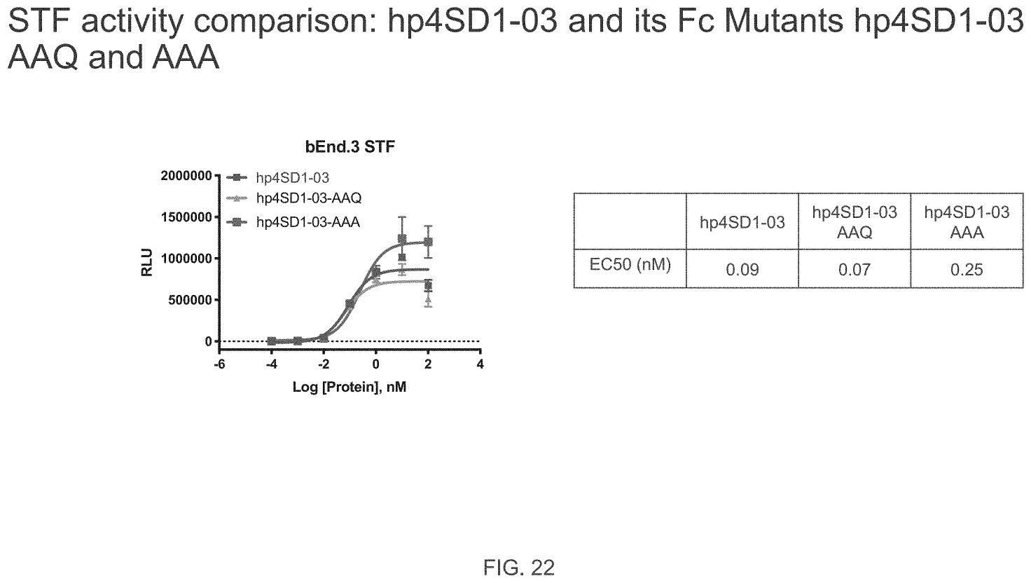

Wnt signaling activity was measured by the STF assay as described in Example 1. Exemplary results are shown in FIG. 22. hp4SD1-03 AAQ showed slightly reduced EC50, and hp4SD1-03 AAA showed increased magnitude in response (Emax), relative to hp4SD1-03.

Binding of hp4SD1-03 AAQ and hp4SD1-03 AAA to FcRn

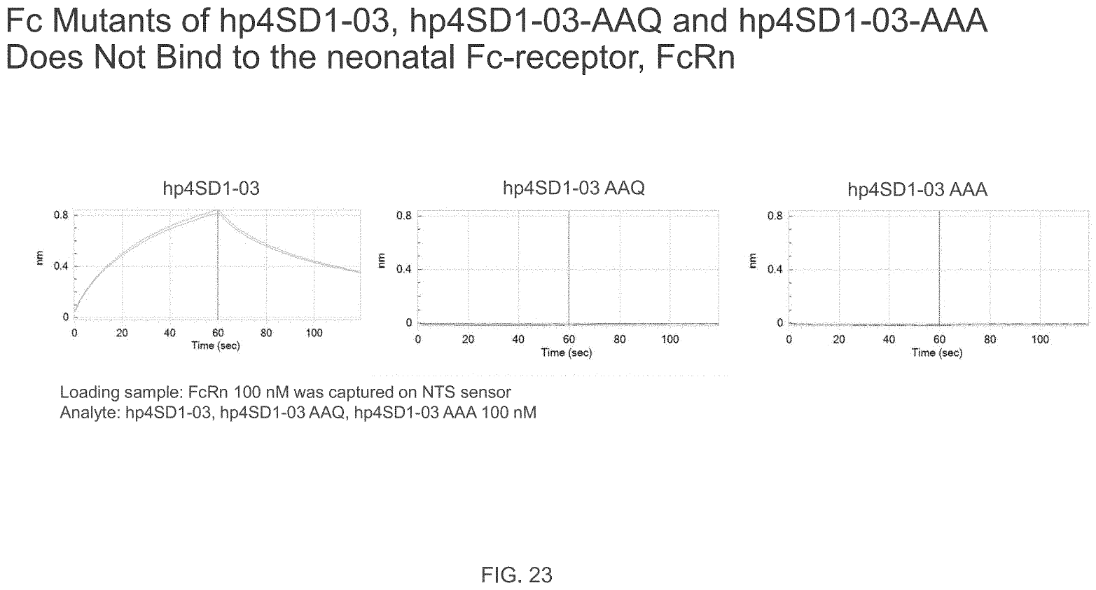

Neither hp4SD1-03 AAQ nor hp4SD1-03 AAA showed any binding to FcRn (see FIG. 23).

Materials and Methods

The binding assay was performed by BLI on the Octet® Red 96 (PALL ForteBio, Fremont, CA) instrument at 25° C., 1000 rpm with NTA biosensors. His-tagged FcRn (Acro Biosystems) was diluted to 100 nM in the running buffer (PBS, 0.05% Tween-20, 0.5% BSA, pH 7.2) and captured to the NTA biosensor (Sartorius), followed by dipping into wells containing the hp4SD1-03, hp4SD1-03 AAQ, and hp4SD1-03 AAA at 100 nM in the running buffer or into a well with only the running buffer as a reference channel. The samples were assayed in duplicates.

### Example 10

### Pharmacokinetics (PK)

Various pharmacokinetic parameters including half-life for hp4SD1-03, hp4SD1-03 AAQ, and hp4SD1-03 AAA will be determined.

hp4SD1-03, hp4SD1-03 AAQ, and hp4SD1-03 AAA will be produced and purified appropriately, e.g., as described above. Prior to dosing, model animals (e.g., rabbits such as New Zealand White rabbits or Chinchilla rabbits, monkeys such as cynomolgus monkeys, etc) will be randomly assigned to different experimental groups designed based on desired doses (e.g., 100 ng, 1 μg, 10 μg, 100 μg, 1 mg, and 10 mg etc), desired routes (e.g., intravitreally, topically using eye drops, intravenously, etc), sample types to harvest, and sample harvest timepoints. Blood samples will be collected for baseline measurements.

On the day of dosing, hp4SD1-03, hp4SD1-03 AAQ, and hp4SD1-03 AAA will be reconstituted to desired concentrations (e.g., 300 nM, 3 μM, 30 M, 300 μM, 3 mM, and 30 mM etc) and administered to model animals at desired doses via desired routes.

For analyses following intravenous administration, an appropriate amount of blood (e.g., 0.2 mL for small animals such as rabbits, 3 mL for larger animals such as monkeys) will be serially collected e.g., via the central ear artery at various timepoints, e.g., 5 and 30 minutes and 1, 2, 4, 8, 24, 32, 48, 72, and 96 hours post dosing. For analyses following intravitreal or topical administration, an appropriate amount of blood will also be serially collected and appropriate tissues such as ocular tissues (e.g., vitreous humor, aqueous humor, vitreous humor, retinal tissue (e.g., including disc, macula, peripheral retina)) will be collected at various pre-selected terminal timepoints.

All blood samples will be collected in serum separator tubes without an anticoagulant, allowed to clot at an ambient temperature e.g., for at least 20 minutes, and centrifuged at 2-8° C. Serum will be harvested within about 20 minutes of centrifugation and stored at −60° C. to −80° C. until PK analyses. All collected tissues will be weighed and stored at −80° C. until analyses.

The concentration of hp4SD1-03, hp4SD1-03 AAQ, or hp4SD1-03 AAA in serum samples will be analyzed, for example by subjecting serum samples to ELISA. The concentration of hp4SD1-03, hp4SD1-03 AAQ, or hp4SD1-03 AAA in tissue samples will be analyzed, for example by homogenizing the tissue samples and subjecting the homogenate to ELISA. Concentrations will be plotted against time to visualize systemic and local pharmacokinetics. Systemic and local half-lives will be calculated. For analyzing PK, fitting to any appropriate PK models may be used (see e.g., Gadkar et al., Invest Ophthalmol Vis Sci. 2015 August; 56(9):5390-400.; Le et al., J Pharmacol Exp Ther. 2015 November; 355(2):288-96.; Li et al., Exp Eye Res. 2012 April; 97(1):154-9.; and Park et al., Invest Ophthalmol Vis Sci. 2016 May 1; 57(6):2612-7.).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### Exemplary Embodiments

Described herein below are some exemplary embodiments according to the present disclosure.

Embodiment P1. An engineered WNT signaling agonist, wherein the WNT signaling agonist comprises: a) two light chains, each comprising a polypeptide sequence having at least 95% identity to SEQ ID NO:1; and 2) two heavy chains, each comprising a polypeptide sequence having at least 95% identity to SEQ ID NO:2, wherein the two heavy chains are bound to each other, and each of the two light chains is bound to a different heavy chain of the WNT signaling agonist.

Embodiment P2. The engineered WNT signaling agonist of Embodiment P1, wherein each of the two light chains comprises a VHH domain comprising one or more of the following amino acid residues: N29Q and/or VHH3-H4 (or hVHH3-H4) DDD substituted with DED.

Embodiment P3. The engineered WNT signaling agonist of Embodiment P1, wherein each of the two heavy chains comprises one or more of the following amino acid residues: a CDR1 S31, a CDR2 E62, and/or CDR3 D108.

Embodiment P4. A nucleic acid comprising a sequence encoding a light chain and/or a heavy chain of the engineered WNT signaling agonist of any one of Embodiments P1-P3.

Embodiment P5. The nucleic acid of Embodiment P4, wherein the nucleic acid is an mRNA, e.g., a modified mRNA.

Embodiment P6. A vector comprising the nucleic acid of Embodiment P4 or Embodiment P5.

Embodiment P7. The vector of Embodiment P6, wherein the vector is an expression vector comprising a promoter operatively linked to the nucleic acid of Embodiment P4 or Embodiment P5.

Embodiment P8. A host cell comprising the nucleic acid of Embodiment P4 or Embodiment P5 or the vector of Embodiment P6 or Embodiment P7.

Embodiment P9. A pharmaceutical composition comprising a pharmaceutically acceptable diluent, excipient or carrier, and: a) the engineered WNT signaling agonist of any one of Embodiments P1-P3; b) the nucleic acid of any one of Embodiments P4-P5; and/or c) the vector of any one of Embodiments P6-P7.

Embodiment P10. A method of treating a retinopathy in a subject, comprising administering to the subject: a) the engineered WNT signaling agonist of any one of Embodiments P1-P3; b) the nucleic acid of any one of Embodiments P4-P5; c) the vector of any one of Embodiments P6-P7; and/or d) the pharmaceutical composition of Embodiment P9.

Embodiment P11. The method of Embodiment P10, wherein the retinopathy is a retinal vascular disease.

Embodiment P12. The method of Embodiment P11, wherein the retinal vascular disease is caused by inhibition of vascular development.

Embodiment P13. The method of Embodiment P10, wherein the retinopathy is caused by excessive angiogenesis.

Embodiment P14. The method of any one of Embodiments P10-P13, wherein the retinal vascular disease is selected from the group consisting of: familiar exudative vitreoretinopathy (FEVR), exudative vitreoretinopathy, Norrie disease, diabetic retinopathy (DR), diabetic macular edema, diabetic macular ischemia, age-related macular degeneration (AMD) (including wet AMD and dry AMD), retinopathy of prematurity (ROP), osteoporosis-pseudoglioma syndrome (OPPG), retinal vein occlusion, and Coats disease.

Embodiment P15. The method of any one of Embodiments P10-P14, wherein the engineered WNT signaling agonist is administered to one or more of the subject's eyes.

Embodiment P16. The method of Embodiment P15, wherein the engineered WNT signaling agonist is administered intravitreally.

Embodiment A1. An anti-Fzd4 antibody or antibody fragment, comprising:
(A) a VH comprising: (i) a CDRH1 comprising an amino acid sequence which comprises or consists of: (i-1) the CDRH1 amino acid sequence contained in any one of SEQ ID NOS: 181-188, optionally according to Kabat; (i-2) the amino acid sequence of any one of SEQ ID NOS: 121-124; and/or (i-3) the amino acid sequence of SEQ ID NO: 120, wherein X₁ is any amino acid, optionally S, A, Q, or N; (ii) a CDRH2 comprising an amino acid sequence which comprises or consists of: (ii-1) the CDRH2 amino acid sequence contained in any one of SEQ ID NOS: 181-188, optionally according to Kabat; (ii-2) the amino acid sequence of any one of SEQ ID NOS: 141-144; and/or (ii-3) the amino acid sequence of SEQ ID NO: 140, wherein X₁ is any amino acid, optionally E, A, S, or D; and (iii) a CDRH3 comprising an amino acid sequence which comprises or consists of: (iii-1) the CDRH3 amino acid sequence contained in any one of SEQ ID NOS: 181-188, optionally according to Kabat; (iii-2) the amino acid sequence of SEQ ID NO: 161; and/or (iii-3) the amino acid sequence of SEQ ID NO: 160, wherein X₁ is any amino acid, optionally D or any amino acid which is not S, E, A, R, T, L, H, Y, Q, or K; and
(B) a VL comprising: (i) a CDRL1 comprising an amino acid sequence which comprises or consists of: (i-1) the CDRL1 amino acid sequence contained in SEQ ID NO: 281, optionally according to Kabat; and/or (i-2) comprises or consists of the amino acid sequence of SEQ ID NO: 221; (ii) a CDRL2 comprising an amino acid sequence which comprises or consists of: (ii-1) the CDRL2 amino acid sequence contained in SEQ ID NO: 281, optionally according to Kabat; and/or (ii-2) comprises or consists of the amino acid sequence of SEQ ID NO: 241; and (iii) a CDRL3 comprising an amino acid sequence which comprises or consists of: (iii-1) the CDRL3 amino acid sequence contained in SEQ ID NO: 281, optionally according to Kabat; and/or (iii-2) comprises or consists of the amino acid sequence of SEQ ID NO: 261.

Embodiment A2. The anti-Fzd4 antibody or antibody fragment of Embodiment A1, wherein:
(A) the VH comprising: (i) a FRH1 comprising an amino acid sequence which comprises or consists of: (i-1) the FRH1 amino acid sequence contained in any one of SEQ ID NOS: 181-188, optionally according to Kabat; (i-2) the amino acid sequence of SEQ ID NO: 111 or 112; and/or (i-3) the amino acid sequence of SEQ ID NO: 110, wherein X₁ is any amino acid, optionally Q or K; (ii) a FRH2 comprising an amino acid sequence which comprises or consists of: (ii-1) the FRH2 amino acid sequence contained in any one of SEQ ID NOS: 181-188, optionally according to Kabat; and/or (ii-2) the amino acid sequence of SEQ ID NO: 130 or 131; (iii) a FRH3 comprising an amino acid sequence which comprises or consists of: (iii-1) the FRH3 amino acid sequence contained in any one of SEQ ID NOS: 181-188, optionally according to Kabat; (iii-2) the amino acid sequence of SEQ ID NO: 151 or 152; and/or (iii-3) the amino acid sequence of SEQ ID NO: 150, wherein X₁ is any amino acid, optionally N or D; X₂ is any amino acid, optionally R or K; and X₃ is any amino acid, optionally E or T; and/or (iv) a FRH4 comprising an amino acid sequence which comprises or consists of: (iv-1) the FRH4 amino acid sequence contained in any one of SEQ ID NOS: 181-188, optionally according to Kabat; and/or (iv-2) the amino acid sequence of SEQ ID NO: 170 or 171; and/or
(B) the VL comprising: (i) a FRL1 comprising an amino acid sequence which comprises or consists of: (i-1) the FRL1 amino acid sequence contained in SEQ ID NO: 281, optionally according to Kabat; and/or (i-2) the amino acid sequence of SEQ ID NO: 221; (ii) a FRL2 comprising an amino acid sequence which comprises or consists of: (ii-1) the FRL2 amino acid sequence contained in SEQ ID NO: 281, optionally according to Kabat; and/or (ii-2) the amino acid sequence of SEQ ID NO: 231; (iii) a FRL3 comprising an amino acid sequence which comprises or consists of: (iii-1) the FRL3 amino acid sequence contained in SEQ ID NO: 281, optionally according to Kabat; and/or (iii-2) the amino acid sequence of SEQ ID NO: 251; (iv) a FRL3 comprising an amino acid sequence which comprises or consists of: (iv-1) the FRL3 amino acid sequence contained in SEQ ID NO: 281, optionally according to Kabat; and/or (iv-2) the amino acid sequence of SEQ ID NO: 271.

Embodiment A3. The anti-Fzd4 antibody or antibody fragment of Embodiment A1 or A2, wherein:
(A) in the VH:
(I) (I-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 181; optionally according to Kabat and/or (ii) the amino acid sequences of SEQ ID NOS: 121, 141, and 161; and (I-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 181;
(II) (II-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 182, optionally according to Kabat; and/or (ii) the amino acid sequences of SEQ ID NOS: 122, 142, and 162; and (II-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 182;
(III) (III-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 183, optionally according to Kabat; and/or (ii) the amino acid sequences of SEQ ID NOS: 124, 142, and 162; and (III-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 183;
(IV) (IV-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 184, optionally according to Kabat; and/or (ii) the amino acid sequences of SEQ ID NOS: 121, 142, and 162; and (IV-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 184;
(V) (V-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 185, optionally according to Kabat; and/or (ii) the amino acid sequences of SEQ ID NOS: 123, 142, and 162; and (V-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 185;
(VI) (VI-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 186, optionally according to Kabat; and/or (ii) the amino acid sequences of SEQ ID NOS: 122, 144, and 162; and (VI-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 186;
(VII) (VII-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 187, optionally according to Kabat; and/or (ii) the amino acid sequences of SEQ ID NOS: 122, 141, and 162; and (VII-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 187;
(VIII) (VIII-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 188, optionally according to Kabat; and/or (ii) the amino acid sequences of SEQ ID NOS: 122, 143, and 162; and (VIII-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 188; and/or
(B) in the VL: (1) the CDRL1, the CDRL2, and the CDRL3 comprise amino acid sequences which comprise or consist of: (i) the CDRL1 amino acid sequence, the CDRL2 amino acid sequence, and the CDRL3 amino acid sequence, respectively, contained in SEQ ID NO: 281, optionally according to Kabat; and/or (ii) the amino acid sequences of SEQ ID NOS: 221, 241, and 261; and (2) optionally the VL comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 281.

Embodiment A4. The anti-Fzd4 antibody or antibody fragment of any one of Embodiments A1-A3, wherein: (A) the VH comprises amino acid sequences which comprise or consist of the amino acid sequence of any one of SEQ ID NOS: 181-188; and/or (B) the VL comprises amino acid sequences which comprise or consist of the amino acid sequence of SEQ ID NO: 281.

Embodiment A5. The anti-Fzd4 antibody or antibody fragment of any one of Embodiments A1-A4, wherein: (A) the VH comprises amino acid sequences which comprise or consist of the amino acid sequence of SEQ ID NO: 181; and (B) the VL comprises amino acid sequences which comprise or consist of the amino acid sequence of SEQ ID NO: 281.

Embodiment A6. The anti-Fzd4 antibody or antibody fragment of any one of Embodiments A1-A5, which is, comprises, or is comprised in:
(i) an IgG, optionally IgG1, IgG2, IgG3, or IgG4, an IgA, an IgE, an IgD, or an IgM comprising said VH and said VL;
(ii) a multispecific antibody or antibody fragment (optionally comprising a structure as depicted in any one of FIGS. 24-35, further optionally FIG. 24) comprising a first antigen-binding region comprising said VH and said VL and a second antigen-binding region specific for a second antigen, optionally wherein the second antigen is LRP5 and/or LRP6; and/or
(iii) a fragment antigen-binding (Fab), a Fab′ fragment, a F(ab′)2, a single-chain Fab (scFab), a variable fragment (Fv), a single-chain Fv (scFv), a diabody, or a triabody comprising said VH and said VL.

Embodiment A7. The anti-Fzd4 antibody or antibody fragment of any one of Embodiments A1-A6, which comprises one or more of the following:
(a) an immunoglobulin heavy chain constant domain 1 (CH1) or a variant thereof, optionally wherein the CH1 or a variant thereof: (a-1) is of an IgG, an IgA, an IgE, an IgD, an IgM, optionally of an IgG1, an IgG4, an IgG2, or an IgG3, further optionally of human; and/or (a-2) comprises an amino acid sequence which comprises or consists of the amino acid sequence of SEQ ID NO: 21 or 22 or which is at least 80%, at least 85%, at least 90%, at least 91%, at least 92%, at least 93%, at least 94%, at least 95%, at least 96%, at least 97%, at least 98%, at least 99%, or 100% identical thereto;
(b) an immunoglobulin hinge or a variant thereof, optionally wherein the hinge or a variant thereof: (b-1) is of an IgG, an IgA, an IgE, an IgD, an IgM, optionally of an IgG1, an IgG4, an IgG2, or an IgG3, further optionally of human; and/or (b-2) comprises an amino acid sequence which comprises or consists of the amino acid sequence of SEQ ID NO: 31 or which is at least 80%, at least 85%, at least 90%, at least 91%, at least 92%, at least 93%, at least 94%, at least 95%, at least 96%, at least 97%, at least 98%, at least 99%, or 100% identical thereto;
(c) an immunoglobulin heavy chain constant domain 2 (CH2) or a variant thereof, optionally wherein the CH2 or a variant thereof: (c-1) is of an IgG, an IgA, an IgE, an IgD, an IgM, optionally of an IgG1, an IgG4, an IgG2, or an IgG3, further optionally of human; and/or (c-2) comprises an amino acid sequence which comprises or consists of the amino acid sequence of any of SEQ ID NOS: 41-46 or which is at least 80%, at least 85%, at least 90%, at least 91%, at least 92%, at least 93%, at least 94%, at least 95%, at least 96%, at least 97%, at least 98%, at least 99%, or 100% identical thereto;
(d) an immunoglobulin heavy chain constant domain 3 (CH3) or a variant thereof, optionally wherein the CH3 or a variant thereof: (d-1) is of an IgG, an IgA, an IgE, an IgD, an IgM, optionally of an IgG1, an IgG4, an IgG2, or an IgG3, further optionally of human; and/or (d-2) comprises an amino acid sequence which comprises or consists of the amino acid sequence of any of SEQ ID NOS: 51-54, 61-64, and 66-69 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or 100% identical thereto; and/or
(e) an immunoglobulin light chain constant domain (CL) or a variant thereof, optionally wherein the CL or a variant thereof: (e-1) is of a kappa light chain constant domain (CLκ), optionally human CLκ and optionally comprising an amino acid sequence which comprises or consists of the amino acid sequence of SEQ ID NO: 11 or which is at least 80%, at least 85%, at least 90%, at least 91%, at least 92%, at least 93%, at least 94%, at least 95%, at least 96%, at least 97%, at least 98%, at least 99%, or 100% identity thereto; or (e-2) is of a lambda light chain constant domain (CLλ), optionally human CLλ, and optionally comprising an amino acid sequence which comprises or consists of the amino acid sequence of SEQ ID NO: 12 or which is at least 80%, at least 85%, at least 90%, at least 91%, at least 92%, at least 93%, at least 94%, at least 95%, at least 96%, at least 97%, at least 98%, at least 99%, or 100% identity thereto, optionally wherein the anti-Fzd4 antibody or antibody fragment comprises the CH2 and CH3 sequences of SEQ ID NOS: 42 and 51, respectively, SEQ ID NOS: 43 and 51, respectively, SEQ ID NOS: 45 and 61, respectively, SEQ ID NO: 45 and 66, respectively, SEQ ID NOS: 46 and 61, respectively, or SEQ ID NO: 46 and 66, respectively.

Embodiment A8. The anti-Fzd4 antibody or antibody fragment of any one of Embodiments A1-A7, which comprises a fragment crystallizable (Fc) region, optionally wherein the Fc region:
(I) is of an IgG, an IgA, an IgE, an IgD, an IgM, optionally of an IgG1, an IgG4, an IgG2, or an IgG3, further optionally of human, or a variant thereof;
(II) is of: (i) an IgG1 or a variant thereof, optionally comprising one or more of the following amino acid substitutions: N297A, N297Q, D265A, L234A, L235A, C226S, C229S, P238S, E233P, L234V, G236-deleted, P238A, A327Q, A327G, P329A, P329G, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, H310A, L328F, M252Y, I253A, S254T, T256E, H435Q, H435A, and/or any combination thereof, according to EU numbering; (ii) an IgG2 or a variant thereof, optionally comprising one or more of the following amino acid substitutions: P238S, V234A, G237A, H268A, H268Q, H268E, V309L, N297A, N297Q, A330S, P331S, C232S, C233S, M252Y, S254T, T256E, and/or any combination thereof, according to EU numbering; (iii) an IgG3 or a variant thereof, optionally comprising the amino acid substitution E235Y, according to EU numbering; or (iv) an IgG4 or a variant thereof, optionally comprising one or more of the following amino acid substitutions: E233P, F234V, L235A, G237A, E318A, S228P, L236E, S241P, L248E, T394D, M252Y, S254T, T256E, N297A, N297Q, and/or any combination thereof, according to EU numbering:
(III) comprises one or more amino acid substitutions that reduce an Fc effector function, optionally wherein the one or more amino acid substitutions are at position(s) selected from the group consisting of positions 234, 235, 236, 237, 265, 297 and 329, according to EU numbering;
(IV) comprises at least the following amino acid substitutions according to EU numbering: (ii-1) L234A, L235A, and P329G; (ii-2) L234A and L235A; (ii-3) I253A, H310A, and H435Q; (ii-4) I253A, H310A, and H435A; (ii-5) D265A and P329A; (ii-6) M252Y, S254T, and T256E; and/or (ii-7) M428L and N434S, optionally relative to a human IgG1 Fc region and/or to any of SEQ ID NOS: 71-74 (optionally not including the C-terminal K); and/or
(V) comprises an amino acid sequence which comprises or consists of the amino acid sequence of any one of SEQ ID NOS: 71-74, 81-84, 86-89, 91-94, 96-99, 471-474, 476-479, 481-484, and 486-489 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or is 100% identical thereto.

Embodiment A9. A nucleic acid or a combination of nucleic acids encoding the anti-Fzd4 antibody or antibody fragment of any one of Embodiments A1-A8, optionally wherein the nucleic acid(s) is/are DNA, cDNA, RNA, mRNA, modified mRNA, or a DNA/RNA hybrid.

Embodiment A10. The nucleic acid or a combination of nucleic acids of Embodiments A9, comprising: (a) a first nucleic acid encoding a first polypeptide comprising said VH; and (b) a second nucleic acid encoding a second polypeptide comprising said VL.

Embodiment A11. A vector or a combination of vectors which comprises the nucleic acid or the combination of nucleic acids of Embodiment A9 or A10, optionally wherein: (i) the vector(s) comprise(s) one or more promoters operably linked to the nucleic acid(s); (ii) the vector(s) is/are an expression vector; and/or (iii) the vector(s) comprise(s) a plasmid, a viral vector (optionally adeno-associated viral, adenoviral, lentiviral, or retroviral), a lipid-based vector, a self-replicating RNA vector, a virus-like particle, a polymer-based vector, and/or a nanoparticle, optionally a lipid-based nanoparticle.

Embodiment A12. The vector or a combination of vectors of Embodiment A11, which encodes the anti-Fzd4 antibody or antibody fragment of any one of Embodiments A1-A8 and comprises: (a) a first vector comprising a first nucleic acid encoding a first polypeptide comprising said VH; and (b) a second vector comprising a second nucleic acid encoding a second polypeptide comprising said VL.

Embodiment A13. A host cell, which comprises: (A) the anti-Fzd4 antibody or antibody fragment of any one of Embodiments A1-A8; (B) the nucleic acid or the combination of nucleic acids of Embodiment A9 or A10; and/or (C) the vector or the combination of vectors of Embodiment A11 or A12, optionally wherein the host cell is: (i) mammalian, optionally human, non-human primate, monkey, rabbit, rodent, hamster, rat, or mouse; or (ii) non-mammalian, optionally plant, bacterial, fungal, yeast, protozoa, or insect, and optionally wherein the host cell is: (i) a human embryonal kidney (HEK) cell, optionally a HEK293 cell, or a variant thereof, further optionally Expi293F™ cell; (ii) a CHO (Chinese Hamster Ovary) cell; (iii) an immune cell or (iv) a hybridoma.

Embodiment A14. A population of cells, which comprises two or more host cells of Embodiment A13.

Embodiment A15. A pharmaceutical composition, which comprises: (I) (A) the anti-Fzd4 antibody or antibody fragment of any one of Embodiments A1-A8; (B) the nucleic acid or the combination of nucleic acids of Embodiment A9 or A10; (C) the vector or the combination of vectors of Embodiment A11 or A12; and/or (D) the host cell of Embodiment A13 or the population of cells of Embodiment A14; and (II) a pharmaceutically acceptable carrier and/or excipient.

Embodiment A16. A method of treating a subject in need of such treatment, comprising administering to the subject an effective amount of: (A) the anti-Fzd4 antibody or antibody fragment of any one of Embodiments A1-A8; (B) the nucleic acid or the combination of nucleic acids of Embodiment A9 or A10; (C) the vector or the combination of vectors of Embodiment A11 or A12; and/or (D) the host cell of Embodiment A13 or the population of cells of Embodiment A14; and/or (E) the pharmaceutical composition of Embodiment A15, optionally wherein: (a) the subject is (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile; (b) the subject comprises or has a risk of developing a disease, disorder, or a condition; and/or (c) the method further comprises administering to the subject an additional agent, optionally an adjuvant or a therapeutic agent.

Embodiment A17. A method of treating or preventing a disease, disorder, or a condition in a subject in need of such treatment, the method comprising administering an effective amount of: (A) the anti-Fzd4 antibody or antibody fragment of any one of Embodiments A1-A8; (B) the nucleic acid or the combination of nucleic acids of Embodiment A9 or A10; (C) the vector or the combination of vectors of Embodiment A11 or A12; and/or (D) the host cell of Embodiment A13 or the population of cells of Embodiment A14; and/or (E) the pharmaceutical composition of Embodiment A15, optionally wherein: (a) the subject is (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile; and/or (b) the method further comprises administering to the subject an additional agent, optionally an adjuvant or a therapeutic agent.

Embodiment A18. A method of manufacturing the anti-Fzd4 antibody or antibody fragment of any one of Embodiments A1-A8, comprising: (a) culturing cells comprising the nucleic acid or the combination of nucleic acids of Embodiment A9 or A10 in a condition that allows for expression of said anti-Fzd4 antibody or antibody fragment, and (b) harvesting and purifying the anti-Fzd4 antibody or antibody fragment from the cell culture from (a).

Embodiment A19. A method of manufacturing the host cell of Embodiment A13 or the population of such cells, comprising introducing the nucleic acid or the combination of nucleic acids of Embodiment A9 or A10 and/or the vector or the combination of vectors of Embodiment A11 or A12 into one or more cells, optionally wherein the introducing occurs in vitro, ex vivo, or in vivo.

Embodiment B1. An anti-LRP5/6 antibody or antibody fragment, comprising: a VH comprising:
(i) a CDRH1 comprising an amino acid sequence which comprises or consists of: (i-1) the CDRH1 amino acid sequence contained in any one of SEQ ID NOS: 381-399, optionally according to IMGT or Kabat; (i-2) the amino acid sequence of any one of SEQ ID NOS: 321-323; and/or (i-3) the amino acid sequence of SEQ ID NO: 320, wherein X₁ is any amino acid, optionally Q, N, or S, or any amino acid that is not A;
(ii) a CDRH2 comprising an amino acid sequence which comprises or consists of: (ii-1) the CDRH2 amino acid sequence contained in any one of SEQ ID NOS: 381-399, optionally according to IMGT or Kabat; (ii-2) the amino acid sequence of SEQ ID NO: 341; and/or (ii-3) the amino acid sequence of SEQ ID NO: 340, wherein X₁ is any amino acid and X₂ is any amino acid, optionally wherein the CDRH2 does not comprise an amino acid sequence consisting of SEQ ID NO: 340, wherein X₁ and X₂ are K and M, respectively, L and M, respectively, I and M, respectively, M and K, respectively, M and L, respectively, or M and I, respectively; and
(iii) a CDRH3 comprising an amino acid sequence which comprises or consists of: (iii-1) the CDRH3 amino acid sequence contained in any one of SEQ ID NOS: 381-399, optionally according to IMGT or Kabat; (iii-2) the amino acid sequence of any one of SEQ ID NOS: 361-369 and 300-303; and/or (iii-3) the amino acid sequence of SEQ ID NO: 360, wherein X₁ is any amino acid, optionally D or E, X₂ is any amino acid, optionally E, D, or S, and X₃ is any amino acid, optionally D, E, S, A, T, or D, optionally wherein the VH is, comprises, or is comprised in a nanobody.

Embodiment B2. The anti-LRP5/6 antibody or antibody fragment of Embodiment B1, wherein the VH comprises:
(i) a FRH1 comprising an amino acid sequence which comprises or consists of: (i-1) the FRH1 amino acid sequence contained in any one of SEQ ID NOS: 381-399, optionally according to IMGT or Kabat; (i-2) the amino acid sequence of SEQ ID NO: 311 or 312; and/or (i-3) the amino acid sequence of SEQ ID NO: 310, wherein X₁ is any amino acid, optionally E or D, X₂ is any amino acid, optionally A or T, and X₃ is any amino acid, optionally S, optionally wherein the FRH1 does not comprise an amino acid sequence consisting of SEQ ID NO: 310, wherein X₁, X₂, and X₃ are E, A, and A, respectively;
(ii) a FRH2 comprising an amino acid sequence which comprises or consists of: (ii-1) the FRH2 amino acid sequence contained in any one of SEQ ID NOS: 381-399, optionally according to IMGT or Kabat; (ii-2) the amino acid sequence of any one of SEQ ID NOS: 331-333; and/or (ii-3) the amino acid sequence of SEQ ID NO: 330, wherein X₁ is any amino acid, optionally Y, X₂ is any amino acid, optionally Q or G, X₃ is any amino acid, optionally R, X₄ is any amino acid, optionally L or W, X₅ is any amino acid, optionally I, and X₆ is any amino acid, optionally A, optionally wherein the FRH2 does not comprise an amino acid sequence consisting of SEQ ID NO: 330, wherein X₁, X₂, X₃, X₄, X₅, and X₆ are V, G, L, W, V, and S, respectively;
(iii) a FRH3 comprising an amino acid sequence which comprises or consists of: (iii-1) the FRH3 amino acid sequence contained in any one of SEQ ID NOS: 381-399, optionally according to IMGT or Kabat; (iii-2) the amino acid sequence of SEQ ID NO: 351 or 352; and/or (iii-3) the amino acid sequence of SEQ ID NO: 350, wherein X₁ is any amino acid, optionally D or G, X₂ is any amino acid, optionally L, X₃ is any amino acid, optionally M, X₄ is any amino acid, optionally T, X₅ is any amino acid, optionally D or E, X₆ is any amino acid, optionally N or S, X₇ is any amino acid, optionally S or A, X₈ is any amino acid, optionally M, X₉ is any amino acid, optionally R or K, and X₁₀ is any amino acid, optionally A or P, optionally wherein the FRH3 does not comprise an amino acid sequence consisting of SEQ ID NO: 350, wherein X₁, X₂, X₃, X₄, X₅, X₆, X₇, X₈, X₉, and X₁₀ are D, V, I, R, D, N, S, L, R, and E, respectively, and/or
(iv) a FRH4 comprising an amino acid sequence which comprises or consists of: (i-1) the FRH4 amino acid sequence contained in any one of SEQ ID NOS: 381-399, optionally according to IMGT or Kabat; (i-2) the amino acid sequence of SEQ ID NO: 371 or 372; and/or (i-3) the amino acid sequence of SEQ ID NO: 370, wherein X₁ is any amino acid, optionally R or W and X₂ is any amino acid, optionally Q or L.

Embodiment B3. The anti-LRP5/6 antibody or antibody fragment of Embodiment B1 or B2, wherein in the VH:
(I) (I-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 381, optionally according to IMGT or Kabat; and/or (ii) the amino acid sequences of SEQ ID NOS: 321, 341, and 361; and (I-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 381;
(II) (II-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in any one of SEQ ID NO: 382-385, optionally according to IMGT or Kabat; and/or (ii) the amino acid sequences of SEQ ID NOS: 322, 341, and 362; and (II-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of any one of SEQ ID NO: 382-385;
(III) (III-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 386, optionally according to IMGT or Kabat; and/or (ii) the amino acid sequences of SEQ ID NOS: 321, 341, and 362; and (III-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 386;
(IV) (IV-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 387, optionally according to IMGT or Kabat; and/or (ii) the amino acid sequences of SEQ ID NOS: 323, 341, and 362; and (IV-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 387;
(V) (V-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 388, optionally according to IMGT or Kabat; and/or (ii) the amino acid sequences of SEQ ID NOS: 322, 341, and 361; and (V-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 388;
(VI) (VI-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 389, optionally according to IMGT or Kabat; and/or (ii) the amino acid sequences of SEQ ID NOS: 322, 341, and 300; and (VI-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 389;
(VII) (VII-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 390, optionally according to IMGT or Kabat; and/or (ii) the amino acid sequences of SEQ ID NOS: 322, 341, and 301; and (VII-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 390;
(VIII) (VIII-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 391, optionally according to IMGT or Kabat; and/or (ii) the amino acid sequences of SEQ ID NOS: 322, 341, and 302; and (VIII-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 391;
(IX) (IX-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 392, optionally according to IMGT or Kabat; and/or (ii) the amino acid sequences of SEQ ID NOS: 322, 341, and 303; and (IX-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 392;
(X) (X-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 393, optionally according to IMGT or Kabat; and/or (ii) the amino acid sequences of SEQ ID NOS: 321, 341, and 363; and (X-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 393;
(XI) (XI-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 394, optionally according to IMGT or Kabat; and/or (ii) the amino acid sequences of SEQ ID NOS: 321, 341, and 364; and (XI-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 394;
(XII) (XII-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 395, optionally according to IMGT or Kabat; and/or (ii) the amino acid sequences of SEQ ID NOS: 321, 341, and 365; and (XII-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 395;
(XIII) (XIII-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 396, optionally according to IMGT or Kabat; and/or (ii) the amino acid sequences of SEQ ID NOS: 321, 341, and 366; and (XIII-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 396;
(XIV) (XIV-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 397, optionally according to IMGT or Kabat; and/or (ii) the amino acid sequences of SEQ ID NOS: 321, 341, and 367; and (XIV-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 397;
(XV) (XV-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 398, optionally according to IMGT or Kabat; and/or (ii) the amino acid sequences of SEQ ID NOS: 321, 341, and 368; and (XV-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 398; or
(XVI) (XVI-1) the CDRH1, the CDRH2, and the CDRH3 comprise amino acid sequences which comprise or consist of: (i) the CDRH1 amino acid sequence, the CDRH2 amino acid sequence, and the CDRH3 amino acid sequence, respectively, contained in SEQ ID NO: 399, optionally according to IMGT or Kabat; and/or (ii) the amino acid sequences of SEQ ID NOS: 321, 341, and 369; and (XVI-2) optionally the VH comprises an amino acid sequence which has at least 80%, at least 85%, at least 90%, at least 91%, at least 92%, at least 93%, at least 94%, at least 95%, at least 96%, at least 97%, at least 98%, at least 99%, or 100% identity to the amino acid sequence of SEQ ID NO: 399.

Embodiment B4. The anti-LRP5/6 antibody or antibody fragment of any one of Embodiments B1-B3, wherein the VH comprises amino acid sequences which comprise or consist of the amino acid sequence of any one of SEQ ID NOS: 381-399.

Embodiment B5. The anti-LRP5/6 antibody or antibody fragment of any one of Embodiments B1-B4, wherein the VH comprises amino acid sequences which comprise or consist of the amino acid sequence of SEQ ID NO: 381.

Embodiment B6. The anti-LRP5/6 antibody or antibody fragment of any one of Embodiments B1-B5, which:
(i) is, comprises, or is comprised in in a nanobody or a Fd fragment;
(ii) is comprised in a multispecific antibody or antibody fragment (optionally comprising a structure as depicted in any one of FIGS. 24-35, further optionally FIG. 24) comprising (ii-1) a first antigen-binding region specific for a first antigen, optionally wherein the first antigen is Fzd4 and a second antigen-binding region comprising said VH.
(iii) further comprises a VL and is, comprises, or is comprised in a Fab, a Fab′ fragment, a F(ab′)2, a scFab, a Fv, a scFv, a diabody, or a triabody comprising said VH and said VL.

Embodiment B7. The anti-LRP5/6 antibody or antibody fragment of any one of Embodiments B1-B6, which comprises one or more of the following:
(a) an immunoglobulin heavy chain constant domain 1 (CH1) or a variant thereof, optionally wherein the CH1 or a variant thereof: (a-1) is of an IgG, an IgA, an IgE, an IgD, an IgM, optionally of an IgG1, an IgG4, an IgG2, or an IgG3, further optionally of human; and/or (a-2) comprises an amino acid sequence which comprises or consists of the amino acid sequence of SEQ ID NO: 21 or 22 or which is at least 80%, at least 85%, at least 90%, at least 91%, at least 92%, at least 93%, at least 94%, at least 95%, at least 96%, at least 97%, at least 98%, at least 99%, or 100% identical thereto;
(b) an immunoglobulin hinge or a variant thereof, optionally wherein the hinge or a variant thereof: (b-1) is of an IgG, an IgA, an IgE, an IgD, an IgM, optionally of an IgG1, an IgG4, an IgG2, or an IgG3, further optionally of human; and/or (b-2) comprises an amino acid sequence which comprises or consists of the amino acid sequence of SEQ ID NO: 31 or which is at least 80%, at least 85%, at least 90%, at least 91%, at least 92%, at least 93%, at least 94%, at least 95%, at least 96%, at least 97%, at least 98%, at least 99%, or 100% identical thereto;
(c) an immunoglobulin heavy chain constant domain 2 (CH2) or a variant thereof, optionally wherein the CH2 or a variant thereof: (c-1) is of an IgG, an IgA, an IgE, an IgD, an IgM, optionally of an IgG1, an IgG4, an IgG2, or an IgG3, further optionally of human; and/or (c-2) comprises an amino acid sequence which comprises or consists of the amino acid sequence of any of SEQ ID NOS: 41-46 or which is at least 80%, at least 85%, at least 90%, at least 91%, at least 92%, at least 93%, at least 94%, at least 95%, at least 96%, at least 97%, at least 98%, at least 99%, or 100% identical thereto;
(d) an immunoglobulin heavy chain constant domain 3 (CH3) or a variant thereof, optionally wherein the CH3 or a variant thereof: (d-1) is of an IgG, an IgA, an IgE, an IgD, an IgM, optionally of an IgG1, an IgG4, an IgG2, or an IgG3, further optionally of human; and/or (d-2) comprises an amino acid sequence which comprises or consists of the amino acid sequence of any of SEQ ID NOS: 51-54, 61-64, and 66-69 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or 100% identical thereto; and/or
(e) an immunoglobulin light chain constant domain (CL) or a variant thereof, optionally wherein the CL or a variant thereof: (e-1) is of a kappa light chain constant domain (CLκ), optionally human CLκ and optionally comprising an amino acid sequence which comprises or consists of the amino acid sequence of SEQ ID NO: 11 or which is at least 80%, at least 85%, at least 90%, at least 91%, at least 92%, at least 93%, at least 94%, at least 95%, at least 96%, at least 97%, at least 98%, at least 99%, or 100% identity thereto; or (e-2) is of a lambda light chain constant domain (CLλ), optionally human CLλ, and optionally comprising an amino acid sequence which comprises or consists of the amino acid sequence of SEQ ID NO: 12 or which is at least 80%, at least 85%, at least 90%, at least 91%, at least 92%, at least 93%, at least 94%, at least 95%, at least 96%, at least 97%, at least 98%, at least 99%, or 100% identity thereto, optionally wherein the anti-LRP5/6 antibody or antibody fragment comprises the CH2 and CH3 sequences of SEQ ID NOS: 42 and 51, respectively, SEQ ID NOS: 43 and 51, respectively, SEQ ID NOS: 45 and 61, respectively, SEQ ID NO: 45 and 66, respectively, SEQ ID NOS: 46 and 61, respectively, or SEQ ID NO: 46 and 66, respectively.

Embodiment B8. The anti-LRP5/6 antibody or antibody fragment of any one of Embodiments B1-B7, which comprises a fragment crystallizable (Fc) region, optionally wherein the Fc region:
(I) is of an IgG, an IgA, an IgE, an IgD, an IgM, optionally of an IgG1, an IgG4, an IgG2, or an IgG3, further optionally of human, or a variant thereof;
(II) is of: (i) an IgG1 or a variant thereof, optionally comprising one or more of the following amino acid substitutions: N297A, N297Q, D265A, L234A, L235A, C226S, C229S, P238S, E233P, L234V, G236-deleted, P238A, A327Q, A327G, P329A, P329G, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, H310A, L328F, M252Y, I253A, S254T, T256E, H435Q, H435A, and/or any combination thereof, according to EU numbering; (ii) an IgG2 or a variant thereof, optionally comprising one or more of the following amino acid substitutions: P238S, V234A, G237A, H268A, H268Q, H268E, V309L, N297A, N297Q, A330S, P331S, C232S, C233S, M252Y, S254T, T256E, and/or any combination thereof, according to EU numbering; (iii) an IgG3 or a variant thereof, optionally comprising the amino acid substitution E235Y, according to EU numbering; or (iv) an IgG4 or a variant thereof, optionally comprising one or more of the following amino acid substitutions: E233P, F234V, L235A, G237A, E318A, S228P, L236E, S241P, L248E, T394D, M252Y, S254T, T256E, N297A, N297Q, and/or any combination thereof, according to EU numbering:
(III) comprises one or more amino acid substitutions that reduce an Fc effector function, optionally wherein the one or more amino acid substitutions are at position(s) selected from the group consisting of positions 234, 235, 236, 237, 265, 297 and 329, according to EU numbering;
(IV) comprises at least the following amino acid substitutions according to EU numbering: (ii-1) L234A, L235A, and P329G; (ii-2) L234A and L235A; (ii-3) I253A, H310A, and H435Q; (ii-4) I253A, H310A, and H435A; (ii-5) D265A and P329A; (ii-6) M252Y, S254T, and T256E; and/or (ii-7) M428L and N434S, optionally relative to a human IgG1 Fc region and/or to any of SEQ ID NOS: 71-74 (optionally not including the C-terminal K); and/or
(V) comprises an amino acid sequence which comprises or consists of the amino acid sequence of any one of SEQ ID NOS: 71-74, 81-84, 86-89, 91-94, 96-99, 471-474, 476-479, 481-484, and 486-489 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or is 100% identical thereto.

Embodiment B9. A nucleic acid or a combination of nucleic acids encoding the anti-LRP5/6 antibody or antibody fragment of any one of Embodiments B1-B8, optionally wherein the nucleic acid(s) is/are DNA, cDNA, RNA, mRNA, modified mRNA, or a DNA/RNA hybrid.

Embodiment B10. The nucleic acid or a combination of nucleic acids of Embodiments B9, comprising: (a) a first nucleic acid encoding a first polypeptide comprising said VH; and (b) a second nucleic acid encoding a second polypeptide comprising said VL.

Embodiment B11. A vector or a combination of vectors which comprises the nucleic acid or the combination of nucleic acids of Embodiment B9 or B10, optionally wherein: (i) the vector(s) comprise(s) one or more promoters operably linked to the nucleic acid(s); (ii) the vector(s) is/are an expression vector; and/or (iii) the vector(s) comprise(s) a plasmid, a viral vector (optionally adeno-associated viral, adenoviral, lentiviral, or retroviral), a lipid-based vector, a self-replicating RNA vector, a virus-like particle, a polymer-based vector, and/or a nanoparticle, optionally a lipid-based nanoparticle.

Embodiment B12. The vector or a combination of vectors of Embodiment B11, which encodes the anti-LRP5/6 antibody or antibody fragment of any one of Embodiments B1-B8 and comprises: (a) a first vector comprising a first nucleic acid encoding a first polypeptide comprising said VH; and (b) a second vector comprising a second nucleic acid encoding a second polypeptide comprising said VL.

Embodiment B13. A host cell, which comprises: (A) the anti-LRP5/6 antibody or antibody fragment of any one of Embodiments B1-B8; (B) the nucleic acid or the combination of nucleic acids of Embodiment B9 or B10; and/or (C) the vector or the combination of vectors of Embodiment B11 or B12, optionally wherein the host cell is: (i) mammalian, optionally human, non-human primate, monkey, rabbit, rodent, hamster, rat, or mouse; or (ii) non-mammalian, optionally plant, bacterial, fungal, yeast, protozoa, or insect, and optionally wherein the host cell is: (i) a human embryonal kidney (HEK) cell, optionally a HEK293 cell, or a variant thereof, further optionally Expi293F™ cell; (ii) a CHO (Chinese Hamster Ovary) cell; (iii) an immune cell or (iv) a hybridoma.

Embodiment B14. A population of cells, which comprises two or more host cells of Embodiment B13.

Embodiment B15. A pharmaceutical composition, which comprises: (I) (A) the anti-LRP5/6 antibody or antibody fragment of any one of Embodiments B1-B8; (B) the nucleic acid or the combination of nucleic acids of Embodiment B9 or B10; (C) the vector or the combination of vectors of Embodiment B11 or B12; and/or (D) the host cell of Embodiment B13 or the population of cells of Embodiment B14; and (II) a pharmaceutically acceptable carrier and/or excipient.

Embodiment B16. A method of treating a subject in need of such treatment, comprising administering to the subject an effective amount of: (A) the anti-LRP5/6 antibody or antibody fragment of any one of Embodiments B1-B8; (B) the nucleic acid or the combination of nucleic acids of Embodiment B9 or B10; (C) the vector or the combination of vectors of Embodiment B11 or B12; and/or (D) the host cell of Embodiment B13 or the population of cells of Embodiment B14; and/or (E) the pharmaceutical composition of Embodiment B15, optionally wherein: (a) the subject is (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile; (b) the subject comprises or has a risk of developing a disease, disorder, or a condition; and/or (c) the method further comprises administering to the subject an additional agent, optionally an adjuvant or a therapeutic agent.

Embodiment B17. A method of treating or preventing a disease, disorder, or a condition in a subject in need of such treatment, the method comprising administering an effective amount of: (A) the anti-LRP5/6 antibody or antibody fragment of any one of Embodiments B1-B8; (B) the nucleic acid or the combination of nucleic acids of Embodiment B9 or B10; (C) the vector or the combination of vectors of Embodiment B11 or B12; and/or (D) the host cell of Embodiment B13 or the population of cells of Embodiment B14; and/or (E) the pharmaceutical composition of Embodiment B15, optionally wherein: (a) the subject is (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile; and/or (b) the method further comprises administering to the subject an additional agent, optionally an adjuvant or a therapeutic agent.

Embodiment B18. A method of manufacturing the anti-LRP5/6 antibody or antibody fragment of any one of Embodiments B1-B8, comprising: (a) culturing cells comprising the nucleic acid or the combination of nucleic acids of Embodiment B9 or B10 in a condition that allows for expression of said anti-LRP5/6 antibody or antibody fragment, and (b) harvesting and purifying the anti-LRP5/6 antibody or antibody fragment from the cell culture from (a).

Embodiment B19. A method of manufacturing the host cell of Embodiment B13 or the population of such cells, comprising introducing the nucleic acid or the combination of nucleic acids of Embodiment B9 or B10 and/or the vector or the combination of vectors of Embodiment B11 or B12 into one or more cells, optionally wherein the introducing occurs in vitro, ex vivo, or in vivo.

Embodiment C1. A multispecific antibody or antibody fragment comprising at least: (A) a first antigen-binding region which specifically binds to Fzd4 and comprises a first heavy chain variable domain (VH1) and a first light chain variable domain (VL1), (B) a second antigen-binding region which specifically binds to LRP5 and/or LRP6 and comprises at least a second heavy chain variable domain (VH2), wherein: (A) the VH1 and the VL1 is according to the VH and VL of any one of the anti-Fzd4 antibody or antibody fragment of any one of Embodiments A1-A8; and/or (B) the VH2 is according to the VH of any one of the anti-LRP5/6 antibody or antibody fragment of any one of Embodiments B1-B8.

Embodiment C2. The multispecific antibody or antibody fragment of Embodiment C1, wherein: (A) the first antigen-binding region is, comprises, or is comprised in a fragment antigen-binding (Fab), a Fab′ fragment, a F(ab′)2, a single-chain Fab (scFab), a variable fragment (Fv), a single-chain Fv (scFv), a diabody, or a triabody; and/or (B) the second antigen-binding region: (i) is, comprises, or is comprised in a nanobody or a Fd fragment; or (ii) further comprises a second light chain variable domain (VL2) and is, comprises, or is comprised in a Fab, a Fab′ fragment, a F(ab′)2, a scFab, a Fv, a scFv, a diabody, or a triabody.

Embodiment C3. The multispecific antibody or antibody fragment of Embodiment C1 or C2, which comprises one or more of the following:
(a) an immunoglobulin heavy chain constant domain 1 (CH1) or a variant thereof, optionally wherein the CH1 or a variant thereof: (a-1) is of an IgG, an IgA, an IgE, an IgD, an IgM, optionally of an IgG1, an IgG4, an IgG2, or an IgG3, further optionally of human; and/or (a-2) comprises an amino acid sequence which comprises or consists of the amino acid sequence of SEQ ID NO: 21 or 22 or which is at least 80%, at least 85%, at least 90%, at least 91%, at least 92%, at least 93%, at least 94%, at least 95%, at least 96%, at least 97%, at least 98%, at least 99%, or 100% identical thereto;
(b) an immunoglobulin hinge or a variant thereof, optionally wherein the hinge or a variant thereof: (b-1) is of an IgG, an IgA, an IgE, an IgD, an IgM, optionally of an IgG1, an IgG4, an IgG2, or an IgG3, further optionally of human; and/or (b-2) comprises an amino acid sequence which comprises or consists of the amino acid sequence of SEQ ID NO: 31 or which is at least 80%, at least 85%, at least 90%, at least 91%, at least 92%, at least 93%, at least 94%, at least 95%, at least 96%, at least 97%, at least 98%, at least 99%, or 100% identical thereto;
(c) an immunoglobulin heavy chain constant domain 2 (CH2) or a variant thereof, optionally wherein the CH2 or a variant thereof: (c-1) is of an IgG, an IgA, an IgE, an IgD, an IgM, optionally of an IgG1, an IgG4, an IgG2, or an IgG3, further optionally of human; and/or (c-2) comprises an amino acid sequence which comprises or consists of the amino acid sequence of any of SEQ ID NOS: 41-46 or which is at least 80%, at least 85%, at least 90%, at least 91%, at least 92%, at least 93%, at least 94%, at least 95%, at least 96%, at least 97%, at least 98%, at least 99%, or 100% identical thereto;
(d) an immunoglobulin heavy chain constant domain 3 (CH3) or a variant thereof, optionally wherein the CH3 or a variant thereof: (d-1) is of an IgG, an IgA, an IgE, an IgD, an IgM, optionally of an IgG1, an IgG4, an IgG2, or an IgG3, further optionally of human; and/or (d-2) comprises an amino acid sequence which comprises or consists of the amino acid sequence of any of SEQ ID NOS: 51-54, 61-64, and 66-69 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or 100% identical thereto; and/or
(e) an immunoglobulin light chain constant domain (CL) or a variant thereof, optionally wherein the CL or a variant thereof: (e-1) is of a kappa light chain constant domain (CLκ), optionally human CLκ and optionally comprising an amino acid sequence which comprises or consists of the amino acid sequence of SEQ ID NO: 11 or which is at least 80%, at least 85%, at least 90%, at least 91%, at least 92%, at least 93%, at least 94%, at least 95%, at least 96%, at least 97%, at least 98%, at least 99%, or 100% identity thereto; or (e-2) is of a lambda light chain constant domain (CLλ), optionally human CLλ, and optionally comprising an amino acid sequence which comprises or consists of the amino acid sequence of SEQ ID NO: 12 or which is at least 80%, at least 85%, at least 90%, at least 91%, at least 92%, at least 93%, at least 94%, at least 95%, at least 96%, at least 97%, at least 98%, at least 99%, or 100% identity thereto, optionally wherein the multispecific antibody or antibody fragment comprises the CH2 and CH3 sequences of SEQ ID NOS: 42 and 51, respectively, SEQ ID NOS: 43 and 51, respectively, SEQ ID NOS: 45 and 61, respectively, SEQ ID NO: 45 and 66, respectively, SEQ ID NOS: 46 and 61, respectively, or SEQ ID NO: 46 and 66, respectively.

Embodiment C4. The multispecific antibody or antibody fragment of any one of Embodiments C1-C3, which comprises a Fc region, optionally wherein the Fc region:
(I) is of an IgG, an IgA, an IgE, an IgD, an IgM, optionally of an IgG1, an IgG4, an IgG2, or an IgG3, further optionally of human, or a variant thereof;
(II) is of: (i) an IgG1 or a variant thereof, optionally comprising one or more of the following amino acid substitutions: N297A, N297Q, D265A, L234A, L235A, C226S, C229S, P238S, E233P, L234V, G236-deleted, P238A, A327Q, A327G, P329A, P329G, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, H310A, L328F, M252Y, I253A, S254T, T256E, H435Q, H435A, and/or any combination thereof, according to EU numbering; (ii) an IgG2 or a variant thereof, optionally comprising one or more of the following amino acid substitutions: P238S, V234A, G237A, H268A, H268Q, H268E, V309L, N297A, N297Q, A330S, P331S, C232S, C233S, M252Y, S254T, T256E, and/or any combination thereof, according to EU numbering; (iii) an IgG3 or a variant thereof, optionally comprising the amino acid substitution E235Y, according to EU numbering; or (iv) an IgG4 or a variant thereof, optionally comprising one or more of the following amino acid substitutions: E233P, F234V, L235A, G237A, E318A, S228P, L236E, S241P, L248E, T394D, M252Y, S254T, T256E, N297A, N297Q, and/or any combination thereof, according to EU numbering:
(III) comprises one or more amino acid substitutions that reduce an Fc effector function, optionally wherein the one or more amino acid substitutions are at position(s) selected from the group consisting of positions 234, 235, 236, 237, 265, 297 and 329, according to EU numbering;
(IV) comprises at least the following amino acid substitutions according to EU numbering: (ii-1) L234A, L235A, and P329G; (ii-2) L234A and L235A; (ii-3) I253A, H310A, and H435Q; (ii-4) I253A, H310A, and H435A; (ii-5) D265A and P329A; (ii-6) M252Y, S254T, and T256E; and/or (ii-7) M428L and N434S, optionally relative to a human IgG1 Fc region and/or to any of SEQ ID NOS: 71-74 (optionally not including the C-terminal K); and/or
(V) comprises an amino acid sequence which comprises or consists of the amino acid sequence of any one of SEQ ID NOS: 71-74, 81-84, 86-89, 91-94, 96-99, 471-474, 476-479, 481-484, and 486-489 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or is 100% identical thereto.

Embodiment C5. The multispecific antibody or antibody fragment of any one of Embodiments C1-C4, which comprises a structure as depicted in any one of FIGS. 24-35, optionally any of the structures depicted in FIG. 24.

Embodiment C6. The multispecific antibody or antibody fragment of any one of Embodiments C1-C5, which comprises: (a) a first polypeptide comprising said VH1; and (b) a second polypeptide comprising said VH2 and said VL1, wherein the first and second polypeptides interact with each other, optionally via one or more disulfide bonds, permitting said VH1 and VL1 to form said first antigen-binding region, optionally wherein:
(a) the first polypeptide comprises the following in the order from the N-terminus to the C-terminus:
(a-1) said VH1 and
(a-2) a CH1 or a variant thereof, optionally wherein the CH1 or a variant thereof: (i) is of an IgG, optionally of an IgG1 or an IgG4, further optionally of human IgG1 or IgG4; and/or (ii) comprises an amino acid sequence which comprises or consists of the amino acid sequence of SEQ ID NO: 21 or 22 or which is at least 80%, at least 85%, at least 90%, at least 91%, at least 92%, at least 93%, at least 94%, at least 95%, at least 96%, at least 97%, at least 98%, at least 99%, or is 100% identical thereto;
(a-3) an immunoglobulin hinge or a variant thereof, optionally wherein the hinge or a variant thereof: (i) is of an IgG, optionally of an IgG1 or an IgG4, further optionally of human IgG1 or IgG4; and/or (ii) comprising an amino acid sequence which comprises or consists of the amino acid sequence of SEQ ID NO: 31 or which is at least 80%, at least 85%, at least 90%, at least 91%, at least 92%, at least 93%, at least 94%, at least 95%, at least 96%, at least 97%, at least 98%, at least 99%, or is 100% identical thereto;
(a-4) a CH2 or a variant thereof, optionally wherein the CH2 or a variant thereof: (i) is of an IgG, optionally of an IgG1 or an IgG4, further optionally of human IgG1 or IgG4; and/or (ii) comprises an amino acid sequence which comprises or consists of the amino acid sequence of any of SEQ ID NOS: 41-46 or which is at least 80%, at least 85%, at least 90%, at least 91%, at least 92%, at least 93%, at least 94%, at least 95%, at least 96%, at least 97%, at least 98%, at least 99%, or is 100% identical thereto; and
(a-5) a CH3 or a variant thereof, optionally wherein the CH3 or a variant thereof: (i) is of an IgG, optionally of an IgG1 or an IgG4, further optionally of human IgG1 or IgG4; and/or (ii) comprises an amino acid sequence which comprises or consists of the amino acid sequence of any of SEQ ID NOS: 51-54, 61-64, and 66-69 (optionally not including the C-terminal K) or which is at least 80%, at least 85%, at least 90%, at least 91%, at least 92%, at least 93%, at least 94%, at least 95%, at least 96%, at least 97%, at least 98%, at least 99%, or is 100% identical thereto; and/or
(b) the second polypeptide comprises the following in the order from the N-terminus to the C-terminus:
(b-1) said VH2;
(b-2) optionally a linker, which is optionally a peptide linker, further optionally a flexible linker, optionally wherein the linker: (i) comprises one or more amino acids, optionally one, two, three, four, five, six, seven, eight, nine, ten, eleven, or twelve amino acids; (ii) consists of small amino acids consisting of G, S, and/or A; (iii) comprises an amino acid sequence which comprises or consists of the amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG; and/or (iv) comprises an amino acid sequence which comprises or consists of multiple repeats of the amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG;
(b-3) said VL1; and (b-4) an CL or a variant thereof, optionally wherein the CL or a variant thereof: (i) is of a CLκ, optionally human CLκ, and optionally comprising an amino acid sequence comprising or consisting of the amino acid sequence of SEQ ID NO: 11 or having at least 80%, at least 85%, at least 90%, at least 91%, at least 92%, at least 93%, at least 94%, at least 95%, at least 96%, at least 97%, at least 98%, at least 99%, or 100% identity thereto; or (ii) is of a CLλ, optionally human CLλ, and optionally comprising an amino acid sequence comprising or consisting of the amino acid sequence of SEQ ID NO: 12 or having at least 80%, at least 85%, at least 90%, at least 91%, at least 92%, at least 93%, at least 94%, at least 95%, at least 96%, at least 97%, at least 98%, at least 99%, or 100% identity thereto, further optionally wherein the multispecific antibody or antibody fragment comprises: two of said first polypeptides forming a dimer with each other optionally via one or more disulfide bonds; and two of said second polypeptides, wherein one of the second polypeptides is interacting with one of the first polypeptides optionally via a disulfide bond and the other of the second polypeptides is interacting with the other of the first polypeptides optionally via a disulfide bond.

Embodiment C7. The multispecific antibody or antibody fragment of any one of Embodiments C1-C6, which comprises a Fc region, wherein the Fc region:
(I) is of an IgG, optionally of an IgG1 or an IgG4, further optionally of human, or a variant thereof,
(II) comprises at least the following amino acid substitutions according to EU numbering: (ii-1) L234A, L235A, and P329G; (ii-2) L234A and L235A; (ii-3) I253A, H310A, and H435Q; and/or (ii-4) I253A, H310A, and H435A, optionally relative to a human IgG1 Fc region and/or to any of SEQ ID NOS: 71-74 (optionally not including the C-terminal K); and/or
(III) comprises an amino acid sequence which comprises or consists of the amino acid sequence of any one of SEQ ID NOS: 71-74, 81-84, 86-89, 91-94, 96-99, 471-474, 476-479, 481-484, and 486-489 (optionally not including the C-terminal K) or comprises an amino acid sequence which is at least 80%, at least 85%, at least 90%, at least 91%, at least 92%, at least 93%, at least 94%, at least 95%, at least 96%, at least 97%, at least 98%, at least 99%, or 100% identical thereto.

Embodiment C8. The multispecific antibody or antibody fragment of any one of Embodiments C1-C7, which comprises: (a) a first polypeptide comprising an amino acid sequence which comprises or consists of the amino acid sequence of SEQ ID NO: 2, 7, 8, 9, 450, 451, 452, and 453 (optionally without the C-terminal K); and (b) a second polypeptide comprising an amino acid sequence which comprises or consists of the amino acid sequence of SEQ ID NO: 1.

Embodiment C9. The multispecific antibody or antibody fragment of Embodiment C8, which comprises: two of said first polypeptides forming a dimer with each other via one or more disulfide bonds; and two of said second polypeptides, wherein one of the second polypeptides is interacting with one of the first polypeptides via a disulfide bond and the other of the second polypeptides is interacting with the other of the first polypeptides via a disulfide bond.

Embodiment C10. A nucleic acid or a combination of nucleic acids encoding the multispecific antibody or antibody fragment of any one of Embodiments C1-C9, optionally wherein the nucleic acid(s) is/are DNA, cDNA, RNA, mRNA, modified mRNA, or a DNA/RNA hybrid.

Embodiment C11. The nucleic acid or a combination of nucleic acids of Embodiment C10, comprising: (a) a first nucleic acid encoding a first polypeptide comprising said VH1; and (b) a second nucleic acid encoding a second polypeptide comprising said VH2 and said VL1.

Embodiment C12. A vector or a combination of vectors which comprises the nucleic acid or the combination of nucleic acids of Embodiment C10 or C11, optionally wherein: (i) the vector(s) comprise(s) one or more promoters operably linked to the nucleic acid(s); (ii) the vector(s) is/are an expression vector; and/or (iii) the vector(s) comprise(s) a plasmid, a viral vector (optionally adeno-associated viral, adenoviral, lentiviral, or retroviral), a lipid-based vector, a self-replicating RNA vector, a virus-like particle, a polymer-based vector, and/or a nanoparticle, optionally a lipid-based nanoparticle.

Embodiment C13. The vector or a combination of vectors of Embodiment C12, which encodes the multispecific antibody or antibody fragment of any one of Embodiments C1-C9 and comprises: (a) a first vector comprising a first nucleic acid encoding a first polypeptide comprising said VH1; and (b) a second vector comprising a second nucleic acid encoding a second polypeptide comprising said VH2 and said VL1.

Embodiment C14. A host cell, which comprises: (A) the multispecific antibody or antibody fragment of any one of Embodiments C1-C9; (B) the nucleic acid or the combination of nucleic acids of Embodiment C10 or C11; and/or (C) the vector or the combination of vectors of Embodiment C12 or C13, optionally wherein the host cell is: (i) mammalian, optionally human, non-human primate, monkey, rabbit, rodent, hamster, rat, or mouse; or (ii) non-mammalian, optionally plant, bacterial, fungal, yeast, protozoa, or insect, and optionally wherein the host cell is: (i) a human embryonal kidney (HEK) cell, optionally a HEK293 cell, or a variant thereof, further optionally Expi293F™ cell; (ii) a CHO (Chinese Hamster Ovary) cell; (iii) an immune cell or (iv) a hybridoma.

Embodiment C15. A population of cells, which comprises two or more host cells of Embodiment B13.

Embodiment C16. A pharmaceutical composition, which comprises: (I) (A) the multispecific antibody or antibody fragment of any one of Embodiments C1-C9; (B) the nucleic acid or the combination of nucleic acids of Embodiment C10 or C11; (C) the vector or the combination of vectors of Embodiment C12 or C13; and/or (D) the host cell of Embodiment C14 or the population of cells of Embodiment C15; and (II) a pharmaceutically acceptable carrier and/or excipient.

Embodiment C17. A method of treating a subject in need of such treatment, comprising administering to the subject an effective amount of: (A) the multispecific antibody or antibody fragment of any one of Embodiments C1-C9; (B) the nucleic acid or the combination of nucleic acids of Embodiment C10 or C11; (C) the vector or the combination of vectors of Embodiment C12 or C13; and/or (D) the host cell of Embodiment C14 or the population of cells of Embodiment C15; and/or (E) the pharmaceutical composition of Embodiment C16, optionally wherein: (a) the subject is (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile; (b) the subject comprises or has a risk of developing a disease, disorder, or a condition; and/or (c) the method further comprises administering to the subject an additional agent, optionally an adjuvant or a therapeutic agent.

Embodiment C18. A method of treating or preventing a disease, disorder, or a condition in a subject in need of such treatment, the method comprising administering an effective amount of: (A) the multispecific antibody or antibody fragment of any one of Embodiments C1-C9; (B) the nucleic acid or the combination of nucleic acids of Embodiment C10 or C11; (C) the vector or the combination of vectors of Embodiment C12 or C13; and/or (D) the host cell of Embodiment C14 or the population of cells of Embodiment C15; and/or (E) the pharmaceutical composition of Embodiment C16, optionally wherein: (a) the subject is (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile; and/or (b) the method further comprises administering to the subject an additional agent, optionally an adjuvant or a therapeutic agent.

Embodiment C19. A method of inducing, promoting, stimulating, enhancing, and/or supporting a Wnt signaling in a target cell of a subject, comprising administering to the subject an effective amount of: (A) the multispecific antibody or antibody fragment of any one of Embodiments C1-C9; (B) the nucleic acid or the combination of nucleic acids of Embodiment C10 or C11; (C) the vector or the combination of vectors of Embodiment C12 or C13; and/or (D) the host cell of Embodiment C14 or the population of cells of Embodiment C15; and/or (E) the pharmaceutical composition of Embodiment C16, optionally wherein: (a) the subject is (i) a mammal, optionally a human, a non-human primate, a monkey, a horse, a cow, sheep, a goat, a pig, a dog, a cat, a rabbit, a rodent, a hamster, a rat, or a mouse; or (ii) a non-mammalian vertebrate, optionally a bird, fish, an amphibian, or a reptile; (b) the subject comprises or has a risk of developing a disease, disorder, or a condition; and/or (c) the method further comprises administering to the subject an additional agent, optionally an adjuvant or a therapeutic agent, and optionally wherein: (i) the Wnt signaling is or comprises Wnt/β-catenin signaling; and/or (ii) the method promotes, stimulates, enhances, and/or supports recruitment of multiple receptor complexes each comprising (ii-1) Fzd4 and (ii-2) LRP5 or LRP6.

Embodiment C20. The method of any one of Embodiments C17-C19, wherein the disease, disorder, or condition comprises one or more of the following:
(a) a retinopathy, optionally a retinal vascular disease (optionally caused by inhibition of vascular development or excessive angiogenesis) and/or optionally selected from the group consisting of exudative vitreoretinopathy, familiar exudative vitreoretinopathy (FEVR), retinopathy of prematurity, Norrie disease, diabetic retinopathy (DR), diabetic macular edema, diabetic macular ischemia, age-related macular degeneration (AMD) (including wet AMD and dry AMD), retinopathy of prematurity (ROP), osteoporosis-pseudoglioma syndrome (OPPG), retinal vein occlusion, and Coats disease;
(b) a vascular disorder, optionally vascular malformation or vascular insufficiency, further optionally associated with ischemia-induced neovascularization;
(c) a bone disease or bone injury, optionally selected from the group consisting of osteoporosis (including osteoporosis pseudoglioma (OPPG) syndrome and juvenile primary osteoporosis), bone mineral density variability, and cancer-associated osteolysis; (d) a muscle wasting disease, optionally selected from the group consisting of sarcopenia, cachexia, and muscular dystrophies; and/or
(e) a neural or neurodegenerative disease, optionally selected from the group consisting of stroke, traumatic brain injury, epilepsy, Alzheimer's disease, Parkinson's disease, Huntington's disease, amyotrophic lateral sclerosis, Friedreich ataxia, Lewy body disease, spinal muscular atrophy, motor neuron disease, multiple sclerosis, Batten disease, and Creutzfeldt-Jakob disease.

Embodiment C21. The method of any one of Embodiments C17-C20, wherein:
(I) the administering is: (i) to one or more of the subject's eyes, optionally intravitreally or via ocular drops; (ii) locally administering, optionally to the eye, ear, nose (optionally intranasally), skin (optionally transdermally or epicutaneously), mucosa, skin, or vagina, or by inhalation; or (iii) parenterally administering, optionally by injection (optionally intravenous, intramuscular, subcutaneous, intradermal, intrathecal, intra-arterial, intraarticular, intraosseous, or intraperitoneal administration) or by inhalation; or (iv) enterally administering, optionally orally, sublingually, buccally, or rectally; and/or
(II) the administering comprising administering the multispecific antibody or antibody fragment of any one of claims 1-20 to one or more of the subject's eyes at about 0.1-100000 ng per eye, about 1-100000 ng per eye, about 1-10000 ng per eye, about 10-10000 ng per eye, about 1-100 ng per eye, about 200-300 ng per eye, about 300-400 ng per eye, about 400-500 ng per eye, about 500-600 ng per eye, about 600-700 ng per eye, about 700-800 ng per eye, about 800-900 ng per eye, about 900-1000 ng per eye, about 1000-2000 ng per eye, about 2000-3000 ng per eye, about 3000-4000 ng per eye, about 4000-5000 ng per eye, about 5000-6000 ng per eye, about 6000-7000 ng per eye, about 7000-8000 ng per eye, about 8000-9000 ng per eye, about 9000-10000 ng per eye, about 10000-20000 ng per eye, about 20000-30000 ng per eye, about 30000-40000 ng per eye, about 40000-50000 ng per eye, about 50000-60000 ng per eye, about 60000-70000 ng per eye, about 70000-80000 ng per eye, about 80000-90000 ng per eye, or about 90000-100000 ng per eye.

Embodiment C22. A method of inducing, promoting, stimulating, enhancing, and/or supporting a Wnt signaling in a target cell, comprising contacting the target cell with an effective amount of: (A) the multispecific antibody or antibody fragment of any one of Embodiments C1-C9; (B) the nucleic acid or the combination of nucleic acids of Embodiment C10 or C11; (C) the vector or the combination of vectors of Embodiment C12 or C13; and/or (D) the host cell of Embodiment C14 or the population of cells of Embodiment C15; and/or (E) the pharmaceutical composition of Embodiment C16, optionally wherein: (I) the contacting occurs in vitro, ex vivo, or in vivo, (II) the method is for (i) preparing a cell and/or tissue for implantation, (ii) differentiation of a stem cell, and/or (iii) preparation of an organoid, (III) the method is for bone formation, vascular formation, and/or neural formation and/or differentiation.

Embodiment C23. A method of manufacturing the multispecific antibody or antibody fragment of any one of Embodiments C1-C9, comprising: (a) culturing cells comprising the nucleic acid or the combination of nucleic acids of Embodiment C10 or C11 in a condition that allows for expression of said multispecific antibody or antibody fragment, and (b) harvesting and purifying the multispecific antibody or antibody fragment from the cell culture from (a).

Embodiment C24. A method of manufacturing the host cell of Embodiment C14 or the population of such cells, comprising introducing the nucleic acid or the combination of nucleic acids of Embodiment C10 or C11 and/or the vector or the combination of vectors of Embodiment C12 or C13 into one or more cells, optionally wherein the introducing occurs in vitro, ex vivo, or in vivo.

Embodiments C25. The multispecific antibody or antibody fragment of any one of Embodiments C1-C9; the nucleic acid or the combination of nucleic acids of Embodiment C10 or C11; the vector or the combination of vectors of Embodiment C12 or C13; the host cell of Embodiment C14 or the population of cells of Embodiment C15; and/or the pharmaceutical composition of Embodiment C16, for use in medicine.

Embodiments C26. The multispecific antibody or antibody fragment of any one of Embodiments C1-C9; the nucleic acid or the combination of nucleic acids of Embodiment C10 or C11; the vector or the combination of vectors of Embodiment C12 or C13; the host cell of Embodiment C14 or the population of cells of Embodiment C15; and/or the pharmaceutical composition of Embodiment C16, for use in treating a disease, disorder, or condition, optionally wherein the disease, disorder, or condition comprises one or more of those recited in Embodiments C20.

Embodiments C27. Use of the multispecific antibody or antibody fragment of any one of Embodiments C1-C9; the nucleic acid or the combination of nucleic acids of Embodiment C10 or C11; the vector or the combination of vectors of Embodiment C12 or C13; the host cell of Embodiment C14 or the population of cells of Embodiment C15; and/or the pharmaceutical composition of Embodiment C16, for the manufacture of a medicament for treatment of a disease, disorder, or condition, optionally wherein the disease, disorder, or condition comprises one or more of those recited in Embodiments C20.

### APPENDIX

APPENDIX TABLE A
Anti-Fzd4 VH CDR and FR sequences defined by Kabat

| SEQ ID | | | |
|---|---|---|---|
| NO: | Name | Sequences | Notes |
| 110 | Anti-Fzd4 FRH1 | EVQLVESGGGLVX₁PG | X₁ is any amino acid. |
| | common sequence | GSLRLSCAASGFTFT | |
| 111 | Anti-Fzd4 FRH1 | EVQLVESGGGLVQPGG | |
| | | SLRLSCAASGFTFT | |
| 112 | Anti-Fzd4 FRH1 | EVQLVESGGGLVKPGG | |
| | | SLRLSCAASGFTFT | |
| 120 | Anti-Fzd4 CDRH1 | X₁YAMS | X₁ is any amino acid. |
| | common sequence | | |
| 121 | Anti-Fzd4 CDRH1 | SYAMS | |
| 122 | Anti-Fzd4 CDRH1 | NYAMS | |
| 123 | Anti-Fzd4 CDRH1 | AYAMS | |
| 124 | Anti-Fzd4 CDRH1 | QYAMS | |
| 130 | Anti-Fzd4 FRH2 | WVRQAPGKGLEWVS | |
| | common sequence | | |
| 131 | Anti-Fzd4 FRH2 | WVRQAPGKGLEWVS | |
| 140 | Anti-Fzd4 CDRH2 | AISGSGGSTYYAX₁SVK | X₁ is any amino acid. |
| | common sequence | G | |
| 141 | Anti-Fzd4 CDRH2 | AISGSGGSTYYAESVK | |
| | | G | |
| 142 | Anti-Fzd4 CDRH2 | AISGSGGSTYYADSVK | |
| | | G | |
| 143 | Anti-Fzd4 CDRH2 | AISGSGGSTYYAASVK | |
| | | G | |
| 144 | Anti-Fzd4 CDRH2 | AISGSGGSTYYASSVK | |
| | | G | |
| 150 | Anti-Fzd4 FRH3 | RFTISRDX₁SKNTLYLQ | X₁ is any amino acid; X₂ is |
| | common sequence | MNSLX₂X₃EDTAVYYC | any amino acid; and X₃ is |
| | | AR | any amino acid. |
| 151 | Anti-Fzd4 FRH3 | RFTISRDNSKNTLYLQ | |
| | | MNSLRAEDTAVYYCA | |
| | | R | |
| 152 | Anti-Fzd4 FRH3 | RFTISRDDSKNTLYLQ | |
| | | MNSLKTEDTAVYYCA | |
| | | R | |
| 160 | Anti-Fzd4 CDRH3 | ATGFGTVVFX₁Y | X₁ is any amino acid. |
| | common sequence | | |
| 161 | Anti-Fzd4 CDRH3 | ATGFGTVVFDY | |
| 170 | Anti-Fzd4 FRH4 | WGQGTLVTVSS | |
| | common sequence | | |
| 171 | Anti-Fzd4 FRH4 | WGQGTLVTVSS | |

APPENDIX TABLE B
Anti-Fzd4 VH sequences

| SEQ ID | | | |
|---|---|---|---|
| NO: | Name | Sequences | Notes |
| 180 | Anti-Fzd4 | EVQLVESGGGLVX₁PGGSLRLSC | X₁ is any amino acid; X₂ is |
| | VH | AASGFTFTX₂YAMSWVRQAPGK | any amino acid; X₃ is any |
| | common | GLEWVSAISGSGGSTYYAX₃SVK | amino acid; X₄ is any |
| | sequence | GRFTISRDX₄SKNTLYLQMNSLX₅ | amino acid; X₅ is any |
| | | X₆EDTAVYYCARATGFGTVVFX₇ | amino acid; X₆ is any |
| | | YWGQGTLVTVSS | amino acid; and X₇ is any |
| | | | amino acid. |
| 181 | Anti-Fzd4 | EVQLVESGGGLVQPGGSLRLSC | |
| | VH | AASGFTFTSYAMSWVRQAPGKG | |
| | | LEWVSAISGSGGSTYYAESVKG | |
| | | RFTISRDNSKNTLYLQMNSLRAE | |
| | | DTAVYYCARATGFGTVVFDYW | |
| | | GQGTLVTVSS | |
| 182 | Anti-Fzd4 | EVQLVESGGGLVKPGGSLRLSC | |
| | VH | AASGFTFTNYAMSWVRQAPGK | |
| | | GLEWVSAISGSGGSTYYADSVK | |
| | | GRFTISRDDSKNTLYLQMNSLKT | |
| | | EDTAVYYCARATGFGTVVFDY | |
| | | WGQGTLVTVSS | |
| 183 | Anti-Fzd4 | EVQLVESGGGLVKPGGSLRLSC | |
| | VH | AASGFTFTQYAMSWVRQAPGK | |
| | | GLEWVSAISGSGGSTYYADSVK | |
| | | GRFTISRDDSKNTLYLQMNSLKT | |
| | | EDTAVYYCARATGFGTVVFDY | |
| | | WGQGTLVTVSS | |
| 184 | Anti-Fzd4 | EVQLVESGGGLVKPGGSLRLSC | |
| | VH | AASGFTFTSYAMSWVRQAPGKG | |
| | | LEWVSAISGSGGSTYYADSVKG | |
| | | RFTISRDDSKNTLYLQMNSLKTE | |
| | | DTAVYYCARATGFGTVVFDYW | |
| | | GQGTLVTVSS | |
| 185 | Anti-Fzd4 | EVQLVESGGGLVKPGGSLRLSC | |
| | VH | AASGFTFTAYAMSWVRQAPGK | |
| | | GLEWVSAISGSGGSTYYADSVK | |
| | | GRFTISRDDSKNTLYLQMNSLKT | |
| | | EDTAVYYCARATGFGTVVFDY | |
| | | WGQGTLVTVSS | |
| 186 | Anti-Fzd4 | EVQLVESGGGLVKPGGSLRLSC | |
| | VH | AASGFTFTNYAMSWVRQAPGK | |
| | | GLEWVSAISGSGGSTYYASSVK | |
| | | GRFTISRDDSKNTLYLQMNSLKT | |
| | | EDTAVYYCARATGFGTVVFDY | |
| | | WGQGTLVTVSS | |
| 187 | Anti-Fzd4 | EVQLVESGGGLVKPGGSLRLSC | |
| | VH | AASGFTFTNYAMSWVRQAPGK | |
| | | GLEWVSAISGSGGSTYYAESVK | |
| | | GRFTISRDDSKNTLYLQMNSLKT | |
| | | EDTAVYYCARATGFGTVVFDY | |
| | | WGQGTLVTVSS | |
| 188 | Anti-Fzd4 | EVQLVESGGGLVKPGGSLRLSC | |
| | VH | AASGFTFTNYAMSWVRQAPGK | |
| | | GLEWVSAISGSGGSTYYAASVK | |
| | | GRFTISRDDSKNTLYLQMNSLKT | |
| | | EDTAVYYCARATGFGTVVFDY | |
| | | WGQGTLVTVSS | |

APPENDIX TABLE C
Anti-Fzd4 VL and VL CDR and FR sequences

| SEQ ID | | |
|---|---|---|
| NO: | Name | Sequences |
| 211 | Anti-Fzd4 | DIQMTQSPSSLSASVGDRVTITC |
| | FRL1 | |
| 221 | Anti-Fzd4 | RASQSISSYLN |
| | CDRL1 | |
| 231 | Anti-Fzd4 | WYQQKPGKAPKLLIY |
| | FRL2 | |
| 241 | Anti-Fzd4 | AASSLQS |
| | CDRL2 | |
| 251 | Anti-Fzd4 | GVPSRFSGSGSGTDFTLTISSLQPEDFATYYC |
| | FRL3 | |
| 261 | Anti-Fzd4 | QQSYSTPLT |
| | CDRL3 | |
| 271 | Anti-Fzd4 | FGGGTKVEIK |
| | FRL4 | |
| 281 | Anti-Fzd4 VL | DIQMTQSPSSLSASVGDRVTITCRASQSISSYLNWYQQ |
| | | KPGKAPKLLIYAASSLQSGVPSRFSGSGSGTDFTLTISSL |
| | | QPEDFATYYCQQSYSTPLTFGGGTKVEIK |

APPENDIX TABLE D
Anti-LRP5/6 VH CDR and FR sequences

| SEQ | | | |
|---|---|---|---|
| ID | | | |
| NO: | Name | Sequences | Notes |
| 310 | Anti-LRP5/6 | X₁VQLVESGGGLVQPGGSLRLS | X₁ is any amino acid; X2 is |
| | FRH1 common | CX₂X₃S | any amino acid; and X3 is |
| | sequence | | any amino acid. |
| 311 | Anti-LRP5/6 | EVQLVESGGGLVQPGGSLRLSC | |
| | FRH1 | ASS | |
| 312 | Anti-LRP5/6 | DVQLVESGGGLVQPGGSLRLSC | |
| | FRH1 | TSS | |
| 320 | Anti-LRP5/6 | ANIX₁SIET | X₁ is any amino acid. |
| | CDRH1 | | |
| | common | | |
| | sequence | | |
| 321 | Anti-LRP5/6 | ANIQSIET | |
| | CDRH1 | | |
| 322 | Anti-LRP5/6 | ANINSIET | |
| | CDRH1 | | |
| 323 | Anti-LRP5/6 | ANISSIET | |
| | CDRH1 | | |
| 330 | Anti-LRP5/6 | LGWX₁RQAPGKX₂X₃EX₄X₅X₆ | X₁ is any amino acid; X₂ is |
| | FRH2 common | | any amino acid; X₃ is any |
| | sequence | | amino acid; X₄ is any amino |
| | | | acid; X₅ is any amino acid; |
| | | | and X₆ is any amino acid. |
| 331 | Anti-LRP5/6 | LGWYRQAPGKQRELIA | |
| | FRH2 | | |
| 332 | Anti-LRP5/6 | LGWYRQAPGKGREWIA | |
| | FRH2 | | |
| 333 | Anti-LRP5/6 | LGWYRQAPGKQREWIA | |
| | FRH2 | | |
| 340 | Anti-LRP5/6 | NX₁RGGGYX₂ | X₁ is any amino acid; and |
| | CDRH2 | | X₂ is any amino acid. |
| | common | | |
| | sequence | | |
| 341 | Anti-LRP5/6 | NMRGGGYM | |
| | CDRH2 | | |
| 350 | Anti-LRP5/6 | KYAX₁SX₂KGRFTX₃SX₄X₅X₆X₇K | X₁ is any amino acid; X₂ is |
| | FRH3 common | NTX₈YLQMNSLX₉X₁₀EDTAVYY | any amino acid; X₃ is any |
| | sequence | C | amino acid; X₄ is any amino |
| | | | acid; X₅ is any amino acid; |
| | | | X₆ is any amino acid; X₇ is |
| | | | any amino acid; X₈ is any |
| | | | amino acid; X₉ is any amino |
| | | | acid; and X₁₀ is any amino |
| | | | acid. |
| 351 | Anti-LRP5/6 | KYADSLKGRFTMSTDNSKNTM | |
| | FRH3 | YLQMNSLRAEDTAVYYC | |
| 352 | Anti-LRP5/6 | KYAGSLKGRFTMSTESAKNTM | |
| | FRH3 | YLQMNSLKPEDTAVYYC | |
| 360 | Anti-LRP5/6 | YVKLRX₁X₂X₃YVY | X₁ is any amino acid; X₂ is |
| | CDRH3 | | any amino acid; and X₃ is |
| | common | | any amino acid. |
| | sequence | | |
| 361 | Anti-LRP5/6 | YVKLRDEDYVY | |
| | CDRH3 | | |
| 362 | Anti-LRP5/6 | YVKLRDDDYVY | |
| | CDRH3 | | |
| 363 | Anti-LRP5/6 | YVKLRDEEYVY | |
| | CDRH3 | | |
| 364 | Anti-LRP5/6 | YVKLRDESYVY | |
| | CDRH3 | | |
| 365 | Anti-LRP5/6 | YVKLRDEAYVY | |
| | CDRH3 | | |
| 366 | Anti-LRP5/6 | YVKLRDETYVY | |
| | CDRH3 | | |
| 367 | Anti-LRP5/6 | YVKLRESEYVY | |
| | CDRH3 | | |
| 368 | Anti-LRP5/6 | YVKLRESTYVY | |
| | CDRH3 | | |
| 369 | Anti-LRP5/6 | YVKLRESSYVY | |
| | CDRH3 | | |
| 300 | Anti-LRP5/6 | YVKLREDEYVY | |
| | CDRH3 | | |
| 301 | Anti-LRP5/6 | YVKLRESDYVY | |
| | CDRH3 | | |
| 302 | Anti-LRP5/6 | YVKLRSDEYVY | |
| | CDRH3 | | |
| 303 | Anti-LRP5/6 | YVKLRSSDYVY | |
| | CDRH3 | | |
| 370 | Anti-LRP5/6 | X₁GQGTX₂VTVSS | X₁ is any amino acid; and |
| | FRH4 common | | X2 is any amino acid. |
| | sequence | | |
| 371 | Anti-LRP5/6 | RGQGTQVTVSS | |
| | FRH4 | | |
| 372 | Anti-LRP5/6 | WGQGTLVTVSS | |
| | FRH4 | | |

APPENDIX TABLE E
Anti-LRP5/6 VH sequences

| SEQ | | | |
|---|---|---|---|
| ID | | | |
| NO: | Name | Sequences | Notes |
| 380 | Anti-LRP5/6 | X₁VQLVESGGGLVQP | X₁ is any amino acid; X₂ is |
| | VH common | GGSLRLSCX₂X₃SANI | any amino acid; and X₃ is any |
| | sequence | X₄SIETLGWX₅RQAPG | amino acid; X₄ is any amino |
| | | KX₆X₇EX₈X₉X₁₀NX₁₁R | acid; X₅ is any amino acid; |
| | | GGGYX₁₂KYAX₁₃SX₁₄ | X₆ is any amino acid; X₇ is |
| | | KGRFTX₁₅SX₁₆X₁₇X₁₈ | any amino acid X₈ is any |
| | | X₁₉KNTX₂₀YLQMNSL | amino acid; X₉ is any amino |
| | | X₂₁X₂₂EDTAVYYCYV | acid; and X₁₀ is any amino |
| | | KLRX₂₃X₂₄X₂₅YVYX₂₆ | acid; X₁₁ is any amino acid; |
| | | GQGTX₂₇VTVSS | X₁₂ is any amino acid; X₁₃ is |
| | | | any amino acid; X₁₄ is any |
| | | | amino acid; X₁₅ is any amino |
| | | | acid; X₁₆ is any amino acid; |
| | | | X₁₇ is any amino acid; X₁₈ is |
| | | | any amino acid; X₁₉ is any |
| | | | amino acid; X₂₀ is any amino |
| | | | acid; X₂₁ is any amino acid; |
| | | | X₂₂ is any amino acid; X₂₃ is |
| | | | any amino acid; X₂₄ is any |
| | | | amino acid; X₂₅ is any amino |
| | | | acid; X₂₆ is any amino acid; |
| | | | and X₂₇ is any amino acid. |
| 381 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDE | |
| | | DYVYRGQGTQVTVS | |
| | | S | |
| 382 | Anti-LRP5/6 | DVQLVESGGGLVQP | |
| | VH | GGSLRLSCTSSANINS | |
| | | IETLGWYRQAPGKQR | |
| | | ELIANMRGGGYMKY | |
| | | AGSLKGRFTMSTESA | |
| | | KNTMYLQMNSLKPE | |
| | | DTAVYYCYVKLRDD | |
| | | DYVYRGQGTQVTVS | |
| | | S | |
| 383 | Anti-LRP5/6 | EVQLVESGGGLVQPG | |
| | VH | GSLRLSCASSANINSI | |
| | | ETLGWYRQAPGKGR | |
| | | EWIANMRGGGYMK | |
| | | YADSLKGRFTMSTD | |
| | | NSKNTMYLQMNSLR | |
| | | AEDTAVYYCYVKLR | |
| | | DDDYVYWGQGTLVT | |
| | | VSS | |
| 384 | Anti-LRP5/6 | EVQLVESGGGLVQPG | |
| | VH | GSLRLSCASSANINSI | |
| | | ETLGWYRQAPGKQR | |
| | | EWIANMRGGGYMK | |
| | | YADSLKGRFTMSTD | |
| | | NSKNTMYLQMNSLR | |
| | | AEDTAVYYCYVKLR | |
| | | DDDYVYWGQGTLVT | |
| | | VSS | |
| 385 | Anti-LRP5/6 | EVQLVESGGGLVQPG | |
| | VH | GSLRLSCASSANINSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDD | |
| | | DYVYRGQGTQVTVS | |
| | | S | |
| 386 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDD | |
| | | DYVYRGQGTQVTVS | |
| | | S | |
| 387 | Anti-LRP5/6 | EVQLVESGGGLVQPG | |
| | VH | GSLRLSCASSANISSIE | |
| | | TLGWYRQAPGKQRE | |
| | | LIANMRGGGYMKYA | |
| | | DSLKGRFTMSTDNSK | |
| | | NTMYLQMNSLRAED | |
| | | TAVYYCYVKLRDDD | |
| | | YVYRGQGTQVTVSS | |
| 388 | Anti-LRP5/6 | EVQLVESGGGLVQPG | |
| | VH | GSLRLSCASSANINSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDE | |
| | | DYVYRGQGTQVTVS | |
| | | S | |
| 389 | Anti-LRP5/6 | EVQLVESGGGLVQPG | |
| | VH | GSLRLSCASSANINSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRED | |
| | | EYVYRGQGTQVTVS | |
| | | S | |
| 390 | Anti-LRP5/6 | EVQLVESGGGLVQPG | |
| | VH | GSLRLSCASSANINSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRES | |
| | | DYVYRGQGTQVTVS | |
| | | S | |
| 391 | Anti-LRP5/6 | EVQLVESGGGLVQPG | |
| | VH | GSLRLSCASSANINSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRSD | |
| | | EYVYRGQGTQVTVS | |
| | | S | |
| 392 | Anti-LRP5/6 | EVQLVESGGGLVQPG | |
| | VH | GSLRLSCASSANINSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRSS | |
| | | DYVYRGQGTQVTVS | |
| | | S | |
| 393 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDE | |
| | | EYVYRGQGTQVTVS | |
| | | S | |
| 394 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDE | |
| | | SYVYRGQGTQVTVSS | |
| 395 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDE | |
| | | AYVYRGQGTQVTVS | |
| | | S | |
| 396 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRDE | |
| | | TYVYRGQGTQVTVS | |
| | | S | |
| 397 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRES | |
| | | EYVYRGQGTQVTVS | |
| | | S | |
| 398 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRES | |
| | | TYVYRGQGTQVTVS | |
| | | S | |
| 399 | Anti-LRP5/6 | EVQLVESGGGLVQPG | |
| | VH | GSLRLSCASSANIQSI | |
| | | ETLGWYRQAPGKQR | |
| | | ELIANMRGGGYMKY | |
| | | ADSLKGRFTMSTDNS | |
| | | KNTMYLQMNSLRAE | |
| | | DTAVYYCYVKLRESS | |
| | | YVYRGQGTQVTVSS | |

APPENDIX TABLE F
Linker unit sequences

| SEQ ID | | |
|---|---|---|
| NO: | Name | Sequences |
| 101 | Linker unit 1 | GGGGS |
| 102 | Linker unit 2 | GGSGS |
| 103 | Linker unit 3 | GSGSG |
| 104 | Linker unit 4 | GGGS |
| 105 | Linker unit 5 | GGGGGS |

APPENDIX TABLE G
Constant domain sequences

| SEQ ID | | |
|---|---|---|
| NO: | Name | Sequences |
| 11 | Human IgG CK | RTVAAPSVFIFPPSDEQLKSGTASVVCLLNNF |
| | | YPREAKVQWKVDNALQSGNSQESVTEQDSK |
| | | DSTYSLSSTLTLSKADYEKHKVYACEVTHQG |
| | | LSSPVTKSFNRGEC |
| 12 | Human IgG CL | GQPKAAPSVTLFPPSSEELQANKATLVCLISDF |
| | | YPGAVTVAWKADSSPVKAGVETTTPSKQSNN |
| | | KYAASSYLSLTPEQWKSHRSYSCQVTHEGST |
| | | VEKTVAPTECS |
| 21 | Human IgG1 CH1, | ASTKGPSVFPLAPSSKSTSGGTAALGCLVKDY |
| | reference 1 | FPEPVTVSWNSGALTSGVHTFPAVLQSSGLYS |
| | | LSSVVTVPSSSLGTQTYICNVNHKPSNTKVDK |
| | | KV |
| 22 | Human IgG1 CH1, | ASTKGPSVFPLAPSSKSTSGGTAALGCLVKDY |
| | reference 2 | FPEPVTVSWNSGALTSGVHTFPAVLQSSGLYS |
| | | LSSVVTVPSSSLGTQTYICNVNHKPSNTKVDK |
| | | RV |
| 31 | Human IgG1 hinge, | EPKSCDKTHTCPPCP |
| | reference | |
| 41 | Human IgG1 CH2, | APELLGGPSVFLFPPKPKDTLMISRTPEVTCVV |
| | reference | VDVSHEDPEVKFNWYVDGVEVHNAKTKPRE |
| | | EQYNSTYRVVSVLTVLHQDWLNGKEYKCKV |
| | | SNKALPAPIEKTISKAK |
| 42 | Human IgG1 CH2, | APEAAGGPSVFLFPPKPKDTLMISRTPEVTCV |
| | reference + LALAPG | VVDVSHEDPEVKFNWYVDGVEVHNAKTKPR |
| | | EEQYNSTYRVVSVLTVLHQDWLNGKEYKCK |
| | | VSNKALGAPIEKTISKAK |
| 43 | Human IgG1 CH2, | APEAAGGPSVFLFPPKPKDTLMISRTPEVTCV |
| | reference + LALA | VVDVSHEDPEVKFNWYVDGVEVHNAKTKPR |
| | | EEQYNSTYRVVSVLTVLHQDWLNGKEYKCK |
| | | VSNKALPAPIEKTISKAK |
| 44 | Human IgG1 CH2, | APELLGGPSVFLFPPKPKDTLMASRTPEVTCV |
| | reference + IAHA | VVDVSHEDPEVKFNWYVDGVEVHNAKTKPR |
| | | EEQYNSTYRVVSVLTVLAQDWLNGKEYKCK |
| | | VSNKALPAPIEKTISKAK |
| 45 | Human IgG1 CH2, | APEAAGGPSVFLFPPKPKDTLMASRTPEVTCV |
| | reference + LALAPG- | VVDVSHEDPEVKFNWYVDGVEVHNAKTKPR |
| | IAHA | EEQYNSTYRVVSVLTVLAQDWLNGKEYKCK |
| | | VSNKALGAPIEKTISKAK |
| 46 | Human IgG1 CH2, | APEAAGGPSVFLFPPKPKDTLMASRTPEVTCV |
| | reference + LALA- | VVDVSHEDPEVKFNWYVDGVEVHNAKTKPR |
| | IAHA | EEQYNSTYRVVSVLTVLAQDWLNGKEYKCK |
| | | VSNKALPAPIEKTISKAK |
| 51 | Human IgG1 CH3, | GQPREPQVYTLPPSREEMTKNQVSLTCLVKGF |
| | reference 1 (356E and | YPSDIAVEWESNGQPENNYKTTPPVLDSDGSF |
| | 358M) | FLYSKLTVDKSRWQQGNVFSCSVMHEALHN |
| | | HYTQKSLSLSPGK |
| 52 | Human IgG1 CH3, | GQPREPQVYTLPPSRDELTKNQVSLTCLVKGF |
| | reference 2 (356D and | YPSDIAVEWESNGQPENNYKTTPPVLDSDGSF |
| | 358L) | FLYSKLTVDKSRWQQGNVFSCSVMHEALHN |
| | | HYTQKSLSLSPGK |
| 53 | Human IgG1 CH3, | GQPREPQVYTLPPSRDELTKNQVSLTCLVKGF |
| | reference 3 (356D, | YPSDIAVEWESNGQPENNYKTTPPVLDSDGSF |
| | 358L, and V422I) | FLYSKLTVDKSRWQQGNIFSCSVMHEALHNH |
| | | YTQKSLSLSPGK |
| 54 | Human IgG1 CH3, | GQPREPQVYTLPPSRDELTKNQVSLTCLVKGF |
| | reference 4 (356D, | YPSDIAVEWESNGQPENNYKTTPPVLDSDGSF |
| | 358L, and A431G) | FLYSKLTVDKSRWQQGNVFSCSVMHEGLHN |
| | | HYTQKSLSLSPGK |
| 61 | Human IgG1 CH3, | GQPREPQVYTLPPSREEMTKNQVSLTCLVKGF |
| | reference 1 + HQ | YPSDIAVEWESNGQPENNYKTTPPVLDSDGSF |
| | | FLYSKLTVDKSRWQQGNVFSCSVMHEALHN |
| | | QYTQKSLSLSPGK |
| 62 | Human IgG1 CH3, | GQPREPQVYTLPPSRDELTKNQVSLTCLVKGF |
| | reference 2+ HQ | YPSDIAVEWESNGQPENNYKTTPPVLDSDGSF |
| | | FLYSKLTVDKSRWQQGNVFSCSVMHEALHN |
| | | QYTQKSLSLSPGK |
| 63 | Human IgG1 CH3, | GQPREPQVYTLPPSRDELTKNQVSLTCLVKGF |
| | reference 3 + HQ | YPSDIAVEWESNGQPENNYKTTPPVLDSDGSF |
| | | FLYSKLTVDKSRWQQGNIFSCSVMHEALHNQ |
| | | YTQKSLSLSPGK |
| 64 | Human IgG1 CH3, | GQPREPQVYTLPPSRDELTKNQVSLTCLVKGF |
| | reference 4 + HQ | YPSDIAVEWESNGQPENNYKTTPPVLDSDGSF |
| | | FLYSKLTVDKSRWQQGNVFSCSVMHEGLHN |
| | | QYTQKSLSLSPGK |
| 66 | Human IgG1 CH3, | GQPREPQVYTLPPSREEMTKNQVSLTCLVKGF |
| | reference 1 + HA | YPSDIAVEWESNGQPENNYKTTPPVLDSDGSF |
| | | FLYSKLTVDKSRWQQGNVFSCSVMHEALHN |
| | | AYTQKSLSLSPGK |
| 67 | Human IgG1 CH3, | GQPREPQVYTLPPSRDELTKNQVSLTCLVKGF |
| | reference 2+ HA | YPSDIAVEWESNGQPENNYKTTPPVLDSDGSF |
| | | FLYSKLTVDKSRWQQGNVFSCSVMHEALHN |
| | | AYTQKSLSLSPGK |
| 68 | Human IgG1 CH3, | GQPREPQVYTLPPSRDELTKNQVSLTCLVKGF |
| | reference 3 + HA | YPSDIAVEWESNGQPENNYKTTPPVLDSDGSF |
| | | FLYSKLTVDKSRWQQGNIFSCSVMHEALHNA |
| | | YTQKSLSLSPGK |
| 69 | Human IgG1 CH3, | GQPREPQVYTLPPSRDELTKNQVSLTCLVKGF |
| | reference 4 + HA | YPSDIAVEWESNGQPENNYKTTPPVLDSDGSF |
| | | FLYSKLTVDKSRWQQGNVFSCSVMHEGLHN |
| | | AYTQKSLSLSPGK |

Underlined residues are differences relative to the corresponding reference sequence.

APPENDIX TABLE H
Fc region sequences

| SEQ | | |
|---|---|---|
| ID | | |
| NO: | Name | Sequences |
| 71 | Human IgG1 Fc, | DKTHTCPPCPAPELLGGPSVFLFPPKPKDTLMISRTPE |
| | reference 1 (356E and | VTCVVVDVSHEDPEVKFNWYVDGVEVHNAKTKPRE |
| | 358M) | EQYNSTYRVVSVLTVLHQDWLNGKEYKCKVSNKAL |
| | | PAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPGK |
| 72 | Human IgG1 Fc, | DKTHTCPPCPAPELLGGPSVFLFPPKPKDTLMISRTPE |
| | reference 2 (356D and | VTCVVVDVSHEDPEVKFNWYVDGVEVHNAKTKPRE |
| | 358L) | EQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPGK |
| 73 | Human IgG1 Fc, | DKTHTCPPCPAPELLGGPSVFLFPPKPKDTLMISRTPE |
| | reference 3 (356D, | VTCVVVDVSHEDPEVKFNWYVDGVEVHNAKTKPRE |
| | 358L, and V422I) | EQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNHYTQK |
| | | SLSLSPGK |
| 74 | Human IgG1 Fc, | DKTHTCPPCPAPELLGGPSVFLFPPKPKDTLMISRTPE |
| | reference 4 (356D, | VTCVVVDVSHEDPEVKFNWYVDGVEVHNAKTKPRE |
| | 358L, and A431G) | EQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNHYTQK |
| | | SLSLSPGK |
| 81 | Human IgG1 Fc, | DKTHTCPPCPAPEAAGGPSVFLFPPKPKDTLMISRTPE |
| | reference 1 + | VTCVVVDVSHEDPEVKFNWYVDGVEVHNAKTKPRE |
| | LALAPG | EQYNSTYRVVSVLTVLHQDWLNGKEYKCKVSNKAL |
| | | GAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPGK |
| 82 | Human IgG1 Fc, | DKTHTCPPCPAPEAAGGPSVFLFPPKPKDTLMISRTPE |
| | reference 2 + | VTCVVVDVSHEDPEVKFNWYVDGVEVHNAKTKPRE |
| | LALAPG | EQYNSTYRVVSVLTVLHQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPGK |
| 83 | Human IgG1 Fc, | DKTHTCPPCPAPEAAGGPSVFLFPPKPKDTLMISRTPE |
| | reference 3 + | VTCVVVDVSHEDPEVKFNWYVDGVEVHNAKTKPRE |
| | LALAPG | EQYNSTYRVVSVLTVLHQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNHYTQK |
| | | SLSLSPGK |
| 84 | Human IgG1 Fc, | DKTHTCPPCPAPEAAGGPSVFLFPPKPKDTLMISRTPE |
| | reference 4 + | VTCVVVDVSHEDPEVKFNWYVDGVEVHNAKTKPRE |
| | LALAPG | EQYNSTYRVVSVLTVLHQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNHYTQK |
| | | SLSLSPGK |
| 86 | Human IgG1 Fc, | DKTHTCPPCPAPEAAGGPSVFLFPPKPKDTLMISRTPE |
| | reference 1 + LALA | VTCVVVDVSHEDPEVKFNWYVDGVEVHNAKTKPRE |
| | | EQYNSTYRVVSVLTVLHQDWLNGKEYKCKVSNKAL |
| | | PAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPGK |
| 87 | Human IgG1 Fc, | DKTHTCPPCPAPEAAGGPSVFLFPPKPKDTLMISRTPE |
| | reference 2 + LALA | VTCVVVDVSHEDPEVKFNWYVDGVEVHNAKTKPRE |
| | | EQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNHYTQK |
| | | SLSLSPGK |
| 88 | Human IgG1 Fc, | DKTHTCPPCPAPEAAGGPSVFLFPPKPKDTLMISRTPE |
| | reference 3 + LALA | VTCVVVDVSHEDPEVKFNWYVDGVEVHNAKTKPRE |
| | | EQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNHYTQK |
| | | SLSLSPGK |
| 89 | Human IgG1 Fc, | DKTHTCPPCPAPEAAGGPSVFLFPPKPKDTLMISRTPE |
| | reference 4 + LALA | VTCVVVDVSHEDPEVKFNWYVDGVEVHNAKTKPRE |
| | | EQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNHYTQK |
| | | SLSLSPGK |
| 91 | Human IgG1 Fc, | DKTHTCPPCPAPELLGGPSVFLFPPKPKDTLMASRTPE |
| | reference 1 + | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNQYTQK |
| | | SLSLSPGK |
| 92 | Human IgG1 Fc, | DKTHTCPPCPAPELLGGPSVFLFPPKPKDTLMASRTPE |
| | reference 2 + | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNQYTQK |
| | | SLSLSPGK |
| 93 | Human IgG1 Fc, | DKTHTCPPCPAPELLGGPSVFLFPPKPKDTLMASRTPE |
| | reference 3 + | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNQYTQK |
| | | SLSLSPGK |
| 94 | Human IgG1 Fc, | DKTHTCPPCPAPELLGGPSVFLFPPKPKDTLMASRTPE |
| | reference 4 + | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNQYTQK |
| | | SLSLSPGK |
| 96 | Human IgG1 Fc, | DKTHTCPPCPAPELLGGPSVFLFPPKPKDTLMASRTPE |
| | reference 1 + | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNAYTQK |
| | | SLSLSPGK |
| 97 | Human IgG1 Fc, | DKTHTCPPCPAPELLGGPSVFLFPPKPKDTLMASRTPE |
| | reference 2 + | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNAYTQK |
| | | SLSLSPGK |
| 98 | Human IgG1 Fc, | DKTHTCPPCPAPELLGGPSVFLFPPKPKDTLMASRTPE |
| | reference 3 + | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNAYTQK |
| | | SLSLSPGK |
| 99 | Human IgG1 Fc, | DKTHTCPPCPAPELLGGPSVFLFPPKPKDTLMASRTPE |
| | reference 4 + | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNAYTQK |
| | | SLSLSPGK |
| 471 | Human IgG1 Fc, | DKTHTCPPCPAPEAAGGPSVFLFPPKPKDTLMASRTPE |
| | reference 1 + | VTCVVVDVSHEDPEVKFNWYVDGVEVHNAKTKPRE |
| | LALAPG-IAHAHQ | EQYNSTYRVVSVLTVLAQDWLNGKEYKCKVSNKAL |
| | | GAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNQYTQK |
| | | SLSLSPGK |
| 472 | Human IgG1 Fc, | DKTHTCPPCPAPEAAGGPSVFLFPPKPKDTLMASRTPE |
| | reference 2 + | VTCVVVDVSHEDPEVKFNWYVDGVEVHNAKTKPRE |
| | LALAPG-IAHAHQ | EQYNSTYRVVSVLTVLAQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNQYTQK |
| | | SLSLSPGK |
| 473 | Human IgG1 Fc, | DKTHTCPPCPAPEAAGGPSVFLFPPKPKDTLMASRTPE |
| | reference 3 + | VTCVVVDVSHEDPEVKFNWYVDGVEVHNAKTKPRE |
| | LALAPG-IAHAHQ | EQYNSTYRVVSVLTVLAQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNQYTQK |
| | | SLSLSPGK |
| 474 | Human IgG1 Fc, | DKTHTCPPCPAPEAAGGPSVFLFPPKPKDTLMASRTPE |
| | reference 4 + | VTCVVVDVSHEDPEVKFNWYVDGVEVHNAKTKPRE |
| | LALAPG-IAHAHQ | EQYNSTYRVVSVLTVLAQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNQYTQK |
| | | SLSLSPGK |
| 476 | Human IgG1 Fc, | DKTHTCPPCPAPEAAGGPSVFLFPPKPKDTLMASRTPE |
| | reference 1 + | VTCVVVDVSHEDPEVKFNWYVDGVEVHNAKTKPRE |
| | LALAPG-IAHAHA | EQYNSTYRVVSVLTVLAQDWLNGKEYKCKVSNKAL |
| | | GAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNAYTQK |
| | | SLSLSPGK |
| 477 | Human IgG1 Fc, | DKTHTCPPCPAPEAAGGPSVFLFPPKPKDTLMASRTPE |
| | reference 2 + | VTCVVVDVSHEDPEVKFNWYVDGVEVHNAKTKPRE |
| | LALAPG-IAHAHA | EQYNSTYRVVSVLTVLAQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNAYTQK |
| | | SLSLSPGK |
| 478 | Human IgG1 Fc, | DKTHTCPPCPAPEAAGGPSVFLFPPKPKDTLMASRTPE |
| | reference 3 + | VTCVVVDVSHEDPEVKFNWYVDGVEVHNAKTKPRE |
| | LALAPG-IAHAHA | EQYNSTYRVVSVLTVLAQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNAYTQK |
| | | SLSLSPGK |
| 479 | Human IgG1 Fc, | DKTHTCPPCPAPEAAGGPSVFLFPPKPKDTLMASRTPE |
| | reference 4 + | VTCVVVDVSHEDPEVKFNWYVDGVEVHNAKTKPRE |
| | LALAPG-IAHAHA | EQYNSTYRVVSVLTVLAQDWLNGKEYKCKVSNKAL |
| | | GAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNAYTQK |
| | | SLSLSPGK |
| 481 | Human IgG1 Fc, | DKTHTCPPCPAPEAAGGPSVFLFPPKPKDTLMASRTPE |
| | reference 1 + LALA- | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNQYTQK |
| | | SLSLSPGK |
| 482 | Human IgG1 Fc, | DKTHTCPPCPAPEAAGGPSVFLFPPKPKDTLMASRTPE |
| | reference 2 + LALA- | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNQYTQK |
| | | SLSLSPGK |
| 483 | Human IgG1 Fc, | DKTHTCPPCPAPEAAGGPSVFLFPPKPKDTLMASRTPE |
| | reference 3 + LALA- | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNQYTQK |
| | | SLSLSPGK |
| 484 | Human IgG1 Fc, | DKTHTCPPCPAPEAAGGPSVFLFPPKPKDTLMASRTPE |
| | reference 4 + LALA- | VTCVVVDVSHEDPEVKFNWYVDGVEVHNAKTKPRE |
| | IAHAHQ | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNQYTQK |
| | | SLSLSPGK |
| 486 | Human IgG1 Fc, | DKTHTCPPCPAPEAAGGPSVFLFPPKPKDTLMASRTPE |
| | reference 1 + LALA- | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSREEMTKNQVSLT |
| | | CLVKGFYPSDIAVEWESNGQPENNYKTTPPVLDSDGS |
| | | FFLYSKLTVDKSRWQQGNVFSCSVMHEALHNAYTQK |
| | | SLSLSPGK |
| 487 | Human IgG1 Fc, | DKTHTCPPCPAPEAAGGPSVFLFPPKPKDTLMASRTPE |
| | reference 2 + LALA- | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNAYTQK |
| | | SLSLSPGK |
| 488 | Human IgG1 Fc, | DKTHTCPPCPAPEAAGGPSVFLFPPKPKDTLMASRTPE |
| | reference 3 + LALA- | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNIFSCSVMHEALHNAYTQK |
| | | SLSLSPGK |
| 489 | Human IgG1 Fc, | DKTHTCPPCPAPEAAGGPSVFLFPPKPKDTLMASRTPE |
| | reference 4 + LALA- | VTCVVVDVSHEDPEVKFNWYVDGVEVHNAKTKPRE |
| | IAHAHA | EQYNSTYRVVSVLTVLAQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEGLHNAYTQK |
| | | SLSLSPGK |

Underlined residues are differences relative to the corresponding reference sequence.

APPENDIX TABLE I
Bispecifics-exemplary common sequences

| SEQ | | | |
|---|---|---|---|
| ID | | | |
| NO: | Name | Sequences | Notes |
| 401 | Bispecific LC | X₁VQLVESGGGLVQ | X₁ is any amino acid; X₂ is any amino |
| | common sequence | PGGSLRLSCX₂X₃SA | acid; and X₃ is any amino acid; X₄ is |
| | | NIX₄SIETLGWX₅RQ | any amino acid; X₅ is any amino acid; |
| | | APGKX₆X₇EX₈X₉X₁₀ | X₆ is any amino acid; X₇ is any amino |
| | | NX₁₁RGGGYX₁₂KYA | acid X₈ is any amino acid; X₉ is any |
| | | X₁₃SX₁₄KGRFTX₁₅SX | amino acid; and X₁₀ is any amino acid; |
| | | ₁₆X₁₇X₁₈X₁₉KNTX₂₀Y | X₁₁ is any amino acid; X₁₂ is any |
| | | LQMNSLX₂₁X₂₂EDT | amino acid; X₁₃ is any amino acid; X₁₄ |
| | | AVYYCYVKLRX₂₃X | is any amino acid; X1₅ is any amino |
| | | ₂₄X25YVYX₂₆GQGTX | acid; X₁₆ is any amino acid; X₁₇ is any |
| | | ₂₇VTVSSGGGGSDIQ | amino acid; X₁₈ is any amino acid; |
| | | MTQSPSSLSASVGD | X₁₉ is any amino acid; X₂₀ is any |
| | | RVTITCRASQSISSY | amino acid; X₂₁ is any amino acid; X₂₂ |
| | | LNWYQQKPGKAPK | is any amino acid; X₂₃ is any amino |
| | | LLIYAASSLQSGVPS | acid; X₂₄ is any amino acid; X₂₅ is any |
| | | RFSGSGSGTDFTLTI | amino acid; X₂₆ is any amino acid; and |
| | | SSLQPEDFATYYCQ | X₂₇ is any amino acid. |
| | | QSYSTPLTFGGGTK | |
| | | VEIKRTVAAPSVFIF | |
| | | PPSDEQLKSGTASV | |
| | | VCLLNNFYPREAKV | |
| | | QWKVDNALQSGNS | |
| | | QESVTEQDSKDSTY | |
| | | SLSSTLTLSKADYE | |
| | | KHKVYACEVTHQG | |
| | | LSSPVTKSFNRGEC | |
| 402 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (LALAPG) | TFTX₂YAMSWVRQ | amino acid; X₅ is any amino acid; X₆ |
| | | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX₇YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPEAAGGPSV | |
| | | FLFPPKPKDTLMISR | |
| | | TPEVTCVVVDVSHE | |
| | | DPEVKFNWYVDGV | |
| | | EVHNAKTKPREEQ | |
| | | YNSTYRVVSVLTVL | |
| | | HQDWLNGKEYKCK | |
| | | VSNKALGAPIEKTIS | |
| | | KAKGQPREPQVYTL | |
| | | PPSREEMTKNQVSL | |
| | | TCLVKGFYPSDIAV | |
| | | EWESNGQPENNYK | |
| | | TTPPVLDSDGSFFLY | |
| | | SKLTVDKSRWQQG | |
| | | NVFSCSVMHEALH | |
| | | NHYTQKSLSLSPGK | |
| 407 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (LALA) | TFTX₂YAMSWVRQ | amino acid; X₅ is any amino acid; X₆ |
| | | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX₇YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPEAAGGPSV | |
| | | FLFPPKPKDTLMISR | |
| | | TPEVTCVVVDVSHE | |
| | | DPEVKFNWYVDGV | |
| | | EVHNAKTKPREEQ | |
| | | YNSTYRVVSVLTVL | |
| | | HQDWLNGKEYKCK | |
| | | VSNKALPAPIEKTIS | |
| | | KAKGQPREPQVYTL | |
| | | PPSREEMTKNQVSL | |
| | | TCLVKGFYPSDIAV | |
| | | EWESNGQPENNYK | |
| | | TTPPVLDSDGSFFLY | |
| | | SKLTVDKSRWQQG | |
| | | NVFSCSVMHEALH | |
| | | NHYTQKSLSLSPGK | |
| 408 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (IAHAHQ) | TFTX₂YAMSWVRQ | amino acid; X₅ is any amino acid; X₆ |
| | | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX₇YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPELLGGPSVF | |
| | | LFPPKPKDTLMASR | |
| | | TPEVTCVVVDVSHE | |
| | | DPEVKFNWYVDGV | |
| | | EVHNAKTKPREEQ | |
| | | YNSTYRVVSVLTVL | |
| | | AQDWLNGKEYKCK | |
| | | VSNKALPAPIEKTIS | |
| | | KAKGQPREPQVYTL | |
| | | PPSREEMTKNQVSL | |
| | | TCLVKGFYPSDIAV | |
| | | EWESNGQPENNYK | |
| | | TTPPVLDSDGSFFLY | |
| | | SKLTVDKSRWQQG | |
| | | NVFSCSVMHEALH | |
| | | NQYTQKSLSLSPGK | |
| 409 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (IAHAHA) | TFTX₂YAMSWVRQ | amino acid; X₅ is any amino acid; X₆ |
| | | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX₇YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPELLGGPSVF | |
| | | LFPPKPKDTLMASR | |
| | | TPEVTCVVVDVSHE | |
| | | DPEVKFNWYVDGV | |
| | | EVHNAKTKPREEQ | |
| | | YNSTYRVVSVLTVL | |
| | | AQDWLNGKEYKCK | |
| | | VSNKALPAPIEKTIS | |
| | | KAKGQPREPQVYTL | |
| | | PPSREEMTKNQVSL | |
| | | TCLVKGFYPSDIAV | |
| | | EWESNGQPENNYK | |
| | | TTPPVLDSDGSFFLY | |
| | | SKLTVDKSRWQQG | |
| | | NVFSCSVMHEALH | |
| | | NAYTQKSLSLSPGK | |
| 460 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (LALAPG- | TFTX₂YAMSWVRQ | amino acid; X₅ is any amino acid; X₆ |
| | IAHAHQ) | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX₇YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPEAAGGPSV | |
| | | FLFPPKPKDTLMAS | |
| | | RTPEVTCVVVDVSH | |
| | | EDPEVKFNWYVDG | |
| | | VEVHNAKTKPREE | |
| | | QYNSTYRVVSVLTV | |
| | | LAQDWLNGKEYKC | |
| | | KVSNKALGAPIEKT | |
| | | ISKAKGQPREPQVY | |
| | | TLPPSREEMTKNQV | |
| | | SLTCLVKGFYPSDIA | |
| | | VEWESNGQPENNY | |
| | | KTTPPVLDSDGSFFL | |
| | | YSKLTVDKSRWQQ | |
| | | GNVFSCSVMHEAL | |
| | | HNQYTQKSLSLSPG | |
| | | K | |
| 461 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (LALAPG- | TFTX₂YAMSWVRQ | amino acid; X₅ is any amino acid; X₆ |
| | IAHAHA) | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX₇YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPEAAGGPSV | |
| | | FLFPPKPKDTLMAS | |
| | | RTPEVTCVVVDVSH | |
| | | EDPEVKFNWYVDG | |
| | | VEVHNAKTKPREE | |
| | | QYNSTYRVVSVLTV | |
| | | LAQDWLNGKEYKC | |
| | | KVSNKALGAPIEKT | |
| | | ISKAKGQPREPQVY | |
| | | TLPPSREEMTKNQV | |
| | | SLTCLVKGFYPSDIA | |
| | | VEWESNGQPENNY | |
| | | KTTPPVLDSDGSFFL | |
| | | YSKLTVDKSRWQQ | |
| | | GNVFSCSVMHEAL | |
| | | HNAYTQKSLSLSPG | |
| | | K | |
| 462 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (LALA- | TFTX₂YAMSWVRQ | amino acid; X₅ is any amino acid; X₆ |
| | IAHAHQ) | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX₇YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPEAAGGPSV | |
| | | FLFPPKPKDTLMAS | |
| | | RTPEVTCVVVDVSH | |
| | | EDPEVKFNWYVDG | |
| | | VEVHNAKTKPREE | |
| | | QYNSTYRVVSVLTV | |
| | | LAQDWLNGKEYKC | |
| | | KVSNKALPAPIEKTI | |
| | | SKAKGQPREPQVYT | |
| | | LPPSREEMTKNQVS | |
| | | LTCLVKGFYPSDIA | |
| | | VEWESNGQPENNY | |
| | | KTTPPVLDSDGSFFL | |
| | | YSKLTVDKSRWQQ | |
| | | GNVFSCSVMHEAL | |
| | | HNQYTQKSLSLSPG | |
| | | K | |
| 463 | Bispecific HC | EVQLVESGGGLVX₁ | X₁ is any amino acid; X₂ is any amino |
| | common sequence | PGGSLRLSCAASGF | acid; X₃ is any amino acid; X₄ is any |
| | (LALA- | TFTX₂YAMSWVRQ | amino acid; X₅ is any amino acid; X₆ |
| | IAHAHA) | APGKGLEWVSAISG | is any amino acid; and X₇ is any |
| | | SGGSTYYAX₃SVKG | amino acid. |
| | | RFTISRDX₄SKNTLY | |
| | | LQMNSLX₅X₆EDTA | |
| | | VYYCARATGFGTV | |
| | | VFX7YWGQGTLVT | |
| | | VSSASTKGPSVFPL | |
| | | APSSKSTSGGTAAL | |
| | | GCLVKDYFPEPVTV | |
| | | SWNSGALTSGVHTF | |
| | | PAVLQSSGLYSLSS | |
| | | VVTVPSSSLGTQTYI | |
| | | CNVNHKPSNTKVD | |
| | | KKVEPKSCDKTHTC | |
| | | PPCPAPEAAGGPSV | |
| | | FLFPPKPKDTLMAS | |
| | | RTPEVTCVVVDVSH | |
| | | EDPEVKFNWYVDG | |
| | | VEVHNAKTKPREE | |
| | | QYNSTYRVVSVLTV | |
| | | LAQDWLNGKEYKC | |
| | | KVSNKALPAPIEKTI | |
| | | SKAKGQPREPQVYT | |
| | | LPPSREEMTKNQVS | |
| | | LTCLVKGFYPSDIA | |
| | | VEWESNGQPENNY | |
| | | KTTPPVLDSDGSFFL | |
| | | YSKLTVDKSRWQQ | |
| | | GNVFSCSVMHEAL | |
| | | HNAYTQKSLSLSPG | |
| | | K | |

APPENDIX TABLE J:
Bispecifics-exemplary HC and LC sequences

| SEQ ID | | |
|---|---|---|
| NO: | Name | Sequences |
| 1 | hp4SD1-3 LC | EVQLVESGGGLVQPGGSLRLSCASSANIQSIETL |
| | | GWYRQAPGKQRELIANMRGGGYMKYADSLK |
| | | GRFTMSTDNSKNTMYLQMNSLRAEDTAVYYC |
| | | YVKLRDEDYVYRGQGTQVTVSSGGGGSDIQM |
| | | TQSPSSLSASVGDRVTITCRASQSISSYLNWYQ |
| | | QKPGKAPKLLIYAASSLQSGVPSRFSGSGSGTD |
| | | FTLTISSLQPEDFATYYCQQSYSTPLTFGGGTKV |
| | | EIKRTVAAPSVFIFPPSDEQLKSGTASVVCLLNN |
| | | FYPREAKVQWKVDNALQSGNSQESVTEQDSK |
| | | DSTYSLSSTLTLSKADYEKHKVYACEVTHQGL |
| | | SSPVTKSFNRGEC |
| 2 | hp4SD1-3 HC | EVQLVESGGGLVQPGGSLRLSCAASGFTFTSYA |
| | (with | MSWVRQAPGKGLEWVSAISGSGGSTYYAESV |
| | LALAPG Fc) | KGRFTISRDNSKNTLYLQMNSLRAEDTAVYYC |
| | | ARATGFGTVVFDYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPEAAGGPSVFLFPPKPKDTLMISRTP |
| | | EVTCVVVDVSHEDPEVKFNWYVDGVEVHNAK |
| | | TKPREEQYNSTYRVVSVLTVLHQDWLNGKEY |
| | | KCKVSNKALGAPIEKTISKAKGQPREPQVYTLP |
| | | PSREEMTKNQVSLTCLVKGFYPSDIAVEWESN |
| | | GQPENNYKTTPPVLDSDGSFFLYSKLTVDKSR |
| | | WQQGNVFSCSVMHEALHNHYTQKSLSLSPGK |
| 7 | hp4SD1-3 | EVQLVESGGGLVQPGGSLRLSCAASGFTFTSYA |
| | variant HC | MSWVRQAPGKGLEWVSAISGSGGSTYYAESV |
| | (with LALA | KGRFTISRDNSKNTLYLQMNSLRAEDTAVYYC |
| | Fc) | ARATGFGTVVFDYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPEAAGGPSVFLFPPKPKDTLMISRTP |
| | | EVTCVVVDVSHEDPEVKFNWYVDGVEVHNAK |
| | | TKPREEQYNSTYRVVSVLTVLHQDWLNGKEY |
| | | KCKVSNKALPAPIEKTISKAKGQPREPQVYTLP |
| | | PSREEMTKNQVSLTCLVKGFYPSDIAVEWESN |
| | | GQPENNYKTTPPVLDSDGSFFLYSKLTVDKSR |
| | | WQQGNVFSCSVMHEALHNHYTQKSLSLSPGK |
| 8 | hp4SD1-3 | EVQLVESGGGLVQPGGSLRLSCAASGFTFTSYA |
| | variant HC | MSWVRQAPGKGLEWVSAISGSGGSTYYAESV |
| | (with | KGRFTISRDNSKNTLYLQMNSLRAEDTAVYYC |
| | IAHAHQ Fc) | ARATGFGTVVFDYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPELLGGPSVFLFPPKPKDTLMASRTP |
| | | EVTCVVVDVSHEDPEVKFNWYVDGVEVHNAK |
| | | TKPREEQYNSTYRVVSVLTVLAQDWLNGKEY |
| | | KCKVSNKALPAPIEKTISKAKGQPREPQVYTLP |
| | | PSREEMTKNQVSLTCLVKGFYPSDIAVEWESN |
| | | GQPENNYKTTPPVLDSDGSFFLYSKLTVDKSR |
| | | WQQGNVFSCSVMHEALHNQYTQKSLSLSPGK |
| 9 | hp4SD1-3 | EVQLVESGGGLVQPGGSLRLSCAASGFTFTSYA |
| | variant HC | MSWVRQAPGKGLEWVSAISGSGGSTYYAESV |
| | (with | KGRFTISRDNSKNTLYLQMNSLRAEDTAVYYC |
| | IAHAHA Fc) | ARATGFGTVVFDYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPELLGGPSVFLFPPKPKDTLMASRTP |
| | | EVTCVVVDVSHEDPEVKFNWYVDGVEVHNAK |
| | | TKPREEQYNSTYRVVSVLTVLAQDWLNGKEY |
| | | KCKVSNKALPAPIEKTISKAKGQPREPQVYTLP |
| | | PSREEMTKNQVSLTCLVKGFYPSDIAVEWESN |
| | | GQPENNYKTTPPVLDSDGSFFLYSKLTVDKSR |
| | | WQQGNVFSCSVMHEALHNAYTQKSLSLSPGK |
| 450 | hp4SD1-3 | EVQLVESGGGLVQPGGSLRLSCAASGFTFTSYA |
| | variant HC | MSWVRQAPGKGLEWVSAISGSGGSTYYAESV |
| | (with | KGRFTISRDNSKNTLYLQMNSLRAEDTAVYYC |
| | LALAPG- | ARATGFGTVVFDYWGQGTLVTVSSASTKGPSV |
| | IAHAHQ Fc) | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPEAAGGPSVFLFPPKPKDTLMASRT |
| | | PEVTCVVVDVSHEDPEVKFNWYVDGVEVHNA |
| | | KTKPREEQYNSTYRVVSVLTVLAQDWLNGKE |
| | | YKCKVSNKALGAPIEKTISKAKGQPREPQVYTL |
| | | PPSREEMTKNQVSLTCLVKGFYPSDIAVEWESN |
| | | GQPENNYKTTPPVLDSDGSFFLYSKLTVDKSR |
| | | WQQGNVFSCSVMHEALHNQYTQKSLSLSPGK |
| 451 | hp4SD1-3 | EVQLVESGGGLVQPGGSLRLSCAASGFTFTSYA |
| | variant HC | MSWVRQAPGKGLEWVSAISGSGGSTYYAESV |
| | (with | KGRFTISRDNSKNTLYLQMNSLRAEDTAVYYC |
| | LALAPG- | ARATGFGTVVFDYWGQGTLVTVSSASTKGPSV |
| | IAHAHA Fc) | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPEAAGGPSVFLFPPKPKDTLMASRT |
| | | PEVTCVVVDVSHEDPEVKFNWYVDGVEVHNA |
| | | KTKPREEQYNSTYRVVSVLTVLAQDWLNGKE |
| | | YKCKVSNKALGAPIEKTISKAKGQPREPQVYTL |
| | | PPSREEMTKNQVSLTCLVKGFYPSDIAVEWESN |
| | | GQPENNYKTTPPVLDSDGSFFLYSKLTVDKSR |
| | | WQQGNVFSCSVMHEALHNAYTQKSLSLSPGK |
| 452 | hp4SD1-3 | EVQLVESGGGLVQPGGSLRLSCAASGFTFTSYA |
| | variant HC | MSWVRQAPGKGLEWVSAISGSGGSTYYAESV |
| | (with LALA- | KGRFTISRDNSKNTLYLQMNSLRAEDTAVYYC |
| | IAHAHQ Fc) | ARATGFGTVVFDYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPEAAGGPSVFLFPPKPKDTLMASRT |
| | | PEVTCVVVDVSHEDPEVKFNWYVDGVEVHNA |
| | | KTKPREEQYNSTYRVVSVLTVLAQDWLNGKE |
| | | YKCKVSNKALPAPIEKTISKAKGQPREPQVYTL |
| | | PPSREEMTKNQVSLTCLVKGFYPSDIAVEWESN |
| | | GQPENNYKTTPPVLDSDGSFFLYSKLTVDKSR |
| | | WQQGNVFSCSVMHEALHNQYTQKSLSLSPGK |
| 453 | hp4SD1-3 | EVQLVESGGGLVQPGGSLRLSCAASGFTFTSYA |
| | variant HC | MSWVRQAPGKGLEWVSAISGSGGSTYYAESV |
| | (with LALA- | KGRFTISRDNSKNTLYLQMNSLRAEDTAVYYC |
| | IAHAHA Fc) | ARATGFGTVVFDYWGQGTLVTVSSASTKGPSV |
| | | FPLAPSSKSTSGGTAALGCLVKDYFPEPVTVSW |
| | | NSGALTSGVHTFPAVLQSSGLYSLSSVVTVPSS |
| | | SLGTQTYICNVNHKPSNTKVDKKVEPKSCDKT |
| | | HTCPPCPAPEAAGGPSVFLFPPKPKDTLMASRT |
| | | PEVTCVVVDVSHEDPEVKFNWYVDGVEVHNA |
| | | KTKPREEQYNSTYRVVSVLTVLAQDWLNGKE |
| | | YKCKVSNKALPAPIEKTISKAKGQPREPQVYTL |
| | | PPSREEMTKNQVSLTCLVKGFYPSDIAVEWESN |
| | | GQPENNYKTTPPVLDSDGSFFLYSKLTVDKSR |
| | | WQQGNVFSCSVMHEALHNAYTQKSLSLSPGK |
| 421 | 4SD1- | DVQLVESGGGLVQPGGSLRLSCTSSANINSIETL |
| | 03_LALAPG | GWYRQAPGKQRELIANMRGGGYMKYAGSLK |
| | LC | GRFTMSTESAKNTMYLQMNSLKPEDTAVYYC |
| | | YVKLRDDDYVYRGQGTQVTVSSGGSGSDIQM |
| | | TQSPSSLSASVGDRVTITCRASQSISSYLNWYQ |
| | | QKPGKAPKLLIYAASSLQSGVPSRFSGSGSGTD |
| | | FTLTISSLQPEDFATYYCQQSYSTPLTFGGGTKV |
| | | EIKRTVAAPSVFIFPPSDEQLKSGTASVVCLLNN |
| | | FYPREAKVQWKVDNALQSGNSQESVTEQDSK |
| | | DSTYSLSSTLTLSKADYEKHKVYACEVTHQGL |
| | | SSPVTKSFNRGEC |
| 422 | 4SD1- | EVQLVESGGGLVKPGGSLRLSCAASGFTFTNY |
| | 03_LALAPG | AMSWVRQAPGKGLEWVSAISGSGGSTYYADS |
| | HC (with | VKGRFTISRDDSKNTLYLQMNSLKTEDTAVYY |
| | LALAPG Fc) | CARATGFGTVVFDYWGQGTLVTVSSASTKGPS |
| | | VFPLAPSSKSTSGGTAALGCLVKDYFPEPVTVS |
| | | WNSGALTSGVHTFPAVLQSSGLYSLSSVVTVPS |
| | | SSLGTQTYICNVNHKPSNTKVDKKVEPKSCDK |
| | | THTCPPCPAPEAAGGPSVFLFPPKPKDTLMISRT |
| | | PEVTCVVVDVSHEDPEVKFNWYVDGVEVHNA |
| | | KTKPREEQYNSTYRVVSVLTVLHQDWLNGKE |
| | | YKCKVSNKALGAPIEKTISKAKGQPREPQVYTL |
| | | PPSREEMTKNQVSLTCLVKGFYPSDIAVEWESN |
| | | GQPENNYKTTPPVLDSDGSFFLYSKLTVDKSR |
| | | WQQGNVFSCSVMHEALHNHYTQKSLSLSPGK |

APPENDIX TABLE K
Miscellaneous sequences

| SEQ | | |
|---|---|---|
| ID | | |
| NO: | Name | Sequences |
| 3 | Human Norrin | MRKHVLAASFSMLSLLVIMGDTDSKTDSSFIMDSDPRRCM |
| | | RHHYVDSISHPLYKCSSKMVLLARCEGHCSQASRSEPLVSF |
| | | STVLKQPFRSSCHCCRPQTSKLKALRLRCSGGMRLTATYR |
| | | YILSCHCEECNS |
| 4 | Human Fzd4 | MAWRGAGPSVPGAPGGVGLSLGLLLQLLLLLGPARGFGDE |
| | | EERRCDPIRISMCQNLGYNVTKMPNLVGHELQTDAELQLT |
| | | TFTPLIQYGCSSQLQFFLCSVYVPMCTEKINIPIGPCGGMCLS |
| | | VKRRCEPVLKEFGFAWPESLNCSKFPPQNDHNHMCMEGPG |
| | | DEEVPLPHKTPIQPGEECHSVGTNSDQYIWVKRSLNCVLKC |
| | | GYDAGLYSRSAKEFTDIWMAVWASLCFISTAFTVLTFLIDS |
| | | SRFSYPERPIIFLSMCYNIYSIAYIVRLTVGRERISCDFEEAAE |
| | | PVLIQEGLKNTGCAIIFLLMYFFGMASSIWWVILTLTWFLA |
| | | AGLKWGHEAIEMHSSYFHIAAWAIPAVKTIVILIMRLVDAD |
| | | ELTGLCYVGNQNLDALTGFVVAPLFTYLVIGTLFIAAGLVA |
| | | LFKIRSNLQKDGTKTDKLERLMVKIGVFSVLYTVPATCVIA |
| | | CYFYEISNWALFRYSADDSNMAVEMLKIFMSLLVGITSGM |
| | | WIWSAKTLHTWQKCSNRLVNSGKVKREKRGNGWVKPGK |
| | | GSETVV |
| 5 | Human LRP5 | MEAAPPGPPWPLLLLLLLLLALCGCPAPAAASPLLLFANRR |
| | | DVRLVDAGGVKLESTIVVSGLEDAAAVDFQFSKGAVYWT |
| | | DVSEEAIKQTYLNQTGAAVQNVVISGLVSPDGLACDWVGK |
| | | KLYWTDSETNRIEVANLNGTSRKVLFWQDLDQPRAIALDP |
| | | AHGYMYWTDWGETPRIERAGMDGSTRKIIVDSDIYWPNGL |
| | | TIDLEEQKLYWADAKLSFIHRANLDGSFRQKVVEGSLTHPF |
| | | ALTLSGDTLYWTDWQTRSIHACNKRTGGKRKEILSALYSP |
| | | MDIQVLSQERQPFFHTRCEEDNGGCSHLCLLSPSEPFYTCA |
| | | CPTGVQLQDNGRTCKAGAEEVLLLARRTDLRRISLDTPDFT |
| | | DIVLQVDDIRHAIAIDYDPLEGYVYWTDDEVRAIRRAYLDG |
| | | SGAQTLVNTEINDPDGIAVDWVARNLYWTDTGTDRIEVTR |
| | | LNGTSRKILVSEDLDEPRAIALHPVMGLMYWTDWGENPKI |
| | | ECANLDGQERRVLVNASLGWPNGLALDLQEGKLYWGDA |
| | | KTDKIEVINVDGTKRRTLLEDKLPHIFGFTLLGDFIYWTDW |
| | | QRRSIERVHKVKASRDVIIDQLPDLMGLKAVNVAKVVGTN |
| | | PCADRNGGCSHLCFFTPHATRCGCPIGLELLSDMKTCIVPE |
| | | AFLVFTSRAAIHRISLETNNNDVAIPLTGVKEASALDFDVSN |
| | | NHIYWTDVSLKTISRAFMNGSSVEHVVEFGLDYPEGMAVD |
| | | WMGKNLYWADTGTNRIEVARLDGQFRQVLVWRDLDNPR |
| | | SLALDPTKGYIYWTEWGGKPRIVRAFMDGTNCMTLVDKV |
| | | GRANDLTIDYADQRLYWTDLDTNMIESSNMLGQERVVIAD |
| | | DLPHPFGLTQYSDYIYWTDWNLHSIERADKTSGRNRTLIQG |
| | | HLDFVMDILVFHSSRQDGLNDCMHNNGQCGQLCLAIPGGH |
| | | RCGCASHYTLDPSSRNCSPPTTFLLFSQKSAISRMIPDDQHS |
| | | PDLILPLHGLRNVKAIDYDPLDKFIYWVDGRQNIKRAKDD |
| | | GTQPFVLTSLSQGQNPDRQPHDLSIDIYSRTLFWTCEATNTI |
| | | NVHRLSGEAMGVVLRGDRDKPRAIVVNAERGYLYFTNMQ |
| | | DRAAKIERAALDGTEREVLFTTGLIRPVALVVDNTLGKLF |
| | | WVDADLKRIESCDLSGANRLTLEDANIVQPLGLTILGKHLY |
| | | WIDRQQQMIERVEKTTGDKRTRIQGRVAHLTGIHAVEEVS |
| | | LEEFSAHPCARDNGGCSHICIAKGDGTPRCSCPVHLVLLQN |
| | | LLTCGEPPTCSPDQFACATGEIDCIPGAWRCDGFPECDDQS |
| | | DEEGCPVCSAAQFPCARGQCVDLRLRCDGEADCQDRSDEA |
| | | DCDAICLPNQFRCASGQCVLIKQQCDSFPDCIDGSDELMCEI |
| | | TKPPSDDSPAHSSAIGPVIGIILSLFVMGGVYFVCQRVVCQR |
| | | YAGANGPFPHEYVSGTPHVPLNFIAPGGSQHGPFTGIACGK |
| | | SMMSSVSLMGGRGGVPLYDRNHVTGASSSSSSSTKATLYP |
| | | PILNPPPSPATDPSLYNMDMFYSSNIPATARPYRPYIIRGMA |
| | | PPTTPCSTDVCDSDYSASRWKASKYYLDLNSDSDPYPPPPT |
| | | PHSQYLSAEDSCPPSPATERSYFHLFPPPPSPCTDSS |
| 6 | Human LRP6 | MGAVLRSLLACSFCVLLRAAPLLLYANRRDLRLVDATNGK |
| | | ENATIVVGGLEDAAAVDFVFSHGLIYWSDVSEEAIKRTEFN |
| | | KTESVQNVVVSGLLSPDGLACDWLGEKLYWTDSETNRIEV |
| | | SNLDGSLRKVLFWQELDQPRAIALDPSSGFMYWTDWGEVP |
| | | KIERAGMDGSSRFIIINSEIYWPNGLTLDYEEQKLYWADAK |
| | | LNFIHKSNLDGTNRQAVVKGSLPHPFALTLFEDILYWTDWS |
| | | THSILACNKYTGEGLREIHSDIFSPMDIHAFSQQRQPNATNP |
| | | CGIDNGGCSHLCLMSPVKPFYQCACPTGVKLLENGKTCKD |
| | | GATELLLLARRTDLRRISLDTPDFTDIVLQLEDIRHAIAIDYD |
| | | PVEGYIYWTDDEVRAIRRSFIDGSGSQFVVTAQIAHPDGIAV |
| | | DWVARNLYWTDTGTDRIEVTRLNGTMRKILISEDLEEPRAI |
| | | VLDPMVGYMYWTDWGEIPKIERAALDGSDRVVLVNTSLG |
| | | WPNGLALDYDEGKIYWGDAKTDKIEVMNTDGTGRRVLVE |
| | | DKIPHIFGFTLLGDYVYWTDWQRRSIERVHKRSAEREVIID |
| | | QLPDLMGLKATNVHRVIGSNPCAEENGGCSHLCLYRPQGL |
| | | RCACPIGFELISDMKTCIVPEAFLLFSRRADIRRISLETNNNN |
| | | VAIPLTGVKEASALDFDVTDNRIYWTDISLKTISRAFMNGS |
| | | ALEHVVEFGLDYPEGMAVDWLGKNLYWADTGTNRIEVSK |
| | | LDGQHRQVLVWKDLDSPRALALDPAEGFMYWTEWGGKP |
| | | KIDRAAMDGSERTTLVPNVGRANGLTIDYAKRRLYWTDL |
| | | DTNLIESSNMLGLNREVIADDLPHPFGLTQYQDYIYWTDW |
| | | SRRSIERANKTSGQNRTIIQGHLDYVMDILVFHSSRQSGWN |
| | | ECASSNGHCSHLCLAVPVGGFVCGCPAHYSLNADNRTCSA |
| | | PTTFLLFSQKSAINRMVIDEQQSPDIILPIHSLRNVRAIDYDP |
| | | LDKQLYWIDSRQNMIRKAQEDGSQGFTVVVSSVPSQNLEI |
| | | QPYDLSIDIYSRYIYWTCEATNVINVTRLDGRSVGVVLKGE |
| | | QDRPRAVVVNPEKGYMYFTNLQERSPKIERAALDGTEREV |
| | | LFFSGLSKPIALALDSRLGKLFWADSDLRRIESSDLSGANRI |
| | | VLEDSNILQPVGLTVFENWLYWIDKQQQMIEKIDMTGREG |
| | | RTKVQARIAQLSDIHAVKELNLQEYRQHPCAQDNGGCSHI |
| | | CLVKGDGTTRCSCPMHLVLLQDELSCGEPPTCSPQQFTCFT |
| | | GEIDCIPVAWRCDGFTECEDHSDELNCPVCSESQFQCASGQ |
| | | CIDGALRCNGDANCQDKSDEKNCEVLCLIDQFRCANGQCI |
| | | GKHKKCDHNVDCSDKSDELDCYPTEEPAPQATNTVGSVIG |
| | | VIVTIFVSGTVYFICQRMLCPRMKGDGETMTNDYVVHGPA |
| | | SVPLGYVPHPSSLSGSLPGMSRGKSMISSLSIMGGSSGPPYD |
| | | RAHVTGASSSSSSSTKGTYFPAILNPPPSPATERSHYTMEFG |
| | | YSSNSPSTHRSYSYRPYSYRHFAPPTTPCSTDVCDSDYAPSR |
| | | RMTSVATAKGYTSDLNYDSEPVPPPPTPRSQYLSAEENYES |
| | | CPPSPYTERSYSHHLYPPPPSPCTDSS |
| 410 | Anti-LRP5/6 | DEDY |
| | CDRH3 | |
| | DEDY motif | |
| 411 | Anti-LRP5/6 | DEEY |
| | CDRH3 | |
| | DEDY motif | |
| | mutant | |
| 412 | Anti-LRP5/6 | DESY |
| | CDRH3 | |
| | DEDY motif | |
| | mutant | |
| 413 | Anti-LRP5/6 | DEAY |
| | CDRH3 | |
| | DEDY motif | |
| | mutant | |
| 414 | Anti-LRP5/6 | DETY |
| | CDRH3 | |
| | DEDY motif | |
| | mutant | |
| 415 | Anti-LRP5/6 | ESEY |
| | CDRH3 | |
| | DEDY motif | |
| | mutant | |
| 416 | Anti-LRP5/6 | ESTY |
| | CDRH3 | |
| | DEDY motif | |
| | mutant | |
| 417 | Anti-LRP5/6 | ESSY |
| | CDRH3 | |
| | DEDY motif | |
| | mutant | |
| 418 | Anti-LRP5/6 | DDDY |
| | CDRH3 | |
| | DDDY motif | |
| 430 | Human | EVQLVESGGGLVQPGGSLRLSCAASGFTVSSNYMSWVRQA |
| | germline 3- | PGKGLEWVSVIYSGGSTYYADSVKGRFTISRDNSKNTLYLQ |
| | 66*01 VH | MNSLRAEDTAVYYCARWGQGTLVTVSS |
| 431 | Anti-LRP5/6 | EVQLVESGGGLVQPGGSLRLSCAASANINSIETLGWVRQAP |
| | VH (h VHH3- | GKGLEWVSNMRGGGYMKYADSVKGRFTISRDNSKNTLYL |
| | H1) | QMNSLRAEDTAVYYCARKLRDDDYVYWGQGTLVTVSS |
| 440 | Human | EVQLVESGGGLVQPGGSLRLSCAASGFTFSSYAMSWVRQA |
| | germline | PGKGLEWVSAISGSGGSTYYADSVKGRFTISRDNSKNTLYL |
| | IGHV3-23*04 | QMNSLRAEDTAVYYCAK |
| 441 | Human | YFDYWGQGTLVTVSS |
| | germline | |
| | IGHJ4*01 | |

APPENDIX TABLE L
Anti-LRP5/6 VH CDR and FR sequences based on IMGT or
Kabat

| SEQ | | |
|---|---|---|
| ID | | |
| NO: | Name | Sequences |
| 311 | Anti-LRP5/6 | EVQLVESGGGLVQPGGSLRLSCASS |
| | FRH1 | |
| | (IMGT) | |
| 321 | Anti-LRP5/6 | ANIQSIET |
| | CDRH1 | |
| | (IMGT) | |
| 531 | Anti-LRP5/6 | LGWYRQAPGKQRELIAN |
| | FRH2 | |
| | (IMGT) | |
| 541 | Anti-LRP5/6 | MRGGGYM |
| | CDRH2 | |
| | (IMGT) | |
| 351 | Anti-LRP5/6 | KYADSLKGRFTMSTDNSKNTMYLQMNSLRAEDTAVYYC |
| | FRH3 | |
| | (IMGT) | |
| 361 | Anti-LRP5/6 | YVKLRDEDYVY |
| | CDRH3 | |
| | (IMGT) | |
| 371 | Anti-LRP5/6 | RGQGTQVTVSS |
| | FRH4 | |
| | (IMGT) | |
| 611 | Anti-LRP5/6 | EVQLVESGGGLVQPGGSLRLSCASSANIQS |
| | FRH1 | |
| | (Kabat) | |
| 621 | Anti-LRP5/6 | IETLG |
| | CDRH1 | |
| | (Kabat) | |
| 631 | Anti-LRP5/6 | WYRQAPGKQRELIA |
| | FRH2 | |
| | (Kabat | |
| 641 | Anti-LRP5/6 | NMRGGGYMKYADSLKG |
| | CDRH2 | |
| | (Kabat) | |
| 651 | Anti-LRP5/6 | RFTMSTDNSKNTMYLQMNSLRAEDTAVYYCYV |
| | FRH3 | |
| | (Kabat) | |
| 661 | Anti-LRP5/6 | KLRDEDYVY |
| | CDRH3 | |
| | (Kabat) | |
| 671 | Anti-LRP5/6 | RGQGTQVTVSS |
| | FRH4 | |
| | (Kabat) | |

What is claimed is:

1. A multispecific antibody or antibody fragment comprising at least:
(A) a first antigen-binding region which specifically binds to frizzled class receptor 4 (Fzd4) and comprises a first heavy chain variable domain (VH1) and a first light chain variable domain (VL1),
(B) a second antigen-binding region which specifically binds to low density lipoprotein receptor-related protein 5 and/or 6 (LRP5 and/or LRP6) and comprises at least a second heavy chain variable domain (VH2),
wherein:
(A) the VH1 comprises:
(i) a heavy chain complementarity-determining region 1 (CDRH1) which has an amino acid sequence which:
(i-1) has at least 80% identity to the amino acid sequence of SEQ ID NO: 121 or to the CDRH1 amino acid sequence contained in SEQ ID NO: 181, optionally defined according to Kabat; or
(i-2) comprises or consists of the amino acid sequence of SEQ ID NO: 121, or comprises or consists of the CDRH1 amino acid sequence contained in SEQ ID NO: 181, optionally defined according to Kabat;
(ii) a heavy chain complementarity-determining region 2 (CDRH2) which has an amino acid sequence which:
(ii-1) has at least 90% identity to the amino acid sequence of SEQ ID NO: 141 or to the CDRH2 amino acid sequence contained in SEQ ID NO: 181, optionally defined according to Kabat;
or
(ii-2) comprises or consists of: the amino acid sequence of SEQ ID NO: 141; or comprises or consists of the CDRH2 amino acid sequence contained in SEQ ID NO: 181, optionally defined by Kabat; and
(iii) a heavy chain complementarity-determining region 3 (CDRH3) which has an amino acid sequence which:
(iii-1) has at least 90% identity to the amino acid sequence of SEQ ID NO: 161 or to the CDRH3 amino acid sequence contained in SEQ ID NO: 181, optionally defined by Kabat; or
(iii-2) comprises or consists of the amino acid sequence of SEQ ID NO: 161, or comprises or consists of the CDRH3 amino acid sequence contained in SEQ ID NO: 181, optionally defined by Kabat; and
the VL1 comprises:
(iv) a light chain complementarity-determining region 1 (CDRL1) which has an amino acid sequence which:
(iv-1) has at least 90% identity to the amino acid sequence of SEQ ID NO: 221 or to the CDRL1 amino acid sequence contained in SEQ ID NO: 281, optionally defined by Kabat; or
(iv-2) comprises or consists of the amino acid sequence of SEQ ID NO: 221, or comprises or consists of the CDRL1 amino acid sequence contained in SEQ ID NO: 281, optionally defined according to Kabat;
(v) a light chain complementarity-determining region 2 (CDRL2) which has an amino acid sequence which comprises or consists of:
(v-1) the amino acid sequence of SEQ ID NO: 241; or
(v-2) the CDRL2 amino acid sequence contained in SEQ ID NO: 281, optionally defined according to Kabat; and
(vi) a light chain complementarity-determining region 3 (CDRL3) which has an amino acid sequence which comprises or consists of:
(vi-1) the amino acid sequence of SEQ ID NO: 261; or
(vi-2) the CDRL3 amino acid sequence contained in SEQ ID NO: 281, optionally defined according to Kabat; and
(B) the VH2 comprises:
(i) a CDRH1 which has an amino acid sequence which comprises or consists of:
(i-1) the amino acid sequence of SEQ ID NO: 321; or
(i-2) the CDRH1 amino acid sequence contained in SEQ ID NO: 381, optionally defined according to IMGT or Kabat;
(ii) a CDRH2 which has an amino acid sequence which:
(ii-1) has at least 90% identity to the amino acid sequence of SEQ ID NO: 341 or to the CDRH3 amino acid sequence contained in SEQ ID NO: 381, optionally defined according to IMGT or Kabat;
(ii-2) comprises or consists of the amino acid sequence of SEQ ID NO: 341; or
(ii-3) comprises or consists of the CDRH2 amino acid sequence contained in SEQ ID NO: 381, optionally defined according to IMGT or Kabat; and
(iii) a CDRH3 which has an amino acid sequence which:
(iii-1) has at least 90% identity to the amino acid sequence of SEQ ID NO: 361 or to the CDRH3 amino acid sequence contained in SEQ ID NO: 381, optionally defined according to IMGT or Kabat; or
(iii-2) comprises or consists of the amino acid sequence of any one of SEQ ID NOs: 361 and 363-366, or comprises or consists of the CDRH3 amino acid sequence contained in any one of SEQ ID NOS: 381 and 393-396, optionally defined according to IMGT or Kabat.

2. The multispecific antibody or antibody fragment of claim 1, wherein the VH2 is, comprises, or is comprised in a nanobody.

3. The multispecific antibody or antibody fragment of claim 1, which is a bispecific antibody or antibody fragment.

4. The multispecific antibody or antibody fragment of claim 1 wherein:
(A) the VH1 comprises:
(i) the CDRH1, the CDRH2, and the CDRH3 of the VH1 comprising the amino acid sequence contained in SEQ ID NO: 181, optionally according to Kabat; or
(ii) a CDRH1, a CDRH2, and a CDRH3 comprising the amino acid sequences of SEQ ID NOS: 121, 141, and 161, respectively, and
the VL1 comprises:
(i) the CDRL1, the CDRL2, and the CDRL3 of the VL1 comprising the amino acid sequence contained in SEQ ID NO: 281, optionally according to Kabat, or
(ii) a CDRL1, a CDRL2, and a CDRL3 comprising the amino acid sequences of SEQ ID NOS: 221, 241, and 261, respectively; and
(B) the VH2 comprises:
(i) the CDRH1, the CDRH2, and the CDRH3 of the VH2 comprising the amino acid sequence contained in any one of SEQ ID NOs: 381 and 393-396, optionally according to IMGT or Kabat; or
(ii) a CDRH1, a CDRH2, and a CDRH3 comprising the amino acid sequences of SEQ ID NOS: 321, 341, and 361, respectively, SEQ ID NOS: 321, 341, and 363, respectively, SEQ ID NOS: 321, 341, and 364, respectively, SEQ ID NOS: 321, 341, and 365, or SEQ ID NOS: 321, 341, and 366, respectively.

5. The multispecific antibody or antibody fragment of claim 1, wherein:
(A) (i) the VH1 comprises an amino acid sequence which has at least 80% identity to the amino acid sequence of SEQ ID NO: 181; and/or
(ii) the VL1 comprises an amino acid sequence which has at least 80% identity to the amino acid sequence of SEQ ID NO: 281; and/or
(B) the VH2 comprises an amino acid sequence which has at least 80% identity to the amino acid sequence contained in any one of SEQ ID NOS: 381 and 393-396.

6. The multispecific antibody or antibody fragment of claim 1, wherein:
(A) the VH1 comprises an amino acid sequence which comprises or consists of the amino acid sequence of SEQ ID NO: 181: and/or
the VL1 comprises an amino acid sequence which comprises or consists of the amino acid sequence of SEQ ID NO: 281; and/or
(B) the VH2 comprises an amino acid sequence which comprises or consists of the amino acid sequence contained in any one of SEQ ID NOS: 381 and 393-396.

7. The multispecific antibody or antibody fragment of claim 1, wherein:
(A) the VH1 comprises an amino acid sequence which consists of the amino acid sequence of SEQ ID NO: 181: and
the VL1 comprises an amino acid sequence which consists of the amino acid sequence of SEQ ID NO: 281; and
(B) the VH2 comprises an amino acid sequence which consists of the amino acid sequence contained in any one of SEQ ID NOS: 381 and 393-396.

8. A multispecific antibody according to claim 1, comprising:
(A) a first antigen-binding region which specifically binds to Fzd4 and comprises a VH1 and a VL1,
(B) a second antigen-binding region which specifically binds to LRP5 and/or LRP6 and comprises at least a VH2,
wherein:
(i) the CDRH1, the CDRH2, and the CDRH3 of the VH1 are identical to those of the heavy chain variable domain (VH) having the amino acid sequence of SEQ ID NO: 181;
(ii) the CDRL1, the CDRL2, and the CDRL3 of the VL1 are identical to those of the light chain variable domain (VL) having the amino acid sequence of SEQ ID NO: 281; and
(iii) the CDRH1, the CDRH2, and the CDRH3 of the VH2 are identical to those of the VH having the amino acid sequence of SEQ ID NO: 381.

9. The multispecific antibody of claim 8, wherein:
(i) the VH1 comprises or consists of the amino acid sequence of SEQ ID NO: 181; and
(ii) the VL1 comprises or consists of the amino acid sequence of SEQ ID NO: 281; and
(iii) the VH2 comprises or consists of the amino acid sequence of SEQ ID NO: 381.

10. The multispecific antibody or antibody fragment of claim 1, which comprises a fragment crystallizable (Fc) region, optionally wherein the Fc region:
(I) is of an IgG, an IgA, an IgE, an IgD, an IgM, optionally of an IgG1, an IgG4, an IgG2, or an IgG3, further optionally of human, or a variant thereof;
(II) comprises one or more amino acid substitutions that reduce an Fc effector function, optionally wherein the one or more amino acid substitutions are at position(s) selected from the group consisting of positions 234, 235, 236, 237, 265, 297 and 329, according to EU numbering;
(III) comprises at least the following amino acid substitutions according to EU numbering:
(ii-1) L234A, L235A, and P329G;
(ii-2) L234A and L235A;
(ii-3) I253A, H310A, and H435Q; and/or
(ii-4) I253A, H310A, and H435A,
optionally relative to a human IgG1 Fc region and/or to any of SEQ ID NOS: 71-74 (optionally not including the C-terminal K); and/or
(IV) comprises an amino acid sequence which comprises or consists of the amino acid sequence of any one of SEQ ID NOS: 71-74, 81-84, 86-89, 91-94, 96-99, 471-474, 476-479, 481-484, and 486-489 (optionally not including the C-terminal K) or which is at least 80% identical thereto.

11. The multispecific antibody or antibody fragment of claim 1, which
comprises:
(a) a first polypeptide comprising said VH1; and
(b) a second polypeptide comprising
the following in the order from the N-terminus to the C-terminus:
(b-1) said VH2;
(b-2) optionally a linker, which is optionally a peptide linker, further optionally a flexible linker, optionally wherein the linker:
(i) comprises one or more amino acids, optionally one, two, three, four, five, six, seven, eight, nine, ten, eleven, or twelve amino acids;
(ii) consists of small amino acids consisting of G, S, and/or A;
(iii) comprising an amino acid sequence which comprises or consists of an amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG; and/or
(iv) comprises or consists of multiple repeats of an amino acid or peptide comprising an amino acid sequence selected from the group consisting of SEQ ID NO: 101, SEQ ID NO: 102, SEQ ID NO: 103, SEQ ID NO: 104, SEQ ID NO: 105, G, GG, GGG, GS, SG, GGS, GSG, SGG, GSS, SGS, and SSG; and
(b-3) said VL1.

12. The multispecific antibody or antibody fragment of claim 11, wherein:
(a) the first polypeptide;
(i) comprises an amino acid sequence which has at least 80% identity to any one of SEQ ID NOS: 2, 7, 8, 9, 450, 451, 452, and 453 (optionally without the C-terminal K); and/or
(ii) comprises an amino acid sequence which comprises or consists of the amino acid sequence of any one of SEQ ID NOS: 2, 7, 8, 9, 450, 451, 452, and 453 (optionally without the C-terminal K); and
(b) the second polypeptide:
(i) comprises an amino acid sequence which has at least 80% identity to SEQ ID NO: 1; and/or
(ii) comprising an amino acid sequence which comprises or consists of the amino acid sequence of SEQ ID NO: 1.

13. The multispecific antibody or antibody fragment of claim 11, wherein:
(a) the first polypeptide comprises an amino acid sequence which comprises or consists of the amino acid sequence of SEQ ID NO: 2, 7, 450, or 451 (optionally without the C-terminal K); and
(b) the second polypeptide comprises an amino acid sequence which comprises or consists of the amino acid sequence of SEQ ID NO: 1.

14. The multispecific antibody or antibody fragment of claim 11, which comprises: two of said first polypeptides forming a dimer with each other via one or more disulfide bonds; and two of said second polypeptides, wherein one of the second polypeptides is interacting with one of the first polypeptides via a disulfide bond and the other of the second polypeptides is interacting with the other of the first polypeptides via a disulfide bond.

15. The multispecific antibody or antibody fragment of claim 1, which comprises:
(A) two or more Fzd4-binding regions, wherein at least one of said Fzd4-binding regions is, comprises, or is comprised in said first antigen-binding region; and/or
(B) two or more LRP5 and/or LRP6-binding regions, wherein at least one of said LRP5 and/or LRP6-binding regions is, comprises, or is comprised in said second antigen-binding region.

16. The multispecific antibody or antibody fragment of claim 15, wherein:
(I) the ratio of the number of said one or more Fzd4-binding regions and the number of said one or more LRP5 and/or LRP6-binding regions comprised in the multispecific antibody or antibody fragment is selected from the group consisting of 2:1, 1:2, 2:2, 3:1, 1:3, 3:2, 2:3, 3:3, 4:1, 1:4, 4:2, 2:4, 4:3, 3:4, and 4:4;
(II) the number of said one or more Fzd4-binding regions and the number of said one or more LRP5 and/or LRP6-binding regions comprised in the multispecific antibody or antibody fragment are: 2 and 1, respectively; 1 and 2, respectively; 2 and 2, respectively; 3 and 1, respectively; 1 and 3, respectively; 3 and 2, respectively; 2 and 3, respectively; 3 and 3, respectively; 4 and 1, respectively; 1 and 4, respectively; 4 and 2, respectively; 2 and 4, respectively; 4 and 3, respectively; 3 and 4, respectively; or 4 and 4 respectively; and/or
(III) the multispecific antibody or antibody fragment comprises:
(a) two identical Fzd4-binding regions, each of which is, comprises, or is comprised in said first antigen-binding region; and one LRP5 and/or LRP6-binding region which is, comprises, or is comprised in said second antigen-binding region;
(b) two Fzd4-binding regions, which are different from each other and (i) at least one of which is, comprises, or is comprised in or (ii) each of which is, comprises, or is comprised in said first antigen-binding region; and one LRP5 and/or LRP6-binding region which is, comprises, or is comprised in said second antigen-binding region;
(c) one Fzd4-binding region which is, comprises, or is comprised in said first antigen-binding region; and two identical LRP5 and/or LRP6-binding regions, each of which is, comprises, or is comprised in said second antigen-binding region;
(d) one Fzd4-binding region which is, comprises, or is comprised in said first antigen-binding region; and two LRP5 and/or LRP6-binding regions, which are different from each other and (i) at least one of which is, comprises, or is comprised in or (ii) each of which is, comprises, or is comprised in said second antigen-binding region;
(e) two identical Fzd4-binding regions, each of which is, comprises, or is comprised in said first antigen-binding region; and two identical LRP5 and/or LRP6-binding region, each of which is, comprises, or is comprised in said second antigen-binding region;
(f) two Fzd4-binding regions, which are different from each other and (i) at least one of which is, comprises, or is comprised in or (ii) each of which is, comprises, or is comprised in said first antigen-binding region; and two identical LRP5 and/or LRP6-binding region, each of which is, comprises, or is comprised in said second antigen-binding region;
(g) two identical Fzd4-binding regions, each of which is, comprises, or is comprised in said first antigen-binding region; and two LRP5 and/or LRP6-binding regions, which are different from each other and (i) at least one of which is, comprises, or is comprised in or (ii) each of which is, comprises, or is comprised in said second antigen-binding region; and/or
(h) two Fzd4-binding regions, which are different from each other and (i) at least one of which is, comprises, or is comprised in or (ii) each of which is, comprises, or is comprised in said first antigen-binding region; and two LRP5 and/or LRP6-binding region, which are different from each other and (i) at least one of which is, comprises, or is comprised in or (ii) each of which is, comprises, or is comprised in said second antigen-binding region.

17. A nucleic acid or a combination of nucleic acids encoding the multispecific antibody or antibody fragment of claim 1, optionally wherein the nucleic acid(s) is/are DNA, cDNA, RNA, mRNA, modified mRNA, or a DNA/RNA hybrid.

18. A nucleic acid or a combination of nucleic acids, which encodes the multispecific antibody or antibody fragment of claim 11 and comprises:
(a) a first nucleic acid encoding the first polypeptide; and
(b) a second nucleic acid encoding the second polypeptide.

19. A vector or a combination of vectors which comprises the nucleic acid or combination of nucleic acids of claim 17, optionally wherein:
(i) the vector(s) comprise(s) one or more promoters operably linked to the nucleic acid(s);
(ii) the vector(s) is/are an expression vector; and/or
(iii) the vector(s) comprise(s) a plasmid, a viral vector (optionally adeno-associated viral, adenoviral, lentiviral, or retroviral), a lipid-based vector, a self-replicating RNA vector, a virus-like particle, a polymer-based vector, and/or a nanoparticle, optionally a lipid-based nanoparticle.

20. The vector or a combination of vectors of claim 19, which encodes a multispecific antibody or antibody fragment, which comprises:
(a) a first vector comprising a first nucleic acid encoding the first polypeptide; and
(b) a second vector comprising a second nucleic acid encoding the second polypeptide.

21. A host cell, which comprises:
(A) a multispecific antibody or antibody fragment according to claim 1;
(B) a nucleic acid or combination of nucleic acids encoding a multispecific antibody or antibody fragment according to claim 1; and/or
(C) a vector or combination of vectors comprising a nucleic acid or combination of nucleic acids encoding at least one multispecific antibody or antibody fragment according to claim 1, which nucleic acid(s) is/are operably linked to one or more promoters; and/or
optionally wherein the host cell is:
(i) mammalian, optionally human, non-human primate, monkey, rabbit, rodent, hamster, rat, or mouse; or
(ii) non-mammalian, optionally plant, bacterial, fungal, yeast, protozoa, or insect,
and optionally wherein the host cell is:
(i) a human embryonal kidney (HEK) cell, optionally a HEK293 cell, or a variant thereof, further optionally Expi293F™ cell;
(ii) a CHO (Chinese Hamster Ovary) cell;
(iii) an immune cell or
(iv) a hybridoma.

22. A population of cells, which comprises two or more host cells of claim 21.

23. A pharmaceutical composition, which comprises:
(I) ((A) a multispecific antibody or antibody fragment according to claim 1;
(B) a nucleic acid or combination of nucleic acids encoding a multispecific antibody or antibody fragment according to claim 1; and/or
(C) a vector or combination of vectors comprising a nucleic acid or combination of nucleic acids encoding at least one multispecific antibody or antibody fragment according to claim 1, which nucleic acid(s) is/are operably linked to one or more promoters, and/or
(D) a host cell or population of cells which comprises any of (A) to (C); and
(II) a pharmaceutically acceptable carrier and/or excipient.

24. A method of treating a subject in need of such treatment, comprising administering to the subject an effective amount of:
(A) a multispecific antibody or antibody fragment according to claim 1;
(B) a nucleic acid or combination of nucleic acids encoding a multispecific antibody or antibody fragment according to claim 1; and/or
(C) a vector or combination of vectors comprising a nucleic acid or combination of nucleic acids encoding at least one multispecific antibody or antibody fragment according to claim 1, which nucleic acid(s) is/are operably linked to one or more promoters; and/or
(D) a host cell or population of cells which comprises any of (A) to (C); and/or
(E) a pharmaceutical composition which comprises any of (A) to (D);
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile;
(b) the subject comprises or has a risk of developing a disease, disorder, or a condition; and/or
(c) the method further comprises administering to the subject an additional agent, optionally an adjuvant or a therapeutic agent.

25. A method of treating or preventing a disease, disorder, or a condition in a subject in need of such treatment, the method comprising administering an effective amount of:
((A) a multispecific antibody or antibody fragment according to claim 1;
(B) a nucleic acid or combination of nucleic acids encoding a multispecific antibody or antibody fragment according to claim 1; and/or
(C) a vector or combination of vectors comprising a nucleic acid or combination of nucleic acids encoding at least one multispecific antibody or antibody fragment according to claim 1, which nucleic acid(s) is/are operably linked to one or more promoters; and/or
(D) a host cell or population of cells which comprises any of (A) to (C); and/or
(E) a pharmaceutical composition which comprises of (A) to (D),
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile; and/or
(b) the method further comprises administering to the subject an additional agent, optionally an adjuvant or a therapeutic agent.

26. A method of inducing, promoting, stimulating, enhancing, and/or supporting a Wnt signaling in a target cell of a subject, comprising administering to the subject an effective amount of:
(A) a multispecific antibody or antibody fragment according to claim 1;
(B) a nucleic acid or combination of nucleic acids encoding a multispecific antibody or antibody fragment according to claim 1; and/or
(C) a vector or combination of vectors comprising a nucleic acid or combination of nucleic acids encoding at least one multispecific antibody or antibody fragment according to claim 1, which nucleic acid(s) is/are operably linked to one or more promoters; and/or
(D) a host cell or population of cells which comprises any of (A) to (C); and/or
(E) a pharmaceutical composition which comprises an of (A) to (D),
optionally wherein:
(a) the subject is
(i) a mammal, optionally a human, a non-human primate, a monkey, a horse, a cow, sheep, a goat, a pig, a dog, a cat, a rabbit, a rodent, a hamster, a rat, or a mouse; or
(ii) a non-mammalian vertebrate, optionally a bird, fish, an amphibian, or a reptile;
(b) the subject comprises or has a risk of developing a disease, disorder, or a condition; and/or
(c) the method further comprises administering to the subject an additional agent, optionally an adjuvant or a therapeutic agent,
and optionally wherein:
(i) the Wnt signaling is or comprises Wnt/β-catenin signaling; and/or
(ii) the method promotes, stimulates, enhances, and/or supports recruitment of multiple receptor complexes each comprising (ii-1) Fzd4 and (ii-2) LRP5 or LRP6.

27. The method of claim 24, wherein the disease, disorder, or condition comprises one or more of the following:
(a) a retinopathy, optionally a retinal vascular disease (optionally caused by inhibition of vascular development or excessive angiogenesis) and/or optionally selected from the group consisting of exudative vitreoretinopathy, familiar exudative vitreoretinopathy (FEVR), retinopathy of prematurity, Norrie disease, diabetic retinopathy (DR), diabetic macular edema, diabetic macular ischemia, age-related macular degeneration (AMD) (including wet AMD and dry AMD), retinopathy of prematurity (ROP), osteoporosis-pseudoglioma syndrome (OPPG), retinal vein occlusion, and Coats disease;
(b) a vascular disorder, optionally vascular malformation or vascular insufficiency, further optionally associated with ischemia-induced neovascularization;
(c) a bone disease or bone injury, optionally selected from the group consisting of osteoporosis (including osteoporosis pseudoglioma (OPPG) syndrome and juvenile primary osteoporosis), bone mineral density variability, and cancer-associated osteolysis;
(d) a muscle wasting disease, optionally selected from the group consisting of sarcopenia, cachexia, and muscular dystrophies; and/or
(e) a neural or neurodegenerative disease, optionally selected from the group consisting of stroke, traumatic brain injury, epilepsy, Alzheimer's disease, Parkinson's disease, Huntington's disease, amyotrophic lateral sclerosis, Friedreich ataxia, Lewy body disease, spinal muscular atrophy, motor neuron disease, multiple sclerosis, Batten disease, and Creutzfeldt-Jakob disease.

28. The method of claim 24, wherein:
(I) the administering is:
(i) to one or more of the subject's eyes, optionally intravitreally or via ocular drops;
(ii) locally administering, optionally to the eye, ear, nose (optionally intranasally), skin (optionally transdermally or epicutaneously), mucosa, skin, or vagina, or by inhalation; or
(iii) parenterally administering, optionally by injection (optionally intravenous, intramuscular, subcutaneous, intradermal, intrathecal, intra-arterial, intraarticular, intraosseous, or intraperitoneal administration) or by inhalation; or
(iv) enterally administering, optionally orally, sublingually, buccally, or rectally; and/or
(II) the multispecific antibody or antibody fragment is administered to one or more of the subject's eyes at about 0.1-100000 ng per eye, about 1-100000 ng per eye, about 1-10000 ng per eye, about 10-10000 ng per eye, about 1-100 ng per eye, about 200-300 ng per eye, about 300-400 ng per eye, about 400-500 ng per eye, about 500-600 ng per eye, about 600-700 ng per eye, about 700-800 ng per eye, about 800-900 ng per eye, about 900-1000 ng per eye, about 1000-2000 ng per eye, about 2000-3000 ng per eye, about 3000-4000 ng per eye, about 4000-5000 ng per eye, about 5000-6000 ng per eye, about 6000-7000 ng per eye, about 7000-8000 ng per eye, about 8000-9000 ng per eye, about 9000-10000 ng per eye, about 10000-20000 ng per eye, about 20000-30000 ng per eye, about 30000-40000 ng per eye, about 40000-50000 ng per eye, about 50000-60000 ng per eye, about 60000-70000 ng per eye, about 70000-80000 ng per eye, about 80000-90000 ng per eye, or about 90000-100000 ng per eye.

29. A method of inducing, promoting, stimulating, enhancing, and/or supporting a Wnt signaling in a target cell, comprising contacting the target cell with an effective amount of:
A) a multispecific antibody or antibody fragment according to claim 1;
(B) a nucleic acid or combination of nucleic acids encoding a multispecific antibody or antibody fragment according to claim 1; and/or
(C) a vector or combination of vectors comprising a nucleic acid or combination of nucleic acids encoding at least one multispecific antibody or antibody fragment according to claim 1, which nucleic acid(s) is/are operably linked to one or more promoters; and/or
(D) a host cell or population of cells which comprise any of (A) to (C); and/or
(E) a pharmaceutical composition which comprises any of (A) to (D),
optionally wherein:
(I) the contacting occurs in vitro, ex vivo, or in vivo,
(II) the method is for (i) preparing a cell and/or tissue for implantation, (ii) differentiation of a stem cell, and/or (iii) the preparation of an organoid,
(III) the method is for bone formation, vascular formation, and/or neural formation and/or differentiation.

30. A method of manufacturing a multispecific antibody or antibody fragment according to claim 1, comprising:
(a) culturing cells comprising a nucleic acid or combination of nucleic acids that encodes said multispecific antibody or antibody fragment under conditions that permit expression of said multispecific antibody or antibody fragment, and
(b) harvesting and purifying the multispecific antibody or antibody fragment from the cell culture from (a).

31. A method of manufacturing a host cell or a population of such cells which express at least one multispecific antibody or antibody fragment according to claim 1, comprising introducing a nucleic acid or combination of nucleic acids which encode for said at least one multispecific antibody or antibody fragment according to claim 1, or one or more vectors comprising said nucleic acid or combination of nucleic acids into one or more cells, optionally wherein the introducing occurs in vitro, ex vivo, or in vivo in a non-human subject.

32-34. (canceled)


SEQUENCE LISTING (671 sequences)

SEQ ID NO: 1 - protein, 336 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDEDYVYRGQGTQVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 4 - protein, 537 aa, Homo sapiens
MAWRGAGPSVPGAPGGVGLSLGLLLQLLLLLGPARGFGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEVPLPHKTPIQPGEECHSVGTNSDQYIWVKRSLNCVLKCGYDAGLYSRSAKEFTDIWMAVWASLCFISTAFTVLTFLIDSSRFSYPERPIIFLSMCYNIYSIAYIVRLTVGRERISCDFEEAAEPVLIQEGLKNTGCAIIFLLMYFFGMASSIWWVILTLTWFLAAGLKWGHEAIEMHSSYFHIAAWAIPAVKTIVILIMRLVDADELTGLCYVGNQNLDALTGFVVAPLFTYLVIGTLFIAAGLVALFKIRSNLQKDGTKTDKLERLMVKIGVFSVLYTVPATCVIACYFYEISNWALFRYSADDSNMAVEMLKIFMSLLVGITSGMWIWSAKTLHTWQKCSNRLVNSGKVKREKRGNGWVKPGKGSETVV

SEQ ID NO: 5 - protein, 1615 aa, Homo sapiens
MEAAPPGPPWPLLLLLLLLLALCGCPAPAAASPLLLFANRRDVRLVDAGGVKLESTIVVSGLEDAAAVDFQFSKGAVYWTDVSEEAIKQTYLNQTGAAVQNVVISGLVSPDGLACDWVGKKLYWTDSETNRIEVANLNGTSRKVLFWQDLDQPRAIALDPAHGYMYWTDWGETPRIERAGMDGSTRKIIVDSDIYWPNGLTIDLEEQKLYWADAKLSFIHRANLDGSFRQKVVEGSLTHPFALTLSGDTLYWTDWQTRSIHACNKRTGGKRKEILSALYSPMDIQVLSQERQPFFHTRCEEDNGGCSHLCLLSPSEPFYTCACPTGVQLQDNGRTCKAGAEEVLLLARRTDLRRISLDTPDFTDIVLQVDDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIALHPVMGLMYWTDWGENPKIECANLDGQERRVLVNASLGWPNGLALDLQEGKLYWGDAKTDKIEVINVDGTKRRTLLEDKLPHIFGFTLLGDFIYWTDWQRRSIERVHKVKASRDVIIDQLPDLMGLKAVNVAKVVGTNPCADRNGGCSHLCFFTPHATRCGCPIGLELLSDMKTCIVPEAFLVFTSRAAIHRISLETNNNDVAIPLTGVKEASALDFDVSNNHIYWTDVSLKTISRAFMNGSSVEHVVEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIPDDQHSPDLILPLHGLRNVKAIDYDPLDKFIYWVDGRQNIKRAKDDGTQPFVLTSLSQGQNPDRQPHDLSIDIYSRTLFWTCEATNTINVHRLSGEAMGVVLRGDRDKPRAIVVNAERGYLYFTNMQDRAAKIERAALDGTEREVLFTTGLIRPVALVVDNTLGKLFWVDADLKRIESCDLSGANRLTLEDANIVQPLGLTILGKHLYWIDRQQQMIERVEKTTGDKRTRIQGRVAHLTGIHAVEEVSLEEFSAHPCARDNGGCSHICIAKGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATARPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 6 - protein, 1613 aa, Homo sapiens
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAVVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 7 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 9 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 10 - skipped

SEQ ID NO: 11 - protein, 107 aa, Homo sapiens [sequence omitted]

SEQ ID NO: 12 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13-20 - skipped (8 entries)

SEQ ID NO: 21 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 22 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 23-30 - skipped (8 entries)

SEQ ID NO: 31 - protein, 15 aa, Homo sapiens
EPKSCDKTHTCPPCP

SEQ ID NO: 32-40 - skipped (9 entries)

SEQ ID NO: 41 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 42 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAK

SEQ ID NO: 43 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 44 - protein, 110 aa, synthetic construct
APELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAK

SEQ ID NO: 45 - protein, 110 aa, synthetic construct
APEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAK

SEQ ID NO: 46 - protein, 110 aa, synthetic construct
APEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAK

SEQ ID NO: 47-50 - skipped (4 entries)

SEQ ID NO: 51 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 54 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 55-60 - skipped (6 entries)

SEQ ID NO: 61 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 62 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 63 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNQYTQKSLSLSPGK

SEQ ID NO: 64 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNQYTQKSLSLSPGK

SEQ ID NO: 65 - skipped

SEQ ID NO: 66 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 67 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 68 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNAYTQKSLSLSPGK

SEQ ID NO: 69 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNAYTQKSLSLSPGK

SEQ ID NO: 70 - skipped

SEQ ID NO: 71 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 74 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 75-80 - skipped (6 entries)

SEQ ID NO: 81 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 84 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 85 - skipped

SEQ ID NO: 86 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 89 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 90 - skipped

SEQ ID NO: 91 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 92 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 93 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNQYTQKSLSLSPGK

SEQ ID NO: 94 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNQYTQKSLSLSPGK

SEQ ID NO: 95 - skipped

SEQ ID NO: 96 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 97 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 98 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNAYTQKSLSLSPGK

SEQ ID NO: 99 - protein, 227 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNAYTQKSLSLSPGK

SEQ ID NO: 100 - skipped

SEQ ID NO: 101 - protein, 5 aa, synthetic construct
GGGGS

SEQ ID NO: 102 - protein, 5 aa, synthetic construct
GGSGS

SEQ ID NO: 103 - protein, 5 aa, synthetic construct
GSGSG

SEQ ID NO: 104 - protein, 4 aa, synthetic construct
GGGS

SEQ ID NO: 105 - protein, 6 aa, synthetic construct
GGGGGS

SEQ ID NO: 106-109 - skipped (4 entries)

SEQ ID NO: 110 - protein, 30 aa, synthetic construct
EVQLVESGGGLVXPGGSLRLSCAASGFTFT

SEQ ID NO: 111 - protein, 30 aa, synthetic construct
EVQLVESGGGLVQPGGSLRLSCAASGFTFT

SEQ ID NO: 112 - protein, 30 aa, synthetic construct
EVQLVESGGGLVKPGGSLRLSCAASGFTFT

SEQ ID NO: 113-119 - skipped (7 entries)

SEQ ID NO: 120 - protein, 5 aa, synthetic construct
XYAMS

SEQ ID NO: 121 - protein, 5 aa, synthetic construct
SYAMS

SEQ ID NO: 122 - protein, 5 aa, synthetic construct
NYAMS

SEQ ID NO: 123 - protein, 5 aa, synthetic construct
AYAMS

SEQ ID NO: 124 - protein, 5 aa, synthetic construct
QYAMS

SEQ ID NO: 125-129 - skipped (5 entries)

SEQ ID NO: 130 - protein, 14 aa, synthetic construct
WVRQAPGKGLEWVS

SEQ ID NO: 131 - protein, 14 aa, synthetic construct
WVRQAPGKGLEWVS

SEQ ID NO: 132-139 - skipped (8 entries)

SEQ ID NO: 140 - protein, 17 aa, synthetic construct
AISGSGGSTYYAXSVKG

SEQ ID NO: 141 - protein, 17 aa, synthetic construct
AISGSGGSTYYAESVKG

SEQ ID NO: 142 - protein, 17 aa, synthetic construct
AISGSGGSTYYADSVKG

SEQ ID NO: 143 - protein, 17 aa, synthetic construct
AISGSGGSTYYAASVKG

SEQ ID NO: 144 - protein, 17 aa, synthetic construct
AISGSGGSTYYASSVKG

SEQ ID NO: 145-149 - skipped (5 entries)

SEQ ID NO: 150 - protein, 32 aa, synthetic construct
RFTISRDXSKNTLYLQMNSLXXEDTAVYYCAR

SEQ ID NO: 151 - protein, 32 aa, synthetic construct
RFTISRDNSKNTLYLQMNSLRAEDTAVYYCAR

SEQ ID NO: 152 - protein, 32 aa, synthetic construct
RFTISRDDSKNTLYLQMNSLKTEDTAVYYCAR

SEQ ID NO: 153-159 - skipped (7 entries)

SEQ ID NO: 160 - protein, 11 aa, synthetic construct
ATGFGTVVFXY

SEQ ID NO: 161 - protein, 11 aa, synthetic construct
ATGFGTVVFDY

SEQ ID NO: 162-169 - skipped (8 entries)

SEQ ID NO: 170 - protein, 11 aa, synthetic construct
WGQGTLVTVSS

SEQ ID NO: 171 - protein, 11 aa, synthetic construct
WGQGTLVTVSS

SEQ ID NO: 172-179 - skipped (8 entries)

SEQ ID NO: 180 - protein, 120 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSS

SEQ ID NO: 181 - protein, 120 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSS

SEQ ID NO: 182 - protein, 120 aa, synthetic construct
EVQLVESGGGLVKPGGSLRLSCAASGFTFTNYAMSWVRQAPGKGLEWVSAISGSGGSTYYADSVKGRFTISRDDSKNTLYLQMNSLKTEDTAVYYCARATGFGTVVFDYWGQGTLVTVSS

SEQ ID NO: 183 - protein, 120 aa, synthetic construct
EVQLVESGGGLVKPGGSLRLSCAASGFTFTQYAMSWVRQAPGKGLEWVSAISGSGGSTYYADSVKGRFTISRDDSKNTLYLQMNSLKTEDTAVYYCARATGFGTVVFDYWGQGTLVTVSS

SEQ ID NO: 184 - protein, 120 aa, synthetic construct
EVQLVESGGGLVKPGGSLRLSCAASGFTFTSYAMSWVRQAPGKGLEWVSAISGSGGSTYYADSVKGRFTISRDDSKNTLYLQMNSLKTEDTAVYYCARATGFGTVVFDYWGQGTLVTVSS

SEQ ID NO: 185 - protein, 120 aa, synthetic construct
EVQLVESGGGLVKPGGSLRLSCAASGFTFTAYAMSWVRQAPGKGLEWVSAISGSGGSTYYADSVKGRFTISRDDSKNTLYLQMNSLKTEDTAVYYCARATGFGTVVFDYWGQGTLVTVSS

SEQ ID NO: 186 - protein, 120 aa, synthetic construct
EVQLVESGGGLVKPGGSLRLSCAASGFTFTNYAMSWVRQAPGKGLEWVSAISGSGGSTYYASSVKGRFTISRDDSKNTLYLQMNSLKTEDTAVYYCARATGFGTVVFDYWGQGTLVTVSS

SEQ ID NO: 187 - protein, 120 aa, synthetic construct
EVQLVESGGGLVKPGGSLRLSCAASGFTFTNYAMSWVRQAPGKGLEWVSAISGSGGSTYYAESVKGRFTISRDDSKNTLYLQMNSLKTEDTAVYYCARATGFGTVVFDYWGQGTLVTVSS

SEQ ID NO: 188 - protein, 120 aa, synthetic construct
EVQLVESGGGLVKPGGSLRLSCAASGFTFTNYAMSWVRQAPGKGLEWVSAISGSGGSTYYAASVKGRFTISRDDSKNTLYLQMNSLKTEDTAVYYCARATGFGTVVFDYWGQGTLVTVSS

SEQ ID NO: 189-210 - skipped (22 entries)

SEQ ID NO: 211 - protein, 23 aa, synthetic construct
DIQMTQSPSSLSASVGDRVTITC

SEQ ID NO: 212-220 - skipped (9 entries)

SEQ ID NO: 221 - protein, 11 aa, synthetic construct
RASQSISSYLN

SEQ ID NO: 222-230 - skipped (9 entries)

SEQ ID NO: 231 - protein, 15 aa, synthetic construct
WYQQKPGKAPKLLIY

SEQ ID NO: 232-240 - skipped (9 entries)

SEQ ID NO: 241 - protein, 7 aa, synthetic construct
AASSLQS

SEQ ID NO: 242-250 - skipped (9 entries)

SEQ ID NO: 251 - protein, 32 aa, synthetic construct
GVPSRFSGSGSGTDFTLTISSLQPEDFATYYC

SEQ ID NO: 252-260 - skipped (9 entries)

SEQ ID NO: 261 - protein, 9 aa, synthetic construct
QQSYSTPLT

SEQ ID NO: 262-270 - skipped (9 entries)

SEQ ID NO: 271 - protein, 10 aa, synthetic construct
FGGGTKVEIK

SEQ ID NO: 272-280 - skipped (9 entries)

SEQ ID NO: 281 - protein, 107 aa, synthetic construct [sequence omitted]

SEQ ID NO: 282-299 - skipped (18 entries)

SEQ ID NO: 300 - protein, 11 aa, synthetic construct
YVKLREDEYVY

SEQ ID NO: 301 - protein, 11 aa, synthetic construct
YVKLRESDYVY

SEQ ID NO: 302 - protein, 11 aa, synthetic construct
YVKLRSDEYVY

SEQ ID NO: 303 - protein, 11 aa, synthetic construct
YVKLRSSDYVY

SEQ ID NO: 304-309 - skipped (6 entries)

SEQ ID NO: 310 - protein, 25 aa, synthetic construct
XVQLVESGGGLVQPGGSLRLSCXXS

SEQ ID NO: 311 - protein, 25 aa, synthetic construct
EVQLVESGGGLVQPGGSLRLSCASS

SEQ ID NO: 312 - protein, 25 aa, synthetic construct
DVQLVESGGGLVQPGGSLRLSCTSS

SEQ ID NO: 313-319 - skipped (7 entries)

SEQ ID NO: 320 - protein, 8 aa, synthetic construct
ANIXSIET

SEQ ID NO: 321 - protein, 8 aa, synthetic construct
ANIQSIET

SEQ ID NO: 322 - protein, 8 aa, synthetic construct
ANINSIET

SEQ ID NO: 323 - protein, 8 aa, synthetic construct
ANISSIET

SEQ ID NO: 324-329 - skipped (6 entries)

SEQ ID NO: 330 - protein, 16 aa, synthetic construct
LGWXRQAPGKXXEXXX

SEQ ID NO: 331 - protein, 16 aa, synthetic construct
LGWYRQAPGKQRELIA

SEQ ID NO: 332 - protein, 16 aa, synthetic construct
LGWYRQAPGKGREWIA

SEQ ID NO: 333 - protein, 16 aa, synthetic construct
LGWYRQAPGKQREWIA

SEQ ID NO: 334-339 - skipped (6 entries)

SEQ ID NO: 340 - protein, 8 aa, synthetic construct
NXRGGGYX

SEQ ID NO: 341 - protein, 8 aa, synthetic construct
NMRGGGYM

SEQ ID NO: 342-349 - skipped (8 entries)

SEQ ID NO: 350 - protein, 38 aa, synthetic construct
KYAXSXKGRFTXSXXXXKNTXYLQMNSLXXEDTAVYYC

SEQ ID NO: 351 - protein, 38 aa, synthetic construct
KYADSLKGRFTMSTDNSKNTMYLQMNSLRAEDTAVYYC

SEQ ID NO: 352 - protein, 38 aa, synthetic construct
KYAGSLKGRFTMSTESAKNTMYLQMNSLKPEDTAVYYC

SEQ ID NO: 353-359 - skipped (7 entries)

SEQ ID NO: 360 - protein, 11 aa, synthetic construct
YVKLRXXXYVY

SEQ ID NO: 361 - protein, 11 aa, synthetic construct
YVKLRDEDYVY

SEQ ID NO: 362 - protein, 11 aa, synthetic construct
YVKLRDDDYVY

SEQ ID NO: 363 - protein, 11 aa, synthetic construct
YVKLRDEEYVY

SEQ ID NO: 364 - protein, 11 aa, synthetic construct
YVKLRDESYVY

SEQ ID NO: 365 - protein, 11 aa, synthetic construct
YVKLRDEAYVY

SEQ ID NO: 366 - protein, 11 aa, synthetic construct
YVKLRDETYVY

SEQ ID NO: 367 - protein, 11 aa, synthetic construct
YVKLRESEYVY

SEQ ID NO: 368 - protein, 11 aa, synthetic construct
YVKLRESTYVY

SEQ ID NO: 369 - protein, 11 aa, synthetic construct
YVKLRESSYVY

SEQ ID NO: 370 - protein, 11 aa, synthetic construct
XGQGTXVTVSS

SEQ ID NO: 371 - protein, 11 aa, synthetic construct
RGQGTQVTVSS

SEQ ID NO: 372 - protein, 11 aa, synthetic construct
WGQGTLVTVSS

SEQ ID NO: 373-379 - skipped (7 entries)

SEQ ID NO: 380 - protein, 117 aa, synthetic construct
XVQLVESGGGLVQPGGSLRLSCXXSANIXSIETLGWXRQAPGKXXEXXXNXRGGGYXKYAXSXKGRFTXSXXXXKNTXYLQMNSLXXEDTAVYYCYVKLRXXXYVYXGQGTXVTVSS

SEQ ID NO: 381 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDEDYVYRGQGTQVTVSS

SEQ ID NO: 382 - protein, 117 aa, synthetic construct
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSS

SEQ ID NO: 383 - protein, 117 aa, synthetic construct
EVQLVESGGGLVQPGGSLRLSCASSANINSIETLGWYRQAPGKGREWIANMRGGGYMKYADSLKGRFTMSTDNSKNTMYLQMNSLRAEDTAVYYCYVKLRDDDYVYWGQGTLVTVSS

SEQ ID NO: 384 - protein, 117 aa, synthetic construct
EVQLVESGGGLVQPGGSLRLSCASSANINSIETLGWYRQAPGKQREWIANMRGGGYMKYADSLKGRFTMSTDNSKNTMYLQMNSLRAEDTAVYYCYVKLRDDDYVYWGQGTLVTVSS

SEQ ID NO: 385 - protein, 117 aa, synthetic construct
EVQLVESGGGLVQPGGSLRLSCASSANINSIETLGWYRQAPGKQRELIANMRGGGYMKYADSLKGRFTMSTDNSKNTMYLQMNSLRAEDTAVYYCYVKLRDDDYVYRGQGTQVTVSS

SEQ ID NO: 386 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDDDYVYRGQGTQVTVSS

SEQ ID NO: 387 - protein, 117 aa, synthetic construct
EVQLVESGGGLVQPGGSLRLSCASSANISSIETLGWYRQAPGKQRELIANMRGGGYMKYADSLKGRFTMSTDNSKNTMYLQMNSLRAEDTAVYYCYVKLRDDDYVYRGQGTQVTVSS

SEQ ID NO: 388 - protein, 117 aa, synthetic construct
EVQLVESGGGLVQPGGSLRLSCASSANINSIETLGWYRQAPGKQRELIANMRGGGYMKYADSLKGRFTMSTDNSKNTMYLQMNSLRAEDTAVYYCYVKLRDEDYVYRGQGTQVTVSS

SEQ ID NO: 389 - protein, 117 aa, synthetic construct
EVQLVESGGGLVQPGGSLRLSCASSANINSIETLGWYRQAPGKQRELIANMRGGGYMKYADSLKGRFTMSTDNSKNTMYLQMNSLRAEDTAVYYCYVKLREDEYVYRGQGTQVTVSS

SEQ ID NO: 390 - protein, 117 aa, synthetic construct
EVQLVESGGGLVQPGGSLRLSCASSANINSIETLGWYRQAPGKQRELIANMRGGGYMKYADSLKGRFTMSTDNSKNTMYLQMNSLRAEDTAVYYCYVKLRESDYVYRGQGTQVTVSS

SEQ ID NO: 391 - protein, 117 aa, synthetic construct
EVQLVESGGGLVQPGGSLRLSCASSANINSIETLGWYRQAPGKQRELIANMRGGGYMKYADSLKGRFTMSTDNSKNTMYLQMNSLRAEDTAVYYCYVKLRSDEYVYRGQGTQVTVSS

SEQ ID NO: 392 - protein, 117 aa, synthetic construct
EVQLVESGGGLVQPGGSLRLSCASSANINSIETLGWYRQAPGKQRELIANMRGGGYMKYADSLKGRFTMSTDNSKNTMYLQMNSLRAEDTAVYYCYVKLRSSDYVYRGQGTQVTVSS

SEQ ID NO: 393 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDEEYVYRGQGTQVTVSS

SEQ ID NO: 394 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDESYVYRGQGTQVTVSS

SEQ ID NO: 395 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDEAYVYRGQGTQVTVSS

SEQ ID NO: 396 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDETYVYRGQGTQVTVSS

SEQ ID NO: 397 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRESEYVYRGQGTQVTVSS

SEQ ID NO: 398 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRESTYVYRGQGTQVTVSS

SEQ ID NO: 399 - protein, 117 aa, synthetic construct
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRESSYVYRGQGTQVTVSS

SEQ ID NO: 400 - skipped

SEQ ID NO: 401 - protein, 336 aa, synthetic construct
XVQLVESGGGLVQPGGSLRLSCXXSANIXSIETLGWXRQAPGKXXEXXXNXRGGGYXKYAXSXKGRFTXSXXXXKNTXYLQMNSLXXEDTAVYYCYVKLRXXXYVYXGQGTXVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 402 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 403-406 - skipped (4 entries)

SEQ ID NO: 407 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 408 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 409 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 410 - protein, 4 aa, synthetic construct
DEDY

SEQ ID NO: 411 - protein, 4 aa, synthetic construct
DEEY

SEQ ID NO: 412 - protein, 4 aa, synthetic construct
DESY

SEQ ID NO: 413 - protein, 4 aa, synthetic construct
DEAY

SEQ ID NO: 414 - protein, 4 aa, synthetic construct
DETY

SEQ ID NO: 415 - protein, 4 aa, synthetic construct
ESEY

SEQ ID NO: 416 - protein, 4 aa, synthetic construct
ESTY

SEQ ID NO: 417 - protein, 4 aa, synthetic construct
ESSY

SEQ ID NO: 418 - protein, 4 aa, synthetic construct
DDDY

SEQ ID NO: 419-420 - skipped (2 entries)

SEQ ID NO: 421 - protein, 336 aa, synthetic construct
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGGSGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 450 aa, synthetic construct
EVQLVESGGGLVKPGGSLRLSCAASGFTFTNYAMSWVRQAPGKGLEWVSAISGSGGSTYYADSVKGRFTISRDDSKNTLYLQMNSLKTEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 423-429 - skipped (7 entries)

SEQ ID NO: 430 - protein, 108 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARWGQGTLVTVSS

SEQ ID NO: 431 - protein, 117 aa, synthetic construct
EVQLVESGGGLVQPGGSLRLSCAASANINSIETLGWVRQAPGKGLEWVSNMRGGGYMKYADSVKGRFTISRDNSKNTLYLQMNSLRAEDTAVYYCARKLRDDDYVYWGQGTLVTVSS

SEQ ID NO: 432-439 - skipped (8 entries)

SEQ ID NO: 440 - protein, 98 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 441 - protein, 15 aa, Homo sapiens
YFDYWGQGTLVTVSS

SEQ ID NO: 442-449 - skipped (8 entries)

SEQ ID NO: 450 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 451 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 452 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 453 - protein, 450 aa, synthetic construct
EVQLVESGGGLVQPGGSLRLSCAASGFTFTSYAMSWVRQAPGKGLEWVSAISGSGGSTYYAESVKGRFTISRDNSKNTLYLQMNSLRAEDTAVYYCARATGFGTVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 454-459 - skipped (6 entries)

SEQ ID NO: 460 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 461 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 462 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 463 - protein, 450 aa, synthetic construct
EVQLVESGGGLVXPGGSLRLSCAASGFTFTXYAMSWVRQAPGKGLEWVSAISGSGGSTYYAXSVKGRFTISRDXSKNTLYLQMNSLXXEDTAVYYCARATGFGTVVFXYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 464-470 - skipped (7 entries)

SEQ ID NO: 471 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 472 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 473 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNQYTQKSLSLSPGK

SEQ ID NO: 474 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNQYTQKSLSLSPGK

SEQ ID NO: 475 - skipped

SEQ ID NO: 476 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 477 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 478 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNAYTQKSLSLSPGK

SEQ ID NO: 479 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNAYTQKSLSLSPGK

SEQ ID NO: 480 - skipped

SEQ ID NO: 481 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 482 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 483 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNQYTQKSLSLSPGK

SEQ ID NO: 484 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNQYTQKSLSLSPGK

SEQ ID NO: 485 - skipped

SEQ ID NO: 486 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 487 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 488 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNAYTQKSLSLSPGK

SEQ ID NO: 489 - protein, 227 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNAYTQKSLSLSPGK

SEQ ID NO: 490-530 - skipped (41 entries)

SEQ ID NO: 531 - protein, 17 aa, synthetic construct
LGWYRQAPGKQRELIAN

SEQ ID NO: 532-540 - skipped (9 entries)

SEQ ID NO: 541 - protein, 7 aa, synthetic construct
MRGGGYM

SEQ ID NO: 542-610 - skipped (69 entries)

SEQ ID NO: 611 - protein, 30 aa, synthetic construct
EVQLVESGGGLVQPGGSLRLSCASSANIQS

SEQ ID NO: 612-620 - skipped (9 entries)

SEQ ID NO: 621 - protein, 5 aa, synthetic construct
IETLG

SEQ ID NO: 622-630 - skipped (9 entries)

SEQ ID NO: 631 - protein, 14 aa, synthetic construct
WYRQAPGKQRELIA

SEQ ID NO: 632-640 - skipped (9 entries)

SEQ ID NO: 641 - protein, 16 aa, synthetic construct
NMRGGGYMKYADSLKG

SEQ ID NO: 642-650 - skipped (9 entries)

SEQ ID NO: 651 - protein, 32 aa, synthetic construct
RFTMSTDNSKNTMYLQMNSLRAEDTAVYYCYV

SEQ ID NO: 652-660 - skipped (9 entries)

SEQ ID NO: 661 - protein, 9 aa, synthetic construct
KLRDEDYVY

SEQ ID NO: 662-670 - skipped (9 entries)

SEQ ID NO: 671 - protein, 11 aa, synthetic construct
RGQGTQVTVSS